(19) United States
(12) Patent Application Publication
(10) Pub. No.: US 2011/0052673 A1
(43) Pub. Date: Mar. 3, 2011

(54) THERAPEUTIC COMPOSITIONS

(76) Inventors: Arthur Tzianabos, Reading, MA (US); Michael W. Heartlein, Boxborough, MA (US)

(21) Appl. No.: 12/865,099

(22) PCT Filed: Jan. 28, 2009
(86) PCT No.: PCT/US09/32239

§ 371 Date: Nov. 9, 2010

Related U.S. Application Data
(60) Provisional application No. 61/024,477, filed on Jan. 29, 2008.

(51) Int. Cl. A61K 38/45; A61K 38/46; A61K 38/47; A61K 9/127; A61P 43/00

(52) U.S. Cl. 424/450; 424/94.5; 424/94.6; 424/94.61

(57) ABSTRACT

Therapeutic compositions containing therapeutic agents and nitrogen-containing lipids are described. These tertiary amine-containing polymers are preferably biodegradable and biocompatible. Nanoparticles, microparticles, and complexes containing lipid/therapeutic agent complexes are also described.

### CROSS REFERENCE TO RELATED APPLICATIONS

This application claims priority to U.S. Application Ser. No. 61/024,477, filed on Jan. 29, 2008. The disclosure of the prior application is considered part of (and is incorporated by reference in) the disclosure of this application.

### BACKGROUND

Deficient or low levels of a protein can lead to a disease or disorder in an individual. The protein at issue can be therapeutically administered to the individual with deficient or low levels of the protein to restore or increase the levels of the protein in the individual, for example, as a method of treating a disease or disorder caused by, or associated with, the deficient or low protein levels.

### SUMMARY

Lipids (e.g., amine-containing lipids) can be used in the preparation of therapeutic compositions that contain a therapeutic protein, e.g., a protein that can be used in replacement therapy. As used herein, “replacement therapy” refers to the use of a protein to reconstitute a deficiency or to increase otherwise low levels of the protein, e.g., in an individual that has a disease or disorder (or has a predisposition for a disease or disorder) cause by, or associated with, a protein deficiency or by low levels of a protein. The therapeutic composition can be contained in a pharmaceutical composition.

The protein deficiency or low levels may be caused, for example, by mutation (e.g., in a gene encoding the protein or in an element controlling expression of the gene (e.g., a regulatory sequence)), misfolding of the protein, or truncation of the protein (e.g., an amino or carboxy terminal truncation). The protein may be an enzyme. Non-limiting examples of proteins (e.g., therapeutic proteins) that can be used in replacement therapy include Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, and N-acetylgalactosamine-4-sulfatase.

In one aspect, the invention features a therapeutic composition comprising a lipid described herein and a therapeutic agent, wherein the therapeutic agent comprises a protein, e.g., a therapeutic protein, e.g., a protein for use in replacement therapy, e.g., a protein described herein.

In one aspect, the disclosure features a therapeutic composition that contains a therapeutic agent (e.g., a therapeutic protein, e.g., a protein for use in replacement therapy, e.g., a protein described herein) and a compound of the formula:

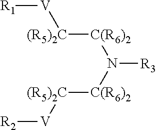

wherein V is selected from the group consisting of C═O, C═S, S═O, and SO2;

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(−0)RA; —NRAC(═O)N(RA)2; —OC(═O)ORA; —OC(═O)RA; —OC(═O)N(RA)2; —NRAC(═O)ORA; or —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(═O)RB; —CO2RB; —CN; —SCN; —SRB; —SORB; —SO2R3; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of R3 is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORC; —C(═O)Rc; —CO2RC; —CN; —SCN; —SRO; —SORc; —SO2RC; —NO2; —N3; —N(Rc)2; —NHC(═O)Rc; —NRCC(═O)N(RC)2; —OC(═O)ORc; —OC(═O)RC; —OC(O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R5 is independently selected from the group consisting of hydrogen and C1-C6 alkyl; each occurrence of R6 is independently selected from the group consisting of hydrogen and C1-C6 alkyl; with the proviso that when all occurrences of R5 and R6 are hydrogen, V is C═O, R1 is —ORA, R2 is —ORB, and R1 and R2 are the same, then R3 is not —CH2CH2ORc′, wherein Rc′ is methyl, ethyl, propyl, isopropyl, butyl, s-butyl, isobutyl, t-butyl, pentyl, cyclopentyl, hexyl, cyclohexyl, decyl, methoxymethyl, 2-methoxyethyl, 1-ethoxyethyl, 2-ethoxyethyl, (2-methoxyethoxy)methyl, 2-tetrahydrofuranyl, 2-tetrahydropyranyl, tetrahydrofurfuryl, fo[pi]nyl, acetyl, propionyl, butyryl, isobutyryl, pivaloyl, valeryl, methoxyacetyl, ethoxyacetyl, acetoxyacetyl, 2-formyloxyethyl, 2-acetoxyethyl, 2-oxopropyl, 2-oxobutyl, 2-oxocyclopentyl, 2-oxo-3-tetrahydrofuranyl, 2-oxo-3-tetrahydropyranyl, methoxycarbonyl, ethoxycarbonyl, and t-butoxycarbonyl; and with the proviso that when all occurrences of R5 and R6 are hydrogen, V is C═O, R1 is —ORA, wherein RA is a straight-chain, branched or cyclic alkyl group of 1 to 20 carbon atoms which may contain an ether, carbonyl, or carbonyloxy group; R2 is —ORB, wherein RB is a straight-chain, branched or cyclic alkyl group of 1 to 20 carbon atoms which may contain an ether, carbonyl, or carbonyloxy group; and R1 and R2 are the same, the R3 is not —CH2CH2ORc″, wherein Rc″ is a straight-chain, branched or cyclic alkyl group of 1 to 20 carbon atoms which may contain an ether, carbonyl, or carbonyloxy group; and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, each occurrence of R5 and R6 is hydrogen.

In some embodiments,

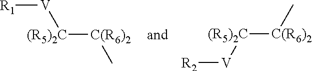

are the same.

In some embodiments,

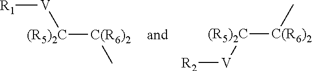

are the same and are different than R3.

In some embodiments

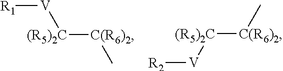

and R3 are all different.

In some embodiments,

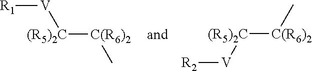

are independently selected from the group consisting of:

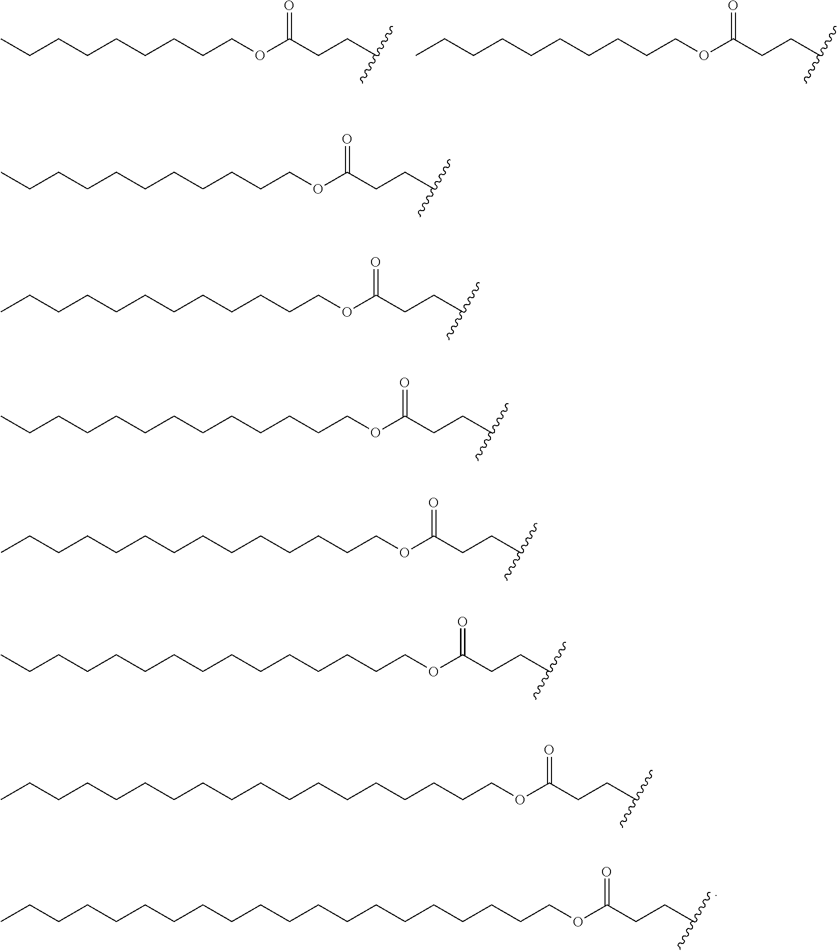

In some embodiments

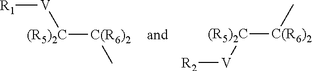

are the same and are selected from the group consisting of:

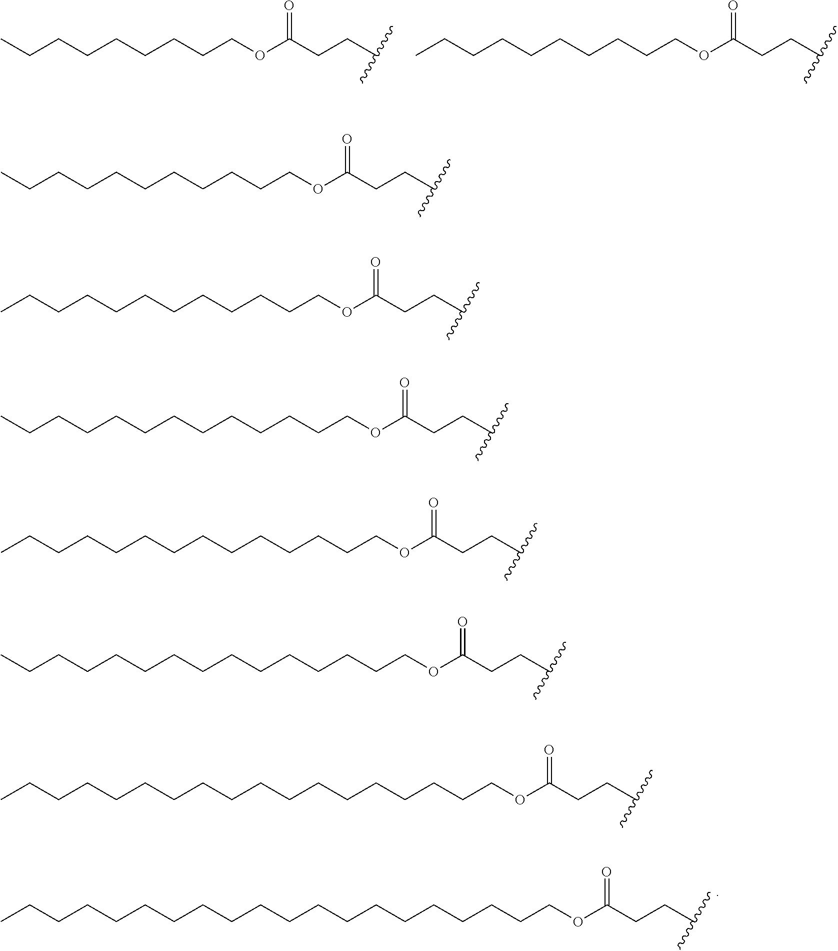

In some embodiments,

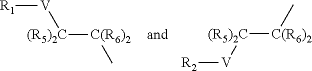

are the same and are selected from the group consisting of:

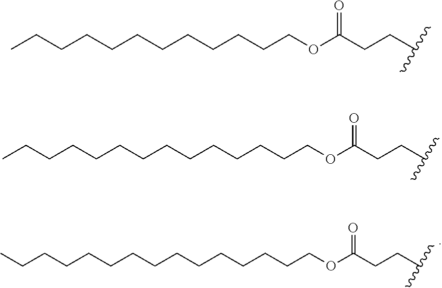

In some embodiments,

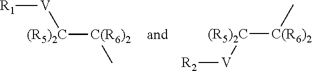

are independently selected from the group consisting of:

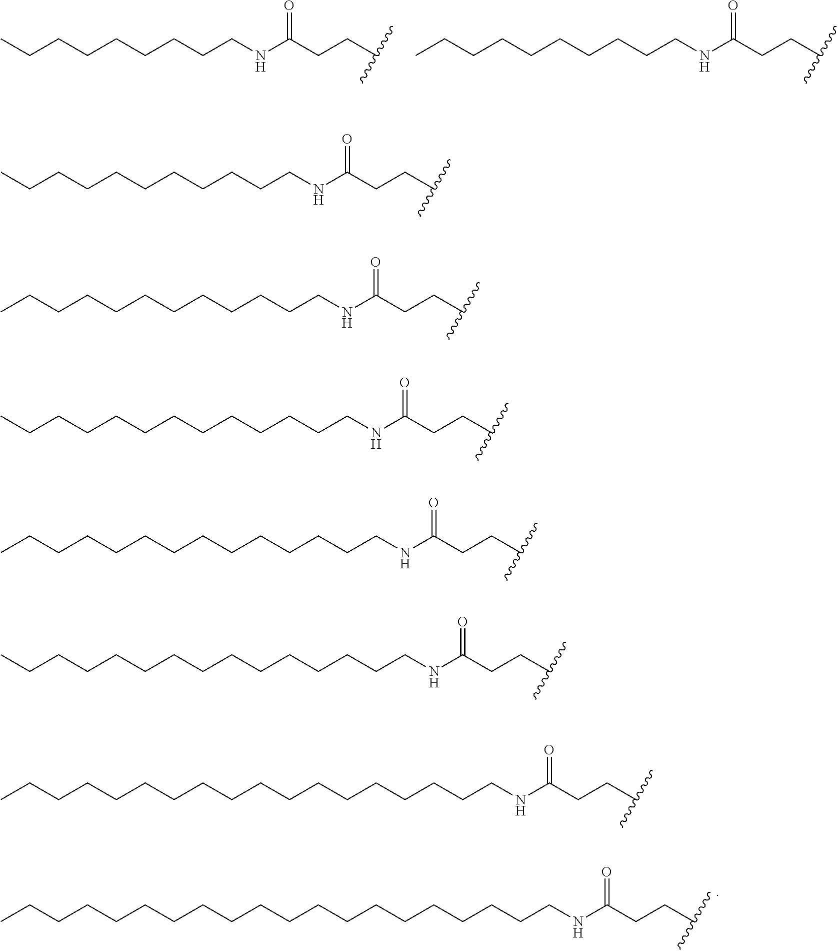

In some embodiments,

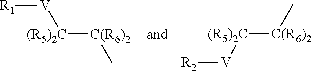

are the same and are selected from the group consisting of:

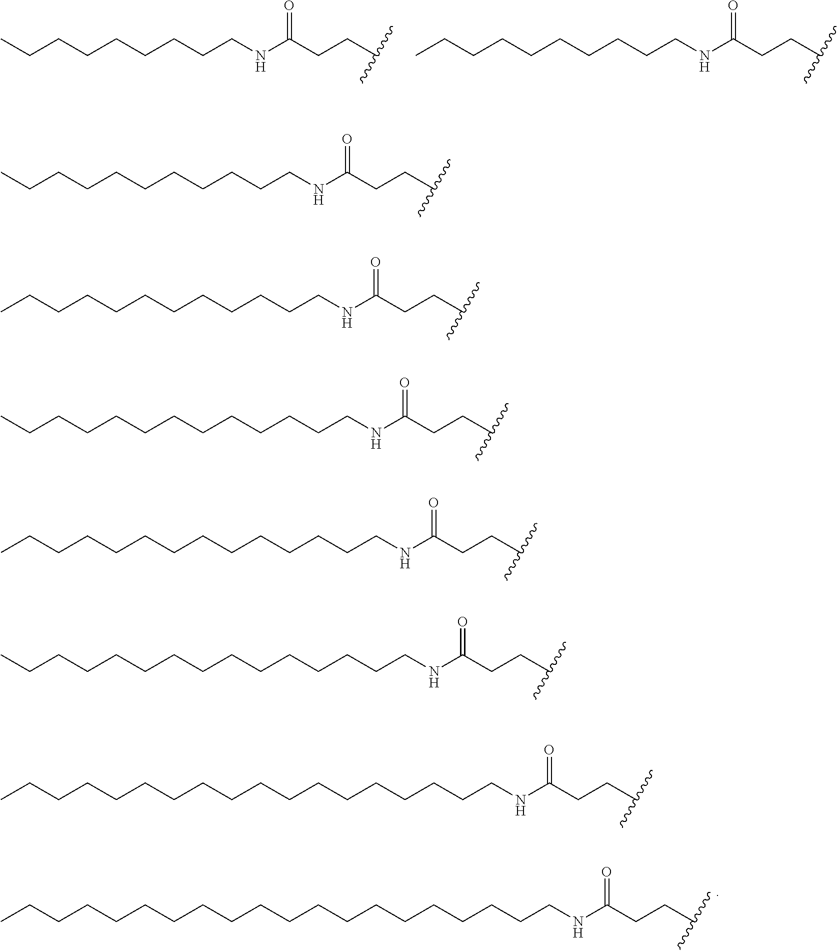

In some embodiments,

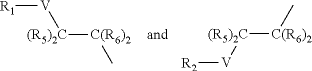

are the same and are selected from the group consisting of:

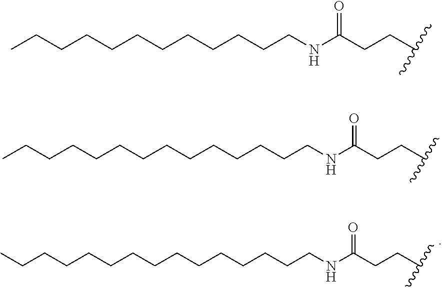

In another aspect, the disclosure features a therapeutic composition that includes a therapeutic agent (e.g., a therapeutic protein, e.g., a protein for use in replacement therapy, e.g., a protein described herein) and a compound of the formula:

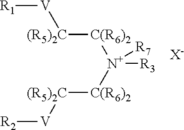

wherein V is selected from the group consisting of C═O, C═S, S═O, and SO2;

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; or —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(═O)RB; —CO2RB; —CN; —SCN; —SRB; —SORB; —SO2RB; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORC; —C(═O)RC; —CO2Rc; —CN; —SCN; —SRC; —SORC; —SO2Rc; —NO2; —N3; —N(Rc)2; —NHC(═O)Rc; —NRcC(═O)N(Rc)2; —OC(═O)ORc; —OC(═O)Rc; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R5 is independently selected from the group consisting of hydrogen and C1-C6 alkyl; each occurrence of R6 is independently selected from the group consisting of hydrogen and C1-C6alkyl;

R7 is hydrogen or C1-C6 aliphatic; and

X is an anion; with the proviso that when all occurrences of R5 and R6 are hydrogen, V is C═O, R1 is —ORA, R2 is —ORB, and R1 and R2 are the same, then R3 is not —CH2CH2ORc′, wherein Rc′ is methyl, ethyl, propyl, isopropyl, butyl, s-butyl, isobutyl, t-butyl, pentyl, cyclopentyl, hexyl, cyclohexyl, decyl, methoxymethyl, 2-methoxyethyl, 1-ethoxyethyl, 2-ethoxyethyl, (2-methoxyethoxy)methyl, 2-tetrahydrofuranyl, 2-tetrahydropyranyl, tetrahydrofurfuryl, formyl, acetyl, propionyl, butyryl, isobutyryl, pivaloyl, valeryl, methoxyacetyl, ethoxyacetyl, acetoxyacetyl, 2-formyloxyethyl, 2-acetoxyethyl, 2-oxopropyl, 2-oxobutyl, 2-oxocyclopentyl, 2-oxo-3-tetrahydrofuranyl, 2-oxo-3-tetrahydropyranyl, methoxycarbonyl, ethoxycarbonyl, and t-butoxycarbonyl; and with the proviso that when all occurrences of R5 and R6 are hydrogen, V is C═O, R1 is —ORA, wherein RA is a straight-chain, branched or cyclic alkyl group of 1 to 20 carbon atoms which may contain an ether, carbonyl, or carbonyloxy group; R2 is —ORB, wherein RB is a straight-chain, branched or cyclic alkyl group of 1 to 20 carbon atoms which may contain an ether, carbonyl, or carbonyloxy group; and R1 and R2 are the same, the R3 is not —CH2CH2ORc″, wherein Rc″ is a straight-chain, branched or cyclic alkyl group of 1 to 20 carbon atoms which may contain an ether, carbonyl, or carbonyloxy group; and salts thereof.

In some embodiments, the therapeutic agent a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, X is an anion selected from the group consisting of fluoride, chloride, bromide, iodide, sulfate, bisulfate, phosphate, nitrate, acetate, fumarate, oleate, citrate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate).

In some embodiments, R7 is C1-C6 alkyl.

In some embodiments, R7 is hydrogen.

In some embodiments, R7 is methyl.

In some embodiments, each occurrence of R5 and R6 is hydrogen.

In some embodiments,

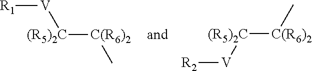

are the same.

In some embodiments

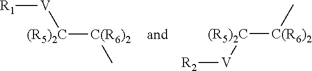

are the same and are different than R3.

In some embodiments,

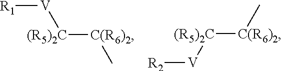

and R3 are all different.

In some embodiments,

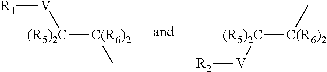

are independently selected from the group consisting of:

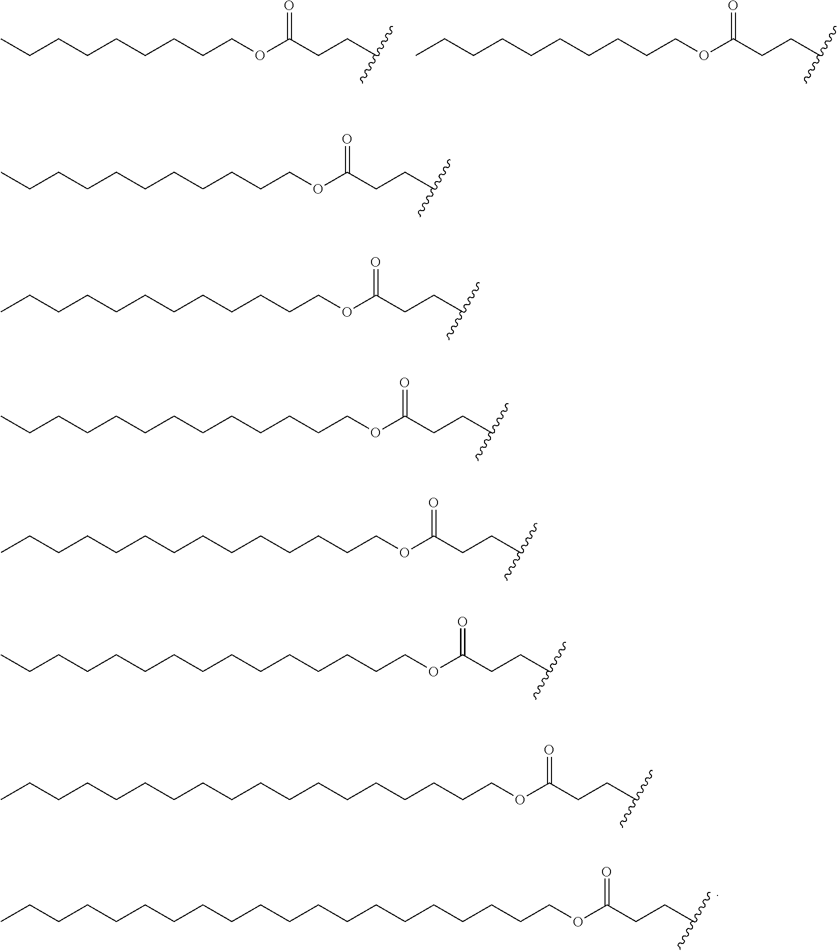

In some embodiments

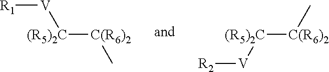

are the same and are selected from the group consisting of:

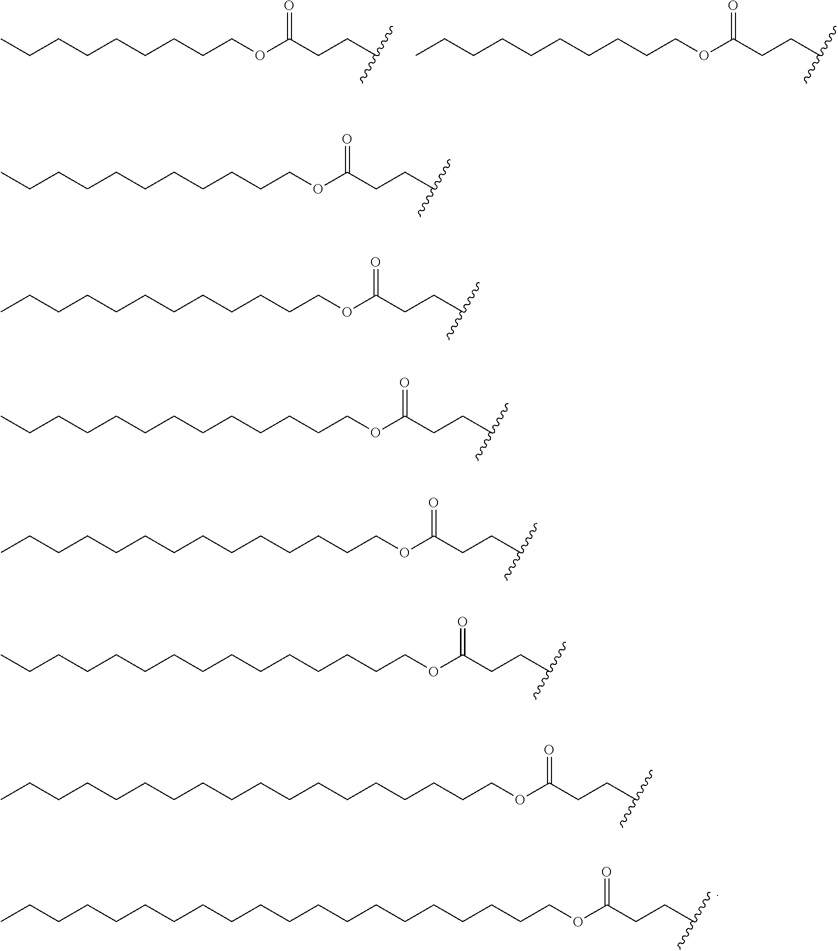

In some embodiments,

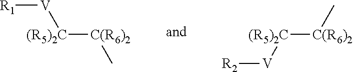

are the same and are selected from the group consisting of:

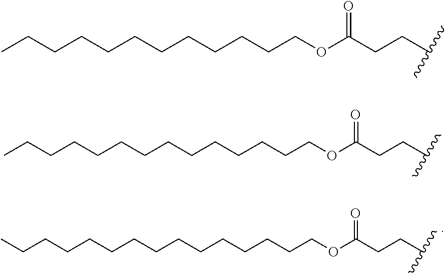

In some embodiments,

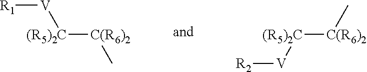

are independently selected from the group consisting of:

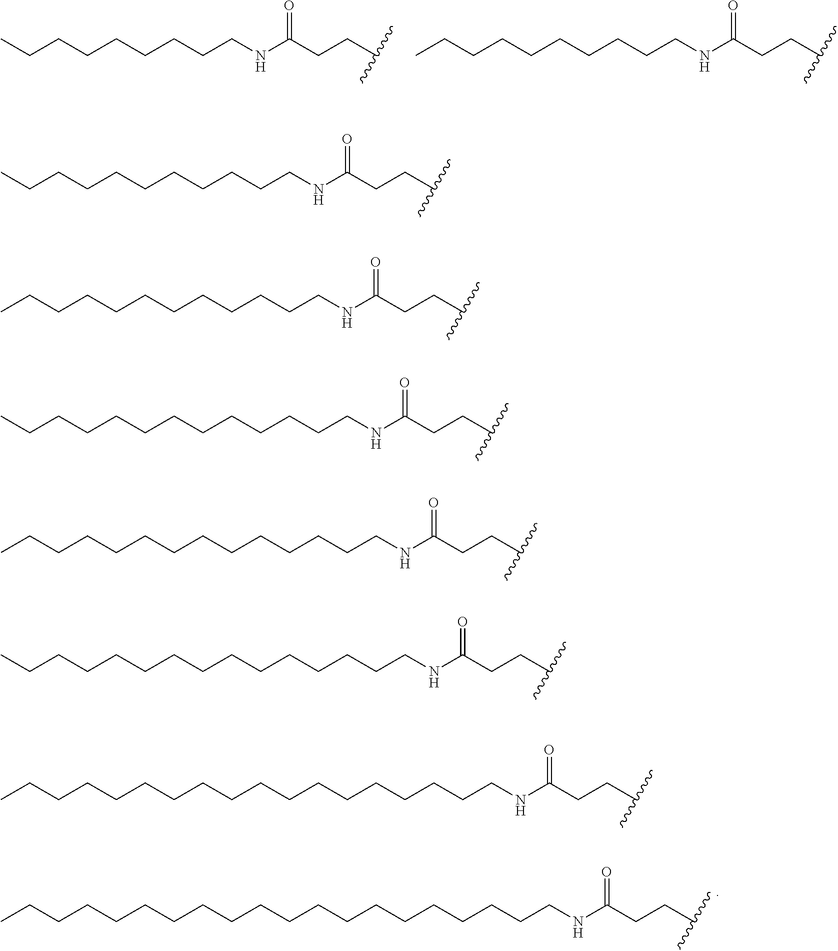

In some embodiments,

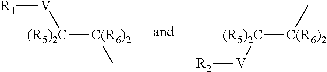

are the same and are selected from the group consisting of:

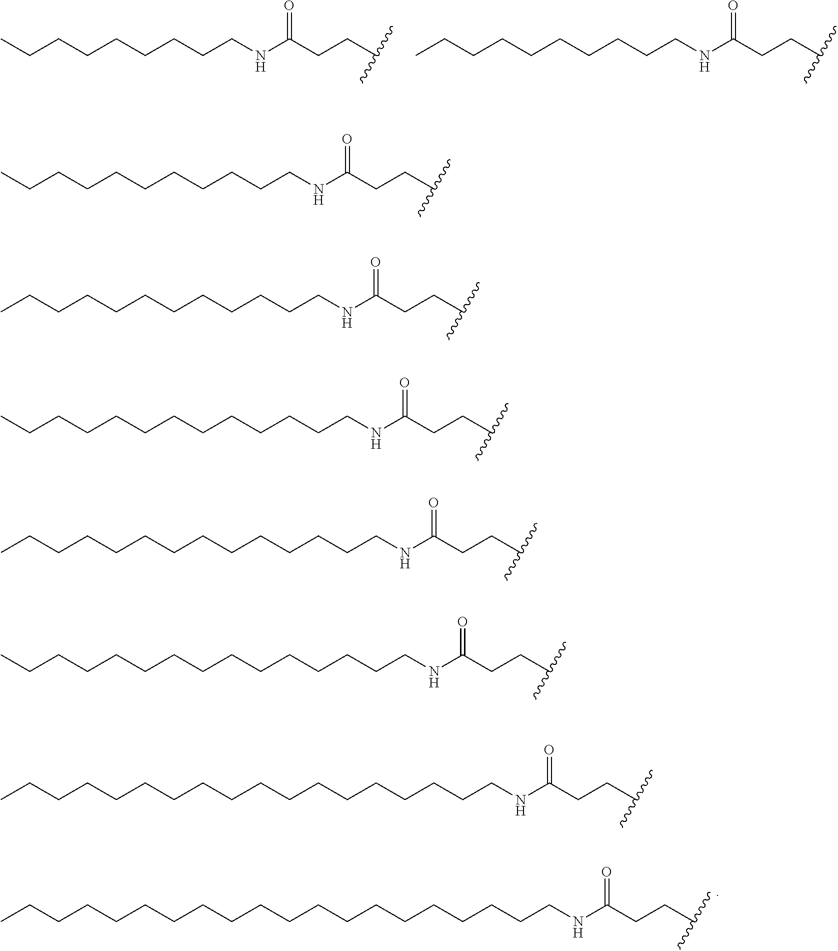

In some embodiments,

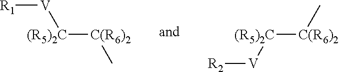

are the same and are selected from the group consisting of:

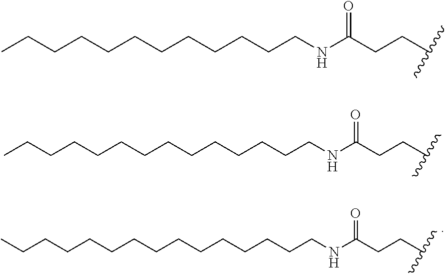

In some embodiments,

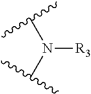

is selected from the group consisting of:

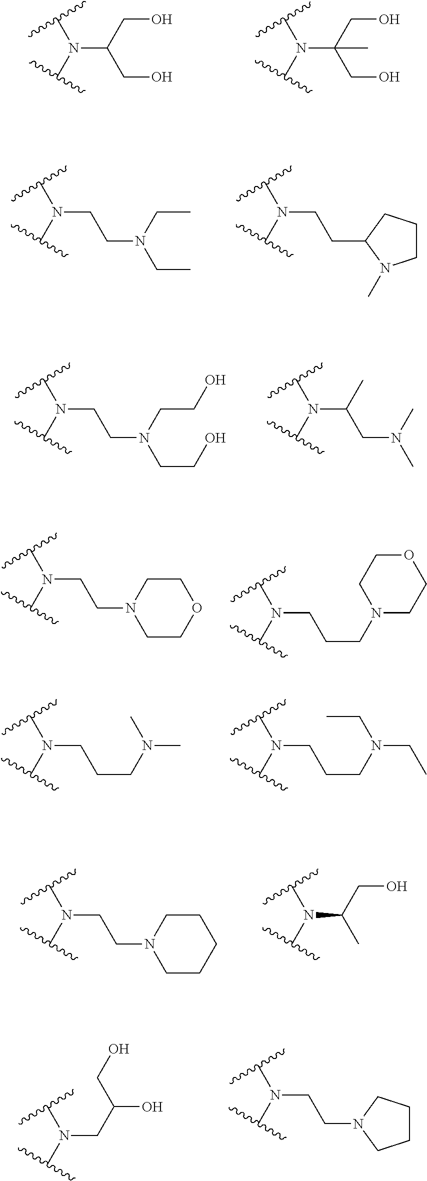

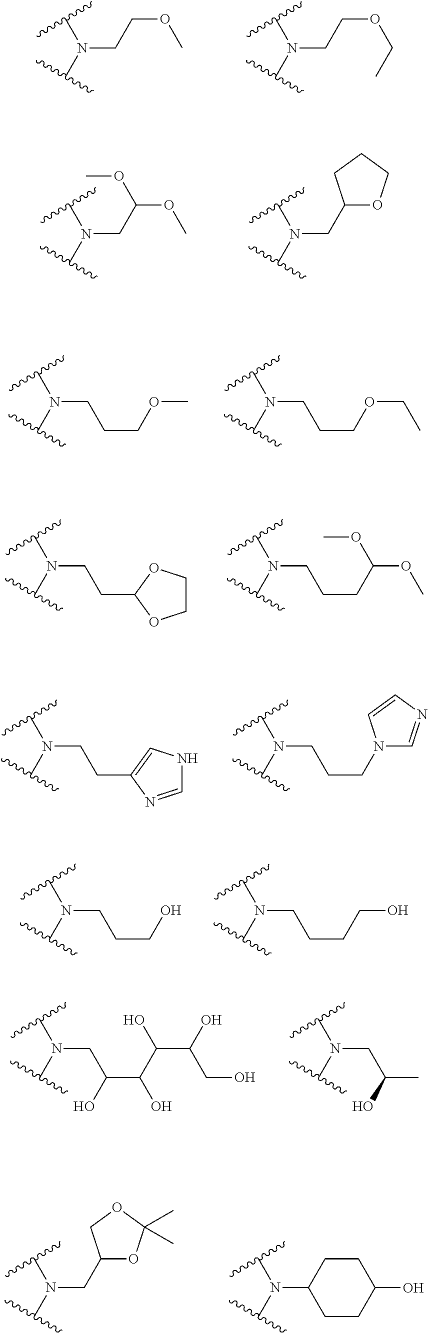

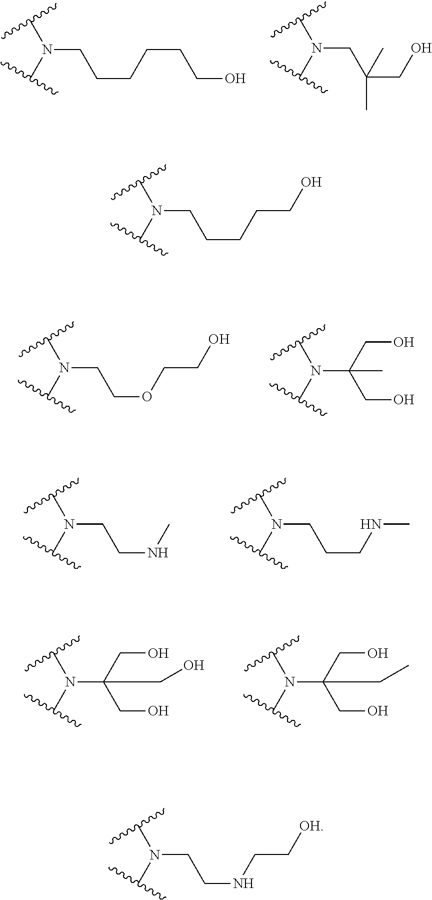

In some embodiments,

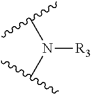

is selected from the group consisting of:

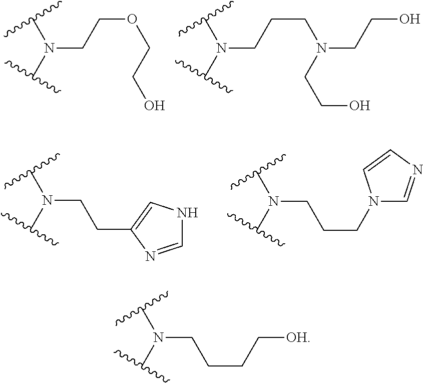

In some embodiments

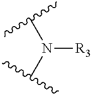

is selected from the group consisting of:

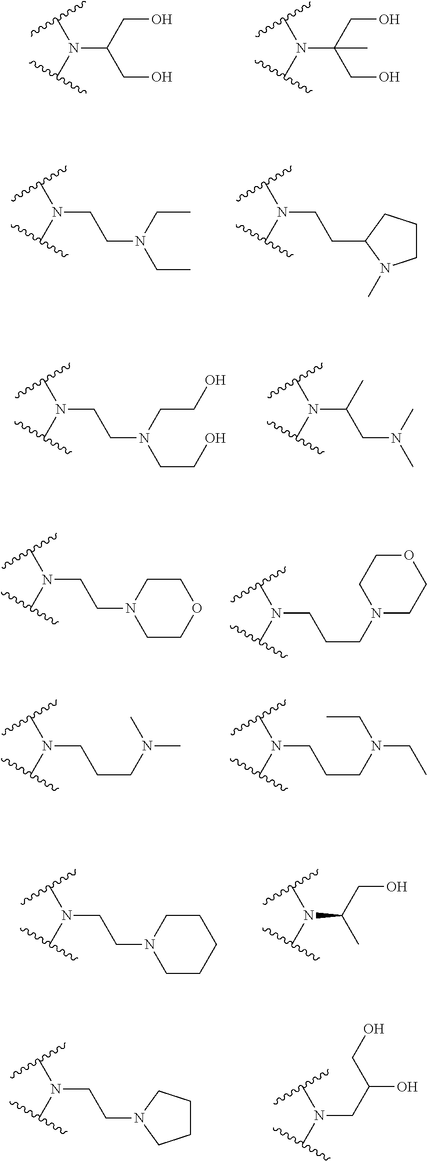

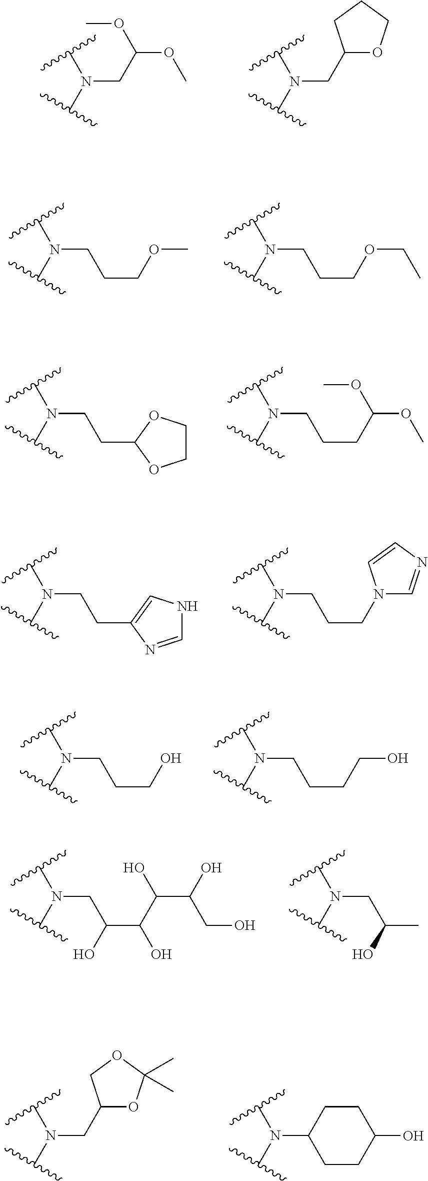

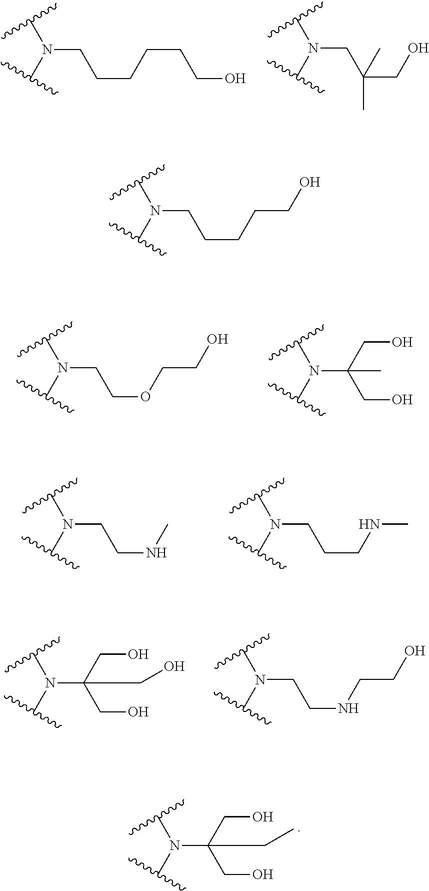

In some embodiments, the compound has the formula:

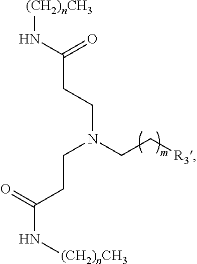

wherein

R3′ is C1-6alkyl, hydroxyl, thiol; C1-6alkoxy; amino, C1-6 alkylamino, diC1-6alkylamino; carbocyclic moiety; heterocyclic moiety; aryl; or heteroaryl moiety; n is an integer between 5 and 20, inclusive; and m is an integer between 1 and 10, inclusive; and pharmaceutically acceptable salts thereof.

In some embodiments, R3′ is hydroxyl.

In some embodiments, R3′ is methoxy or ethoxy.

In some embodiments, R3′ is heteroaryl.

In some embodiments, R3′ is histidinyl.

In some embodiments, R3′ is a heterocyclic moiety.

In some embodiments, the compound is selected from the group consisting of:

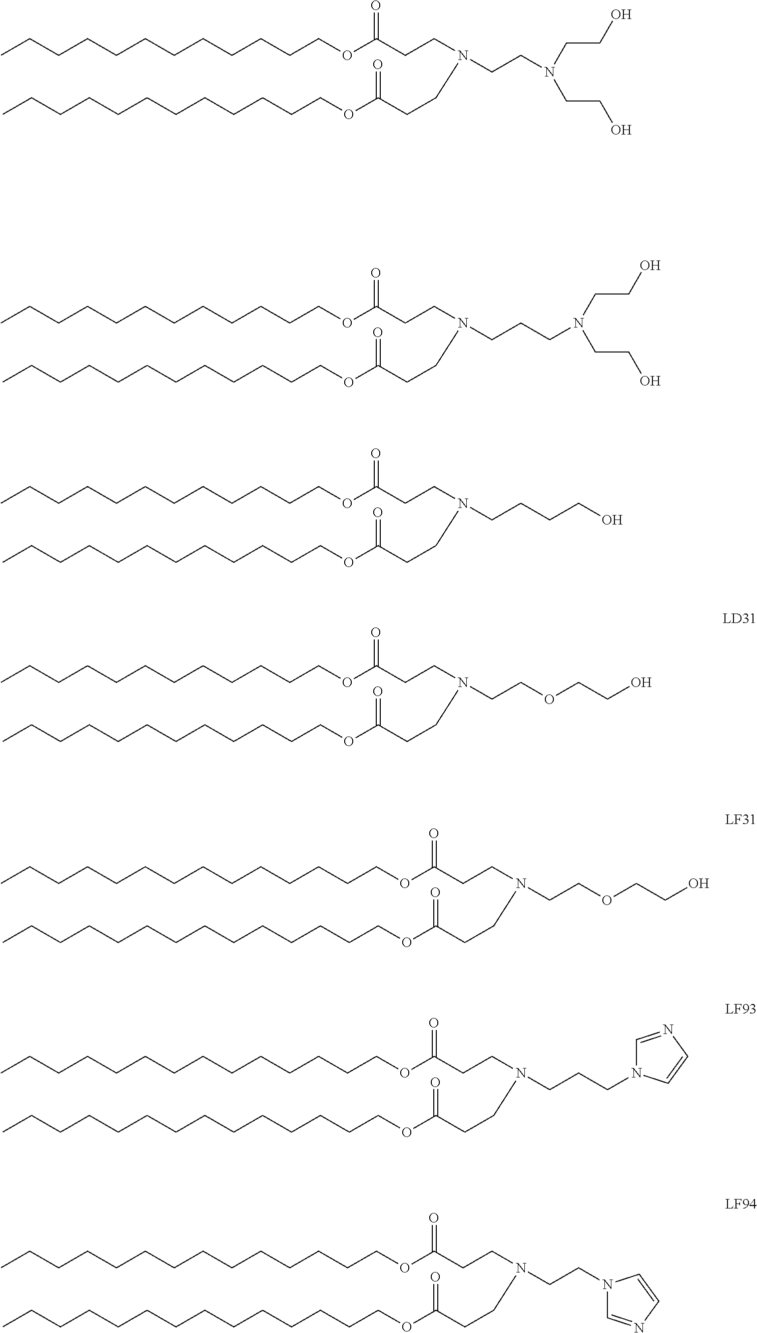

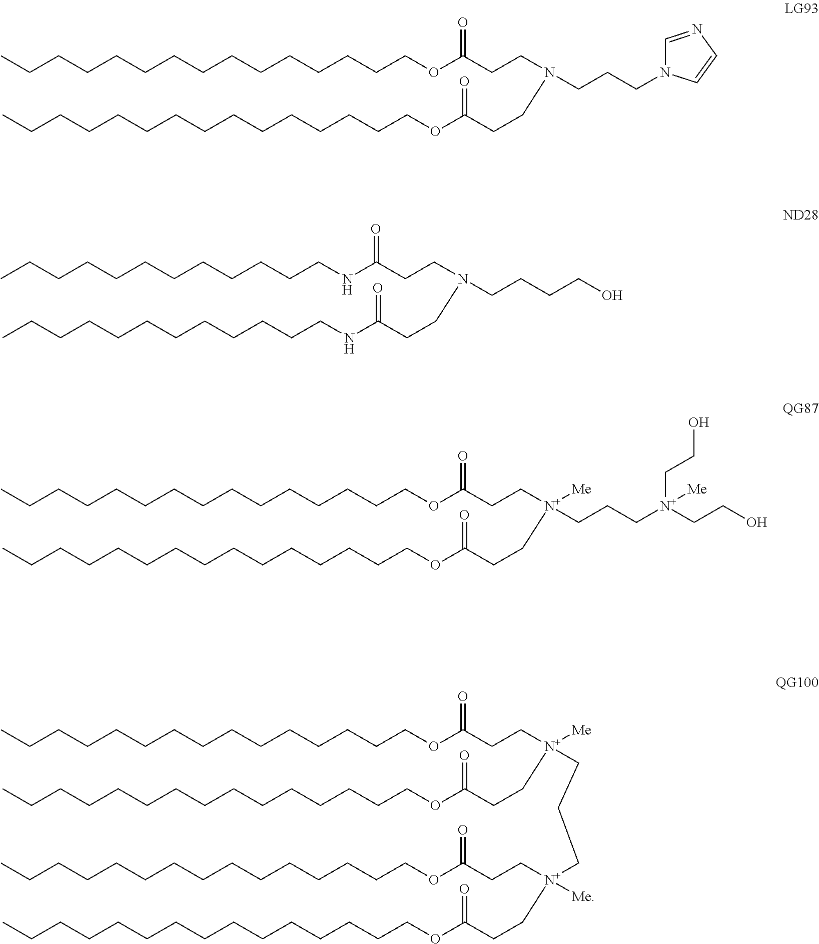

In some embodiments, the compound has the formula:

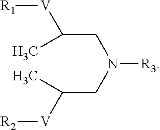

In some embodiments, the compound has the formula:

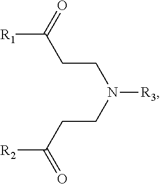

wherein V is C═O.

In some embodiments, the compound has the formula:

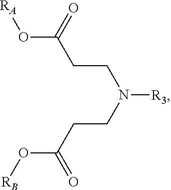

wherein R1 is —ORA; and R2 is —ORB.

In some embodiments, RA and RB are the same.

In some embodiments, RA and RB are C6-C30 straight chain alkyl groups.

In some embodiments, RA and RB are C9-C20 straight-chain alkyl groups.

In some embodiments, RA and RB are C21-C30 straight-chain alkyl groups.

In some embodiments, RA and RB are C6-C30 straight chain alkenyl groups.

In some embodiments, RA and RB are C6-C30 straight chain alkynyl groups.

In some embodiments, RA and RB each comprise at least 4 carbon atoms.

In some embodiments, RA and RB each comprise at least 5 carbon atoms.

In some embodiments, RA and RB each comprise at least 6 carbon atoms.

In some embodiments, RA and RB are independently selected from the group consisting of a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety.

In some embodiments, RA and RB are cyclic or acyclic, substituted or unsubstituted, branched or unbranched aliphatic moities.

In some embodiments, RA and RB are the same.

In some embodiments, RA and RB are acyclic, substituted or unsubstituted, branched or unbranched aliphatic moieties.

In some embodiments, RA and RB are acyclic, unsubstituted, unbranched aliphatic moeities.

In some embodiments, RA and RB are alkyl groups.

In some embodiments, RA and RB are C1-C30 alkyl groups.

In some embodiments, RA and RB are C5-C20 alkyl groups.

In some embodiments, RA and RB are C5-C12 alkyl groups.

In some embodiments, RA and RB are polyethylene glycol groups.

In some embodiments, R3 is a cyclic or acyclic, substituted or unsubstituted, branched or unbranched aliphatic moiety; a cyclic or acyclic, substituted or unsubstituted, branched or unbranched heteroaliphatic moiety; a substituted or unsubstitued, branched or unbranched aryl moiety; or a substituted or unsubstituted, branched or unbranched heteroaryl moiety.

In some embodiments, R3 is not

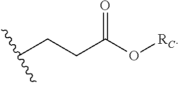

In some embodiments, the compound has the formula:

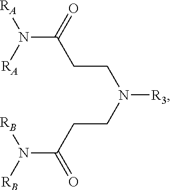

wherein R1 is —N(RA)25 and R2 is —N(RB)2-

In some embodiments, the compound has the formula:

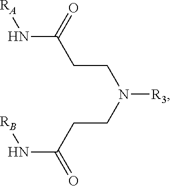

wherein R1 is —NHRA; and R2 is —NHRB.

In some aspects, the disclosure features a therapeutic composition that contains a therapeutic agent (e.g., a therapeutic protein, e.g., a protein for use in replacement therapy, e.g., a protein described herein) and a compound of the formula:

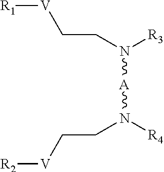

wherein A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; and substituted or unsubstituted, branched or unbranched heteroaryl;

V is selected from the group consisting of C═O, C═S, S═O, and SO2;

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(═O)RB; —CO2RB; —CN; —SCN; —SRB; —SORB; —SO2RB; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORC; —C(═O)RC; —CO2RC; —CN; —SCN; —SRC; —SORO; —SO2RC; —NO2; —N3; —N(Rc)2; —NHC(═O)RC; —NRCC(═O)N(RC)2; —OC(═O)ORC; —OC(═O)RC; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORD; —C(═O)RD; —CO2RO; —CN; —SCN; —SRO; —SORD; —SO2RO; —NO2; —N3; —N(RD)2; —NHC(═O)RD; —NRCC(═O)N(RO)2; —OC(═O)ORD; —OC(═O)RO; —OC(═O)N(RO)2; —NRcC(═O)ORD; or —C(RO)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a therapeutic composition that contains a therapeutic agent (e.g., a therapeutic protein, e.g., a protein for use in replacement therapy, e.g., a protein described herein) and a compound of the formula:

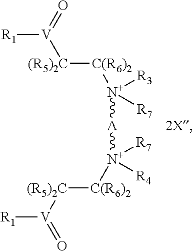

wherein A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; and substituted or unsubstituted, branched or unbranched heteroaryl;

V is selected from the group consisting of C═O, C═S, S═O, and SO2;

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(O)RA; —CO2RA; —CN; —SCN; —SRA; —SORA; —SO2RA; —NO2; —N3; —N(RA)2; —NHC(═O)RA; —NRAC(O)N(RA)2; —OC(═O)ORA; —OC(O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(O)R8; —CO2RB; —CN; —SCN; —SRB; —SORB; —SO2RB; —NO2; —N3; —N(RB)2; —NHC(O)RO; —NRBC(O)N(RB)2; —OC(O)OR8; —OC(O)RB; —OC(O)N(RB)2; —NRBC(O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORO; —C(O)RO; —CO2RC; —CN; —SCN; —SRC; —SORO; —SO2RC; —NO2; —N3; —N(Rc)2; —NHC(O)RO; —NROC(O)N(Rc)2; —OC(O)ORO; —OC(O)RO; —OC(O)N(Rc)2; —NRcC(O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORD; C(O)Rn; —CO2RO; —CN; —SCN; —SRD; —SORD; —SO2RO; —NO2; —N3; —N(RD)2; —NHC(O)RO; —NRCC(O)N(RD)2; —OC(O)ORO; —OC(O)RO; —OC(O)N(RD)2; —NROC(O)ORD; or —C(RD)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R7 is C1-C6 aliphatic; X is an anion; and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, X is an anion selected from the group consisting of fluoride, chloride, bromide, iodide, sulfate, bisulfate, phosphate, nitrate, acetate, fumarate, oleate, citrate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate).

In some embodiments, R7 is hydrogen.

In some embodiments, R7 is C1-C6 alkyl.

In some embodiments, R7 is methyl.

In some embodiments, A is a cyclic or acyclic, substituted or unsubstituted, branched or unbranched aliphatic moiety; or a cyclic or acyclic, substituted or unsubstituted, branched or unbranched heteroaliphatic moiety.

In some embodiments, A is an acyclic, unsubstituted, unbranched aliphatic moiety.

In some embodiments, A is an acyclic, unsubstituted, unbranched alkyl group.

In some embodiments, A is an acyclic, unsubstituted, unbranched C1-C6 alkyl group.

In some embodiments, A is an acyclic, unsubstituted, unbranched heteroaliphatic moiety.

In some embodiments, A is a polyethylene glycol moiety.

In some embodiments, R3 and R4 are the same.

In some embodiments, R3 and R4 are C1-C6 alkyl group.

In some embodiments, R3 or R4 is

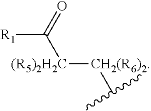

In some embodiments,

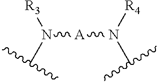

is selected from the group consisting of:

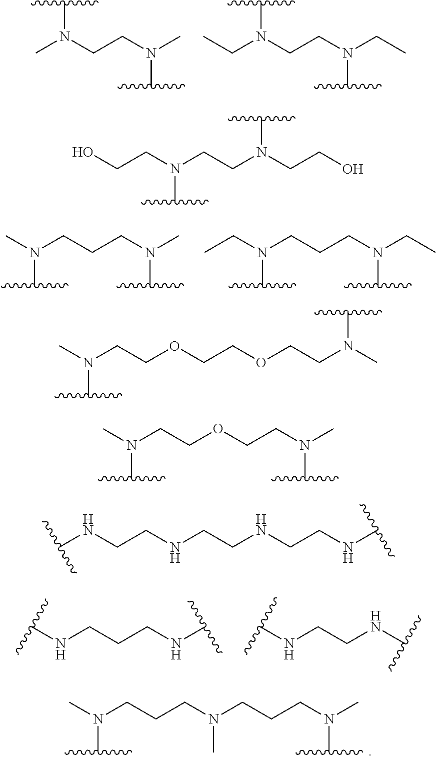

In some embodiments,

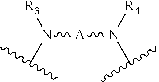

is:

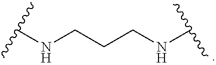

In some embodiments,

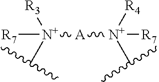

is selected from the group consisting of:

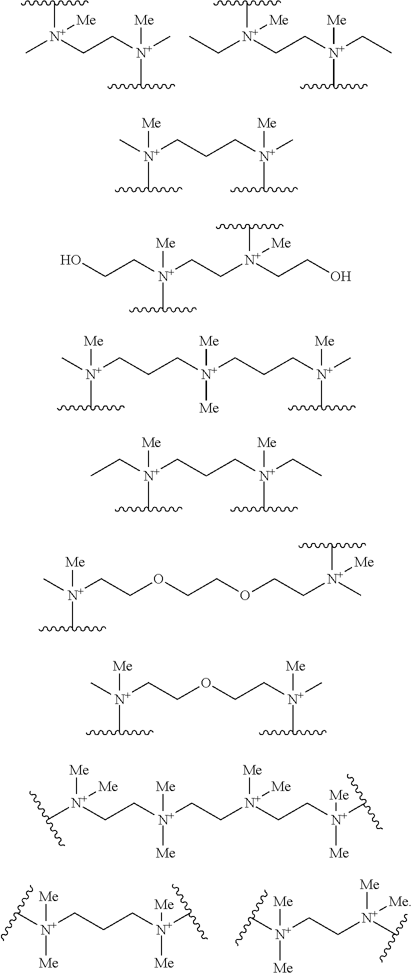

In some embodiments

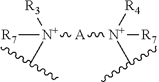

is:

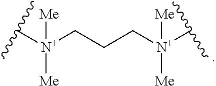

In some aspects, the disclosure features a therapeutic composition that contains a therapeutic agent (e.g., a therapeutic protein, e.g., a protein for use in replacement therapy, e.g., a protein described herein) and a compound of the formula:

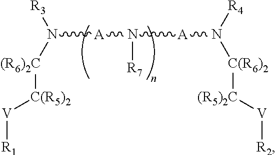

wherein

A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; and substituted or unsubstituted, branched or unbranched heteroaryl;

V is selected from the group consisting of C═O, C═S, S═O, and SO2;

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(K))ORA; —OC(O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(═O)RB; —CO2R3; —CN; —SCN; —SRB; —SORB; —SO2RB; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORC; —C(═O)Rc; —CO2RC; —CN; —SCN; —SRC; —SORC; —SO2RC; —NO2; —N3; —N(Rc)2; —NHC(═O)Rc; —NRcC(═O)N(Rc)2; —OC(═O)ORc; —OC(═O)RO; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORD; —C(═O)RO; —CO2RO; —CN; —SCN; —SR; —SORD; —SO2RD; —NO2; —N3; —N(RD)2; —NHC(═O)RD; —NRCC(═O)N(RD)2; —OC(═O)ORD; —OC(═O)RD; —OC(═O)N(RD)2; —NRCC(═O)ORD; or —C(RD)3; wherein each occurrence of Ro is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R5 is independently selected from the group consisting of hydrogen and C1-C6 alkyl; each occurrence of R6 is independently selected from the group consisting of hydrogen and C1-C6alkyl;

R7 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORG; —CC(═O)RG; —CO2RG; —CN; —SCN; —SRG; —SORG; —SO2RG; —NO2; —N3; —N(RG)2; —NHC(═O)RG; —NRGC(═O)N(RG)2; —OC(═O)ORG; —OC(═O)RO; —OCC(═O)N(RG)2; —NRGC(═O)ORG; and —C(RG)3; wherein each occurrence of Ro is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety and salts thereof.

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In other aspects, the disclosure features a therapeutic composition that includes a therapeutic agent (e.g., a therapeutic protein, e.g., a protein for use in replacement therapy, e.g., a protein described herein) and a compound of the formula:

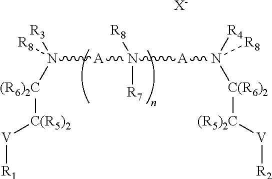

wherein

A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; and substituted or unsubstituted, branched or unbranched heteroaryl;

V is selected from the group consisting of C═O, C═S, S═O, and SO2;

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(O)R3; —CO2RB; —CN; —SCN; —SRB; —SORB; —SO2R8; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORC; —C(O)Rc; —CO2RC; —CN; —SCN; —SRO; —SORC; —SO2Rc; —NO2; —N3; —N(Rc)2; —NHC(═O)RC; —NRCC(═O)N(RC)2; —OC(═O)ORc; —OC(═O)Rc; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORD; —C(═O)RD; —CO2RO; —CN; —SCN; —SRD; —SORD; —SO2RO; —NO2; —N3; —N(RD)2; —NHC(═O)RD; —NRCC(═O)N(RD)2; —OC(═O)ORD; —OC(═O)RD; —OC(═O)N(RD)2; —NRCC(═O)ORD; or —C(RD)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R5 is independently selected from the group consisting of hydrogen and C1-C6 alkyl; each occurrence of R6 is independently selected from the group consisting of hydrogen and C1-C6alkyl;

R7 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORG; —C(═O)RG; —CO2RG; —CN; —SCN; —SRG; —SORG; —SO2RG; —NO2; —N3; —N(RG)2; —NHC(═O)RG; —NRGC(═O)N(RG)2; —OC(═O)ORG; —OC(═O)RO; —OC(═O)N(RG)2; —NRGC(═O)ORG; and —C(RG)3; wherein each occurrence of RG is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R8 is hydrogen or C1-C6 aliphatic; each dashed line represents a bond or the absence of a bond, wherein when the dashed line represents a bond, the attached nitrogen is positively charged; and X is any anion.

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, each occurrence of R8 is hydrogen or methyl.

In some embodiments,

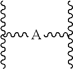

is selected from the group consisting of:

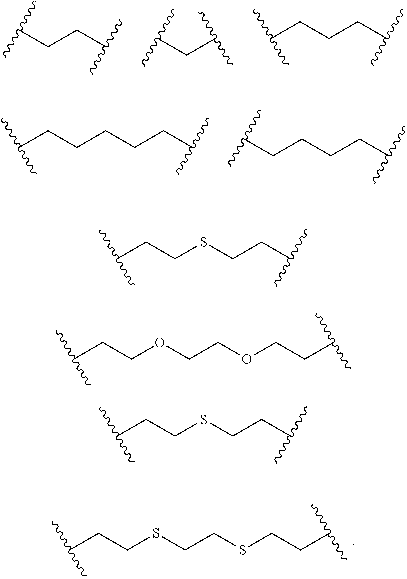

In some embodiments, the compound has the formula:

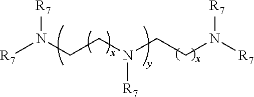

wherein each occurrence of x is an integer between 1 and 10, inclusive; y is an integer between 0 and 10, inclusive;

each occurrence of R7 is hydrogen or

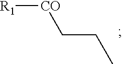

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; and salts thereof.

In some embodiments, x is an integer between 1 and 6, inclusive.

In some embodiments, y is an integer between 1 and 3, inclusive.

In some embodiments, R1 is —ORA.

In some embodiments, R1 is —ONHRA.

In some embodiments, R7 is hydrogen or

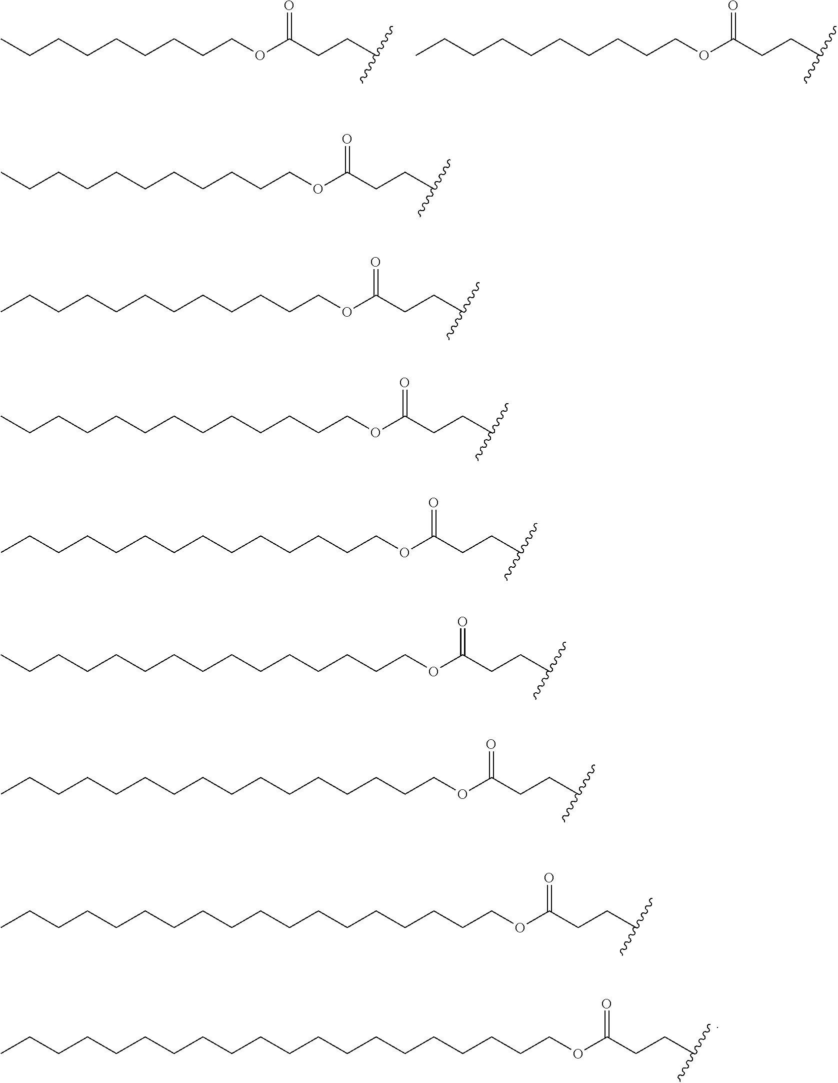

In some embodiments, R7 is hydrogen or

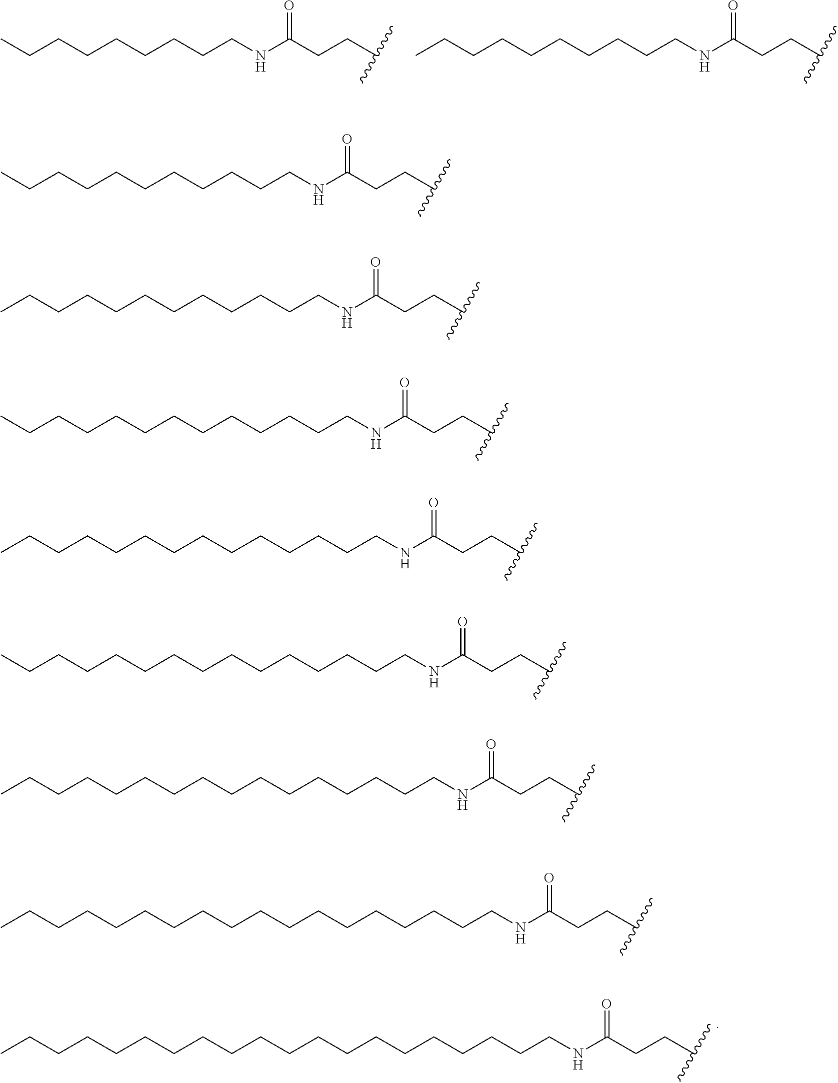

In other aspects, the disclosure features a therapeutic composition that contains a therapeutic agent (e.g., a therapeutic protein, e.g., a protein for use in replacement therapy, e.g., a protein described herein) and a compound of the formula:

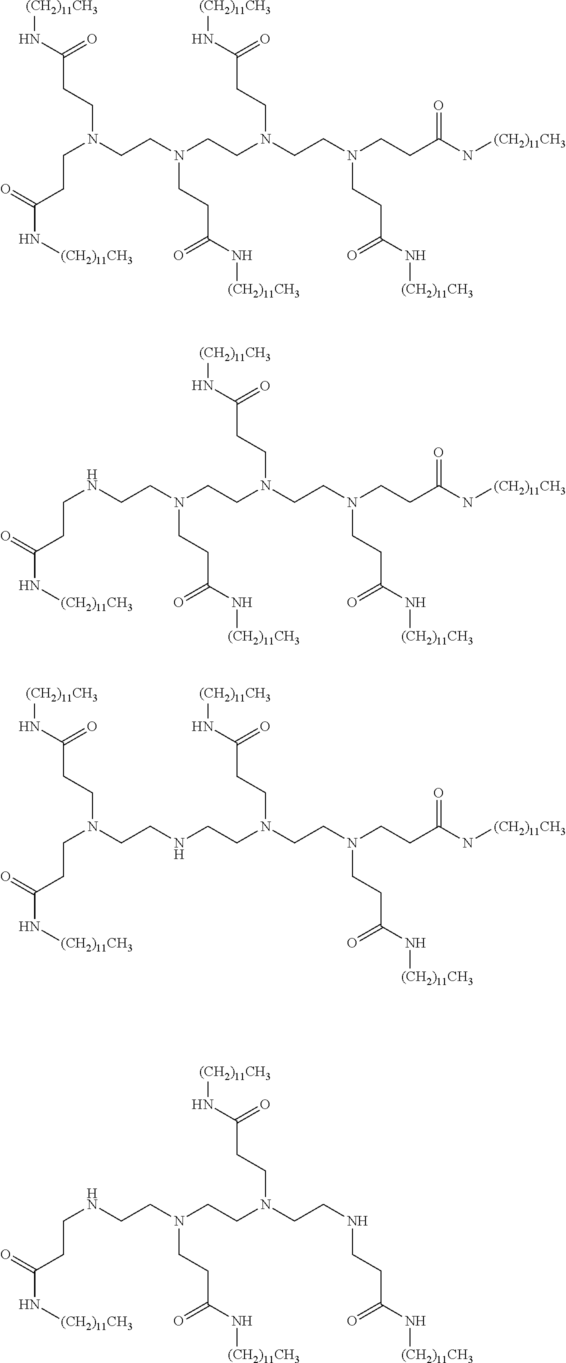

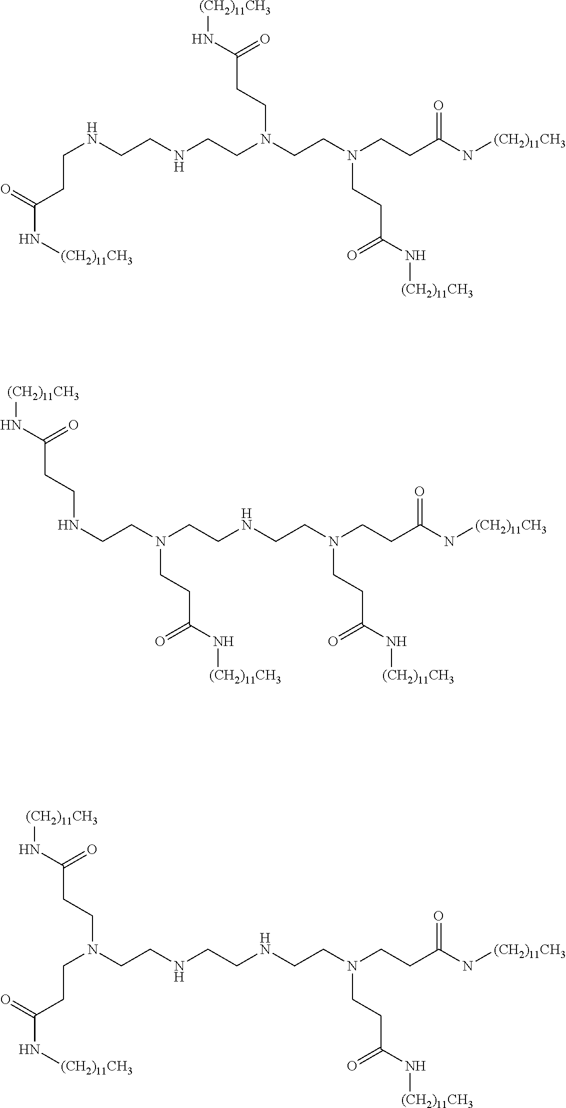

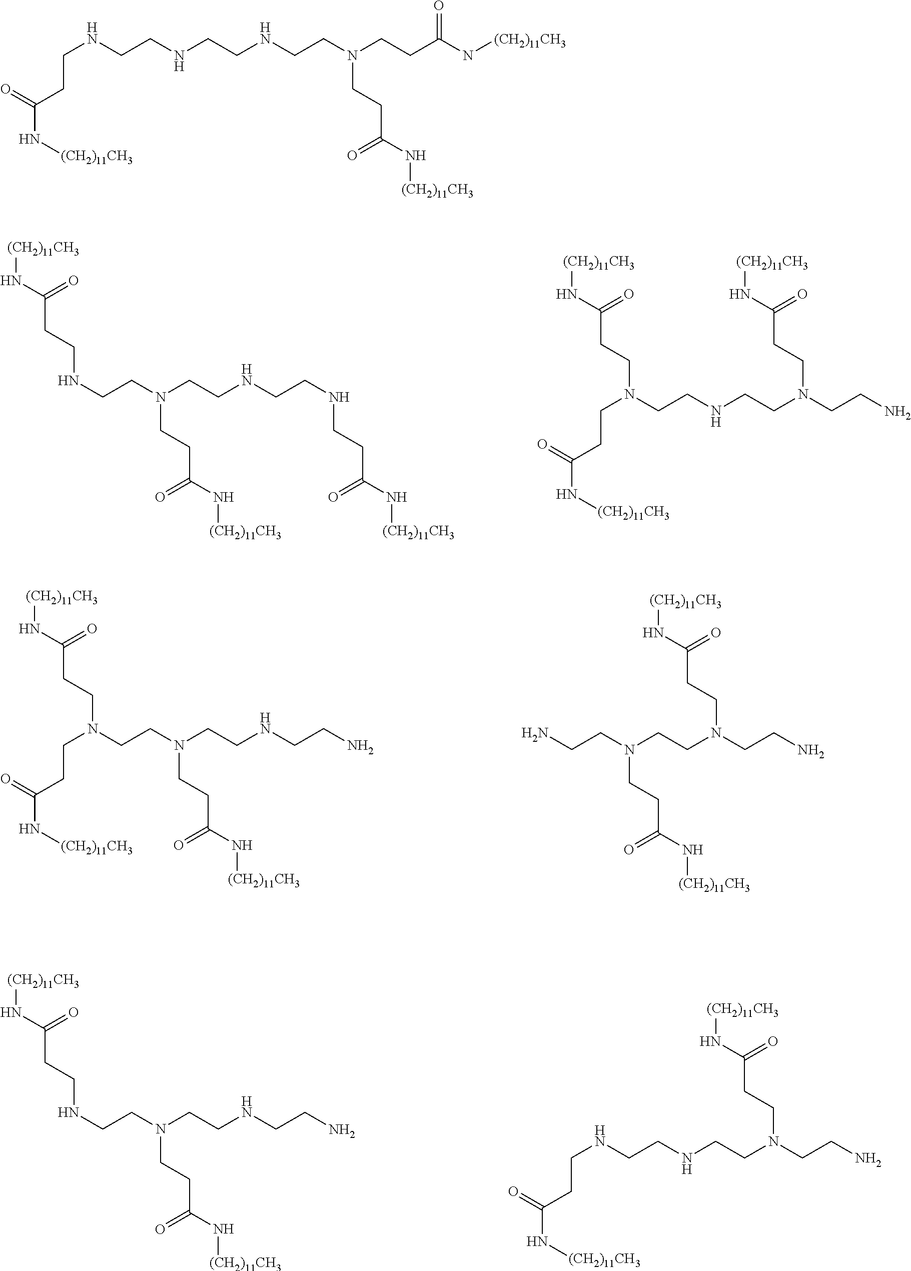

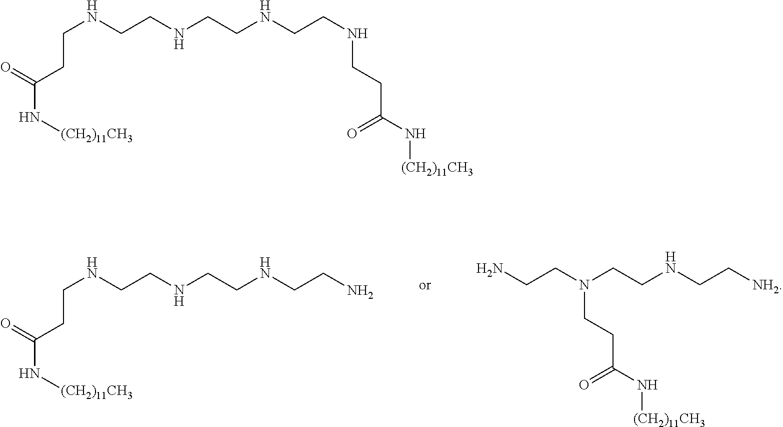

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, the compound is of formula:

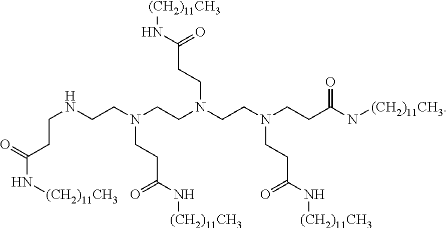

In some embodiments, the compound is of formula:

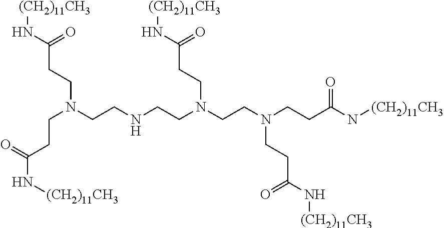

In some aspects, the disclosure features a therapeutic composition that contains a therapeutic agent (e.g., a therapeutic protein, e.g., a protein for use in replacement therapy, e.g., a protein described herein) and one or more of the compounds of formula:

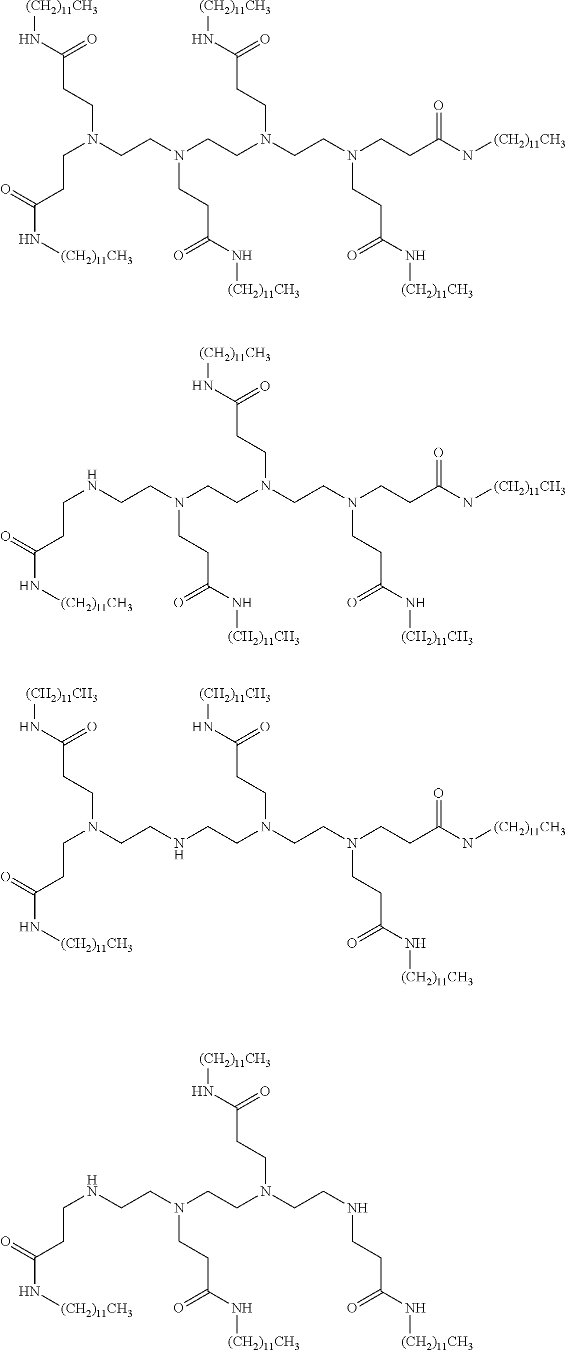

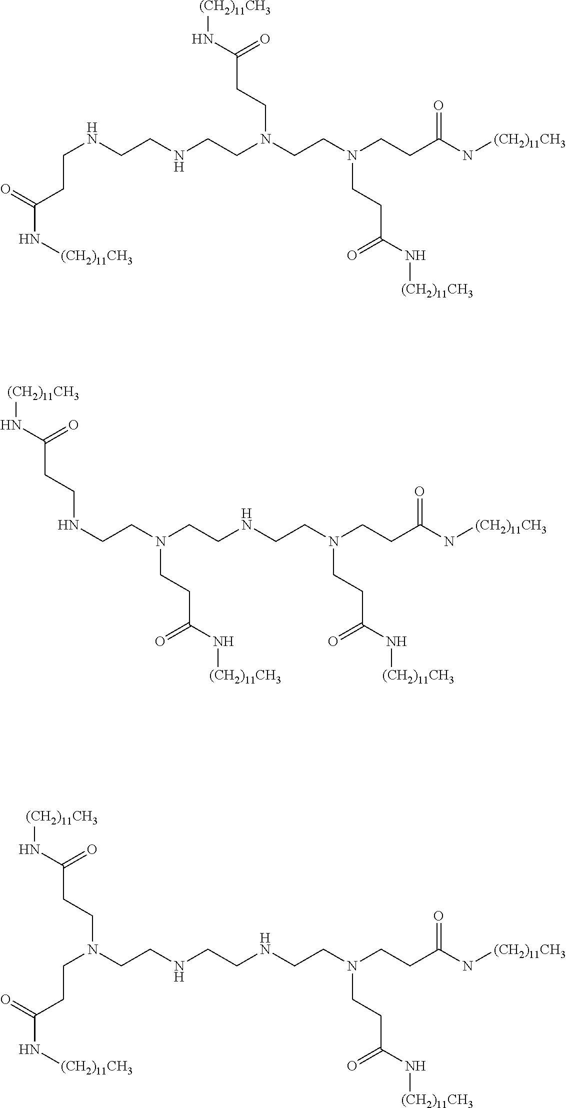

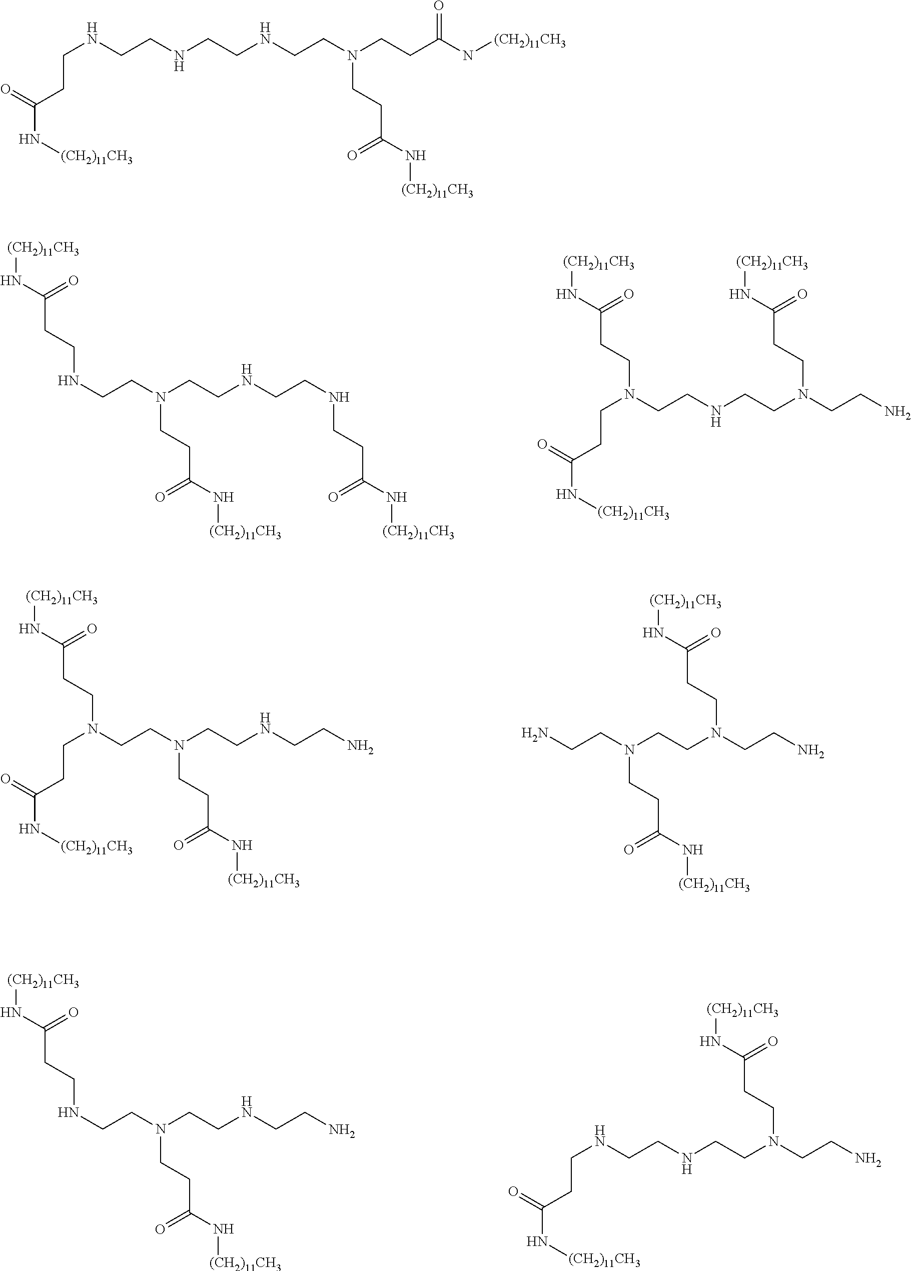

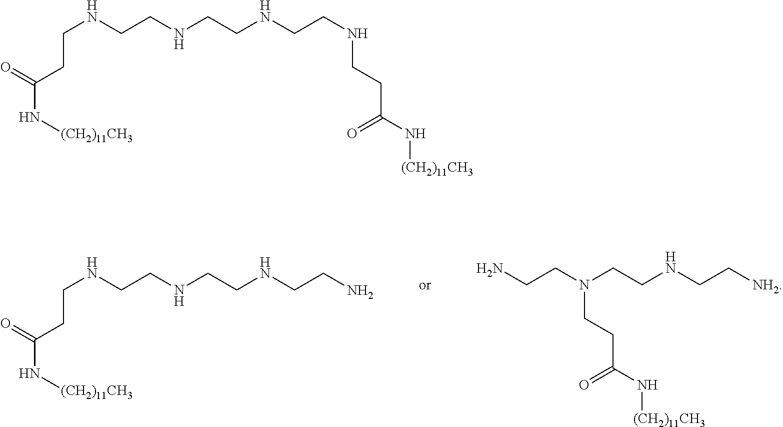

In some embodiments, the therapeutic agent is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In one aspect, the disclosure features a microparticle comprising a therapeutic composition described herein.

In another aspect, the disclosure features a liposome comprising a therapeutic composition described herein.

In some embodiments, the liposome further contains cholesterol.

In some embodiments, the liposome further contains PEG-ceramide.

In some embodiments, the liposome contains a therapeutic composition described herein; cholesterol; and PEG-ceramide

In one aspect, the disclosure features a micelle comprising a therapeutic composition described herein.

In one aspect, the disclosure features a pharmaceutical composition comprising a therapeutic composition described herein and pharmaceutical agent.

In one aspect, the disclosure features a method of making a therapeutic composition, the method includes:
providing a lipid described herein;
providing a therapeutic agent described herein (e.g., a therapeutic protein, e.g., a protein for use in replacement therapy, e.g., a protein described herein); and
combining the lipid and the therapeutic agent, thereby making a therapeutic composition.

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In one aspect, the disclosure features a method of preparing microparticles, the method includes:

contacting a therapeutic agent described herein (e.g., a therapeutic protein, e.g., a protein for use in replacement therapy, e.g., a protein described herein) with a lipid described herein in the presence of a solvent to form a mixture; and spray drying the mixture, thereby preparing microparticles.

In some embodiments, the therapeutic agent is a therapeutic protein that includes Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In one aspect, the disclosure features a method of treating an individual, the method includes administering a therapeutic composition described herein to an individual in need of such treatment (e.g., an individual in need of replacement therapy).

In some embodiments, the therapeutic composition includes a therapeutic agent that is a therapeutic protein that contains Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

In some embodiments, the invention features the use of a therapeutic composition described herein for use in therapy.

In some embodiments, the invention features the use of a therapeutic composition described herein for the preparation of a medicament for replacement therapy.

The present invention provides therapeutic compositions that contain lipids of the formula (I):

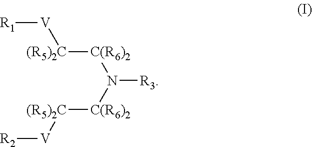

These lipids may be prepared by the addition of a primary amine to a double bond conjugated with an electron withdrawing groups such as a carbonyl moiety. Two equivalents of an [alpha], [beta]-unsaturated ketone such as an acrylate are reacted with one equivalent of a primary amine to prepare the lipids as shown in the scheme below:

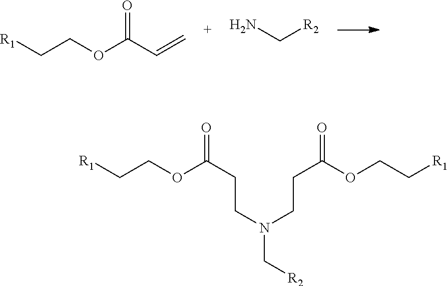

These lipids typically have a hydrophobic half and a hydrophilic half. The hydrophobic portion is typically provided by fatty acid moieties attached to the acrylate, and the hydrophilic portion is provided by the esters, amines, and side chain of the amine. The fatty acid groups may be straight chain alkyl groups (C1-C30) with no substitutions. In certain embodiments, the fatty acid groups are substituted and/or branched. The amine may be protonated or alkylated thereby forming a positively charged amine. These lipids may be used in the delivery of therapeutic agents to a subject. The lipids are particularly useful in delivering negatively charged agents given the tertiary amine available for protonation thus forming a positive charge. As would be appreciated by one of skill in the art, the above reaction may result in a mixture with some lipids have one acrylate tail and other having two acrylate tails. Also, two different acrylates may be used in the reaction mixture to prepare a lipid with two different acrylate tails.

In another aspect, the invention provides therapeutic compositions that contain lipids of the formula (II):

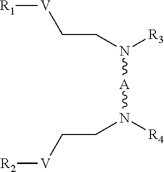

Lipids of the formula (II) are prepared by the addition of a primary or secondary diamine to a double bond conjugated to an electron-withdrawing group such as a carbonyl. The lipids of formula (II) have two amines per lipid molecule as compared to the one amine per lipid molecule in the lipids of formula (I). These amines may be protonated or alkylated to form positively charged amino groups. As with the primary amine, the acrylate tails may be the same or different. Also, the lipid may include any where from one acrylate tail to as many acrylate tails as is chemically possible.

In another aspect, the invention provides therapeutic compositions that contain lipids of the formula (III) or (IV):

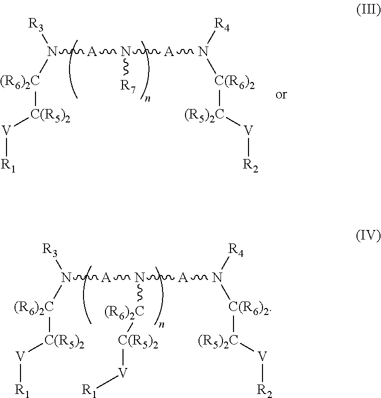

Lipids of the formula (III) or (IV) are prepared by the addition of primary or secondary amino groups to a double bond conjugated to an electron-withdrawing groups as a carbonyl. The lipids of formula (III) and (IV) have multiple amino groups per lipid molecule. In certain embodiments, the number of amino groups per lipid molecule is 3, 4, 5, 6, 7, 8, 9, or 10. These amines may be protonated or alkylated to form positively charged amino groups. The acrylate tails may all be the same or they may be different. Any number of acrylate tails may be present on the molecule.

In one aspect of the invention, the lipids described herein are combined with a therapeutic agent (e.g., a protein, e.g., a therapeutic protein, e.g., a protein for use in replacement therapy, e.g., a protein described herein) to form microparticles, liposomes, or micelles. The therapeutic agent to be delivered by the microparticles, liposomes, or micelles may be in the form of a gas, liquid, or solid. The lipids may be combined with other lipids, polymers, surfactants, cholesterol, carbohydrates, proteins, etc. to form the particles. These particles may be combined with a pharmaceutically excipient to form pharmaceutical compositions.

The invention also provides methods of making therapeutic compositions that contain the lipids.

One or more equivalents of an acrylate are allowed to react with one equivalent of a primary amine, diamine, or polyamine under suitable conditions to form a lipid of the formula (I), (II), (III), or (IV). In certain embodiments, all the amino groups of the amine are fully reacted with acrylates to form tertiary amines. In other embodiments, all the amino groups of the amine are not fully reacted with acrylate to form tertiary amines thereby resulting in primary or secondary amines in the lipid molecule. These primary or secondary amines are left as is or may be reacted with another electrophile such as a different acrylate. As will be appreciated by one of skill in this art, reacting an amine with less than an excess of acrylate will result in a plurality of different lipid amines. Certain molecules may include a full complement of acrylate moieties while other molecules will not include a full complement of acrylates. For example, a diamine or polyamine may include only one, two, three, four, five, or six acrylate moieties off the various amino moieties of the molecule resulting in primary, secondary, and tertiary amines, hi certain embodiments, it is preferred that all the amino groups not be fully functionalized. In certain embodiments, the two of the same type of acrylate are used. In other embodiments, two or more different acrylates are used. The synthesis of the lipid may be performed with or without solvent, and the synthesis may be performed at temperatures ranging from 25° C. to 100° C., preferably approximately 95° C. The prepared lipids may be optionally purified. For example, the mixture of lipids may be purified to yield a lipid with a certain number of acrylate moieties. The lipids may also be alkylated using an alkyl halide (e.g., methyl iodide) or other alkylating agent.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999.

Certain compounds of the present invention may exist in particular geometric or stereoisomeric forms. The present invention contemplates all such compounds, including cis- and toms-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. AU such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-A0M), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, [alpha]-naphthyldiphenylmethyl, p-methoxyphenyldiphenylmethyl, di(p-niethoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxy acetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, [alpha]-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, [alpha]-methoxybenzylidene ortho ester, 1-(N,N-dimethylamino)ethylidene derivative, [alpha]-([N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydroth[iota]oxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocimiamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), [beta]-trimethylsilylethanesulfonamide (SES), 9-anthracenesulfonamide, 4-(4′,8′-dimethoxynaphthylmethyl)benzenesulfonamide (DNMB S), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH2-cyclopropyl, vinyl, allyl, n-butyl, sec-butyl, isobutyl, tert-butyl, cyclobutyl, —CH2-cyclobutyl, n-pentyl, sec-pentyl, isopentyl, tert-pentyl, cyclopentyl, —CH2-cyclopentyl, n-hexyl, sec-hexyl, cyclohexyl, —CH2-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-pro[rho]ynyl(propargyl), 1-propynyl, and the like.

The term “alkyl” as used herein refers to saturated, straight- or branched-chain hydrocarbon radicals derived from a hydrocarbon moiety containing between one and twenty carbon atoms by removal of a single hydrogen atom. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkenyl” denotes a monovalent group derived from a hydrocarbon moiety having at least one carbon-carbon double bond by the removal of a single hydrogen atom. Alkenyl groups include, for example, ethenyl, propenyl, butenyl, 1-methyl-2-buten-1-yl, and the like.

The term “alkynyl” as used herein refers to a monovalent group derived form a hydrocarbon having at least one carbon-carbon triple bond by the removal of a single hydrogen atom. Representative alkynyl groups include ethynyl, 2-propynyl(propargyl), 1-propynyl, and the like.

The term “alkoxy”, or “thioalkyl” as used herein refers to an alkyl group, as previously defined, attached to the parent molecule through an oxygen atom or through a sulfur atom. In certain embodiments, the alkyl, alkenyl, and alkynyl groups contain 1-20 alipahtic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “carboxylic acid” as used herein refers to a group of formula —CO2H.

The term “dialkylamino” refers to a group having the structure —NRR′ wherein R and R′ are each an aliphatic group, as defined herein. R and R′ may be the same or different in an dialkyamino moiety. In certain embodiments, the aliphatic groups contain 1-20 aliphatic carbon atoms. In certain other embodiments, the aliphatic groups contain 1-10 aliphatic carbon atoms. In yet other embodiments, the aliphatic groups employed in the invention contain 1-8 aliphatic carbon atoms. In still other embodiments, the aliphatic groups contain 1-6 aliphatic carbon atoms. In yet other embodiments, the aliphatic groups contain 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino 5 di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(RX)2; —OC(O)Rx; —OCO2Rx; —0C0N(Rx)2; —N(Rx)2; —S(O)2Rx; —NRx(CO)Rx wherein each occurrence of Rx independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(Rx)2; —OC(O)Rx; —OCO2Rx; —0C0N(Rx)2; —N(RX)2; —S(O)2Rx; —NRx(CO)Rx, wherein each occurrence of Rx independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —C0N(Rx)2; —OC(O)Rx; —OCO2Rx; —OCON(Rx)2; —N(RX)2; —S(O)2Rx; —NRx(CO)Rx, wherein each occurrence of Rx independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(RX)2; —OC(O)Rx; —OCO2Rx; —OCON(RX)2; —N(RxJ2; —S(O)2Rx; —NRx(CO)Rx, wherein each occurrence of Rx independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO2; —CN; —CF3; —CH2CF3; —CHCl2; —CH2OH; —CH2CH2OH; —CH2NH2; —CH2SO2CH3; —C(O)Rx; —CO2(Rx); —CON(RX)2; —OC(O)Rx; —OCO2Rx; —0C0N(Rx)2; —N(RX)2; —S(O)2Rx; —NRx(CO)Rx, wherein each occurrence of Rx independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Carbocycle”: The term “carbocycle”, as used herein, refers to an aromatic or non-aromatic ring in which each atom of the ring is a carbon atom.

“Independently selected”: The term “independently selected” is used herein to indicate that the R groups can be identical or different.

“Labeled”: As used herein, the term “labeled” is intended to mean that a compound has at least one element, isotope, or chemical compound attached to enable the detection of the compound. In general, labels typically fall into three classes: a) isotopic labels, which may be radioactive or heavy isotopes, including, but not limited to, ²H, ³H, ³²P, ³⁵S, ⁶⁷Ga, ^{99m}Tc (Tc-99m), ¹¹¹In, ¹²³I, ¹²⁵I, ¹⁶⁹Yb and ¹⁸⁶Re; b) immune labels, which may be antibodies or antigens, which may be bound to enzymes (such as horseradish peroxidase) that produce detectable agents; and c) colored, luminescent, phosphorescent, or fluorescent dyes. It will be appreciated that the labels may be incorporated into the compound at any position that does not interfere with the biological activity or characteristic of the compound that is being detected. In certain embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (See, Bayley, H., Photogenerated Reagents in Biochemistry and Molecular Biology (1983), Elsevier, Amsterdam.). In certain embodiments of the invention, the photoaffinity labels employed are o-, m- and p-azidobenzoyls, substituted with one or more halogen moieties, including, but not limited to 4-azido-2,3,5,6-tetrafluorobenzoic acid.

The terms halo and halogen as used herein refer to an atom selected from fluorine, chlorine, bromine, and iodine.

The term “heterocyclic”, as used herein, refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

The term “heteroaryl”, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, A-(phenylmethyl)piperazine, 4-(1-phenylethyl)piperazine, 4-(1,1-dimethylethoxycarbonyl)piperazine, 4-(2-(bis-(2-propenyl)amino)ethyl)pi[rho]erazine, A-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, A-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The terms “substituted,” whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted {e.g., an aryl group substituent may have another substituent off it, such as another aryl group, which is further substituted with fluorine at one or more positions).

The following are more general terms used throughout the present application:

“Animal”: The term animal, as used herein, refers to humans as well as non-human animals, including, for example, mammals, birds, reptiles, amphibians, and fish. Preferably, the non-human animal is a mammal {e.g., a rodent, a mouse, a rat, a rabbit, a monkey, a dog, a cat, a primate, or a pig). An animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

“Effective amount”: In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, etc. For example, the effective amount of microparticles containing an antigen to be delivered to immunize an individual is the amount that results in an immune response sufficient to prevent infection with an organism having the administered antigen.

“Isolated composition”: An “isolated composition” refers to a composition that is removed from at least 90% of at least one component of a natural sample from which the isolated composition can be obtained. Compositions produced artificially or naturally can be “compositions of at least” a certain degree of purity if the species or population of species of interest is at least 5, 10, 25, 50, 75, 80, 90, 95, 96, 97, 98, or 99% pure on a weight-weight basis. For example, a therapeutic protein described herein can be an isolated therapeutic protein, or a therapeutic protein of at least a certain degree of purity. Similarly, a therapeutic composition (e.g., containing a therapeutic protein and an amine-containing lipid described herein can be an isolated therapeutic composition, or a therapeutic composition of at least a certain degree of purity.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should—substantially interfere with the desired biological activity of the peptide.

All cited publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments are set forth in the accompanying drawings and the description below. Other features, objects, and advantages of the invention will be apparent from the description and drawings, and from the claims.

### DESCRIPTION OF DRAWINGS

FIGS. 1A and 1B show acrylates and amines that can be used in the synthesis of exemplary amine-containing lipids.

### DETAILED DESCRIPTION

The present invention provides therapeutic compositions that contain a lipid (e.g., an amine-containing lipid) and a therapeutic agent (e.g., a therapeutic protein, e.g., a protein for use in replacement therapy, e.g., a protein described herein). Also described are delivery systems based on the use of lipids. The systems may be used to prepare pharmaceutical compositions, e.g., that contain a therapeutic agent (e.g., a therapeutic protein, e.g., a protein for use in replacement therapy, e.g., a protein described herein), that can be administered to individuals in need of such administration (e.g., an individual with a protein deficiency or with low levels of the protein).

The amino lipids provide for several different uses in the drug delivery art. For example, the lipids with their amine-containing hydrophilic portion may be used to complex a therapeutic agent (e.g., a therapeutic protein, e.g., a protein for use in replacement therapy, e.g., a protein described herein) and thereby enhance the delivery of the therapeutic agent and prevent its degradation. The lipids may also be used in the formation of nanoparticles, microparticles, liposomes, and micelles containing the therapeutic agent to be delivered. Preferably, the lipids are biocompatible and biodegradable, and the formed particles are also biodegradable and biocompatible and may be used to provide controlled, sustained release of the agent. These lipids and their corresponding particles may also be responsive to pH changes given that these lipids are protonated at lower pH.

Therapeutic Proteins

The lipids described herein can be used to prepare a therapeutic composition that contains a therapeutic protein, e.g., a protein for replacement therapy. The protein can be, e.g., an enzyme. Examples of proteins that can be used for replacement therapy include: Bruton's tyrosine kinase (BTK), ornithine transcarbamylase (OTC), survival motor neuron protein (SMN1 or SMN2), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, N-acetylgalactosamine-4-sulfatase, protein C, protein S, CLN2 (ceroid lipofuscinosis, neuronal 2) protein, alpha-1 antitrypsin, Factor VIII, Factor IX, albumin, pancrelipase (e.g., CREON, Cotazym, Pancrease, Pancrease MT, ULTRASE®).

Preferred proteins for the compositions described herein include Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, and N-acetylgalactosamine-4-sulfatase.

Bruton's Tyrosine Kinase (BTK)

Bruton's tyrosine kinase is a type of kinase enzyme implicated in the primary immunodeficiency disease X-linked agammaglobulinemia (XLA). It plays a crucial role in B cell maturation as well as mast cell activation through the high-affinity IgE receptor. Patients with XLA have normal pre-B cell populations in their bone marrow but these cells fail to mature and enter the circulation. The BTK gene is located on the X chromosome. At least 24 mutations of the BTK gene have been identified.

BTK contains a PH domain which binds Phosphatidylinositol (3,4,5)-trisphosphate (PIP3). PIP3 binding induces BTK to phosphorylate phospholipase C, which in turn hydrolyzes PIP2 into two second messengers, inositol triphosphate (IP3) and diacylglycerol (DAG), which then go on to modulate the activity of downstream proteins during B-cell signalling.

A therapeutic composition that contains BTK and a lipid described herein, and the methods described herein, can be used in the treatment of an individual with a need for BTK replacement therapy.

Ornithine Transcarbamylase (OTC)

Ornithine transcarbamylase deficiency (OTC) is a rare metabolic disorder. It is a genetic disorder resulting in a mutated and ineffective form of the enzyme ornithine transcarbamylase.

OTC affects the body's ability to get rid of ammonia. As a result, ammonia accumulates in the blood causing hyperammonemia. This ammonia travels to the various organs of the body including the brain, causing coma, brain damage, liver damage, and death.

Ornithine transcarbamylase deficiency often becomes evident in the first few days of life. An infant with ornithine transcarbamylase deficiency may be lethargic or unwilling to eat, and have poorly-controlled breathing rate or body temperature. Some babies with this disorder may experience seizures or unusual body movements, or go into a coma. Complications from ornithine transcarbamylase deficiency may include developmental delay and mental retardation. Progressive liver damage, skin lesions, and brittle hair may also be seen. Other symptoms include irrational behavior (caused by encephalitis), mood swings, and poor performance in school.

Mutations in the OTC gene cause ornithine transcarbamylase deficiency. Ornithine transcarbamylase deficiency is an X-linked disorder caused by a number of different mutations. Since the gene is on the X chromosome, females are primarily carriers while males with nonconservative mutations rarely survive past 72 hours of birth. Half of those survivors die in the first month, and half of the remaining by age 5.

A therapeutic composition that contains OTC and a lipid described herein, and the methods described herein, can be used in the treatment of an individual with a need for OTC replacement therapy.

Survival Motor Neuron Protein (SMN)

Loss of the SMN1 gene causes Spinal Muscular Atrophy (SMA), which manifests as weakness, due to loss of the motor neurons of the spinal cord and brainstem.

The region of chromosome 5 that contains the SMN (Survival Motor Neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1. Often there are additional copies of SMN2, and an increasing number of SMN2 copies causes less severe disease.

Infantile SMA is the most severe form. Some of the symptoms include: muscle weakness, poor muscle tone, weak cry, limpness or a tendency to flop, difficulty sucking or swallowing, accumulation of secretions in the lungs or throat, the legs tend to be weaker than the arms, feeding difficulties, increased susceptibility to respiratory tract infections, developmental milestones, such as lifting the head or sitting up, can't be reached. Although SMA often results in death during childhood, some people with SMA survive into adulthood and even old age. Actual lifespan depends greatly on the severity of SMA in each individual.

In general, the earlier the symptoms appear, the shorter the life span. The onset is sudden and dramatic. Once symptoms appear the motor neuron cells quickly deteriorate shortly after. The disease can be fatal and there is no cure for SMA yet known. The major management issue in Type 1 SMA is the prevention and early treatment of respiratory infections; pneumonia is the cause of death in the majority of the cases. Infants with Type 1 SMA have a life expectancy of less than two years, however, some grow to be adults. Intellectual and later, sexual functions, are unaffected by SMA.

A therapeutic composition that contains SMN and a lipid described herein, and the methods described herein, can be used in the treatment of an individual with a need for SMN replacement therapy.

Galactocerebrosidase (GALC)

Krabbe's Leukodystrophy is a rare inherited lipid storage disorder caused by a deficiency of the enzyme galactocerebrosidase (GALC), which is necessary for the metabolism of the sphingolipids galactosylceremide and psychosine. Failure to break down these sphingolipids results in degeneration of the myelin sheath surrounding nerves in the brain (demyelination). Characteristic globoid cells appear in affected areas of the brain. This metabolic disorder is characterized by progressive neurological dysfunction such as mental retardation, paralysis, blindness, deafness and paralysis of certain facial muscles (pseudobulbar palsy). Krabbe's Leukodystrophy is inherited as an autosomal recessive trait.

A therapeutic composition that contains GALC and a lipid described herein, and the methods described herein, can be used in the treatment of an individual with a need for GALC replacement therapy.

N-Sulfoglucosamine Sulfohydrolase

MPS type III-A (Sanfilipo A syndrome) is a lysosomal storage disorder caused by deficiency or malfunction of N-sulfoglucosamine sulfohydrolase (also referred to as heparin sulfate sulfatase or sulfamidase), which is required for the degradation of heparan sulphate. Patients develop severe central nervous system degeneration resulting in progressive dementia often combined with delayed speech, sleep disturbance, hirsutism, diarrhoea, hyperactivity and aggressive behavior. Clinical features can also include severe mental defect with relatively mild somatic features (moderately severe claw hand and visceromegaly, little or no corneal clouding or skeletal, e.g., vertebral, change). The presenting problem may be marked overactivity, destructive tendencies, and other behavioral aberrations in a child of 4 to 6 years of age. Clinical onset in severely affected patients usually occurs following 2-3 years of apparently normal development. Mild skeletal pathology, joint stiffness and hepatosplenomegaly are often present in older patients. Patients may present and develop within a wide spectrum of clinical severity. The radiologic findings in the skeleton are relatively mild and include persistent biconvexity of the vertebral bodies and very thick calvaria.

A therapeutic composition that contains N-sulfoglucosamine sulfohydrolase and a lipid described herein, and the methods described herein, can be used in the treatment of an individual with a need for N-sulfoglucosamine sulfohydrolase replacement therapy.

N-Acetylglucosaminidase

The Sanfilippo syndrome type B is a lysosomal storage disorder caused by deficiency of alpha-N-acetylglucosaminidase; it is characterized by profound mental deterioration in childhood and death in the second decade. The cDNA sequence was found to encode a protein of 743 amino acids, with a 20- to 23-aa signal peptide immediately preceding the amino terminus of the tissue enzyme and with six potential N-glycosylation sites. The 8.5-kb gene (NAGLU), interrupted by 5 introns, was localized to the 5′-flanking sequence of a known gene, EDH17B, on chromosome 17q21. Five mutations have been identified in cells of patients with Sanfilippo syndrome type B: 503del10, R297X, R626X, R643H, and R674H. The occurrence of a frameshift and a nonsense mutation in homozygous form confirms the identity of the NAGLU gene.

A therapeutic composition that contains N-acetylglucosaminidase and a lipid described herein, and the methods described herein, can be used in the treatment of an individual with a need for N-acetylglucosaminidase replacement therapy.

Iduronate-2-Sulfatase

Hunter syndrome, or mucopolysaccharoidosis Type II (MPS II), is a lysosomal storage disease caused by a deficiency of iduronate-2-sulfatase (I2S). The I2S gene is located on the X chromosome. Hunter syndrome is a serious genetic disorder that primarily affects males. It interferes with the body's ability to break down and recycle specific glycosaminoglycans or GAG Hunter syndrome is one of several related lysosomal storage diseases.

In Hunter syndrome, GAG build up in cells throughout the body due to a deficiency or absence of the enzyme iduronate-2-sulfatase (I2S). This buildup interferes with the way certain cells and organs in the body function and leads to a number of serious symptoms. As the buildup of GAG continues throughout the cells of the body, signs of Hunter syndrome become more visible. Physical manifestations for some people with Hunter syndrome include distinct facial features, a large head, and an enlarged abdomen. People with Hunter syndrome may also experience hearing loss, thickening of the heart valves leading to a decline in cardiac function, obstructive airway disease, sleep apnea, and enlargement of the liver and spleen. Range of motion and mobility may also be affected. In some cases of Hunter syndrome, central nervous system involvement leads to developmental delays and nervous system problems. Not all people with Hunter syndrome are affected by the disease in exactly the same way, and the rate of symptom progression varies widely. However, Hunter syndrome is always severe, progressive, and life-limiting.

ELAPRASE™ is a synthetic version of I2S that was approved by the United States Food and Drug Administration for enzyme replacement treatment for Hunter syndrome.

A therapeutic composition that contains I2S and a lipid described herein, and the methods described herein, can be used in the treatment of an individual with a need for I2S replacement therapy.

Alpha-Glucosidase

Pompe disease (acid maltase deficiency (AMD), glycogen storage disease type II) is a rare, inherited and often fatal disorder that disables the heart and muscles. It is caused by mutations in a gene that makes alpha-glucosidase (GAA). Normally, the body uses GAA to break down glycogen. But in Pompe disease, mutations in the GAA gene reduce or completely eliminate this essential enzyme. Excessive amounts of glycogen accumulate everywhere in the body, but the cells of the heart and skeletal muscles are the most seriously affected. Researchers have identified up to 70 different mutations in the GAA gene that cause the symptoms of Pompe disease, which can vary widely in terms of age of onset and severity. The severity of the disease and the age of onset are related to the degree of enzyme deficiency.

Early onset (or infantile) Pompe disease is the result of complete or near complete deficiency of GAA. Symptoms begin in the first months of life, with feeding problems, poor weight gain, muscle weakness, floppiness, and head lag. Respiratory difficulties are often complicated by lung infections. The heart is grossly enlarged. More than half of all infants with Pompe disease also have enlarged tongues. Most babies with Pompe disease die from cardiac or respiratory complications before their first birthday.

Late onset (or juvenile/adult) Pompe disease is the result of a partial deficiency of GAA. The onset can be as early as the first decade of childhood or as late as the sixth decade of adulthood. The primary symptom is muscle weakness progressing to respiratory weakness and death from respiratory failure after a course lasting several years. The heart may be involved but it will not be grossly enlarged. A diagnosis of Pompe disease can be confirmed by screening for the common genetic mutations or measuring the level of GAA enzyme activity in a blood sample—a test that has 100 percent accuracy. Once Pompe disease is diagnosed, testing of all family members and consultation with a professional geneticist is recommended. Carriers are most reliably identified via genetic mutation analysis.

A therapeutic composition that contains GAA and a lipid described herein, and the methods described herein, can be used in the treatment of an individual with a need for GAA replacement therapy.

Sulfatase-Modifying Factor 1 (SUMF1)

Sulfatase modifying factor 1 (SUMF1) is the gene mutated in multiple sulfatase deficiency (MSD) and encodes the formylglycine-generating enzyme, an essential activator of all the sulfatases. The disorder combines features of metachromatic leukodystrophy and of a mucopolysaccharidosis. Increased amounts of acid mucopolysaccharides are found in several tissues. In contrast to the classic form of metachromatic leukodystrophy, arylsulfatases A, B, and C are absent in the Austin type of juvenile sulfatidosis. The ‘gargoylism’ features are mild. Neurologic deterioration is rapid. Both mucopolysaccharide and sulfatide are found in the urine in excess. Cerebrospinal fluid protein is increased. Peripheral nerves show metachromatic degeneration of myelin on biopsy. The disease is associated with ichthyosis, dysostosis multiplex, psychomotor retardation, and coarse facies.

A therapeutic composition that contains SUMF1 and a lipid described herein, and the methods described herein, can be used in the treatment of an individual with a need for SUMF1 replacement therapy.

Glucocerebrosidase (GCB)

Gaucher patients exhibit a glucocerebrosidase deficiency. Glucocerebrosidase (also known as glucoceramidase) is involved in the breakdown and recycling of glucocerebroside. Gaucher disease is an inherited metabolic disorder in which harmful quantities of a fatty substance called glucocerebroside accumulate in the spleen, liver, lungs, bone marrow, and sometimes in the brain. There are three types of Gaucher disease. The first category, called type 1, is by far the most common. Patients in this group usually bruise easily and experience fatigue due to anemia and low blood platelets. They also have an enlarged liver and spleen, skeletal disorders, and, in some instances, lung and kidney impairment. There are no signs of brain involvement. Symptoms can appear at any age. In type 2 Gaucher disease, liver and spleen enlargement are apparent by 3 months of age. Patients have extensive and progressive brain damage and usually die by 2 years of age. In the third category, called type 3, liver and spleen enlargement is variable, and signs of brain involvement such as seizures gradually become apparent. The buildup of this fatty material within cells prevents the cells and organs from functioning properly.

Examples of GCB protein that can be used in the therapeutic compositions described herein include CEREZYME® (imiglucerase for injection; Genzyme Corporation), and the proteins described in, e.g., WO02/15927, WO2005/089047, WO03/056897, WO01/77307, WO01/07078, and WO90/07573; European Published App. No. EP1392826; U.S. Published Application Nos. 2005-0026249, 2005-0019861, 2002-0168750, 2005-0265988, 2004-0043457, 2003-0215435, and 2003-0133924; and U.S. patent application Ser. No. 10/968,870; U.S. Pat. Nos. 7,138,262, 6,451,600, 6,074,864, 5,879,680, 5,549,892, 5,236,838, and 3,910,822.

A therapeutic composition that contains GCB and a lipid described herein, and the methods described herein, can be used in the treatment of an individual with a need for GCB replacement therapy.

Alpha Galactosidase

An alpha galactosidase mutation causes Fabry disease. A mutation in the gene that controls this enzyme causes insufficient breakdown of lipids, which build up to harmful levels in the eyes, kidneys, autonomic nervous system, and cardiovascular system. Since the gene that is altered is carried on a mother's X chromosome, her sons have a 50 percent chance of inheriting the disorder and her daughters have a 50 percent chance of being a carrier. Symptoms usually begin during childhood or adolescence and include burning sensations in the hands that gets worse with exercise and hot weather and small, raised reddish-purple blemishes on the skin. Some boys will also have eye manifestations, especially cloudiness of the cornea. Lipid storage may lead to impaired arterial circulation and increased risk of heart attack or stroke. The heart may also become enlarged and the kidneys may become progressively involved. Other symptoms include decreased sweating, fever, and gastrointestinal difficulties, particularly after eating. Some female carriers may also exhibit symptoms. Fabry disease is one of several lipid storage disorders.

Examples of alpha-galactosidase protein that can be used in the therapeutic compositions described herein include those described in, e.g., WO98/11206 and WO00/53730.

A therapeutic composition that contains alpha-galactosidase and a lipid described herein, and the methods described herein, can be used in the treatment of an individual with a need for alpha-galactosidase replacement therapy.

Alpha Iduronidase

Alpha iduronidase (IDUA) deficiency is responsible for Hurler syndrome (also known as mucopolysaccharidosis type I (MPS I), Hurler's disease and gargoylism). Alpha-L iduronidase functions to break down mucopolysaccharides. Without this enzyme, the buildup of heparan sulfate and dermatan sulfate occurs in the body (the heart, liver, brain etc.). Symptoms appear during childhood and early death can occur due to organ damage.

MPS I is divided into three subtypes based on severity of symptoms. All three types result from an absence of, or insufficient levels of, the enzyme alpha-L-iduronidase. MPS I H or Hurler syndrome is the most severe of the MPS I subtypes. The other two types are MPS I S or Scheie syndrome and MPS I H-S or Hurler-Scheie syndrome. Hurler syndrome is marked by progressive deterioration, hepatosplenomegaly, dwarfism and gargoyle-like faces. There is a progressive mental retardation, with death occurring by the age of 10 years.

Developmental delay is evident by the end of the first year, and patients usually stop developing between ages 2 and 4. This is followed by progressive mental decline and loss of physical skills. Language may be limited due to hearing loss and an enlarged tongue. In time, the clear layers of the cornea become clouded and retinas may begin to degenerate. Carpal tunnel syndrome (or similar compression of nerves elsewhere in the body) and restricted joint movement are common.

Hurler's Syndrome is often classified as a lysosomal storage disease and is mechanistically related to Hunter's Syndrome (X-linked recessive). Children born to an MPS I parent carry a defective IDUA gene, which has been mapped to the 4p16.3 site on chromosome 4. As of 2001, 52 different mutations in the IDUA gene have been shown to cause Hurler syndrome. Because Hurler syndrome is an autosomal recessive disorder.

Therapeutic forms if IDUA include laronidase, e.g., ALDURAZYME®.

A therapeutic composition that contains IDUA and a lipid described herein, and the methods described herein, can be used in the treatment of an individual with a need for IDUA replacement therapy.

Beta Glucuronidase

Sly syndrome belongs to a group of disorders known as the mucopolysaccharidoses, which are lysosomal storage diseases. It is characterized by a deficiency of the enzyme β-glucuronidase, a lysosomal enzyme. In Sly syndrome, the deficiency in β-glucuronidase leads to the accumulation of certain complex carbohydrates (mucopolysaccharides) in many tissues and organs of the body. The defective gene lies on chromosome 7.

Sly syndrome is also known as Mucopolysaccahridosis Type VII (MPS), β-glucurondinase deficiency, β-glucurondinase deficiency mucopolysaccahridosis, GUSB deficiency, mucopolysaccahride storage disease VII, MCA, and MR.

The symptoms of Sly syndrome are similar to those of Hurler syndrome (MPS I). The symptoms include: in the head, neck, and face: coarse (Hurler-like) facies and macrocephaly, frontal prominence, premature closure of sagittal lambdoid sutures, and J-shaped sella turcica, in the eyes: corneal opacity and iris colobmata, in the nose: anteverted nostrils and a depressed nostril bridge, in the mouth and oral areas: prominent alveolar processes and cleft palate, in the thorax: usually pectus carinatum or exacavatum and oar-shaped ribs; also a protruding abdomen and inguinal or umbilical hernia, in the extremities: talipes, an underdeveloped ilium, aseptic necrosis of femoral head, and shortness of tubular bones occurs, in the spine: kyphosis or scoliosis and hook-like deformities in thoracic and lumbar vertebrate, in the bones: dysotosis multiplex. In addition, recurrent pulmonary infections occur. Hepatomegaly occurs in the gastrointestinal system. Splenomegaly occurs in the hematopoietic system. Inborn mucopolysaccharide metabolic disorders due to β-glucuronidase deficiency with granular inclusions in granulocytes occurs in the biochemical and metabolic systems. Growth and motor skills are affected, and mental retardation also occurs.

A therapeutic composition that contains beta glucuronidase and a lipid described herein, and the methods described herein, can be used in the treatment of an individual with a need for beta glucuronidase replacement therapy.

N-Acetylgalactosamine-4-Sulfatase (Arylsulfatase B)

Mucopolysacchariodosis type VI (MPS VI, Maroteaux-Lamy syndrome) is a lysosomal storage disorder caused by the deficient activity of N-acetylgalactosamine-4-sulfatase (arylsulfatase B) and the subsequent accumulation of the glycosaminoglycan (GAG) dermatan sulfate. Children with MPS VI usually have normal intellectual development but share many of the physical symptoms found in Hurler syndrome. The condition has a variable spectrum of severe symptoms. Neurological complications include clouded corneas, deafness, thickening of the dura (the membrane that surrounds and protects the brain and spinal cord), and pain caused by compressed or traumatized nerves and nerve roots. MPS VI is characterized by short stature, dysotosis multiplex, coarse facial features, cardiac valve anomalies, thickening of the tracheal wall.

Growth in children with the disorder is normal at first but stops suddenly around age 8. By age 10 children have developed a shortened trunk, crouched stance, and restricted joint movement. In more severe cases, children also develop a protruding abdomen and forward-curving spine. Skeletal changes (particularly in the pelvic region) are progressive and limit movement. Many children also have umbilical or inguinal hernias. Nearly all children have some form of heart disease, usually involving valve dysfunction.

A therapeutic composition that contains N-acetylgalactosamine-4-sulfatase and a lipid described herein, and the methods described herein, can be used in the treatment of an individual with a need for N-acetylgalactosamine-4-sulfatase replacement therapy.

Lipids

The therapeutic compositions of the present disclosure include a lipid and a therapeutic agent. The lipids of the present invention are lipids containing primary, secondary, or tertiary amines and salts thereof. Such lipids are also described in WO 2006/138380. In a particularly preferred embodiment, the lipids are relatively non-cytotoxic. In another particularly preferred embodiment, the lipids are biocompatible and biodegradable. In a particularly preferred embodiment, the lipids of the present invention have pKa's in the range of 5.5 to 7.5, more preferably between 6.0 and 7.0. In another particularly preferred embodiment, the lipid may be designed to have a desired pKa between 3.0 and 9.0, more preferably between 5.0 and 8.0. The lipids are particularly attractive for drug delivery for several reasons: 1) they contain amino groups for interacting with negatively charged agents, for buffering the pH, for causing endosomolysis, etc.; 2) they can be synthesized from commercially available starting materials; and 3) they are pH responsive and can be engineered with a desired pKa.

In certain embodiments, the lipids of the present invention are of the formula (I):

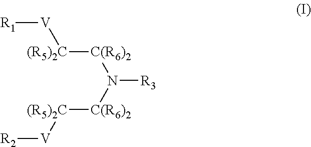

wherein each occurrence of V is independently selected from the group consisting of C═O, C═S, S═O, and SO2; R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; or —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(═O)RB; —CO2RB; —CN; —SCN; —SRB; —SORB; —SO2RB; —NO2; —N3; —N(RB)2; —NHC(=0)RB; —NRBC(=0)N(RB)2; -0C(=0)0RB; —OC(═O)RB; —OC(═O)N(RB)2; —NRBC(=0)0RB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR0; —C(═O)Rc; —CO2RC; —CN; —SCN; —SR0; —SORC; —SO2Rc; —NO2; —N3; —N(Rc)2; —NHC(=0)Rc; —NRCC(═O)N(RC)2; —OCOO)ORc; -0C(=0)Rc; -0C(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R5 is independently selected from the group consisting of hydrogen and C1-C6 alkyl; each occurrence of R6 is independently selected from the group consisting of hydrogen and C1-C6 alkyl; and salts thereof.

In certain embodiments, the tertiary amine of formula (I) is protonated or alkylated to form a compound of formula (Ia):

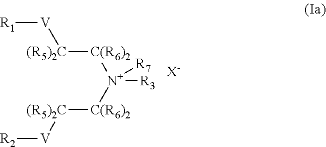

wherein R1, R2, R3, R5, R6, and V are defined above;

R7 is hydrogen or C1-C6 aliphatic, preferably C1-C6 alkyl, more preferably hydrogen or methyl; and

X is any anion. Possible anions include fluoride, chloride, bromide, iodide, sulfate, bisulfate, phosphate, nitrate, acetate, fumarate, oleate, citrate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate).

In certain embodiments, V is C═O. In other embodiments, V is C═S. In yet other embodiments, V is S═O. In still other embodiments, V is SO2.

In certain embodiments, R1 is hydrogen. In other embodiments, R1 is a cyclic or acyclic, substituted or unsubstituted, branched or un branched aliphatic or heteroaliphatic moiety. In certain embodiments, R1 is a substituted or unsubstituted aryl or heteroaryl moiety. Preferably, the aryl or heteroaryl moiety is a monocyclic 5- or 6-membered ring system. In certain embodiments, R1 is —ORA, —SRA, —N(RA)2, or —NHRA- In certain embodiments, R1 is —ORA. In other embodiments, R1 is —N(RA)2 or —NHRA- In certain embodiments, RA is hydrogen. In other embodiments, RA is a cyclic or acyclic, substituted or unsubstituted, branched or unbranched aliphatic or heteroaliphatic moiety. In certain embodiments, RA is an acyclic, substituted or unsubstituted aliphatic moiety. In certain other embodiments, RA is an acyclic, unsubstituted, unbranched aliphatic moiety, preferably C6-C30, more preferably C10-C20. In certain embodiments, RA is an unsubstituted, straight chain alkyl group with at least 5 carbons. In certain embodiments, RA is an unsubstituted, straight chain alkyl group, preferably C6-C30, more preferably 10-C20. In certain embodiments, R1 is —ORA, wherein RA is an unsubstituted, unbranched C9 alkyl chain. In certain embodiments, R1 is —ORA, wherein RA is an unsubstituted, unbranched Ci0 alkyl chain. In certain embodiments, R1 is —ORA5 wherein RA is an unsubstituted, unbranched C11 alkyl chain. In certain embodiments, R1 is —ORA, wherein RA is an unsubstituted, unbranched C12 alkyl chain. In certain embodiments, R1 is —ORA, wherein RA is an unsubstituted, unbranched C13 alkyl chain. In certain embodiments, R1 is —ORA, wherein RA is an unsubstituted, unbranched C14 alkyl chain. In certain embodiments, R1 is —ORA, wherein RA is an unsubstituted, unbranched Cj5 alkyl chain. In certain embodiments, R1 is —ORA, wherein RA is an unsubstituted, unbranched C16 alkyl chain. In certain embodiments, R1 is —ORA, wherein RA is an unsubstituted, unbranched C17 alkyl chain. In certain embodiments, R1 is —ORA, wherein RA is an unsubstituted, unbranched C18 alkyl chain. In certain embodiments, R1 is —ORA, wherein RA is an unsubstituted, unbranched C19 alkyl chain. In certain embodiments, R1 is —ORA, wherein RA is an unsubstituted, unbranched C20 alkyl chain. In yet other embodiments, RA is a substituted or unsubstituted aryl or heteroaryl moiety.

In certain embodiments, R2 is hydrogen. In other embodiments, R2 is a cyclic or acyclic, substituted or unsubstituted, branched or un branched aliphatic or heteroaliphatic moiety. In certain embodiments, R2 is a substituted or unsubstituted aryl or heteroaryl moiety. Preferably, the aryl or heteroaryl moiety is a monocylic 5- or 6-membered ring system. In certain embodiments, R2 is —ORB, —SRB, —N(RB)2, or —NHRB. In certain embodiments, R2 is —ORB. In other embodiments, R2 is —N(RB)2 or —NHRB— In certain embodiments, RB is hydrogen. In other embodiments, RB is a cyclic or acyclic, substituted or unsubstituted, branched or unbranched aliphatic or heteroaliphatic moiety. In certain embodiments, RB is an acyclic, substituted or unsubstituted aliphatic moiety. In certain embodiments, RB is an unsubstituted, straight chain alkyl group with at least 5 carbons. In certain other embodiments, RB is an acyclic, unsubstituted, unbranched aliphatic moiety, preferably C6-C30, more preferably C10-C20. In certain embodiments, RB is an unsubstituted, straight chain alkyl group, preferably C6-C30, more preferably C10-C20. In certain embodiments, R2 is —ORB, wherein RB is an unsubstituted, unbranched C9 alkyl chain. In certain embodiments, R2 is —ORB, wherein RB is an unsubstituted, unbranched C10 alkyl chain. In certain embodiments, R2 is —ORB, wherein RB is an unsubstituted, unbranched Cn alkyl chain. In certain embodiments, R2 is —ORB, wherein RB is an unsubstituted, unbranched C12 alkyl chain. In certain embodiments, R2 is —ORB, wherein RB is an unsubstituted, unbranched C13 alkyl chain. In certain embodiments, R2 is —ORB, wherein RB is an unsubstituted, unbranched C14 alkyl chain. In certain embodiments, R2 is —ORB, wherein RB is an unsubstituted, unbranched C15 alkyl chain. In certain embodiments, R2 is —ORA, wherein RB is an unsubstituted, unbranched C16 alkyl chain. In certain embodiments, R2 is —ORA, wherein RB is an unsubstituted, unbranched C17 alkyl chain. In certain embodiments, R2 is —ORA, wherein RB is an unsubstituted, unbranched C18 alkyl chain. In certain embodiments, R2 is —ORB, wherein RB is an unsubstituted, unbranched C 19 alkyl chain. In certain embodiments, R2 is —ORB, wherein RB is an unsubstituted, unbranched C20 alkyl chain. In yet other embodiments, RB is a substituted or unsubstituted aryl or heteroaryl moiety.

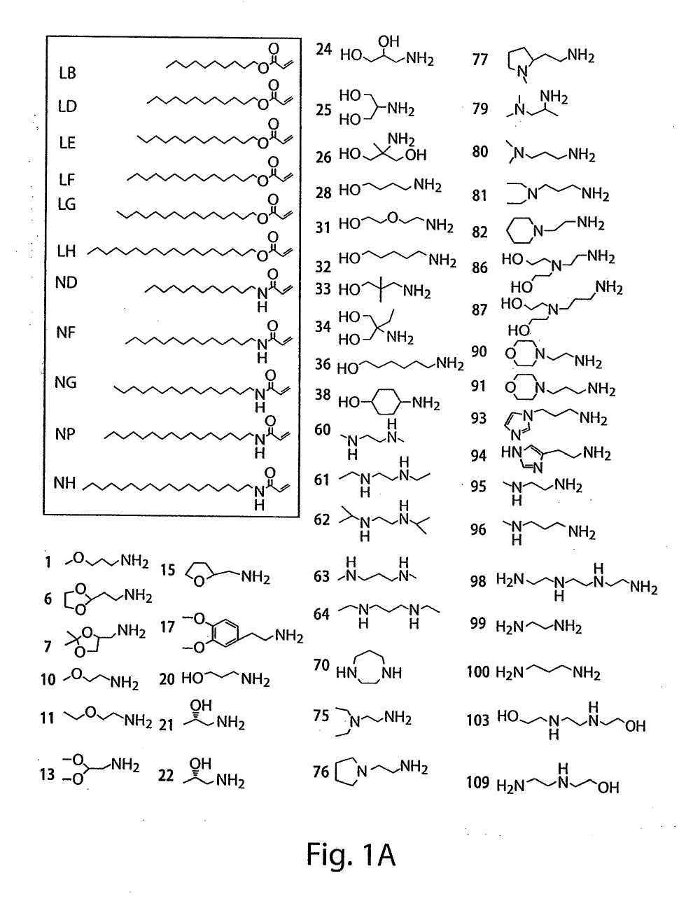

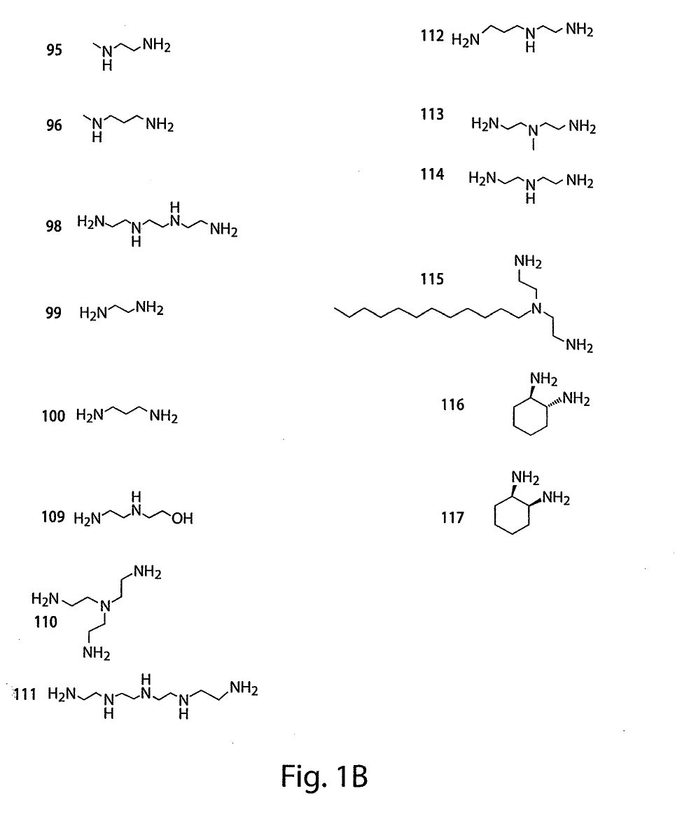

In certain embodiments, R3 is a cyclic or acyclic, substituted or unsubstituted, branched or unbranched aliphatic moiety. In other embodiments, R3 is a cyclic or acyclic, substituted or unsubstituted, branched or unbranched heteroaliphatic moiety. In certain embodiments, R3 is a polyethylene glycol moiety. In certain embodiments, R3 is an aliphatic moiety substituted with one or more hydroxyl groups. In other embodiments, R3 is an aliphatic moiety substituted with one or more amino, alkylamino, or dialkylamino groups. In certain embodiments, R3 is a heteroaliphatic moiety. In certain embodiments, R3 is cyclic aliphatic, preferably a monocylic ring system with a 5- or 6-membered ring. In other embodiments, R3 is aryl or heteroaryl, preferably a monocyclic ring system with a 5- or 6-membered ring. In certain embodiments, the lipids are prepared from the primary amines 1, 11, 20, 24, 25, 28, 31, 32, 36, 76, 77, 80, 86, 87, 93, 94, 95, 96, 99, or 100 shown in FIGS. 1A and 1B. In certain other embodiments the lipids are prepared from the primary amines 31, 93, or 94 as shown in FIGS. 1A and 1B.

In certain embodiments, each occurrence of R5 is hydrogen. In certain embodiments, at least one occurrence of R5 is methyl and the other occurrences are hydrogen. In certain embodiments, at least two occurrences of R5 are methyl, and the other occurrences are hydrogen. In other embodiments, at least two occurrences of R5 are hydrogen.

In certain embodiments, each occurrence of R6 is hydrogen. In certain other embodiments, at least two occurrences of R6 are hydrogen. In certain embodiments, at least one occurrence of R6 is methyl, and the other occurrences are hydrogen. In certain embodiments, at least two occurrences of R6 are methyl, and the other occurrences are hydrogen.

In certain embodiment

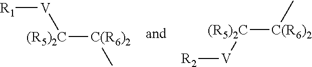

are attached to N, are the same.

In other embodiments,

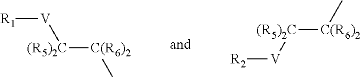

which are attached to N are the same and are different than R3.

In yet other embodiments,

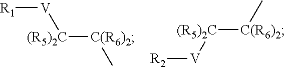

and R3 are all different.

In certain subclasses of lipids, the lipids are of the formula:

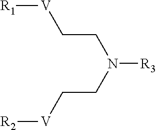

wherein V, R1, R2, and R3 are defined as above; and all occurrences of R5 and R6 are hydrogen. In certain embodiments, R1 and R2 are the same. In other embodiments, R1 and R2 are different. In certain embodiments, V is C═O as shown in the formula:

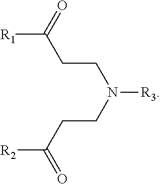

In certain embodiments, R1 and R2 are the same. In other embodiments, R1 and R2 are different. In certain embodiments, R1 is —ORA and R2 is —ORB, as shown in the formula below:

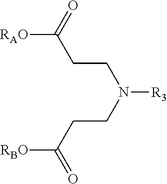

In certain embodiments, RA and RB are the same. In other embodiments, RA and RB are different. In certain embodiments, at least one of RA and RB is an unsubstituted, straight chain alkyl group with at least 5 carbons. In certain embodiments, both of RA and RB are an unsubstituted, straight chain alkyl group with at least 5 carbons. In certain embodiments, RA and RB are C6-C30 straight chain alkyl groups, or C21-C30 straight chain alkyl groups, preferably Cg-C20 straight chain alkyl groups. In certain embodiments, RA and RB are C6-C30 straight chain alkenyl groups, or C21-C30 straight chain alkenyl groups, preferably C9-C20 straight chain alkenyl groups. In certain embodiments, RA and RB are C6-C30 straight chain alkynyl groups, or C21-C30 straight chain alkynyl groups, preferably C9-C20 straight chain alkynyl groups. In certain embodiments, when RA and RB are the same, RA and RB are not methyl, ethyl, n-propyl,

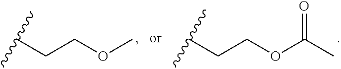

In other embodiments, when RA and RB are the same, RA and RB each comprise at least 4 carbon atoms. In other embodiments, when RA and RB are the same, RA and RB each comprise at least 5 carbon atoms. In other embodiments, when RA and RB are the same, RA and RB each comprise at least 6 carbon atoms. In other embodiments, RA and RB each comprise at least 4 carbon atoms. In other embodiments, RA and RB each comprise at least 5 carbon atoms. In other embodiments, RA and RB each comprise at least 6 carbon atoms. Exemplary classes of the above formula include:

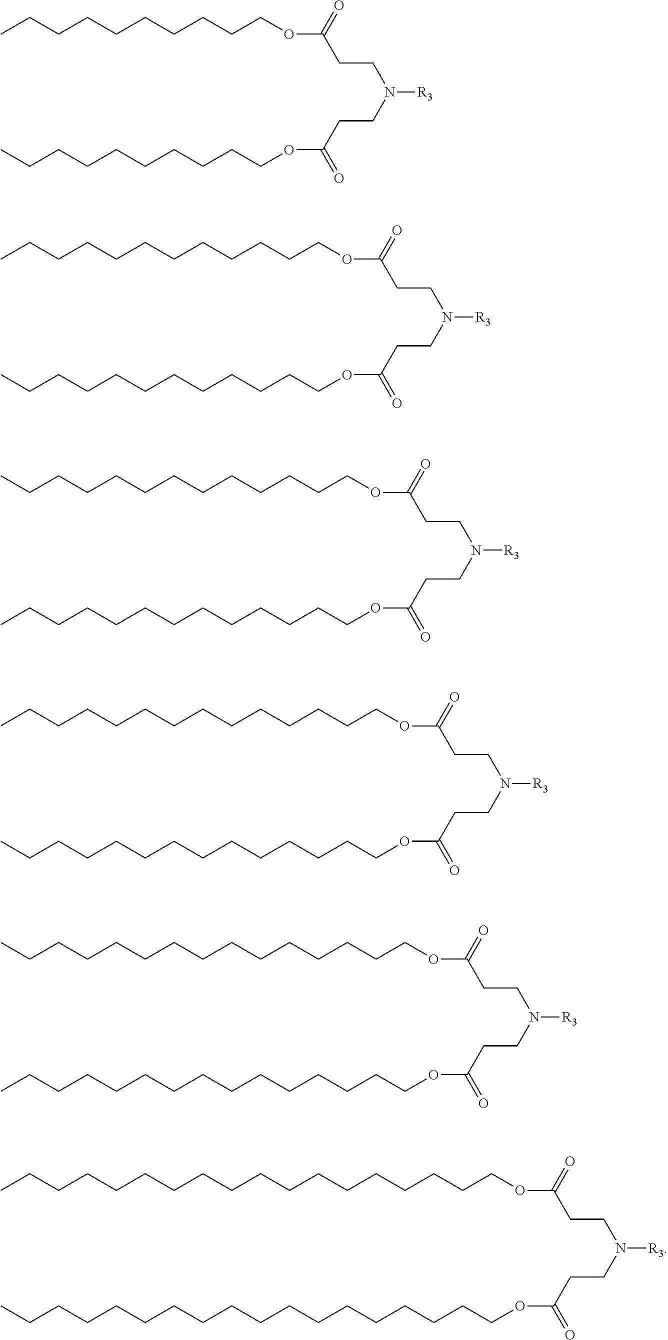

In certain embodiments, the acrylate used in the synthesis of the lipid is acrylate LD, LF, or LG in FIG. 1A. In certain embodiments the acrylate is acrylate LF in FIG. 1A. In certain embodiments the acrylate is acrylate LG in FIG. 1A. In certain embodiments, R3 is not

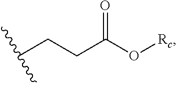

wherein Rc is defined as above. In certain embodiments, R3 is not —CH2CH2ORc′, wherein Rc′ is methyl, ethyl, propyl, isopropyl, butyl, s-butyl, isobutyl, t-butyl, pentyl, cyclopentyl, hexyl, cyclohexyl, decyl, methoxymethyl, 2-methoxyethyl, 1-ethoxyethyl, 2-ethoxyethyl, (2-methoxyethoxy)methyl, 2-tetrahydrofuranyl, 2-tetrahydropyranyl, tetrahydrofurfuryl, formyl, acetyl, propionyl, butyryl, isobutyryl, pivaloyl, valeryl, methoxyacetyl, ethoxyacetyl, acetoxyacetyl, 2-formyloxyethyl, 2-acetoxyethyl, 2-oxopropyl, 2-oxobutyl, 2-oxocyclopentyl, 2-oxo-3-tetrahydrofuranyl, 2-oxo-3-tetrahydropyranyl, methoxycarbonyl, ethoxycarbonyl, and t-butoxycarbonyl. In yet other embodiments, R3 is not —CH2CH2ORc″, wherein Rc″ is a straight chain, branched or cyclic alkyl group of 1 to 20 carbons atoms, which may contain an ether, carbonyl, or carbonyloxy group. In yet other embodiments, R3 is not —CH2CH2ORc″, wherein Rc″ is a straight chain, branched or cyclic alkyl group of 1 to 10 carbons atoms, which may contain an ether, carbonyl, or carbonyloxy group. In certain particular embodiments, R3 is not —CH2CH2ORc″, wherein Rc″ is formyl; acetyl; or methyl group.

In other embodiments, R1 is —NRA and R2 is —NRB, as shown in the formula below:

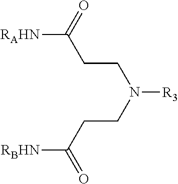

In certain embodiments, RA and RB are the same. In other embodiments, RA and RB are different. In certain embodiments, RA and RB are C6-C30 straight chain alkyl groups, or C21-C30 straight chain alkyl groups, preferably Cg-C20 straight chain alkyl groups. In certain embodiments, RA and RB are C6-C30 straight chain alkenyl groups, or C21-C30 straight chain alkenyl groups, preferably Cg-C20 straight chain alkenyl groups. In certain embodiments, RA and RB are C6-C30 straight chain alkynyl groups, or C21-C30 straight chain alkynyl groups, preferably C9-C20 straight chain alkynyl groups. In certain embodiments, when RA and RB are the same, RA and RB are not methyl, ethyl, n-propyl,

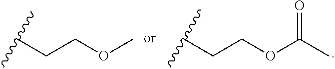

In other embodiments, when RA and RB are the same, RA and RB each comprise at least 4 carbon atoms. In other embodiments, when RA and RB are the same, RA and RB each comprise at least 5 carbon atoms. In other embodiments, when RA and RB are the same, RA and RB each comprise at least 6 carbon atoms. In other embodiments, RA and RB each comprise at least 4 carbon atoms. In other embodiments, RA and RB each comprise at least 5 carbon atoms. In other embodiments, RA and RB each comprise at least 6 carbon atoms. Exemplary classes of the above formula include:

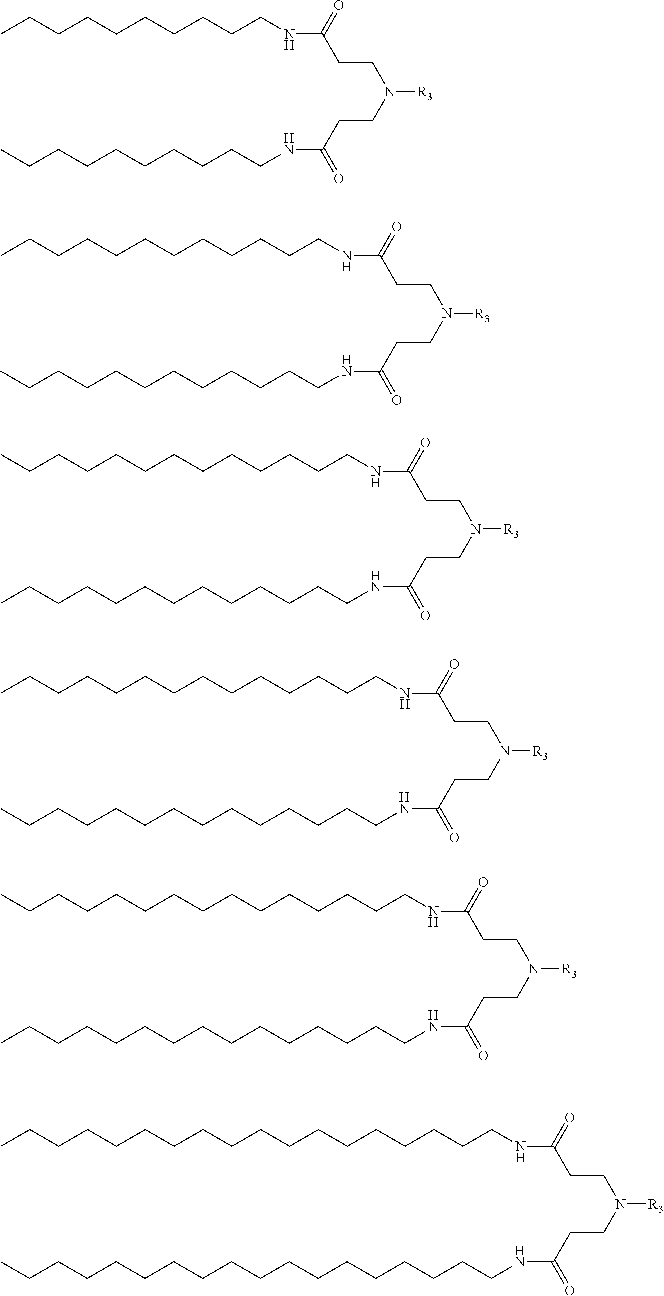

In certain embodiments, the acrylate used in the synthesis of the lipid is acrylate ND, NF, NG, or NP in FIG. 1A. In certain embodiments the acrylate is acrylate ND in FIG. 1A. In certain embodiments the acrylate is acrylate NF in FIG. 1A. In certain embodiments the acrylate is acrylate NP in FIG. 1A.

Particular exemplary compounds include:

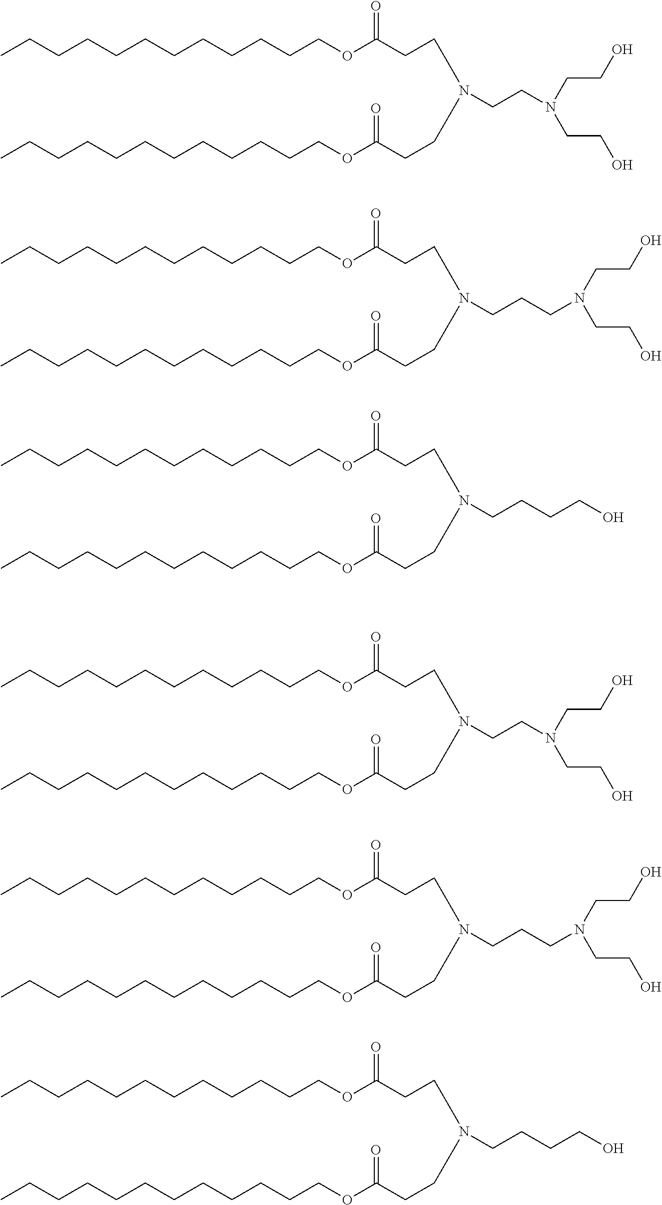

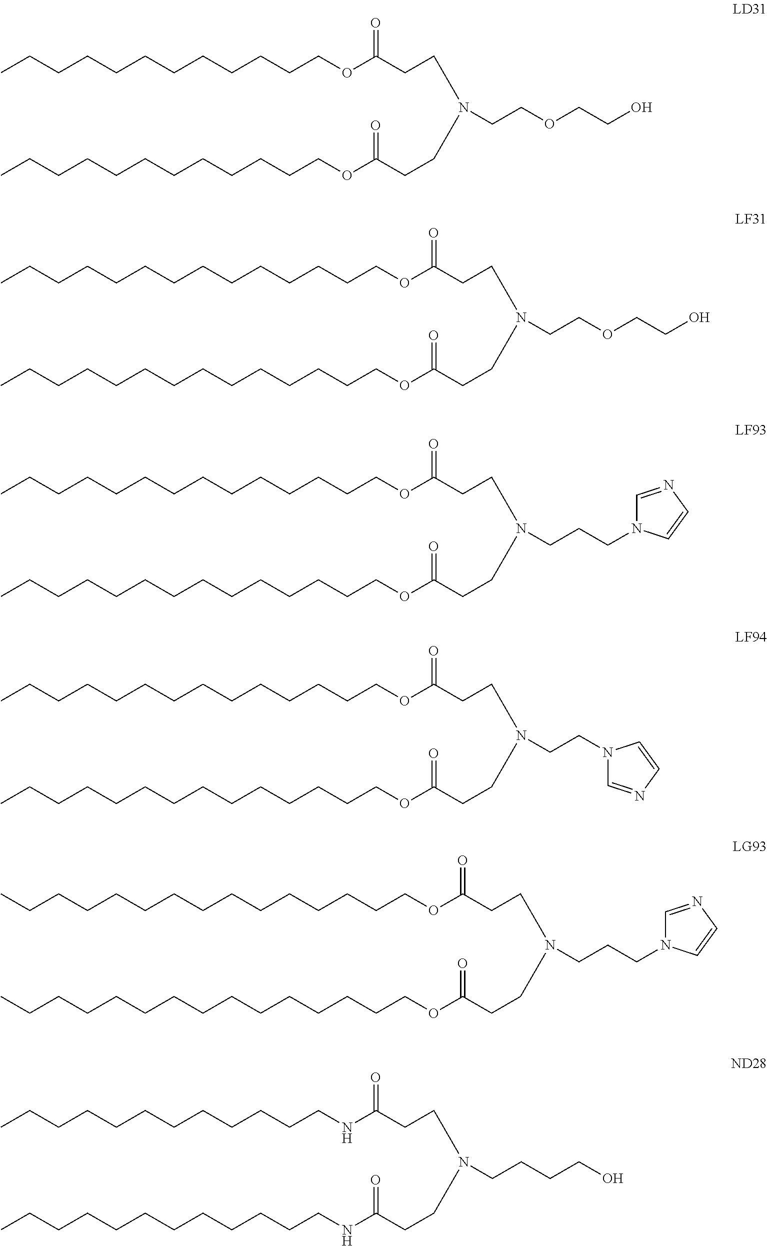

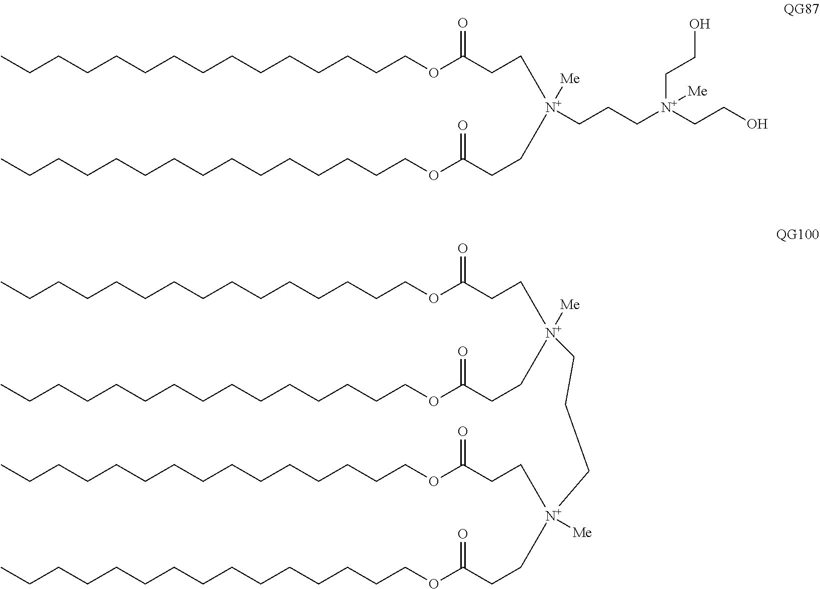

In other subclasses of lipids, the lipids are of the formula:

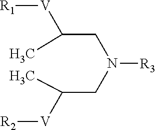

wherein V, R1, and R3 are defined as above; all occurrences of R6 are hydrogen; and R5 is defined as in the formula. In certain embodiments, R1 and R2 are the same. In certain embodiments, V is C═O as shown in the formula:

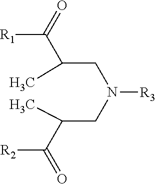

preferably R1 and R2 are the same.

In certain embodiments, R1 is —ORA and R2 is —ORB, as shown in the formula below:

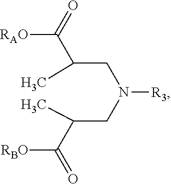

preferably RA and RB are the same. In certain embodiments,

RA and RB are C6-C30 straight chain alkyl groups, preferably C9-C20 straight chain alkyl groups.

In other embodiments, R1 is —NRA and R2 is —NRB, as shown in the formula below:

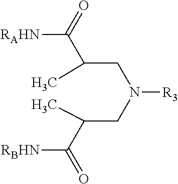

preferably RA and RB are the same. In certain embodiments, RA and RB are C6-C30 straight chain alkyl groups, preferably C9-C20 straight chain alkyl groups.

In certain embodiments,

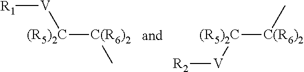

in formulae (I) and (Ia) are selected from the group consisting of:

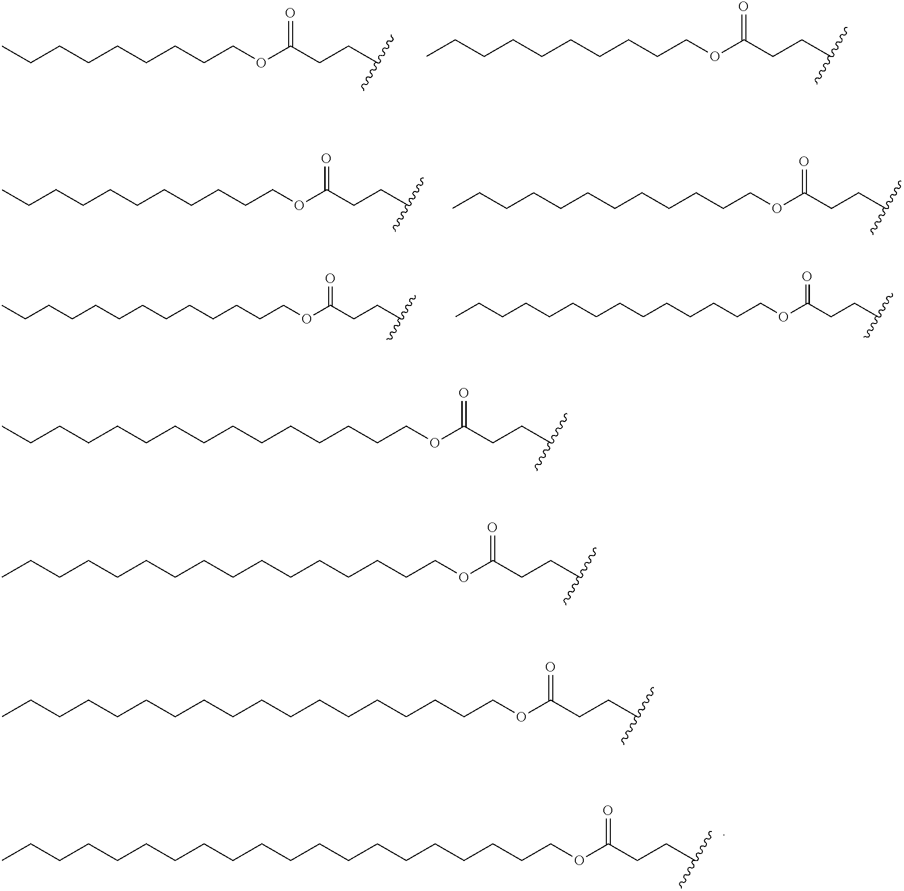

In certain embodiments, the lipids are prepared using acrylates LC, LD, LE, LF, and LG in FIG. 1A.

In certain embodiments

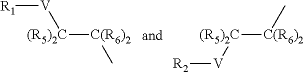

in formulae (I) and (Ia) are selected from the group consisting of:

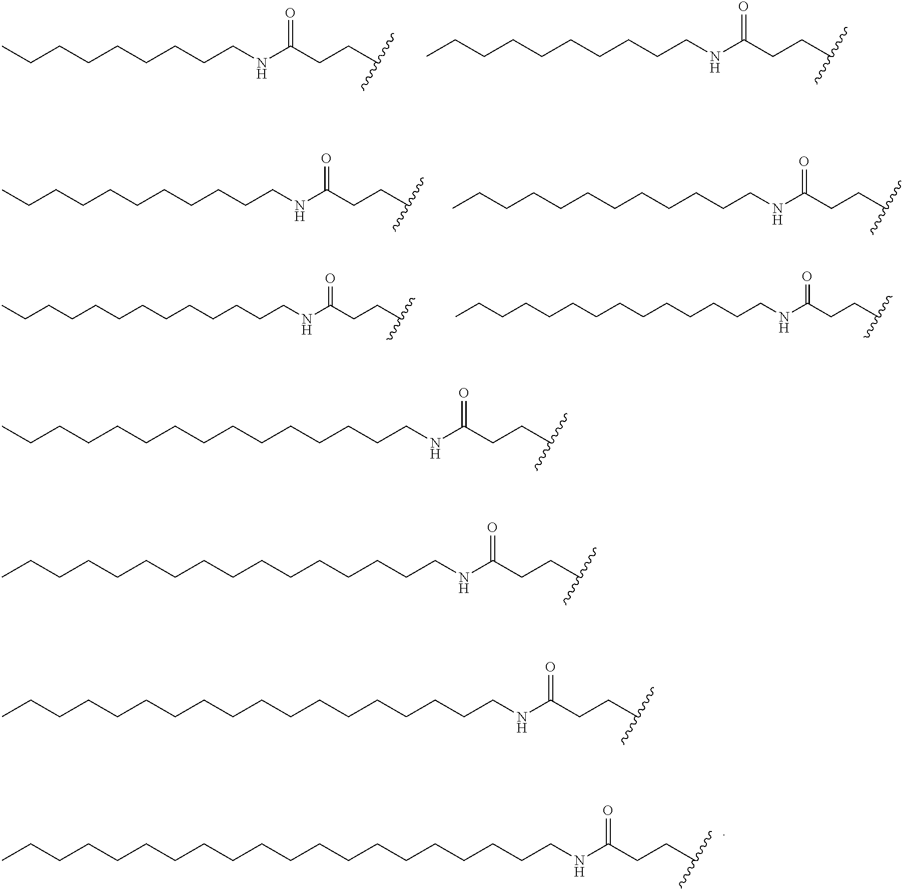

In certain embodiments, the lipids are prepared using acrylates NC, ND, NF, NG, or NP in FIG. 1A. In certain embodiments, the lipids are prepared using acrylate ND. In other embodiments, the lipids are prepared using acrylate NF.

In certain embodiments,

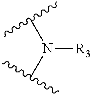

is selected from the group consisting of:

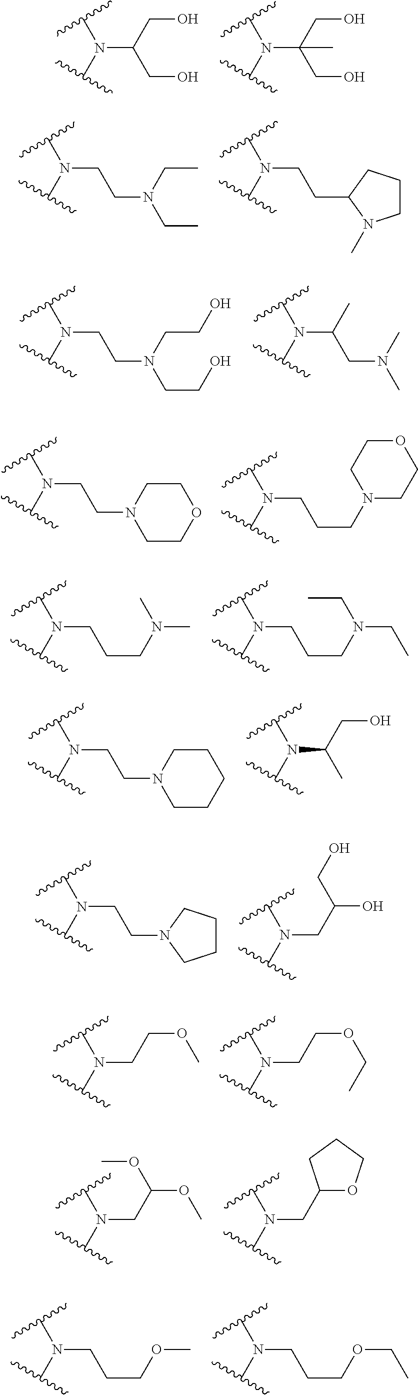

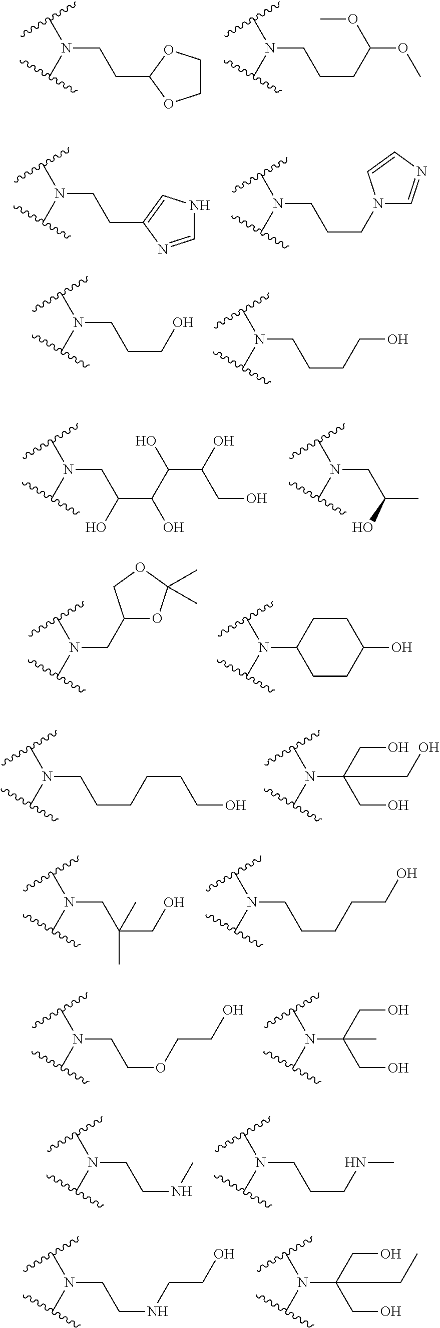

In certain embodiments,

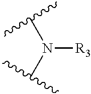

is selected from the group consisting of:

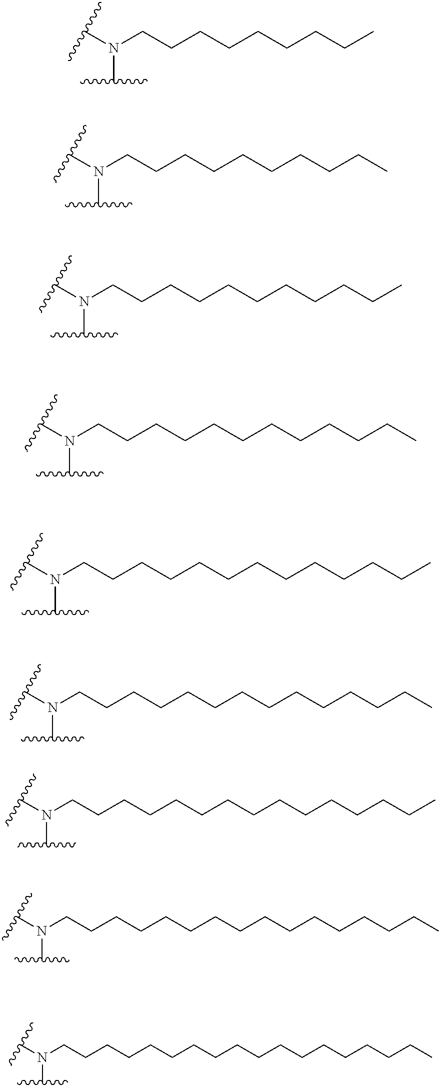

In certain embodiments,

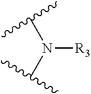

is

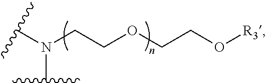

wherein n is an interger between 0 and 10, inclusive; and R3′ is hydrogen, aliphatic, heteroaliphatic, carbocyclic, heterocyclic, aryl, acyl, or heteroaryl. In certain embodiments, R3′ is hydrogen, In other embodiments, R3′ is C1-C6 alkyl. In yet other embodiments, R3′ is acyl (e.g., acetyl).

In certain embodiments, the lipid is of formula:

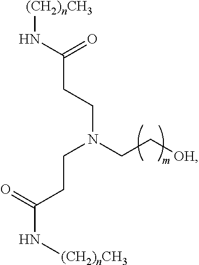

wherein n is an integer between 5 and 20, inclusive; and m is an integer between 1 and 10, inclusive; and pharmaceutically acceptable salts thereof. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

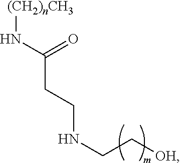

wherein n is an integer between 5 and 20, inclusive; and m is an integer between 1 and 10, inclusive; and pharmaceutically acceptable salts thereof. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

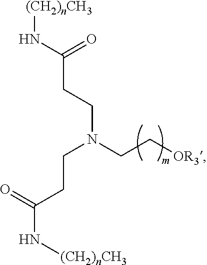

wherein

R3′ is C1-6alkyl; n is an integer between 5 and 20, inclusive; and m is an integer between 1 and 10, inclusive; and pharmaceutically acceptable salts thereof. In certain embodiments, R3′ is methyl. In other embodiments, R3′ is ethyl. In other embodiments, R3′ is n-propyl. In still other embodiments, R3′ is iso-propyl. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

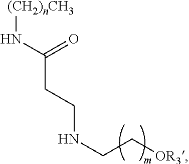

wherein R3′ is C1-6alkyl; n is an integer between 5 and 20, inclusive; and m is an integer between 1 and 10, inclusive; and pharmaceutically acceptable salts thereof. In certain embodiments, R3′ is methyl. In other embodiments, R3′ is ethyl. In other embodiments, R3′ is n-propyl. In still other embodiments, R3′ is iso-propyl. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

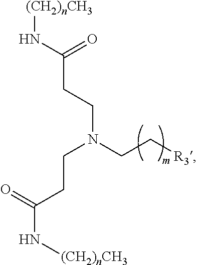

wherein R3′ is carbocyclic; heterocyclic; aryl or heteroaryl; n is an integer between 5 and 20, inclusive; and m is an integer between 1 and 10, inclusive; and pharmaceutically acceptable salts thereof. In certain embodiments, R3′ is phenyl. In other embodiments, R3′ is heteroaryl. In other embodiments, R3′ is aryl. In still other embodiments, R3′ is histidine. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

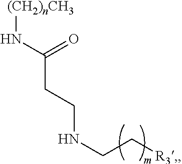

wherein R3′ is carbocyclic; heterocyclic; aryl or heteroaryl; n is an integer between 5 and 20, inclusive; and m is an integer between 1 and 10, inclusive; and pharmaceutically acceptable salts thereof. In certain embodiments, R3′ is phenyl. In other embodiments, R3′ is heteroaryl. In other embodiments, R3′ is aryl. In still other embodiments, R3′ is histidine. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

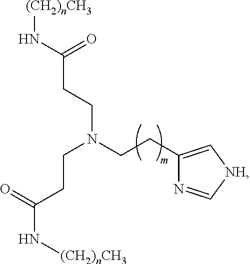

wherein n is an integer between 5 and 20, inclusive; and m is an integer between 1 and 10, inclusive; and pharmaceutically acceptable salts thereof. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

In certain embodiments, the lipid is of formula:

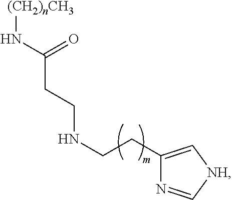

wherein n is an integer between 5 and 20, inclusive; and m is an integer between 1 and 10, inclusive; and pharmaceutically acceptable salts thereof. In certain embodiments, n is 11. In other embodiments, n is 12. In yet other embodiments, n is 13. In still other embodiments, n is 14. In certain embodiments, m is 1. In other embodiments, m is 2. In other embodiments, m is 3. In other embodiments, m is 4. In other embodiments, m is 5. In other embodiments, m is 6.

The present invention also provides amino lipids prepared from reacting acrylates with diamines, triamines, or polyamines. The amino moieties are completely or partially reacted with acrylate or acrylamides. Also, as would be appreciated by one of skill in this art, amino lipids with different number of acrylate or acrylamide tails will result in various isomers. These various forms of the lipids are prepared individually, or the lipid is prepared as a mixture and then purified from the other forms. A single form may be used in a composition, or a mixture of forms may be used.

The tails of the amino lipids may also be the same or different.

Non-exhaustively reacted amino groups may be reacted with a second acrylate, second acrylamide, or other electrophiles to created a mixed amino lipid. Again, various isomeric forms may be prepared and may optionally be purified.

In certain embodiments, the lipids of the present invention are of the formula (II):

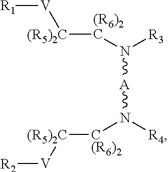

wherein A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; and substituted or unsubstituted, branched or unbranched heteroaryl; V is selected from the group consisting of C═O, C═S, S═O, and SO2; R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(═O)RB; —CO2RB; —CN; —SCN; —SRB; —SOR3; —SO2RB; —NO2; —N3; —N(RB)2; —NHC(═O)RB; —NRBC(═O)N(RB)2; —OC(═O)ORB; —OC(═O)RB; -0C(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;

R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORC; —C(═O)Rc; —CO2Rc; —CN; —SCN; —SRC; —SOR0; —SO2RO; —NO2; —N3; —N(Rc)2; —NHC(═O)Rc; —NRcC(═O)N(Rc)2; —OC(═O)ORc; —OC(═O)Rc; —OCC═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;

R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORO; —C(O)RO; —CO2RO; —CN; —SCN; —SRO; —SORO; —SO2RD; —NO2; —N3; —N(RD)2; —NHC(═O)RD; —NRCC(═O)N(RD)2; —OC(═O)ORD; —OC(═O)RD; —OC(═O)N(RD)2; —NRcC(═O)ORD; or —C(RO)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R5 is independently selected from the group consisting of hydrogen and C1-C6 alkyl; each occurrence of R6 is independently selected from the group consisting of hydrogen and C1-C6 alkyl; and salts thereof. In certain embodiments, the lipid is prepared using amine 95, 96, 99, 100, 103, and 109 in FIGS. 1A and 1B. In certain embodiments, the lipid is prepared using amine 99 in FIGS. 1A and 1B. In certain embodiments, the lipid is prepared using amine 100 in FIGS. 1A and 1B. In certain embodiments, the lipid is prepared using acrylate ND, NF, NP, LF, and LG in FIG. 1A. In certain embodiments, the lipid is prepared using acrylate ND in FIG. 1A. In certain embodiments, the lipid is prepared using acrylate NF in FIG. 1A. In certain embodiments, the lipid is prepared using acrylate NP in FIG. 1A.

In certain embodiments, the tertiary amine of formula (II) is protonated or alkylated to form a compound of formula (IIa):

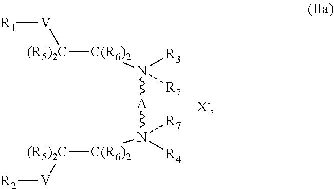

wherein R1, R2, R3, R4, R5, R6, and V are defined above; each occurrence of R7 is hydrogen or C1-C6 aliphatic, preferably C1-C6 alkyl, more preferably hydrogen or methyl; each dashed line represents a bond or the absence of a bond, wherein when the dashed line represents a bond, the attached nitrogen is positively charged; and

X is any anion. Possible anions include fluoride, chloride, bromide, iodide, sulfate, bisulfate, phosphate, nitrate, acetate, fumarate, oleate, citrate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate). In certain embodiments, both dashed lines presents bonds, and both nitrogen atoms are positively charged.

In certain embodiments, A is a cyclic or acyclic, substituted or unsubstituted, branched or unbranched aliphatic or heteroaliphatic group. In certain embodiments, A is a substituted or unsubstituted, branched or unbranched aliphatic group. In certain particular embodiments, A is a substituted or unsubstituted, branched or unbranched alkyl group. In certain embodiments, A is an unsubstituted, C1-C6 straight chain alkyl group. In other embodiments, A is a polyethylene group. In yet other embodiments, A is a polyethylene glycol moiety. In certain embodiments, A, the two nitrogen atoms attached to A, R3 and R4 form a heterocyclic ring. In certain embodiments, the ring is aromatic. In other embodiments, the ring is non-aromatic. In certain embodiments,

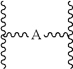

is selected from the group consisting of:

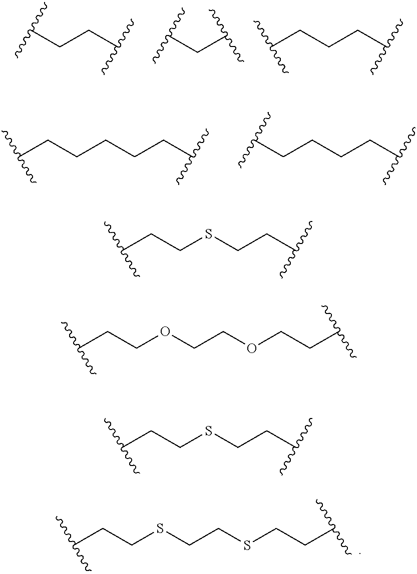

In certain particular embodiments,

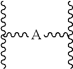

is

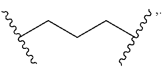

In certain embodiments,

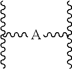

is

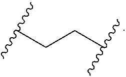

In certain embodiments, A is

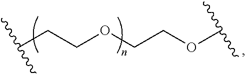

wherein n is an interger between O and 10, inclusive.

In certain embodiments, V is C═O. In other embodiments, V is C═S. In yet other embodiments, V is S═O. In still other embodiments, V is SO2.

In certain embodiments, R1 is hydrogen. In other embodiments, R1 is a cyclic or acyclic, substituted or unsubstituted, branched or un branched aliphatic or heteroaliphatic moiety. In certain embodiments, R1 is a substituted or unsubstituted aryl or heteroaryl moiety. Preferably, the aryl or heteroaryl moiety is a monocylic 5- or 6-membered ring system. In certain embodiments, R1 is —ORA, —SRA, —N(RA)2, or —NHRA. In certain embodiments, R1 is —ORA. In other embodiments, R1 is —N(RA)2 or —NHRA. In certain embodiments, RA is hydrogen. In certain embodiments, RA is not hydrogen. In other embodiments, RA is a cyclic or acyclic, substituted or unsubstituted, branched or unbranched aliphatic or heteroaliphatic moiety. In certain embodiments, RA is an acyclic, substituted or unsubstituted aliphatic moiety. In certain embodiments, RA is an unsubstituted, straight chain alkyl group with at least 5 carbons. In certain other embodiments, RA is an acyclic, unsubstituted, unbranched aliphatic moiety, preferably C6-C30, more preferably C10-C20. In certain embodiments, RA is an unsubstituted, straight chain alkyl group, preferably C6-C30, more preferably C10-C20. In certain embodiments, R1 is —ORA, wherein RA is an unsubstituted, unbranched C9 alkyl chain. In certain embodiments, R1 is —ORA, wherein RA is an unsubstituted, unbranched C10 alkyl chain. In certain embodiments, R1 is —ORA, wherein RA is an unsubstituted, unbranched C11 alkyl chain. In certain embodiments, R1 is —ORA, wherein RA is an unsubstituted, unbranched C12 alkyl chain. In certain embodiments, R1 is —ORA, wherein RA is an unsubstituted, unbranched C13 alkyl chain. In certain embodiments, R1 is —ORA, wherein RA is an unsubstituted, unbranched C14 alkyl chain. In certain embodiments, R1 is —ORA, wherein RA is an unsubstituted, unbranched C15 alkyl chain. In certain embodiments, R1 is —ORA, wherein RA is an unsubstituted, unbranched C16 alkyl chain. In certain embodiments, R1 is —ORA, wherein RA is an unsubstituted, unbranched C17 alkyl chain. In certain embodiments, R1 is —ORA, wherein RA is an unsubstituted, unbranched C18 alkyl chain. In certain embodiments, R1 is —ORA, wherein RA is an unsubstituted, unbranched C1Q alkyl chain. In certain embodiments, R1 is —ORA, wherein RA is an unsubstituted, unbranched C20 alkyl chain. In yet other embodiments, RA is a substituted or unsubstituted aryl or heteroaryl moiety.

In certain embodiments, R2 is hydrogen. En other embodiments, R2 is a cyclic or acyclic, substituted or unsubstituted, branched or unbranched aliphatic or heteroaliphatic moiety. In certain embodiments, R2 is a substituted or unsubstituted aryl or heteroaryl moiety. Preferably, the aryl or heteroaryl moiety is a monocylic 5- or 6-membered ring system. In certain embodiments, R2 is —ORB, —SRB, —N(RB)2, or —NHRB. In certain embodiments, R2 is —ORB. In other embodiments, R2 is —N(RB)2 or —NHRB. In certain embodiments, RB is hydrogen. In certain embodiments, RB is not hydrogen. In other embodiments, RB is a cyclic or acyclic, substituted or unsubstituted, branched or unbranched aliphatic or heteroaliphatic moiety. In certain embodiments, RB is an acyclic, substituted or unsubstituted aliphatic moiety. In certain embodiments, RB is an unsubstituted, straight chain alkyl group with at least 5 carbons. In certain other embodiments, RB is an acyclic, unsubstituted, unbranched aliphatic moiety, preferably C6-C30, more preferably C10-C20. In certain embodiments, RB is an unsubstituted, straight chain alkyl group, preferably C6-C30, more preferably C10-C20. In certain embodiments, R2 is —ORB, wherein RB is an unsubstituted, unbranched C9 alkyl chain. In certain embodiments, R2 is —ORB, wherein RB is an unsubstituted, unbranched C10 alkyl chain. In certain embodiments, R2 is —ORB, wherein RB is an unsubstituted, unbranched C11 alkyl chain. In certain embodiments, R2 is —ORB, wherein RB is an unsubstituted, unbranched C12 alkyl chain. In certain embodiments, R2 is —ORB, wherein RB is an unsubstituted, unbranched C13 alkyl chain. In certain embodiments, R2 is —ORB, wherein RB is an unsubstituted, unbranched C14 alkyl chain. In certain embodiments, R2 is —ORB, wherein RB is an unsubstituted, unbranched C15 alkyl chain. In certain embodiments, R2 is —ORA, wherein RB is an unsubstituted, unbranched C16 alkyl chain. In certain embodiments, R2 is —ORA, wherein RB is an unsubstituted, unbranched C17 alkyl chain. In certain embodiments, R2 is —ORA, wherein RB is an unsubstituted, unbranched C18 alkyl chain. In certain embodiments, R2 is —ORB, wherein RB is an unsubstituted, unbranched C19 alkyl chain. In certain embodiments, R2 is —ORB, wherein RB is an unsubstituted, unbranched C20 alkyl chain. In yet other embodiments, RB is a substituted or unsubstituted aryl or heteroaryl moiety.

In certain embodiments, R3 is a cyclic or acyclic, substituted or unsubstituted, branched or unbranched aliphatic moiety. In other embodiments, R3 is a cyclic or acyclic, substituted or unsubstituted, branched or unbranched heteroaliphatic moiety. In certain embodiments, R3 is an aliphatic moiety substituted with one or more hydroxyl groups. In other embodiments, R3 is an aliphatic moiety substituted with one or more amino, alkylamino, or dialkylamino groups. In certain embodiments, R3 is C1-C6 alkyl. In certain embodiments, R3 is methyl. In certain embodiments, R3 is ethyl. In other embodiments, R3 is n-propyl. In other embodiments, R3 is iso-propyl. In certain embodiments, R3 is hydrogen. In certain embodiments, R3 is a heteroaliphatic moiety. In certain embodiments, R3 is cyclic aliphatic, preferably a monocylic ring system with a 5- or 6-membered ring. In other embodiments, R3 is aryl or heteroaryl, preferably a monocyclic ring system with a 5- or 6-membered ring. In certain embodiments, R3 is

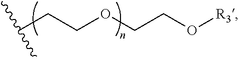

wherein n is an interger between 0 and 10, inclusive; and R3′ is hydrogen, aliphatic, heteroaliphatic, carbocyclic, heterocyclic, aryl, acyl, or heteroaryl. In certain embodiments, R3′ is hydrogen, In other embodiments, R3′ is C1-C6 alkyl. In yet other embodiments, R3′ is acyl (e.g., acetyl). In certain embodiments, R3 is

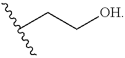

In other embodiments, R3 is

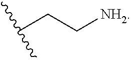

In certain embodiments, R4 is a cyclic or acyclic, substituted or unsubstituted, branched or unbranched aliphatic moiety. In other embodiments, R4 is a cyclic or acyclic, substituted or unsubstituted, branched or unbranched heteroaliphatic moiety. In certain embodiments, R4 is an aliphatic moiety substituted with one or more hydroxyl groups. In other embodiments, R4 is an aliphatic moiety substituted with one or more amino, alkylamino, or dialkylamino groups. In certain embodiments, R4 is C1-C6 alkyl. In certain embodiments, R3 is methyl. In certain embodiments, R3 is ethyl. In other embodiments, R3 is n-propyl. In other embodiments, R3 is iso-propyl. In certain embodiments, R4 is hydrogen. In certain embodiments, R4 is a heteroaliphatic moiety. In certain embodiments, R3 is cyclic aliphatic, preferably a monocylic ring system with a 5- or 6-membered ring. In other embodiments, R4 is aryl or heteroaryl, preferably a monocyclic ring system with a 5- or 6-membered ring. In certain embodiments, R4 is

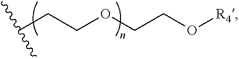

wherein n is an interger between 0 and 10, inclusive; and R4′ is hydrogen, aliphatic, heteroaliphatic, carbocyclic, heterocyclic, aryl, acyl, or heteroaryl. In certain embodiments, R4′ is hydrogen. In other embodiments, R4′ is C1-C6 alkyl. In yet other embodiments, R4′ is acyl (e.g., acetyl). In certain embodiments, R4 is

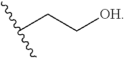

In other embodiments, R4 is

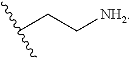

In certain embodiments, R3 and R4 are the same. In other embodiments,

R3 and R4 are different.

In certain embodiments, each occurrence of R5 is hydrogen. In certain embodiments, at least one occurrence of R5 is methyl and the other occurrences are hydrogen. In certain embodiments, at least two occurrences of R5 are methyl, and the other occurrences are hydrogen. In other embodiments, at least two occurrences of R5 are hydrogen.

In certain embodiments, each occurrence of R6 is hydrogen. In certain other embodiments, at least two occurrences of R6 are hydrogen. In certain embodiments, at least one occurrence of R6 is methyl, and the other occurrences are hydrogen. In certain embodiments, at least two occurrences of R6 are methyl, and the other occurrences are hydrogen.

In certain embodiments

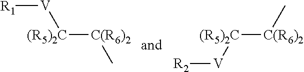

which are attached to N are the same. In other embodiments,

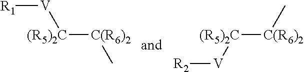

which are attached to N are the same and are different than R3. In yet other embodiments,

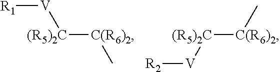

and R3 are all different.

In certain embodiments,

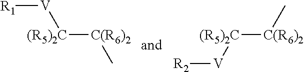

in formulae (II) and (IIa) are selected from the group consisting of:

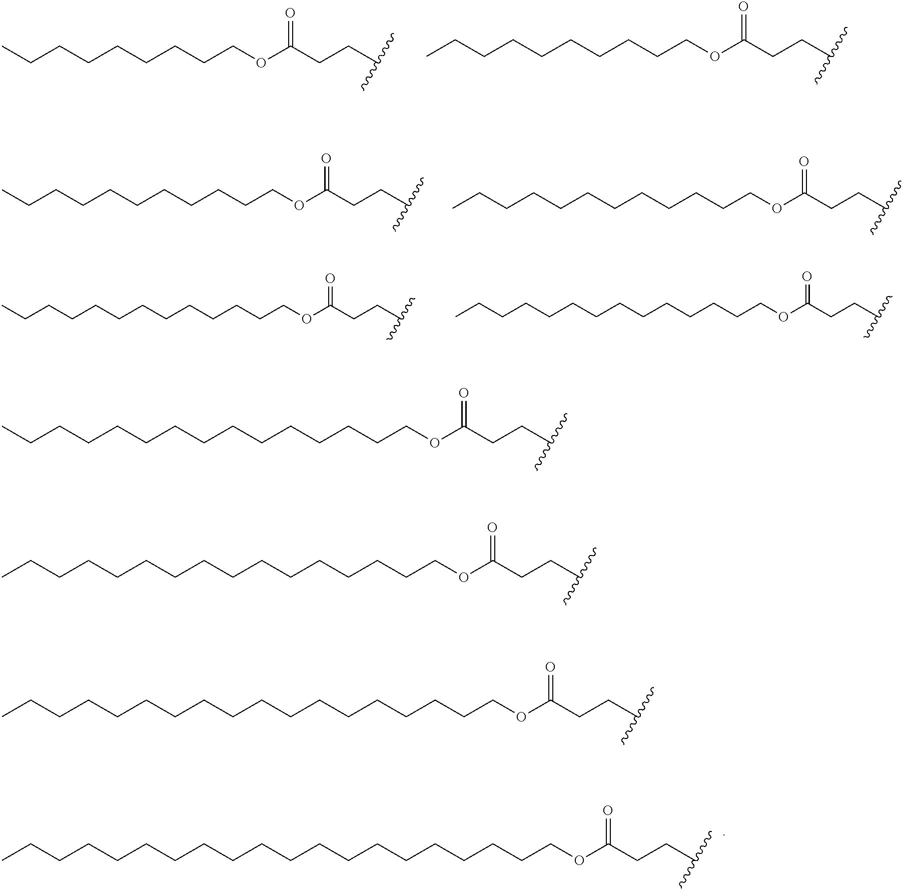

In certain embodiments, the lipids are prepared using acrylates LC, LD, LE, LF, and LG in FIG. 1A.

In certain embodiments,

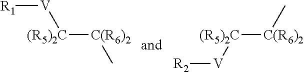

in formulae (II) and (IIa) are selected from the group consisting of:

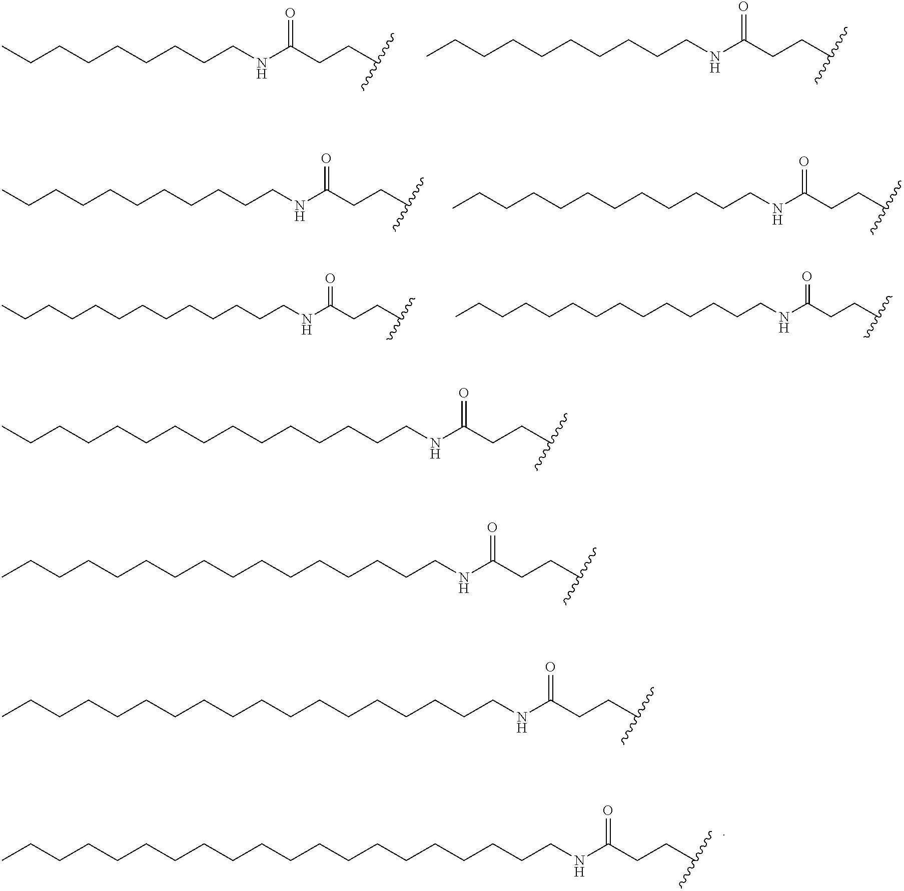

In certain embodiments, the lipids are prepared using acrylates NC, ND, NF, NG, or NP in FIG. 1A. In certain embodiments, the lipids are prepared using acrylate ND. In other embodiments, the lipids are prepared using acrylate NF. In other embodiments, the lipids are prepared using acrylate NP.

In certain embodiments,

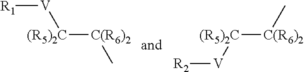

in formulae (II) and (IIa) are the same.

In other embodiments,

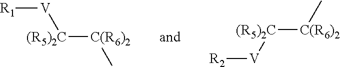

in formulae (II) and (IIa) are different.

In certain embodiments,

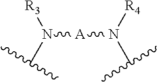

is selected from the group consisting of:

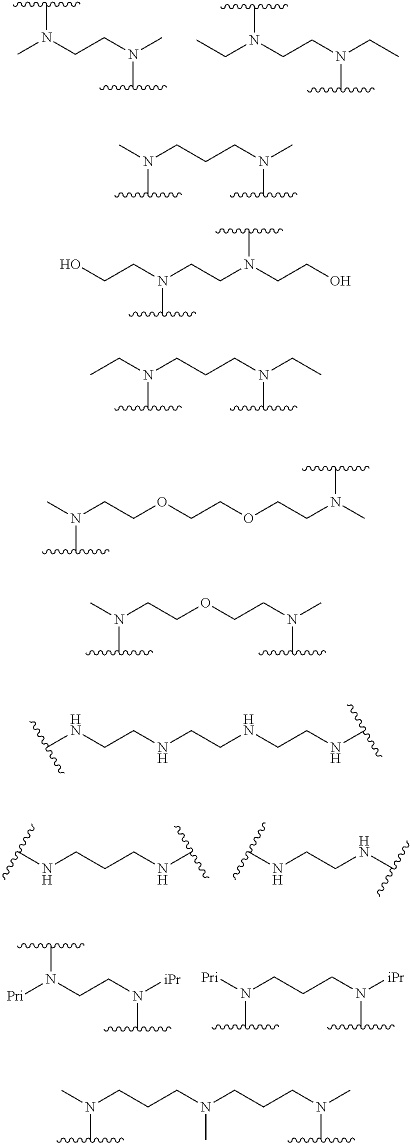

In certain embodiments,

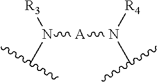

is selected from the group consisting of:

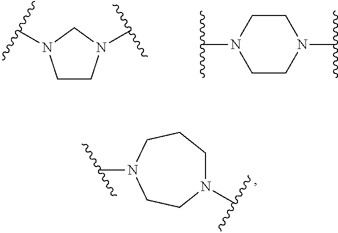

wherein R3 and R4 form a cyclic structure.

In other embodiments, the lipids of the present invention are of the formula (III):

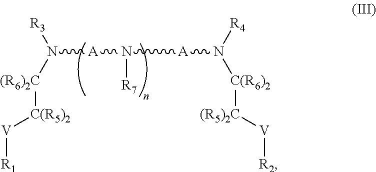

wherein A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; and substituted or unsubstituted, branched or unbranched heteroaryl;

V is selected from the group consisting of C═O, C═S, S═O, and SO2; n is an integer between 0 and 10, inclusive; R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(═O)RB; —CO2RB; —CN; —SCN; —SRB; —SORB; —SO2RB; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure; R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORC; —C(O)Rc; —CO2RC; —CN; —SCN; —SRC; —SORC; —SO2Rc; —NO2; —N3; —N(Rc)2; —NHC(═O)RC; —NRCC(═O)N(RC)2; —OC(═O)ORC; —OC(═O)RC; -0C(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORD; —C(═O)RD; —CO2RO; —CN; —SCN; —SRD; —SORD; —SO2RD; —NO2; —N3; —N(RD)2; —NHC(═O)RD; —NRCC(═O)N(RD)2; —OC(O)ORD; —OC(═O)RD; —OC(═O)N(RD)2; —NRcC(═O)ORD; or —C(RD)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R5 is independently selected from the group consisting of hydrogen and C1-C6 alkyl; each occurrence of R6 is independently selected from the group consisting of hydrogen and C1-C6 alkyl; R7 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORG; —C(O)RG; —CO2RG; —CN; —SCN; —SRG; —SORG; —SO2RG; —NO2; —N3; —N(RG)2; —NHC(═O)RG; —NRGC(═O)N(RG)2; —OC(═O)ORG; —OC(═O)RG; —OC(═O)N(RG)2; —NRGC(═O)ORG; and —C(RG)3; wherein each occurrence of RG is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety and salts thereof. In certain embodiments, n is 0. In other embodiments, n is 1. In still other embodiments, n is 2. In other embodiments, n is 3. In yet other embodiments, n is 4. In other embodiments, n is 5. In other embodiments, n is 6. In certain embodiments, the lipid is prepared using amine 98. In other embodiments, the lipid is prepared using amine 100.

In certain embodiments, the tertiary amine of formula (III) is protonated or alkylated to form a compound of formula (IIIa):

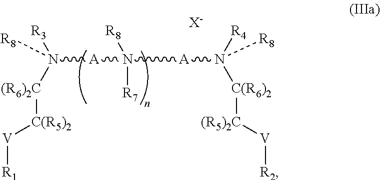

wherein R1, R2, R3, R4, R5, R6, R7, n, and V are defined above; each occurrence of R8 is hydrogen or C1-C6 aliphatic, preferably C1-C6 alkyl, more preferably hydrogen or methyl; each dashed line represents a bond or the absence of a bond, wherein when the dashed line represents a bond, the attached nitrogen is positively charged; and X is any anion. Possible anions include fluoride, chloride, bromide, iodide, sulfate, bisulfate, phosphate, nitrate, acetate, fumarate, oleate, citrate, valerate, maleate, oxalate, isonicotinate, lactate, salicylate, tartrate, tannate, pantothenate, bitartrate, ascorbate, succinate, gentisinate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate). In certain embodiments, both dashed lines presents bonds, and both nitrogen atoms are positively charged.

In certain embodiments, A is a cyclic or acyclic, substituted or unsubstituted, branched or unbranched aliphatic or heteroaliphatic group. In certain embodiments, A is a substituted or unsubstituted, branched or unbranched aliphatic group. In certain particular embodiments, A is a substituted or unsubstituted, branched or unbranched alkyl group. In certain embodiments, A is an unsubstituted, C1-C6 straight chain alkyl group. In other embodiments, A is a polyethylene group. In yet other embodiments, A is a polyethylene glycol moiety. In certain embodiments, A, the two nitrogen atoms attached to A, R3 and R4 form a heterocyclic ring. In certain embodiments, the ring is aromatic. In other embodiments, the ring is non-aromatic.

In certain embodiments, V is C═O. In other embodiments, V is C═S. In yet other embodiments, V is S═O. In still other embodiments, V is SO2.

In certain embodiments, R1 is hydrogen. In other embodiments, R1 is a cyclic or acyclic, substituted or unsubstituted, branched or un branched aliphatic or heteroaliphatic moiety. In certain embodiments, R1 is a substituted or unsubstituted aryl or heteroaryl moiety. Preferably, the aryl or heteroaryl moiety is a monocylic 5- or 6-membered ring system. In certain embodiments, R1 is —ORA, —SRA, —N(RA)2, or —NHRA- In certain embodiments, R1 is —ORA. In other embodiments, R1 is —N(RA)2 or —NHRA. In certain embodiments, RA is hydrogen. In other embodiments, RA is a cyclic or acyclic, substituted or unsubstituted, branched or unbranched aliphatic or heteroaliphatic moiety. In certain embodiments, RA is an acyclic, substituted or unsubstituted aliphatic moiety. In certain other embodiments, RA is an acyclic, unsubstituted, unbranched aliphatic moiety, preferably C6-C30, more preferably C10-C20. In certain embodiments, RA is an unsubstituted, straight chain alkyl group, preferably C6-C30, more preferably C10-C20. In certain embodiments, R1 is —ORA, wherein RA is an unsubstituted, unbranched C9 alkyl chain. In certain embodiments, R1 is —ORA, wherein RA is an unsubstituted, unbranched C10 alkyl chain. In certain embodiments, R1 is —ORA, wherein RA is an unsubstituted, unbranched Cn alkyl chain. In certain embodiments, R1 is —ORA, wherein RA is an unsubstituted, unbranched C12 alkyl chain. In certain embodiments, R1 is —ORA, wherein RA is an unsubstituted, unbranched C13 alkyl chain. In certain embodiments, R1 is —ORA, wherein RA is an unsubstituted, unbranched C14 alkyl chain. In certain embodiments, R1 is —ORA, wherein RA is an unsubstituted, unbranched C15 alkyl chain. In certain embodiments, R1 is —ORA, wherein RA is an unsubstituted, unbranched C16 alkyl chain. In certain embodiments, R1 is —ORA, wherein RA is an unsubstituted, unbranched C17 alkyl chain. In certain embodiments, R1 is —ORA, wherein RA is an unsubstituted, unbranched C18 alkyl chain. In certain embodiments, R1 is —ORA, wherein RA is an unsubstituted, unbranched C19 alkyl chain. In certain embodiments, R1 is —ORA, wherein RA is an unsubstituted, unbranched C20 alkyl chain. In yet other embodiments, RA is a substituted or unsubstituted aryl or heteroaryl moiety.

In certain embodiments, R2 is hydrogen. In other embodiments, R2 is a cyclic or acyclic, substituted or unsubstituted, branched or un branched aliphatic or heteroaliphatic moiety. In certain embodiments, R2 is a substituted or unsubstituted aryl or heteroaryl moiety. Preferably, the aryl or heteroaryl moiety is a monocylic 5- or 6-membered ring system. In certain embodiments, R2 is —ORB, —SRB, —N(RB)2, or —NHRB. In certain embodiments, R2 is —ORB. In other embodiments, R2 is —N(RB)2 or —NHRB. In certain embodiments, RB is hydrogen. In other embodiments, RB is a cyclic or acyclic, substituted or unsubstituted, branched or unbranched aliphatic or heteroaliphatic moiety. In certain embodiments, RB is an acyclic, substituted or unsubstituted aliphatic moiety. In certain other embodiments, RB is an acyclic, unsubstituted, unbranched aliphatic moiety, preferably C6-C30, more preferably C10-C20. In certain embodiments, RB is an unsubstituted, straight chain alkyl group, preferably C6-C30, more preferably C10-C20. In certain embodiments, R2 is —ORB, wherein RB is an unsubstituted, unbranched C9 alkyl chain. In certain embodiments, R2 is —ORB, wherein RB is an unsubstituted, unbranched C10 alkyl chain. In certain embodiments, R2 is —ORB, wherein RB is an unsubstituted, unbranched C11 alkyl chain. In certain embodiments, R2 is —ORB, wherein RB is an unsubstituted, unbranched C12 alkyl chain. In certain embodiments, R2 is —ORB, wherein RB is an unsubstituted, unbranched C13 alkyl chain. In certain embodiments, R2 is —ORB, wherein RB is an unsubstituted, unbranched C14 alkyl chain. In certain embodiments, R2 is —ORB, wherein RB is an unsubstituted, unbranched C15 alkyl chain. In certain embodiments, R2 is —ORA, wherein RB is an unsubstituted, unbranched C16 alkyl chain. In certain embodiments, R2 is —ORA, wherein RB is an unsubstituted, unbranched C17 alkyl chain. In certain embodiments, R2 is —ORA, wherein RB is an unsubstituted, unbranched C18 alkyl chain. In certain embodiments, R2 is —ORB, wherein RB is an unsubstituted, unbranched C19 alkyl chain. In certain embodiments, R2 is —ORB, wherein RB is an unsubstituted, unbranched C20 alkyl chain. In yet other embodiments, RB is a substituted or unsubstituted aryl or heteroaryl moiety.

In certain embodiments, R3 is hydrogen. In certain embodiments, R3 is a cyclic or acyclic, substituted or unsubstituted, branched or unbranched aliphatic moiety. In other embodiments, R3 is a cyclic or acyclic, substituted or unsubstituted, branched or unbranched heteroaliphatic moiety. In certain embodiments, R3 is an aliphatic moiety substituted with one or more hydroxyl groups. In other embodiments, R3 is an aliphatic moiety substituted with one or more amino, alkylamino, or dialkylamino groups. In certain embodiments, R3 is C1-C6 alkyl. In certain embodiments, R3 is hydrogen. In certain embodiments, R3 is a heteroaliphatic moiety. In certain embodiments, R3 is cyclic aliphatic, preferably a monocylic ring system with a 5- or 6-membered ring. In other embodiments, R3 is aryl or heteroaryl, preferably a monocyclic ring system with a 5- or 6-membered ring. In certain embodiments, R3 is

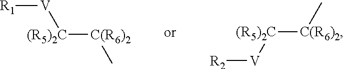

wherein R1, R2, R5, R6, and V are defined as above.

In other embodiments, R4 is hydrogen. In certain embodiments, R4 is a cyclic or acyclic, substituted or unsubstituted, branched or unbranched aliphatic moiety. In other embodiments, R4 is a cyclic or acyclic, substituted or unsubstituted, branched or unbranched heteroaliphatic moiety. In certain embodiments, R4 is an aliphatic moiety substituted with one or more hydroxyl groups. In other embodiments, R4 is an aliphatic moiety substituted with one or more amino, alkylamino, or dialkylamino groups. In certain embodiments, R4 is C1-C6 alkyl. In certain embodiments, R4 is hydrogen. In certain embodiments, R4 is a heteroaliphatic moiety. In certain embodiments, R3 is cyclic aliphatic, preferably a monocylic ring system with a 5- or 6-membered ring. In other embodiments, R4 is aryl or heteroaryl, preferably a monocyclic ring system with a 5- or 6-membered ring. In certain embodiments, R4 is

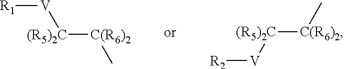

wherein R1, R2, R5, R6, and V are defined as above.

In certain embodiments, R3 and R4 are the same. In other embodiments, R3 and R4 are different. In certain embodiments, both R3 and R4 are hydrogen. In certain embodiments, only one of R3 and R4 is hydrogen. In certain embodiments, both R3 and R4 are

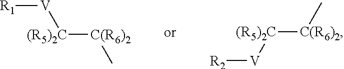

wherein R1, R2, R5, R6, and V are defined as above. In certain embodiments, one of R3 and R4 is

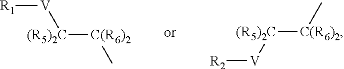

wherein R1, R2, R5, R6, and V are defined as above; and the other is hydrogen. In certain embodiments, both R3 and R4 are

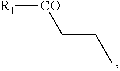

wherein R1 is as defined above.

In certain embodiments, one of R3 and R4 is

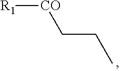

wherein R1 is defined as above; and the other is hydrogen.

In certain embodiments, each occurrence of R5 is hydrogen. In certain embodiments, at least one occurrence of R5 is methyl and the other occurrences are hydrogen. In certain embodiments, at least two occurrences of R5 are methyl, and the other occurrences are hydrogen. In other embodiments, at least two occurrences of R5 are hydrogen.

In certain embodiments, each occurrence of R6 is hydrogen. In certain other embodiments, at least two occurrences of R6 are hydrogen. In certain embodiments, at least one occurrence of R6 is methyl, and the other occurrences are hydrogen. In certain embodiments, at least two occurrences of R6 are methyl, and the other occurrences are hydrogen.

In certain embodiments, R7 is

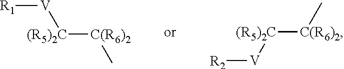

wherein R1, R2, R5, R6, and V are defined as above. In certain embodiments, R7,

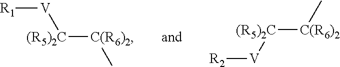

are the same. In other embodiments, R7,

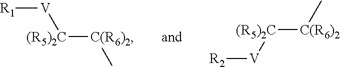

are different. In certain embodiments, R7 and

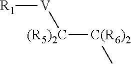

are the same. In other embodiments, R7 and

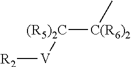

are the same. In certain embodiments, all R7 are the same.
In certain embodiments,

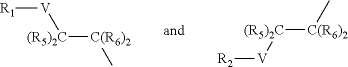

which are attached to N are the same. In other embodiments,

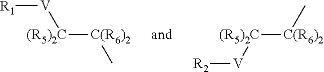

which are attached to N are the same and are different than R3 or R4.

In yet other embodiments,

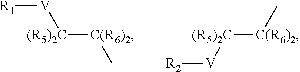

R3, and R4 are all different. In certain embodiments, R3 and R4 are the same. In other embodiments, R3 and R4 are different.

In certain embodiments,

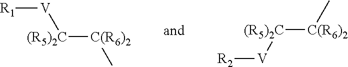

in formulae (III) and (IIIa) are selected from the group consisting of:

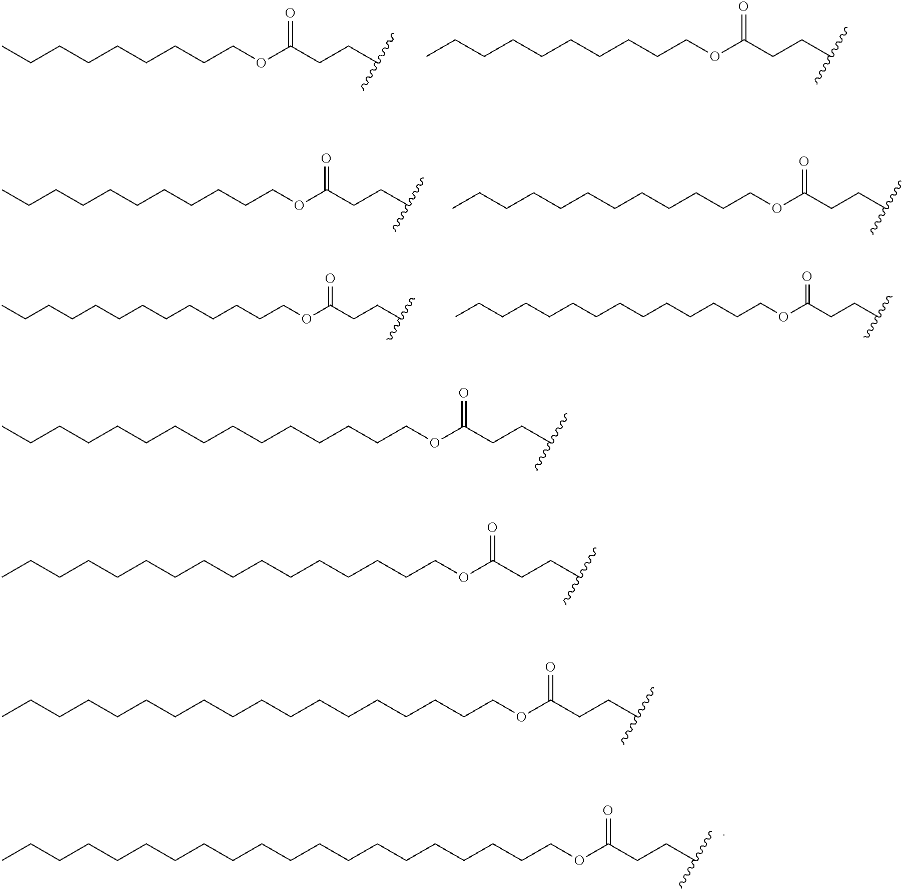

In certain embodiments, the lipids are prepared using acrylates LC, LD, LE, LF, and LG in FIG. 1A.

In certain embodiments

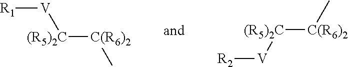

in formulae (III) and (IIIa) are selected from the group consisting of:

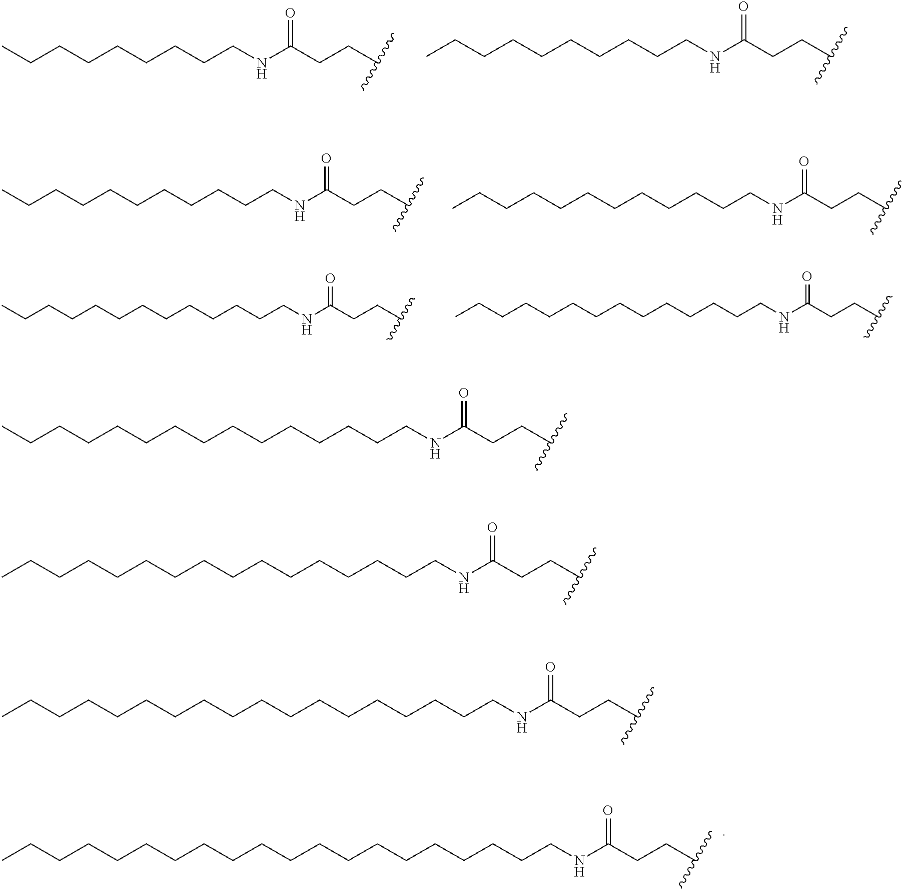

In certain embodiments, the lipids are prepared using acrylates NC, ND, NF, NG, and NP in FIG. 1A. In certain embodiments, the lipids are prepared using acrylate ND. In other embodiments, the lipids are prepared using acrylate NF.

In certain embodiments

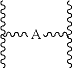

is selected from the group consisting of:

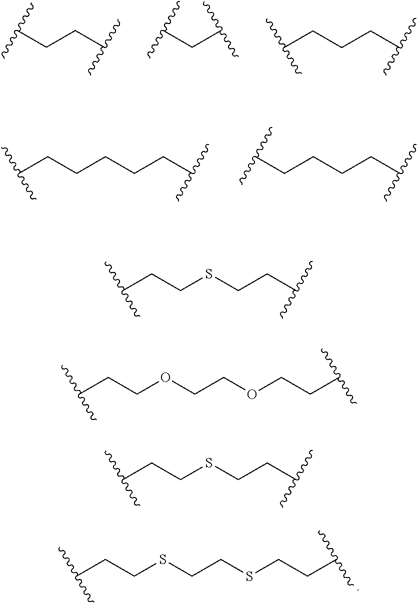

In certain particular embodiments,

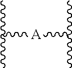

is

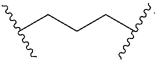

In certain particular embodiments,

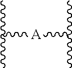

is

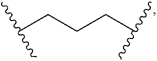

and n is 0, 1, 2, 3, 4, 5, or 6. In certain particular embodiments,

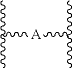

is

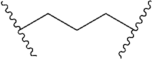

and n is 2.

In certain embodiments,

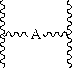

is

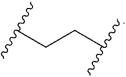

In certain embodiments,

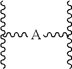

is

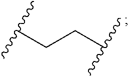

and n is 0, 1, 2, 3, 4, 5, or 6. In certain embodiments,

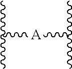

is

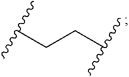

and n is 2.

In certain embodiments, the lipid is of the formula (IV):

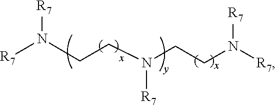

wherein each occurrence of x is an integer between 1 and 10, inclusive; preferably, between 1 and 6, inclusive; y is an integer between 0 and 10, inclusive; preferably, between 0 and 6, inclusive; each occurrence of R7 is hydrogen; substituted or unsubstituted, branched or unbranched aliphatic; substituted or unsubstituted, branched or unbranched heteroaliphatic; substituted or unsubstituted aryl; substituted or unsubstituted heteroaryl; or

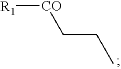

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; and salts thereof. In certain embodiments, x is 1, 2, 3, 4, or 5. In certain particular embodiments, x is 1. In other particular embodiments, x is 2. In certain embodiments, y is 0. In certain embodiments, y is 1. In other embodiments, y is 2. In yet other embodiments, y is 3. In still other embodiments, y is 4. In certain embodiments, R1 is —ORA. In other embodiments, R1 is —NHRA. In certain embodiments, at least one R1 is C1-C20 alkyl. In certain embodiments, all R7 are of the formula

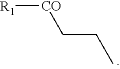

In certain embodiments, at least one R7 is branched or unbranched, substituted or unsubstituted aliphatic. In certain embodiments, at least one R7 is C1-C20 alkyl. In certain embodiments, at least one R7 is C1-C12 alkyl. In certain embodiments, at least one R7 is branched or unbranched, substituted or unsubstituted heteroaliphatic. In certain embodiments, at least one R7 is

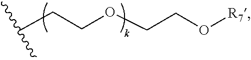

wherein k is an interger between 0 and 10, inclusive, and R7′ is hydrogen or C1-6alkyl. In certain embodiments, at least one R7 is

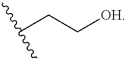

In other embodiments, at least one R7 is

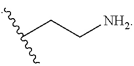

In other embodiments, at least one R7 is a hydrogen. In other embodiments, at least two R7 are each hydrogen. In still other embodiments, at least three R7 are each hydrogen. In still further embodiments, at least four R7 are each hydrogen.

In certain embodiments, each R7 in formulae (IV) is independently selected from the group consisting of hydrogen and

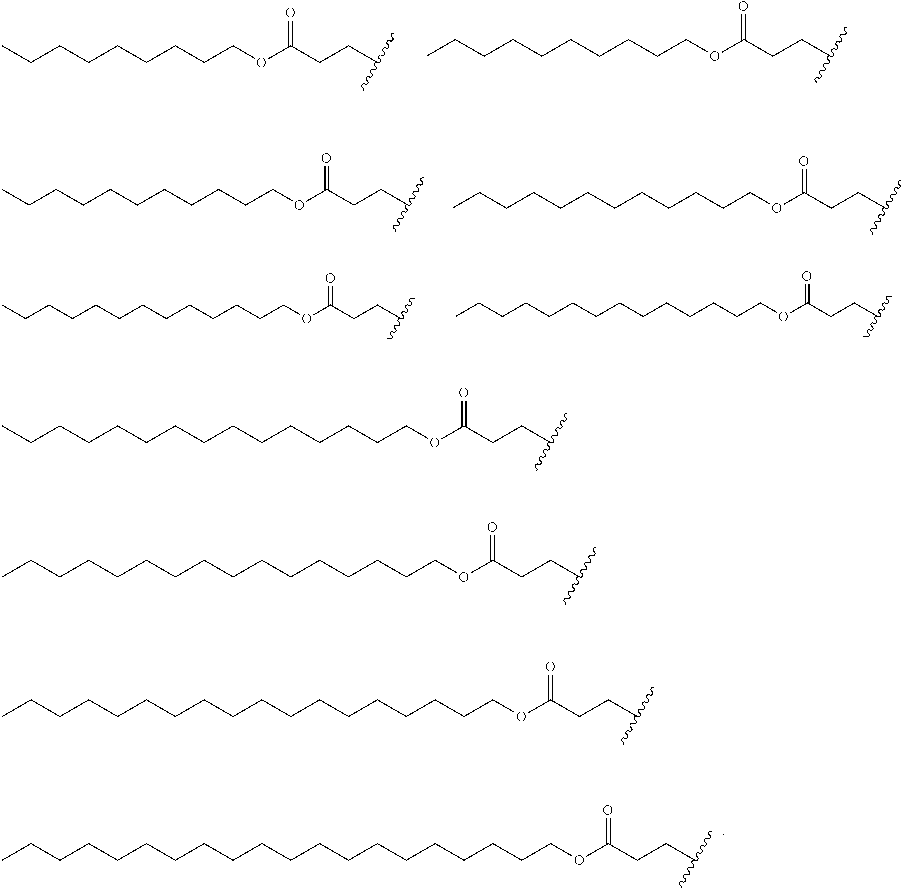

In certain embodiments, each R7 in formulae (IV) is independently selected from the group consisting of hydrogen and

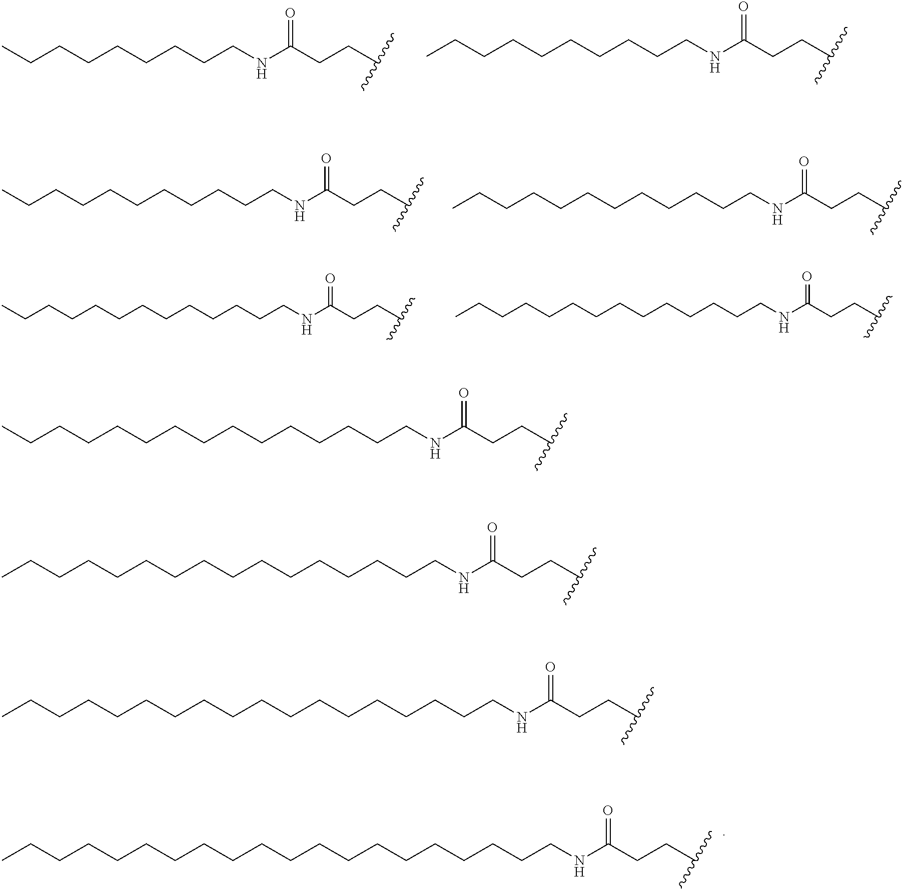

In certain embodiments, the lipid is of the formula (V), (VI), or (VII):

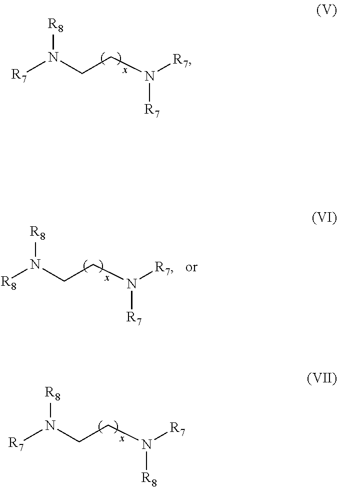

wherein x is an integer between 1 and 10, inclusive; preferably, between 1 and 6, inclusive; more preferably, between 1 and 3, inclusive; each occurrence of R7 is hydrogen or

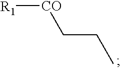

R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R8 is independently hydrogen, C1-C6 alkyl, hydroxy-C1-C6alkyl; or

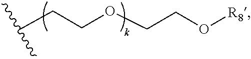

wherein k is an interger between 0 and 10, inclusive, and R8′ is hydrogen or C1-6alkyl; and salts thereof. In certain embodiments, x is 1, 2, 3, 4, or 5. In certain particular embodiments, x is 1. In other particular embodiments, x is 2. In other embodiments, x is 3. In certain embodiments, R1 is —ORA. In other embodiments, R1 is —NHRA. In certain embodiments, all R7 are of the formula

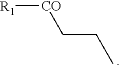

In other embodiments, at least one R7 is a hydrogen. In other embodiments, at least two R7 are each hydrogen. In still other embodiments, at least three R7 are each hydrogen. In still further embodiments, at least four R7 are each hydrogen. In certain embodiments, all R8 are the same. In certain particular embodiments, R8 is hydrogen. In certain embodiments, R8 is methyl. In other embodiments, R8 is ethyl. In yet other embodiments, R8 is hydroxymethyl. In still other embodiments, R8 is hydroxyethyl.

In certain embodiments, each R7 in formula (V), (VI), or (VII) is independently selected from the group consisting of hydrogen and

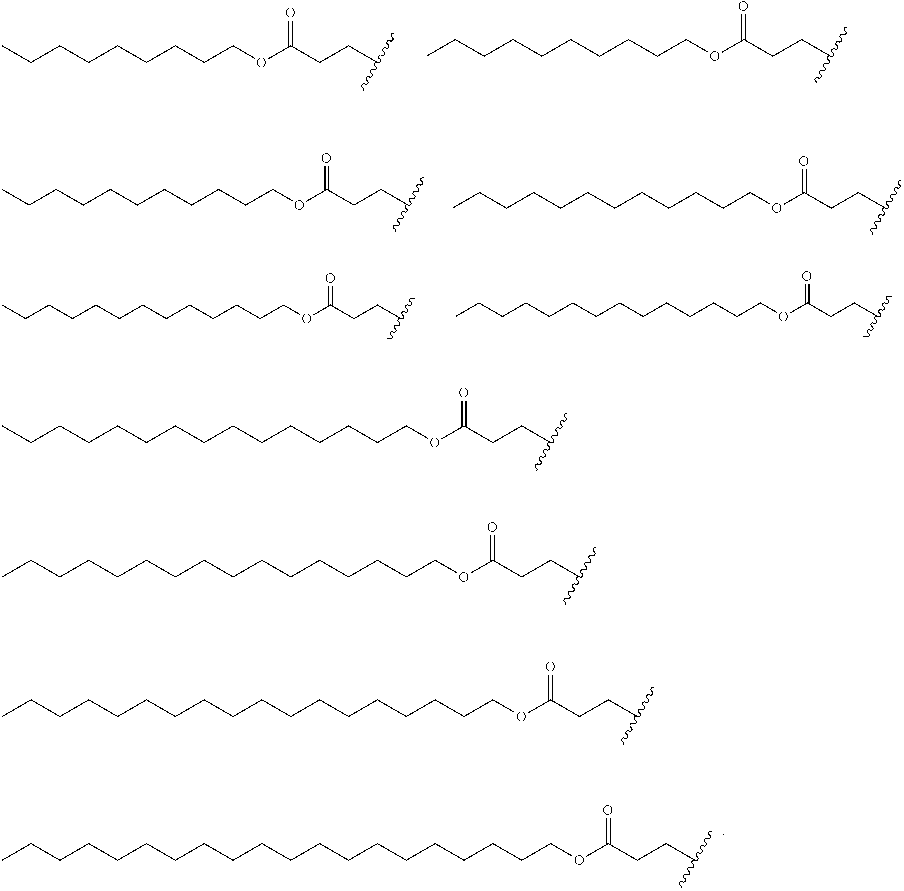

In certain embodiments, each R7 in formula (V), (VI), or (VII) is independently selected from the group consisting of hydrogen and

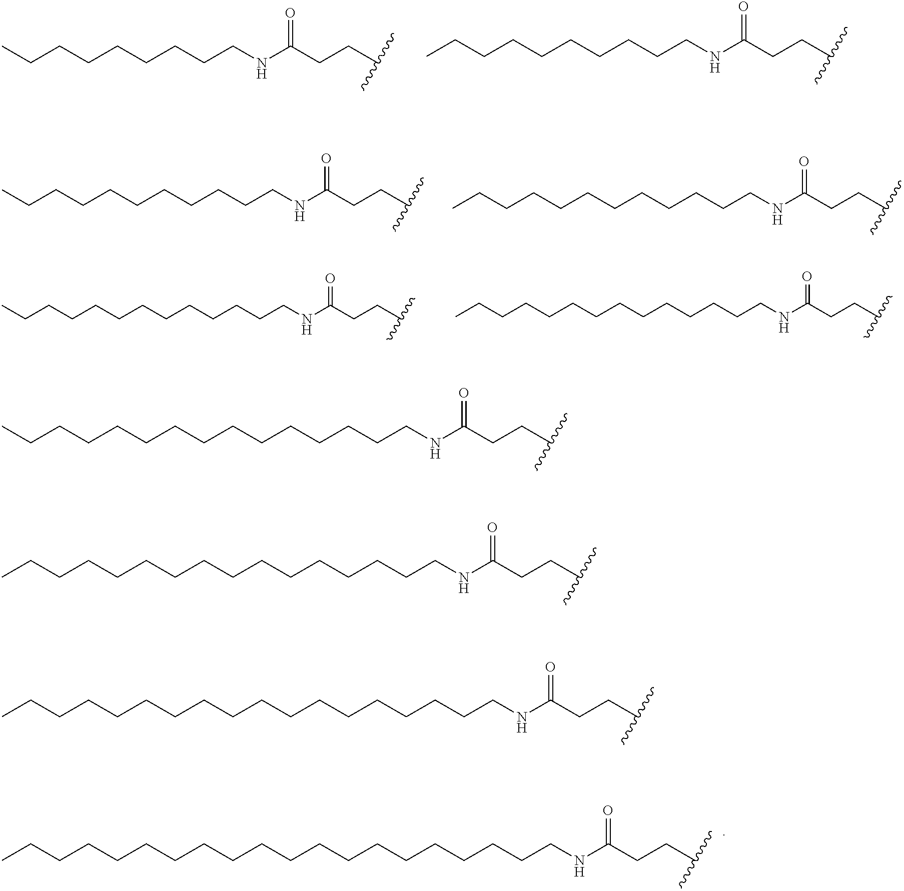

Exemplary compounds of the formulae (V), (VI), and (VII) are of the formula:

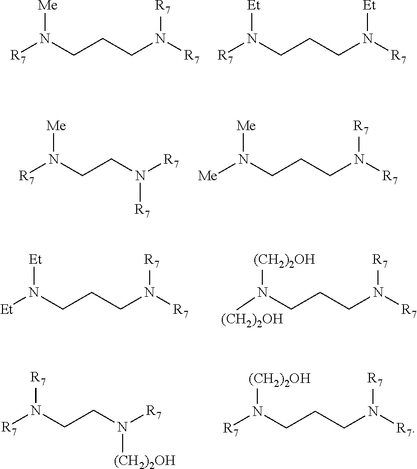

In certain embodiments, the lipid is one of the formulae:

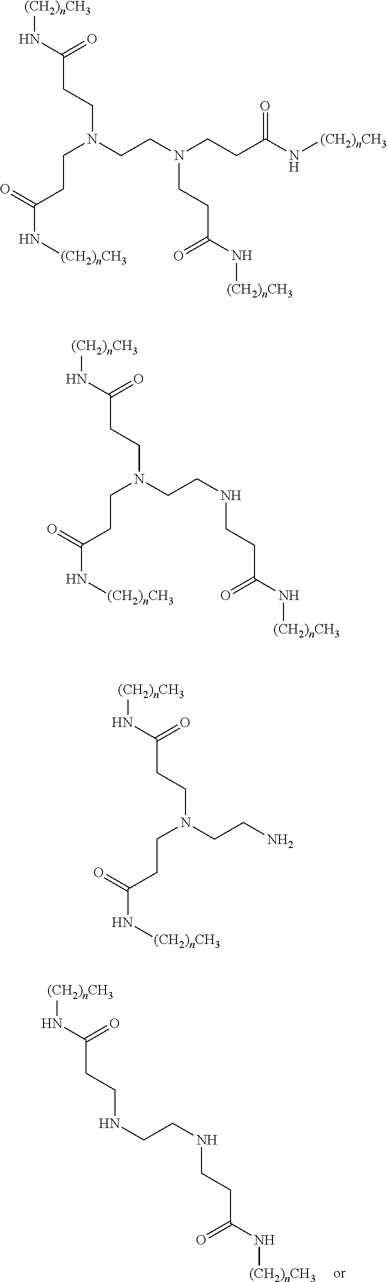

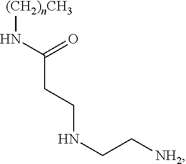

wherein n is an integer ranging from 1 to 15, inclusive; prefererably, n is an integer ranging from 6 to 12, inclusive, or 1 to 6, inclusive. In certain embodiments, n is 1, 2, 3, 4, 5, 6, 7, 8, 9, 10, 11, 12, 13, 14, or 15. In certain particular embodiments, n is 10, 11, or 12. In certain embodiments, n is 11. In other emboidments, n is 10. In certain embodiments, each n is independently an integer ranging from 1 to 15, inclusive. In other embodiments, all n are the same integer. In certain embodiments, one n is different from the other n in the compound.

In other embodiments, the compound is of one of the formulae:

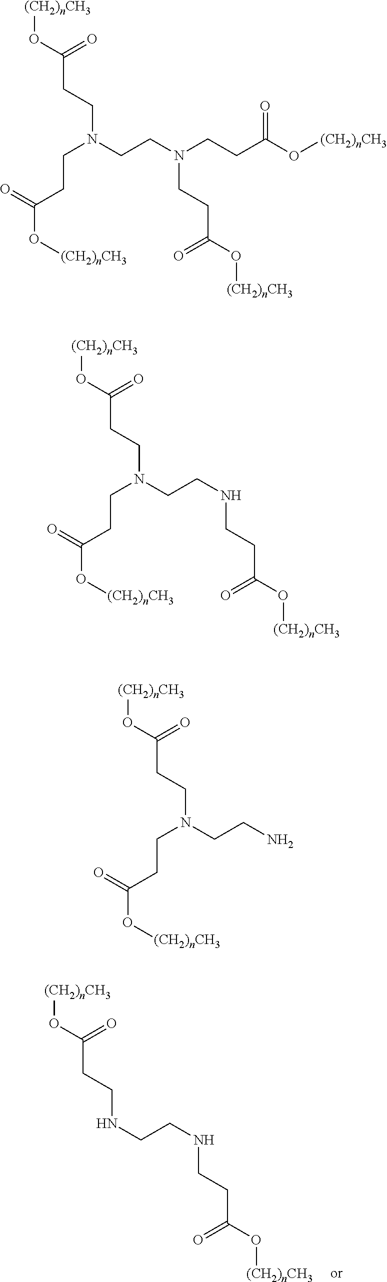

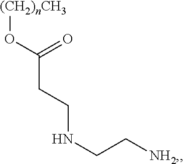

wherein n is an integer ranging from 1 to 15, inclusive; prefererably, n is an integer ranging from 6 to 12, inclusive, or 1 to 6, inclusive. In certain embodiments, n is 1, 2, 3, 4, 5, 6, 7, 8, 9, 10, 11, 12, 13, 14, or 15. In certain particular embodiments, n is 10, 11, or 12. In certain embodiments, n is 11. In other emboidments, n is 10. In certain embodiments, each n is independently an integer ranging from 1 to 15, inclusive. In other embodiments, all n are the same integer. In certain embodiments, one n is different from the other n in the compound.

In certain embodiments, the lipid is of one of the formulae:

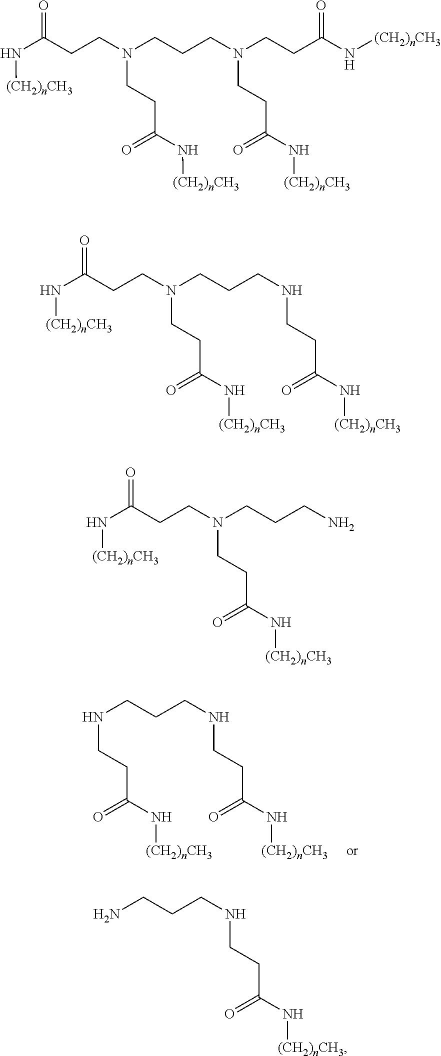

wherein n is an integer ranging from 1 to 15, inclusive; prefererably, n is an integer ranging from 6 to 12, inclusive, or 1 to 6, inclusive. In certain embodiments, n is 1, 2, 3, 4, 5, 6, 7, 8, 9, 10, 11, 12, 13, 14, or 15. In certain particular embodiments, n is 10, 11, or 12. In certain embodiments, n is 11. In other emboidments, n is 10. In certain embodiments, each n is independently an integer ranging from 1 to 15, inclusive. In other embodiments, all n are the same integer. In certain embodiments, one n is different from the other n in the compound.

In certain embodiments, the lipid is of one of the formulae:

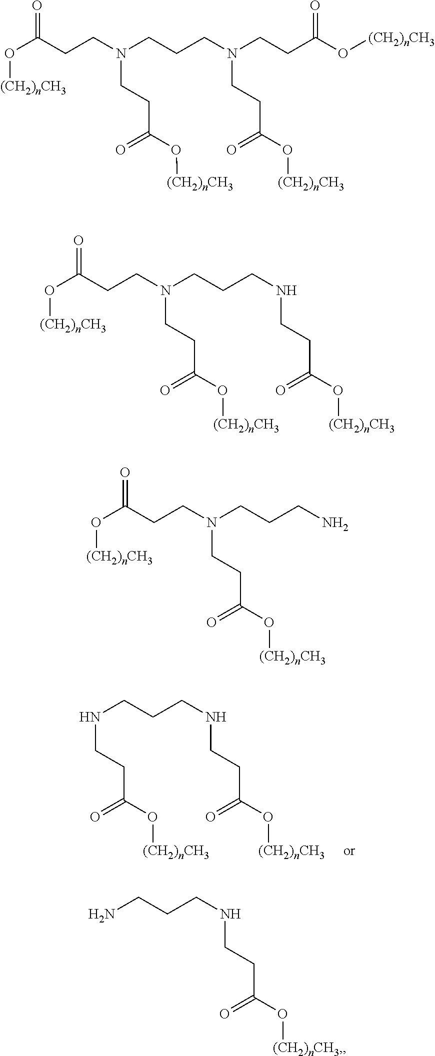

wherein n is an integer ranging from 1 to 15, inclusive; prefererably, n is an integer ranging from 6 to 12, inclusive, or 1 to 6, inclusive. In certain embodiments, n is 1, 2, 3, 4, 5, 6, 7, 8, 9, 10, 11, 12, 13, 14, or 15. In certain particular embodiments, n is 10, 11, or 12. In certain embodiments, n is 11. In other emboidments, n is 10. In certain embodiments, each n is independently an integer ranging from 1 to 15, inclusive. In other embodiments, all n are the same integer. In certain embodiments, one n is different from the other n in the compound.

In certain embodiments, the lipid is of one of the formulae:

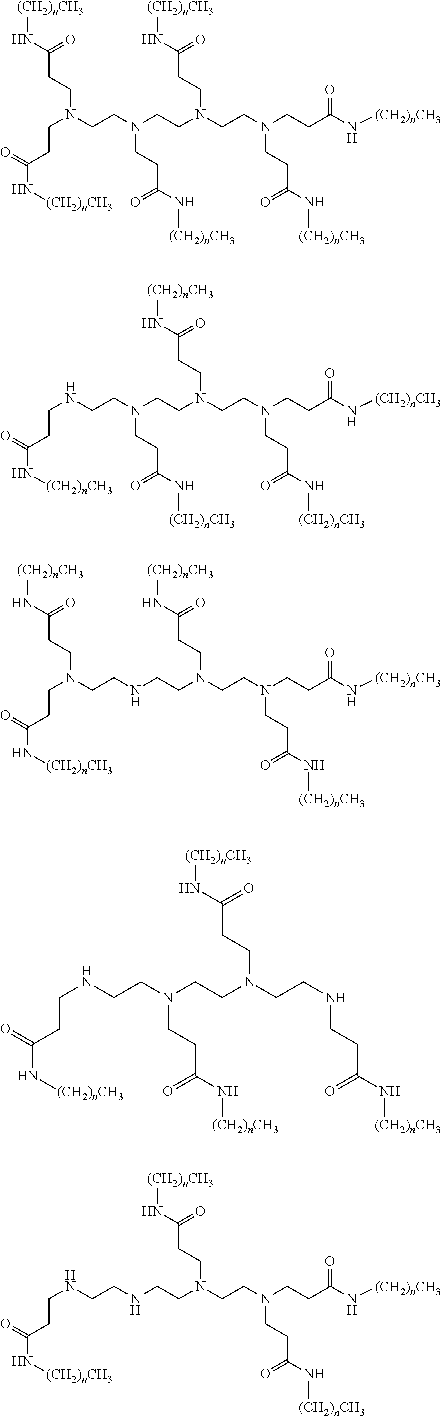

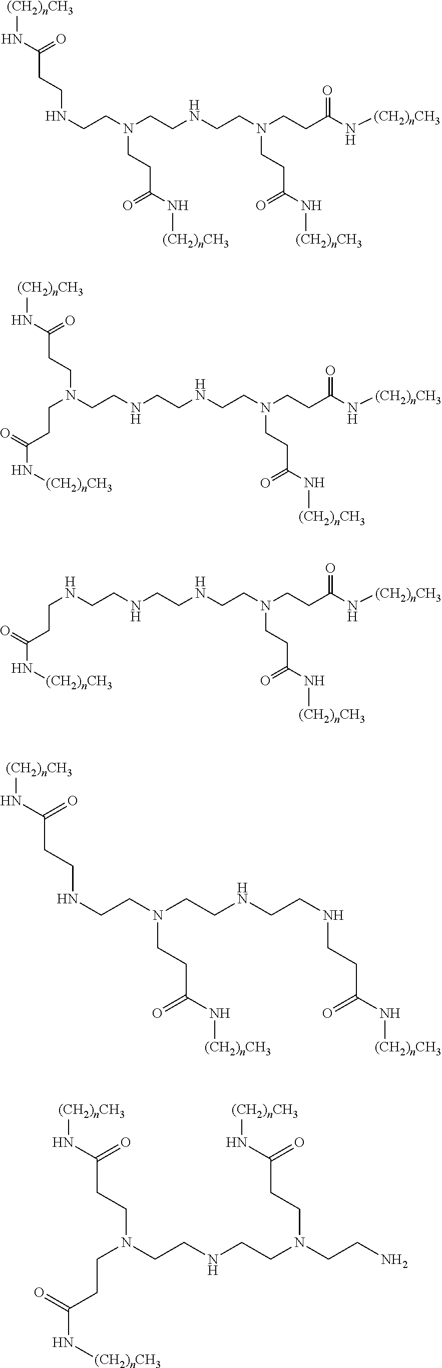

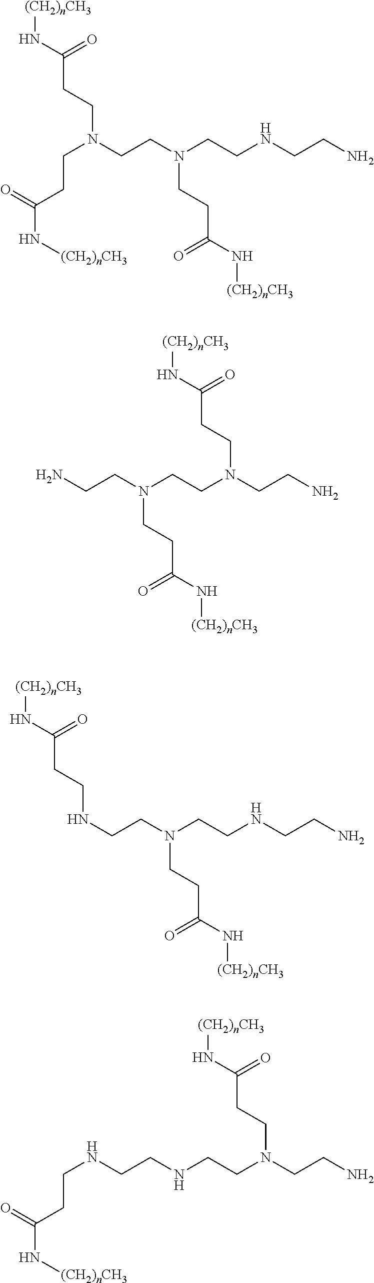

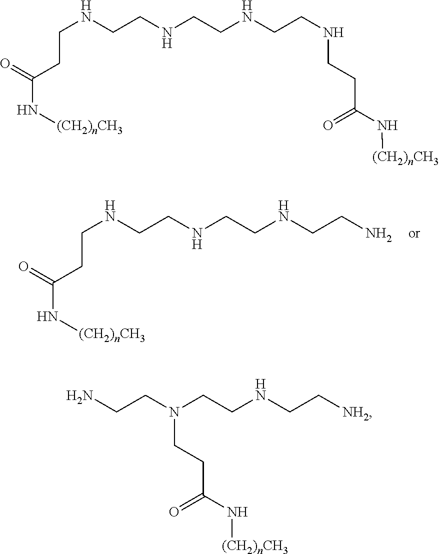

wherein n is an integer ranging from 1 to 15, inclusive; prefererably, n is an integer ranging from 6 to 12, inclusive, or 1 to 6, inclusive. In certain embodiments, n is 1, 2, 3, 4, 5, 6, 7, 8, 9, 10, 11, 12, 13, 14, or 15. In certain particular embodiments, n is 10, 11, or 12. In certain embodiments, n is 11. In other emboidments, n is 10. In certain embodiments, each n is independently an integer ranging from 1 to 15, inclusive. In other embodiments, all n are the same integer. In certain embodiments, one n is different from the other n in the compound.

In certain embodiments, the lipid is of one of the formulae:

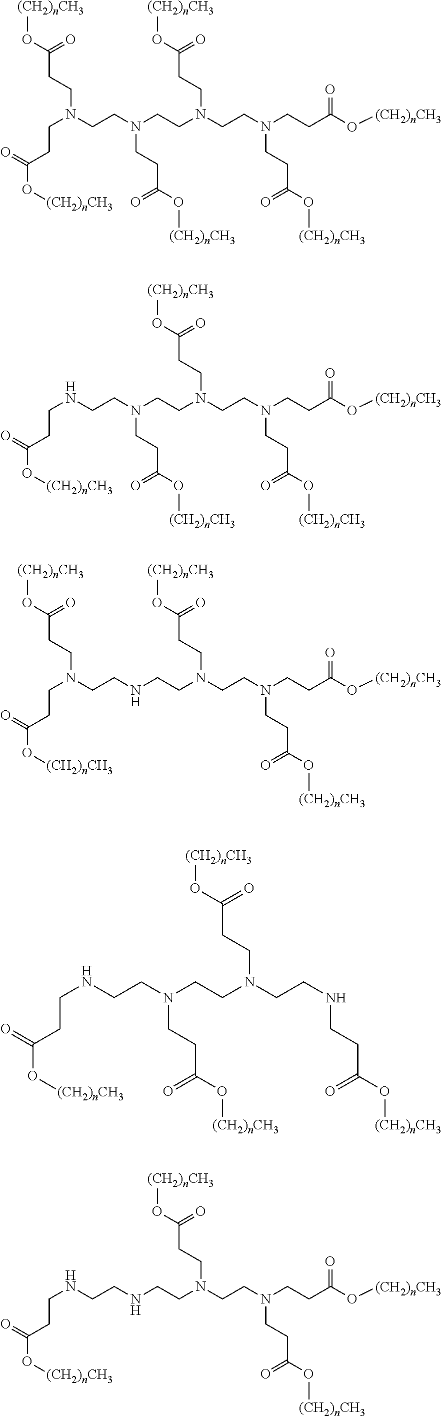

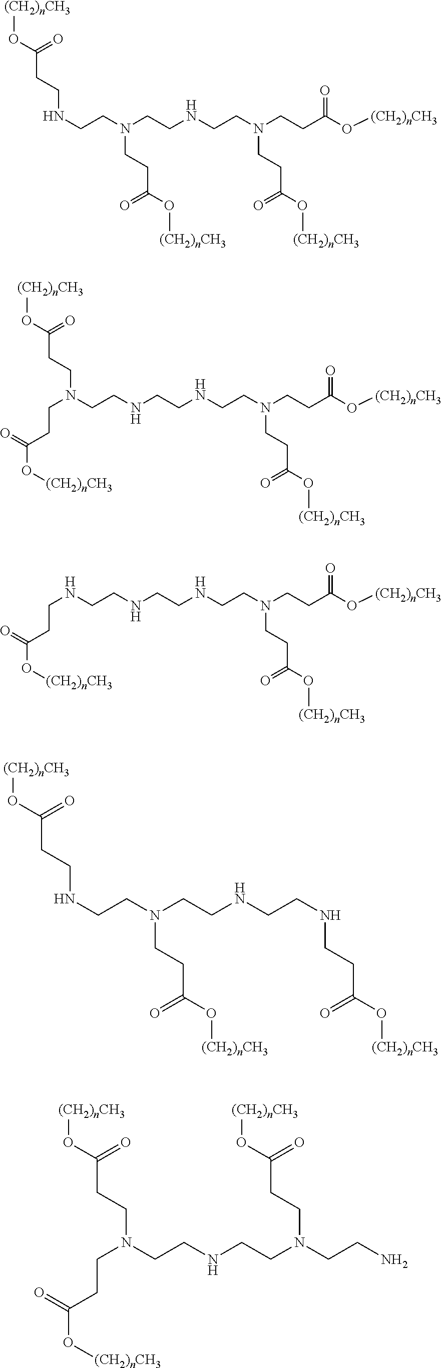

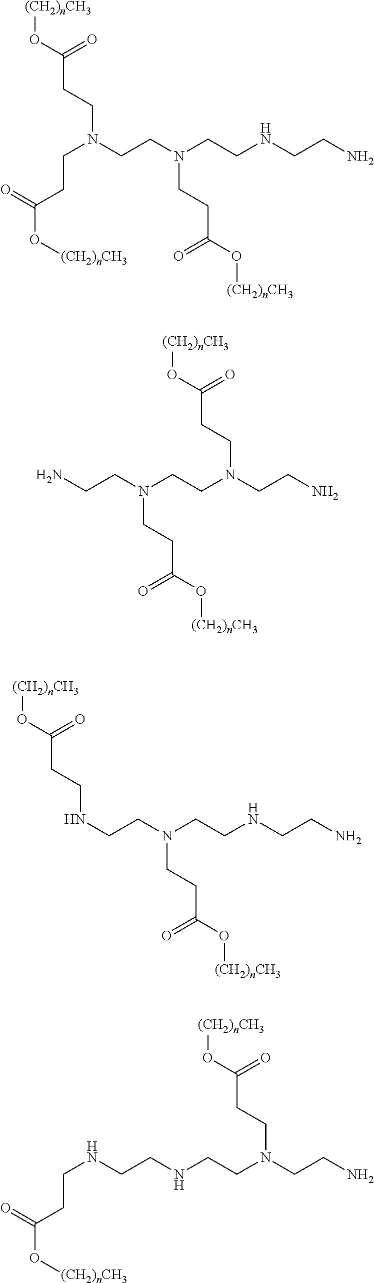

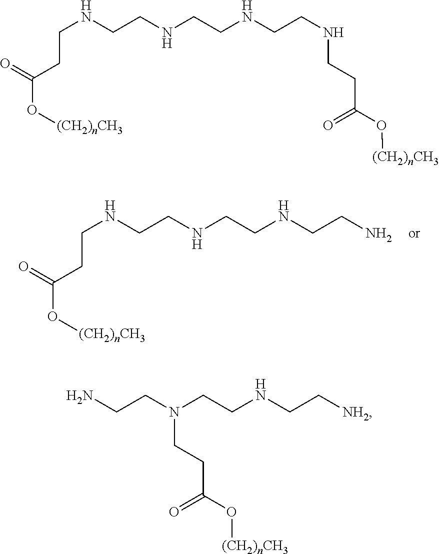

wherein n is an integer ranging from 1 to 15, inclusive; prefererably, n is an integer ranging from 6 to 12, inclusive, or 1 to 6, inclusive. In certain embodiments, n is 1, 2, 3, 4, 5, 6, 7, 8, 9, 10, 11, 12, 13, 14, or 15. In certain particular embodiments, n is 10, 11, or 12. In certain embodiments, n is 11. In other emboidments, n is 10. In certain embodiments, each n is independently an integer ranging from 1 to 15, inclusive. In other embodiments, all n are the same integer. In certain embodiments, one n is different from the other n in the compound.

In another aspect of the invention, the lipid or composition of lipids of the invention is lipid or composition prepared by reacting an amine of one of the formula (I-117):

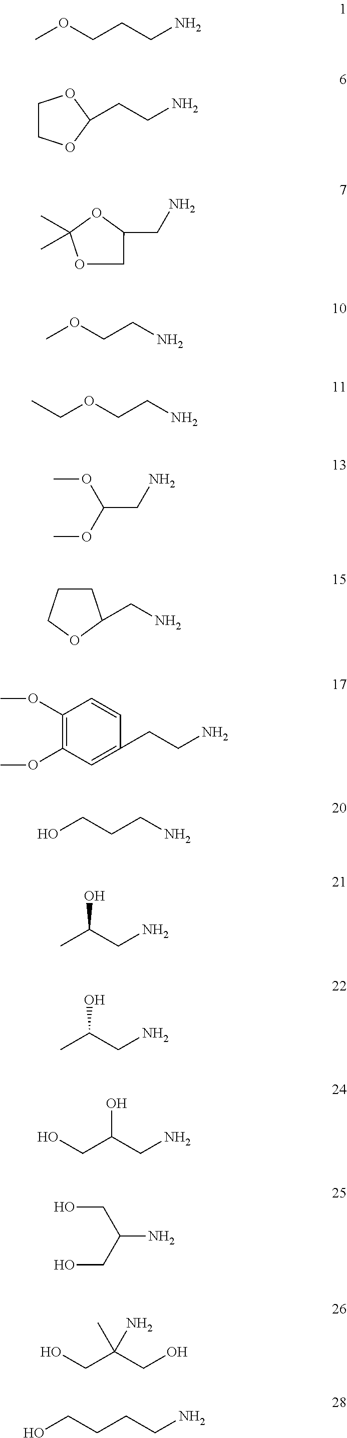

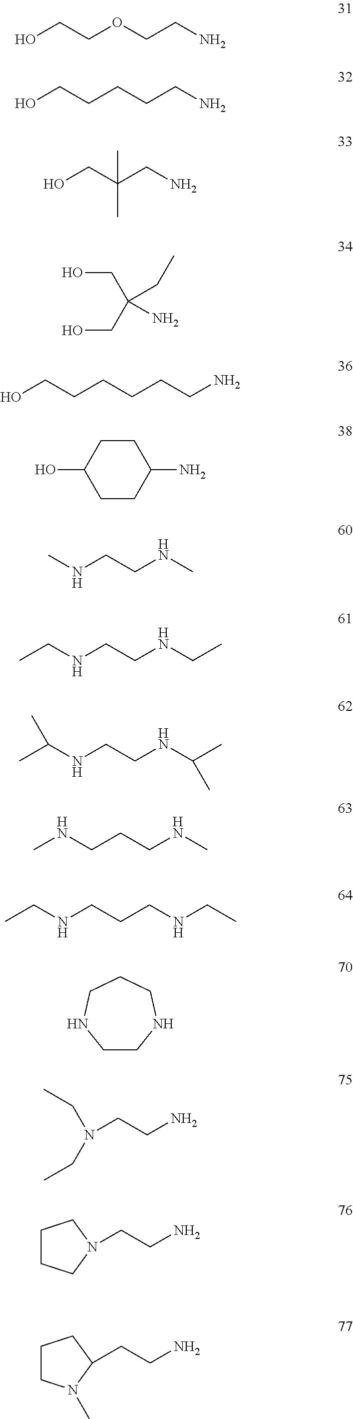

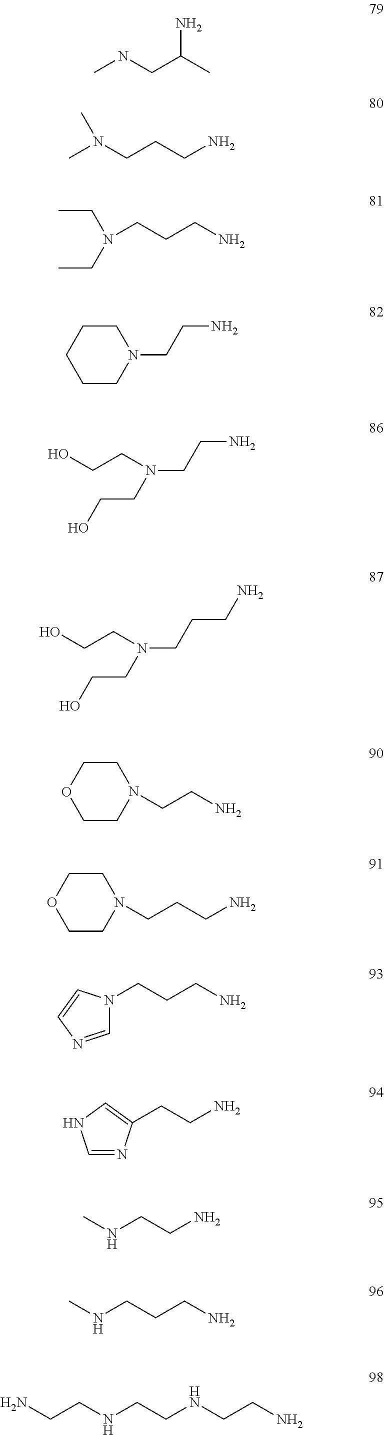

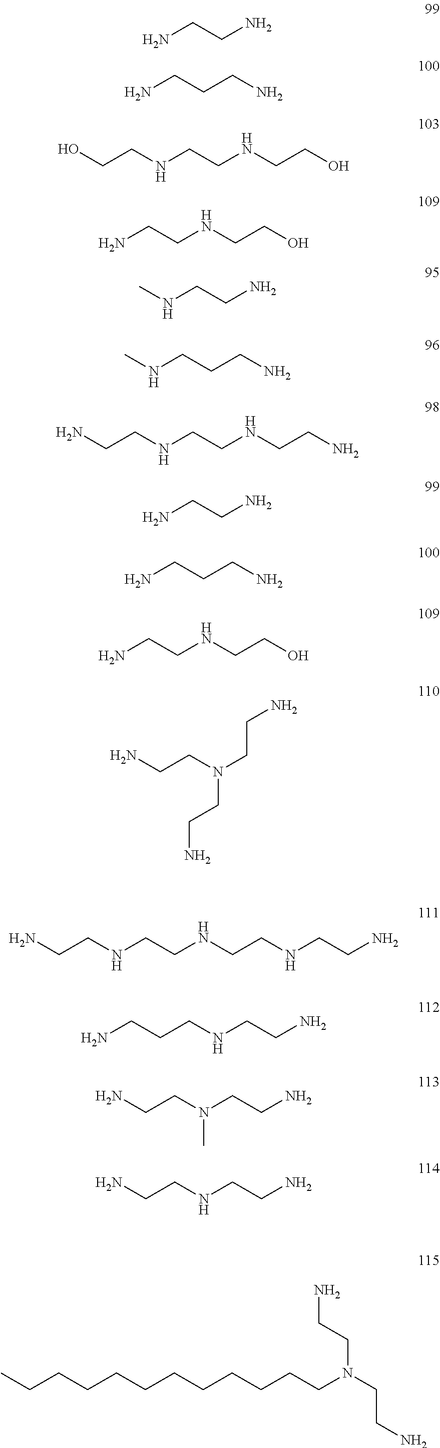

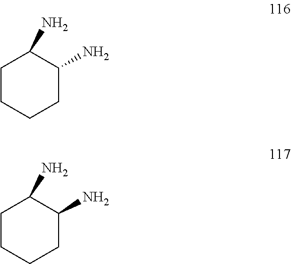

with an acrylate of formula:

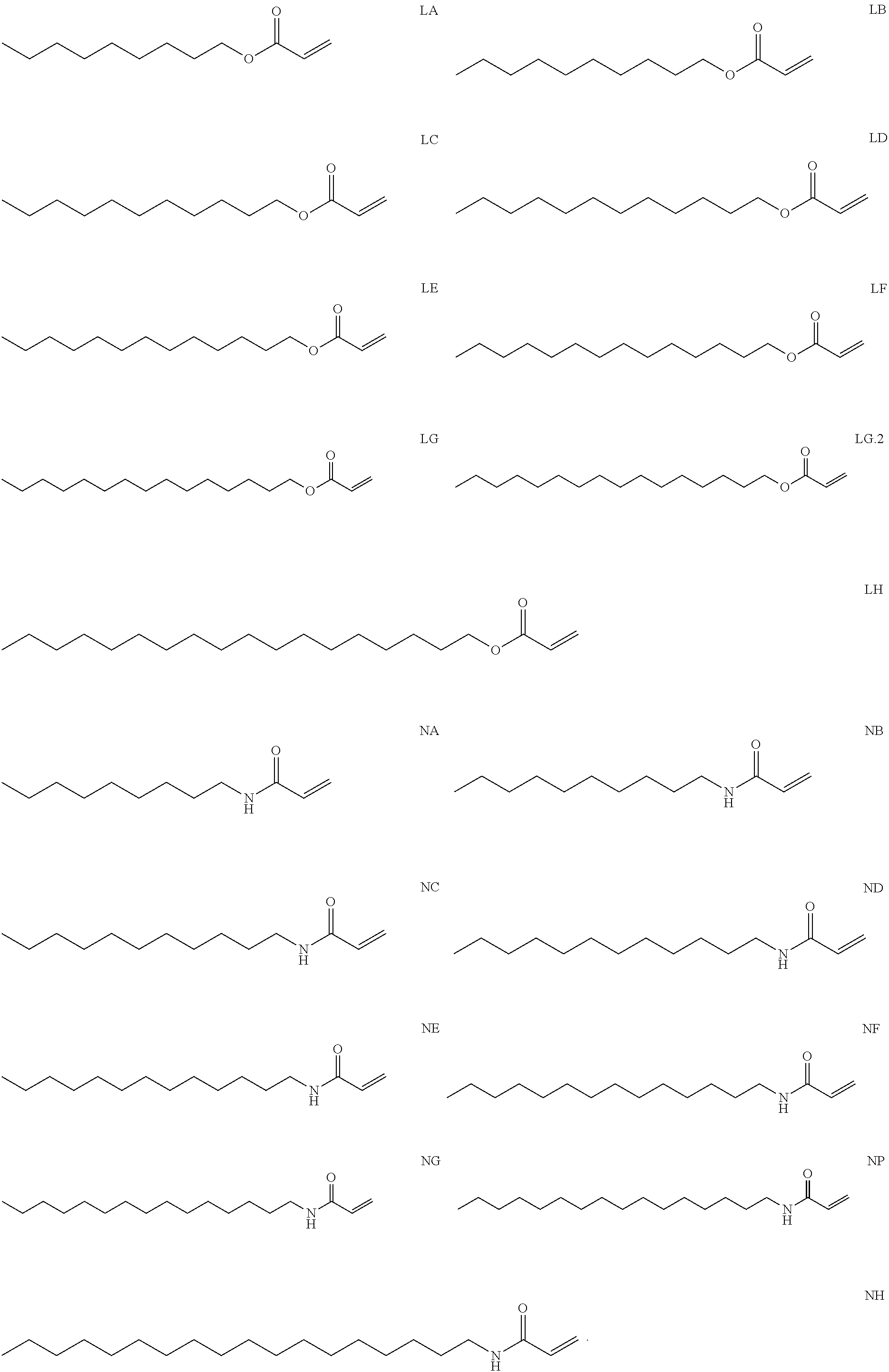

In certain embodiments, one equivalent of amine is reacted with one equivalent of acrylate. In certain embodiments, one equivalent of amine is reacted with one, two, three, four, five, six, or more equivalents of acrylate. In certain embodiments, the amount of acrylate is limiting to prevent the functionalization of all amino groups. The resulting lipid or lipid composition in these instances contain secondary amino groups or primary amino groups. Lipids having secondary amines are particular useful in certain instances. In certain embodiments, amine-containing lipids that have not been fully functionalize are further reacted with another electrophile (e.g., an acrylate, acrylamide, alkylating agent, acylating agent, etc.). Such further functionalization of the amines of the lipid results in lipids with different tails. One, two, three, four, five, or more tails may be different from the other tails of the lipid.

In certain embodiments, the amine and acrylate are reacted together neat. In other embodiments, the reaction is done in a solvent (e.g., THF, CH2Cl2, MeOH, EtOH, CHCl3, hexanes, toluene, benzene, CCl4, glyme, diethyl ether, etc.). In certain embodiments, the reaction mixture is heated. In a particularly preferred embodiment, the reaction mixture is heated to temperature ranging from 50-150° C. In another particularly preferred embodiment, the reaction mixture is heated to approximately 95° C. The reaction may also be catalyzed. For example, the reaction may be catalyzed by the addition of an acid, base, or metal. The reaction may be allowed to proceed for hours, days, or weeks. In certain embodiments, the reaction is allowed to proceed for 1-7 days, preferably 7 days. The resulting composition may be used with or without purification. In certain embodiments, the lipids are subsequently subjected to an alkylation step (e.g., reaction with methyl iodide) to form quanternary amine salts. Optionally, various salt forms of the lipids may be prepared. In certain embodiments, the salts are pharmaceutically acceptable salts.

In certain embodiments, the lipid is prepared by reacting amine 98 with acrylate NC to form lipid NC98. In certain embodiments, the lipid NC98 is of one of the formulae below:

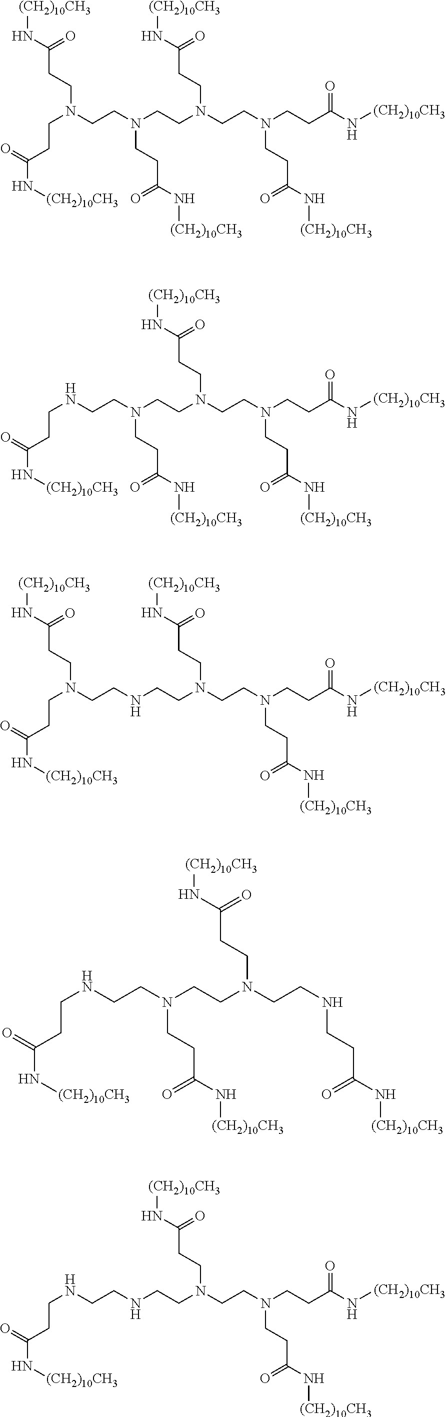

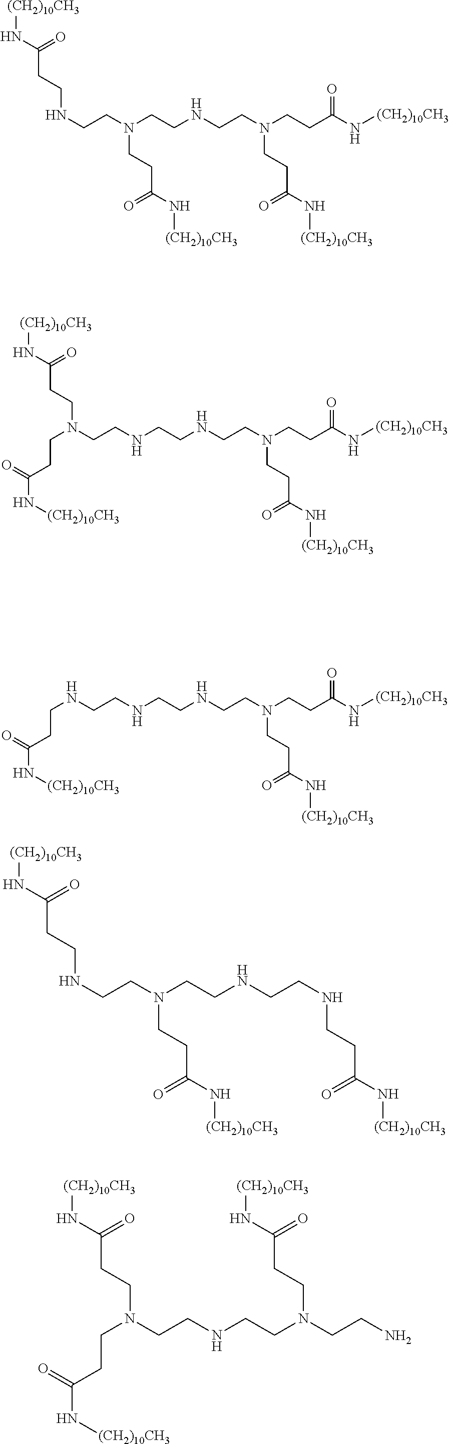

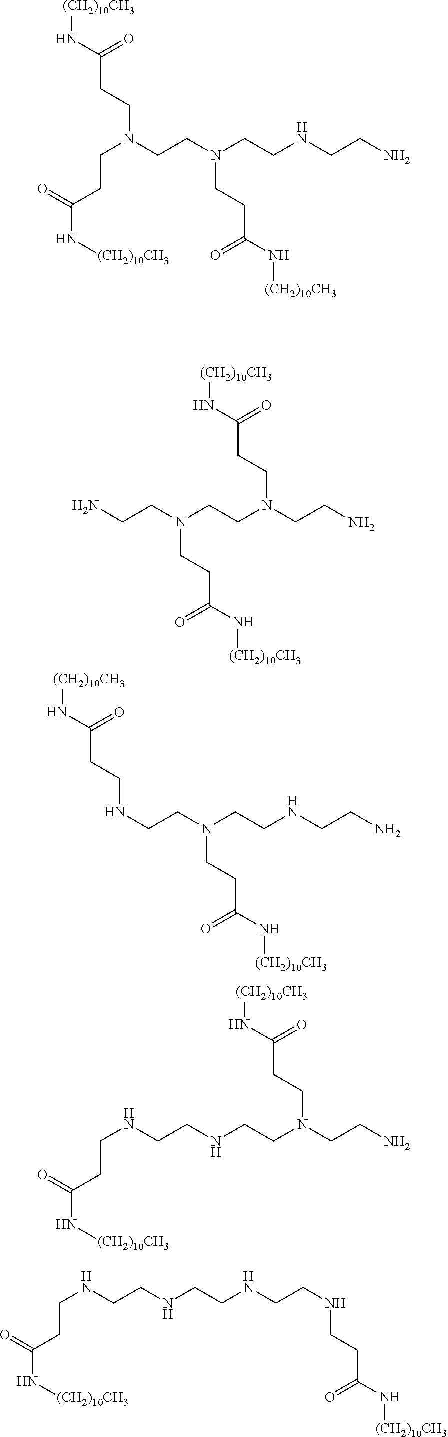

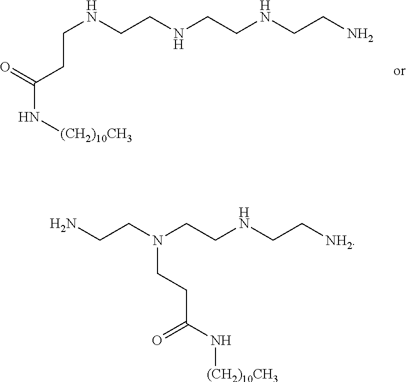

In other embodiments, the lipid is a composition of one or more of the above lipids.

In certain embodiments, the lipid is prepared by reacting amine 99 with acrylate NC to form lipid NC99. In certain embodiments, the lipid NC99 is of one of the formulae below:

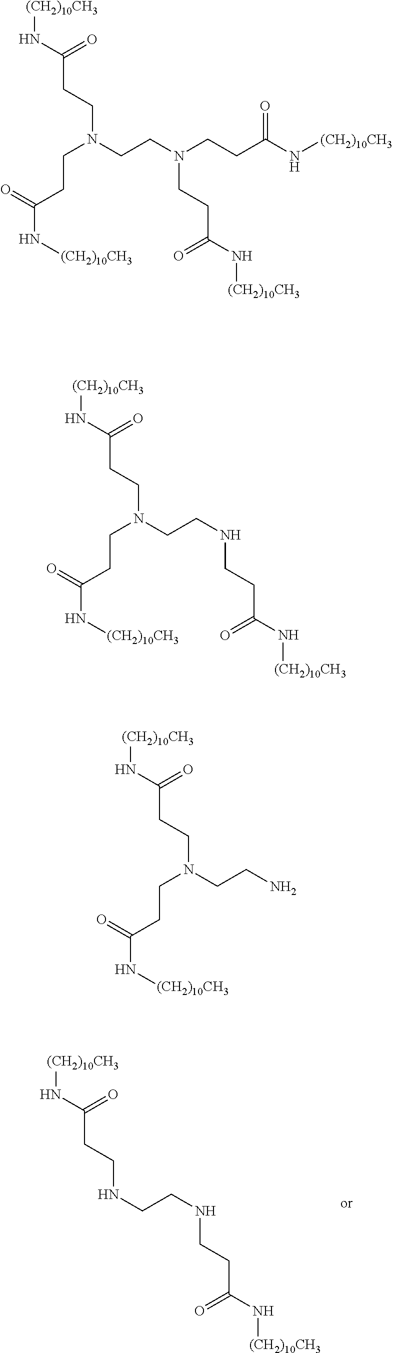

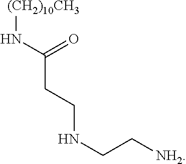

In other embodiments, the lipid is a composition of one or more of the above lipids.

In certain embodiments, the lipid is prepared by reacting amine 100 with acrylate NC to form lipid NC100. In certain embodiments, the lipid NC100 is of one of the formulae below:

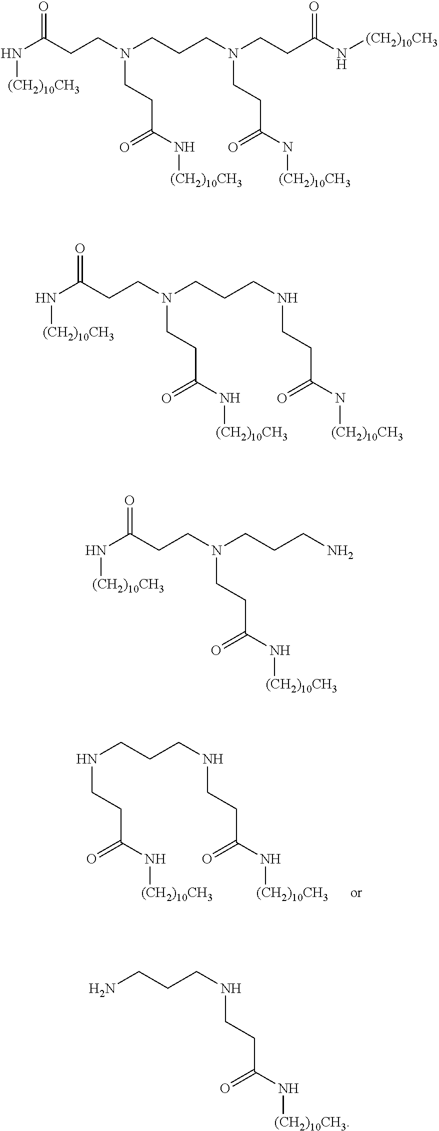

In other embodiments, the lipid is a composition of one or more of the above lipids.

In certain embodiments, the lipid is prepared by reacting amine 20 with acrylate ND to form lipid ND20. In certain embodiments, the lipid ND20 is of one of the formulae below:

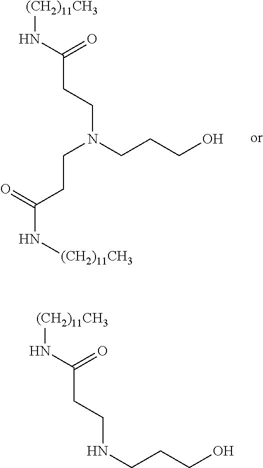

In other embodiments, the lipid is a composition of one or more of the above lipids.

In certain embodiments, the lipid is prepared by reacting amine 24 with acrylate ND to form lipid ND24. In certain embodiments, the lipid ND24 is of one of the formulae below:

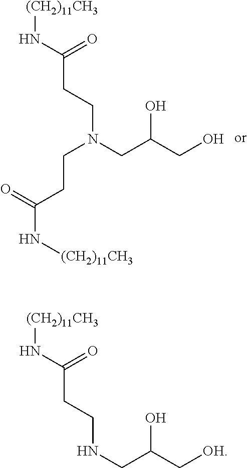

In other embodiments, the lipid is a composition of one or more of the above lipids.

In certain embodiments, the lipid is prepared by reacting amine 25 with acrylate ND to form lipid ND25. In certain embodiments, the lipid ND25 is of one of the formulae below:

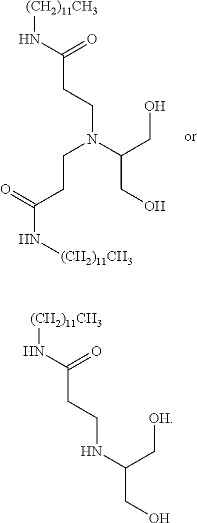

In other embodiments, the lipid is a composition of one or more of the above lipids.

In certain embodiments, the lipid is prepared by reacting amine 28 with acrylate ND to form lipid ND28. In certain embodiments, the lipid ND28 is of one of the formulae below:

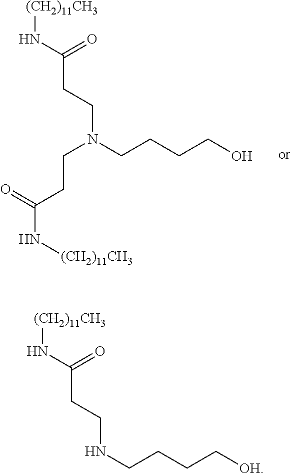

In other embodiments, the lipid is a composition of one or more of the above lipids.

In certain embodiments, the lipid is prepared by reacting amine 32 with acrylate ND to form lipid ND32. In certain embodiments, the lipid ND32 is of one of the formulae below:

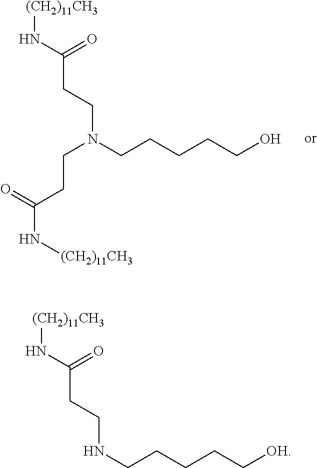

In other embodiments, the lipid is a composition of one or more of the above lipids.

In certain embodiments, the lipid is prepared by reacting amine 36 with acrylate ND to form lipid ND36. In certain embodiments, the lipid ND36 is of one of the formulae below:

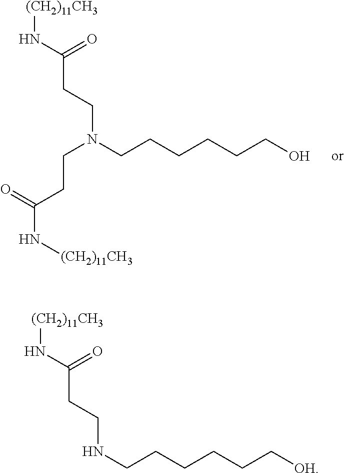

In other embodiments, the lipid is a composition of one or more of the above lipids.

In certain embodiments, the lipid is prepared by reacting amine 98 with acrylate ND to form lipid ND98. In certain embodiments, the lipid ND98 is of one of the formulae below:

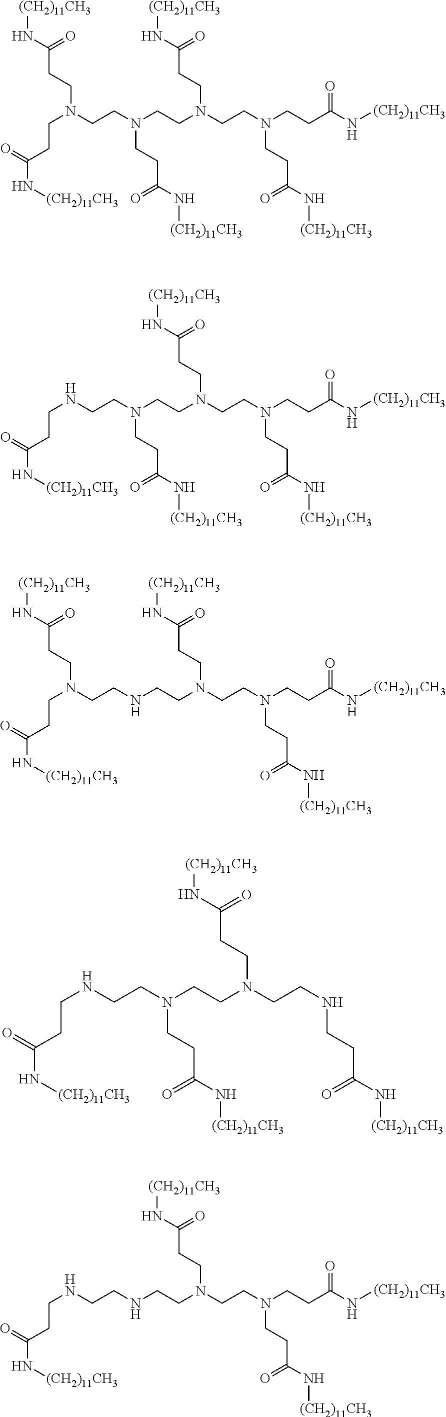

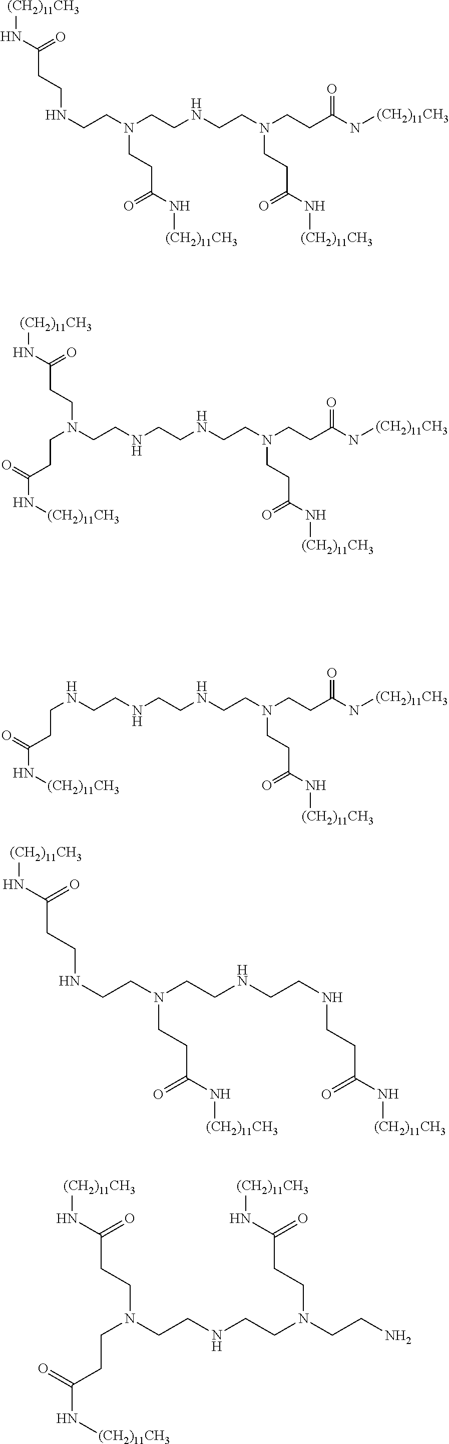

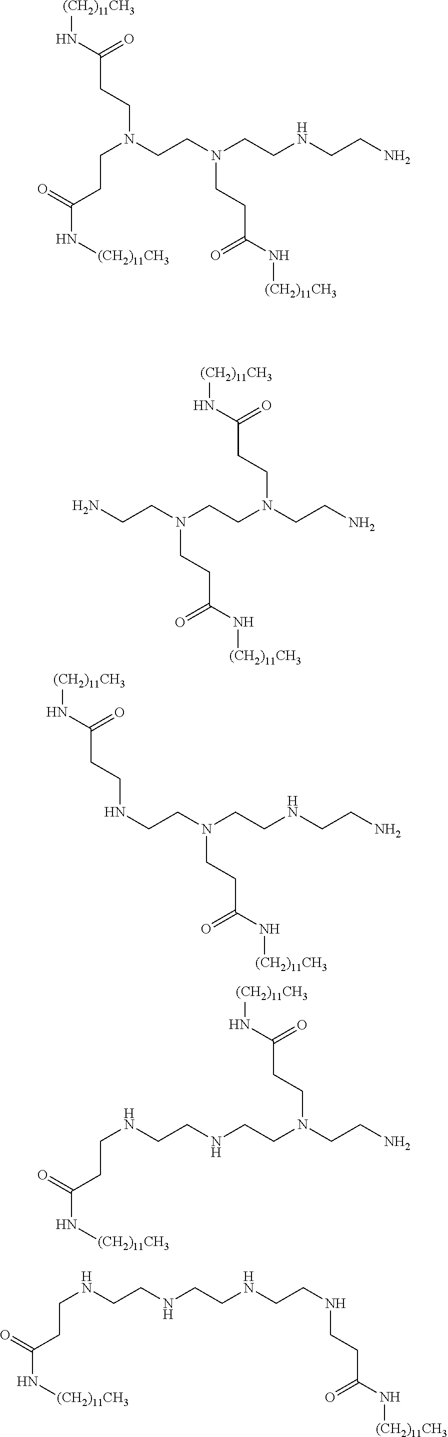

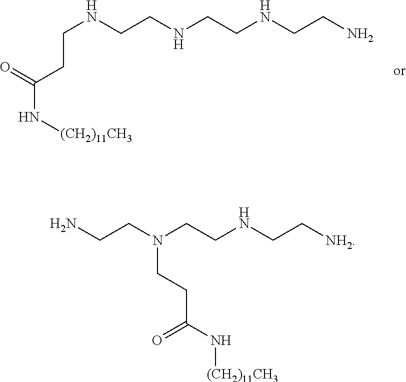

In other embodiments, the lipid is a composition of one or more of the above lipids.

In certain embodiments, the lipid is prepared by reacting amine 94 with acrylate ND to form lipid ND94. In certain embodiments, the lipid ND94 is of one of the formulae below:

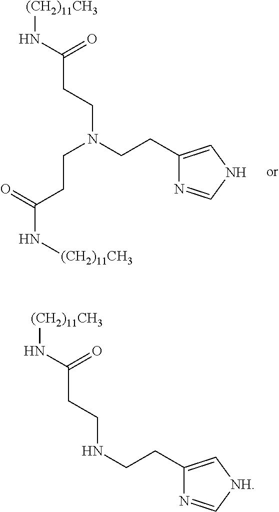

In other embodiments, the lipid is a composition of one or more of the above lipids.

In certain embodiments, the lipid is prepared by reacting amine 95 with acrylate ND to form lipid ND95. In certain embodiments, the lipid ND95 is of one of the formulae below:

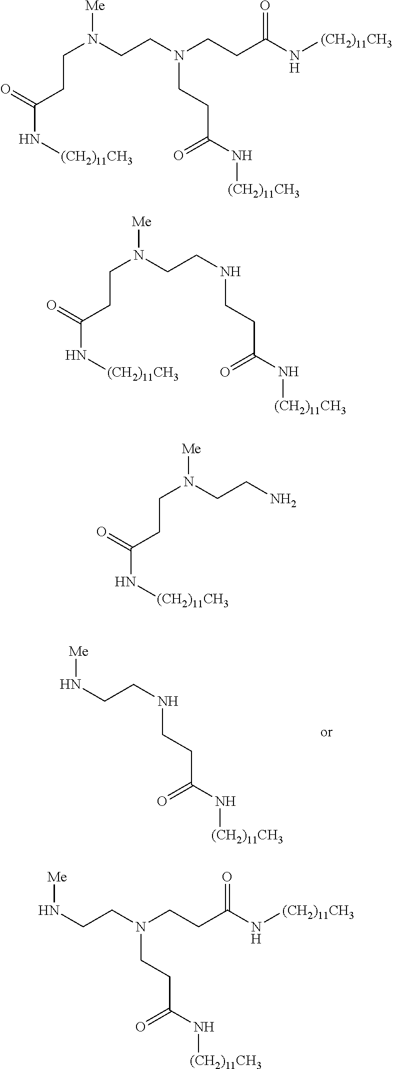

In other embodiments, the lipid is a composition of one or more of the above lipids.

In certain embodiments, the lipid is prepared by reacting amine 96 with acrylate ND to form lipid ND96. In certain embodiments, the lipid ND96 is of one of the formulae below: or

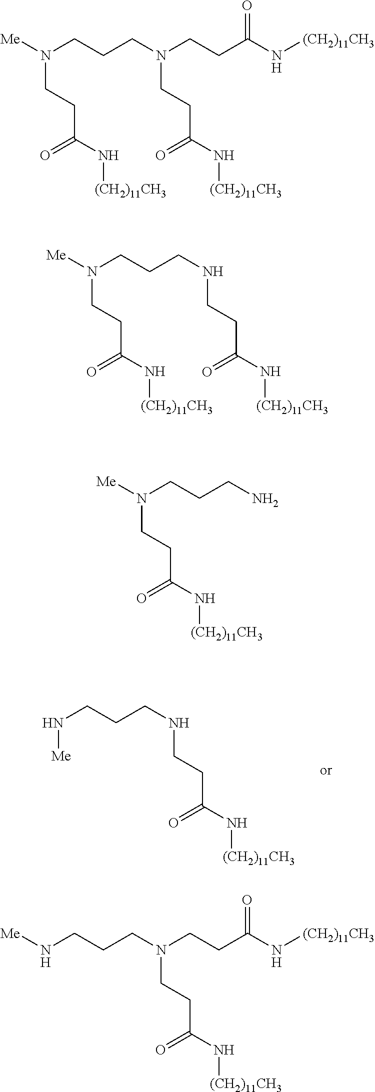

In other embodiments, the lipid is a composition of one or more of the above lipids.

In certain embodiments, the lipid is prepared by reacting amine 99 with acrylate ND to form lipid ND99. In certain embodiments, the lipid ND99 is of one of the formulae below:

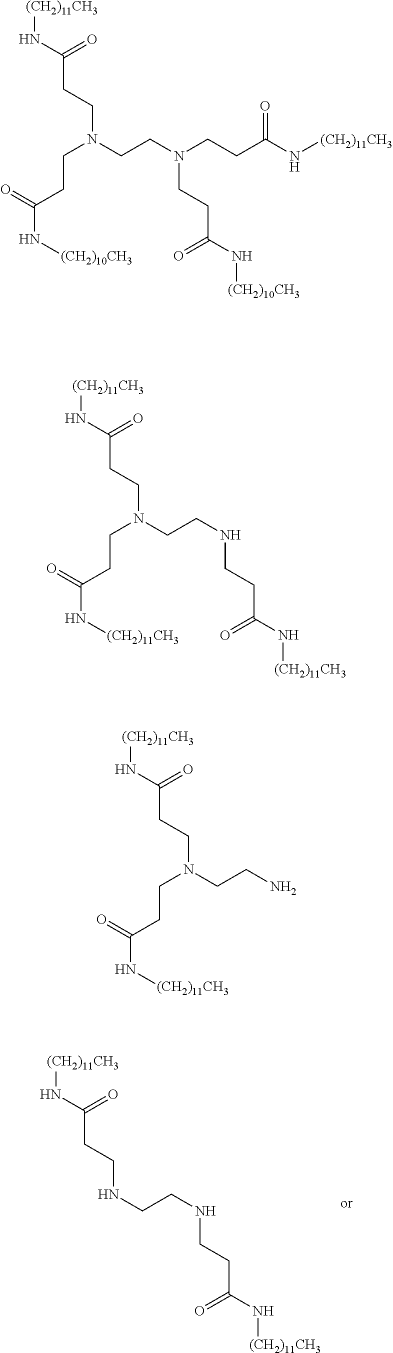

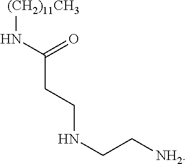

In other embodiments, the lipid is a composition of one or more of the above lipids. In certain embodiments, ND99 is treated with MeI or another alkylating agent to form lipids of the formulae:

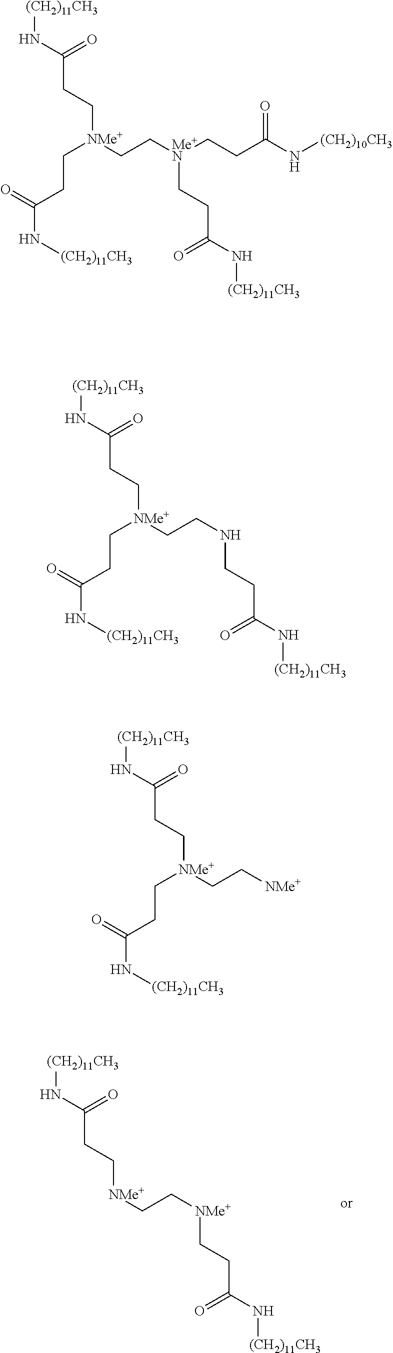

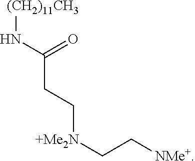

In certain embodiments, the lipid is prepared by reacting amine 100 with acrylate ND to form lipid ND100. In certain embodiments, the lipid ND100 is of one of the formulae below:

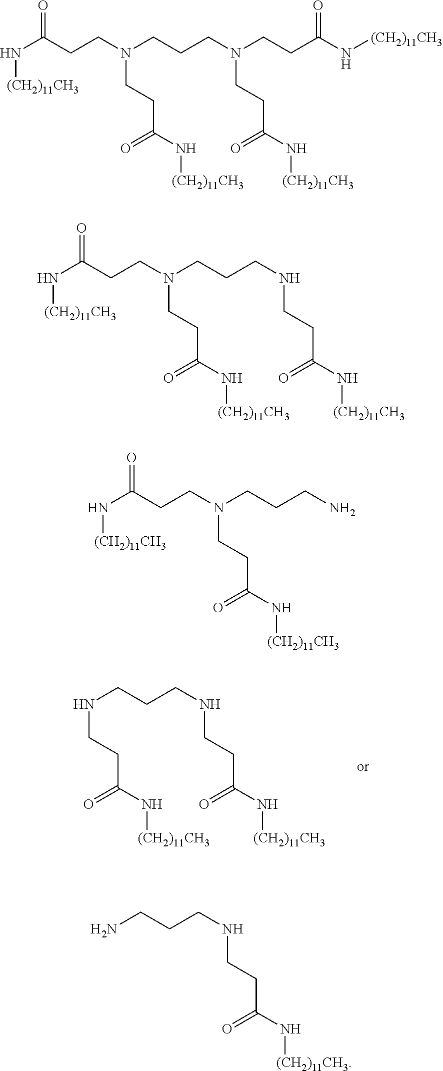

In other embodiments, the lipid is a composition of one or more of the above lipids.

In certain embodiments, the lipid is prepared by reacting amine 103 with acrylate ND to form lipid ND 103. In certain embodiments, the lipid ND 103 is of one of the formulae below:

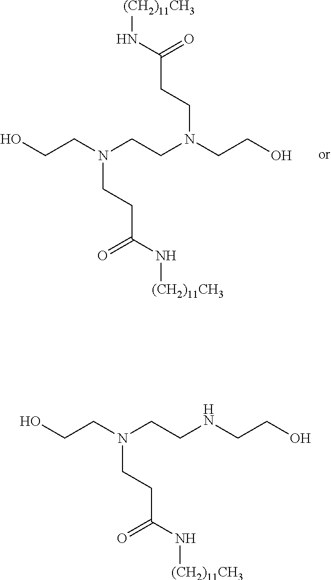

In other embodiments, the lipid is a composition of one or more of the above lipids.

In certain embodiments, the lipid is prepared by reacting amine 109 with acrylate ND to form lipid ND 109. In certain embodiments, the lipid ND 109 is of one of the formulae below:

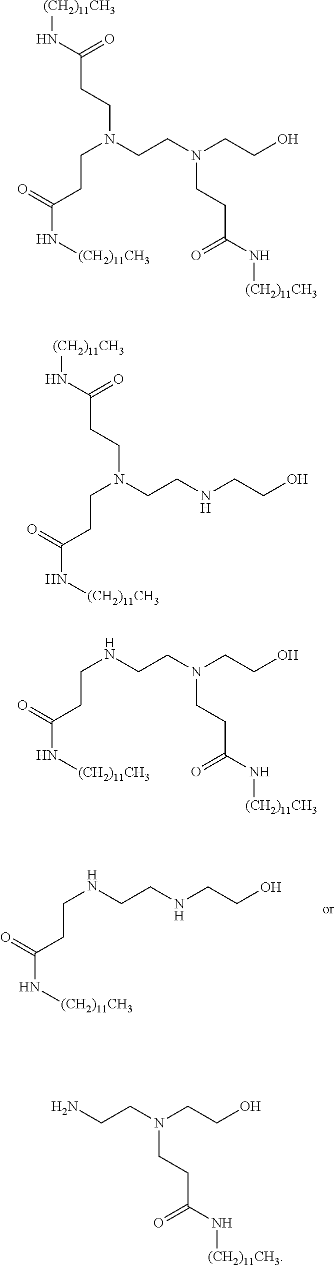

In other embodiments, the lipid is a composition of one or more of the above lipids.

In certain embodiments, the lipid is prepared by reacting amine 98 with acrylate NE to form lipid NE98. In certain embodiments, the lipid NE98 is of one of the formulae below:

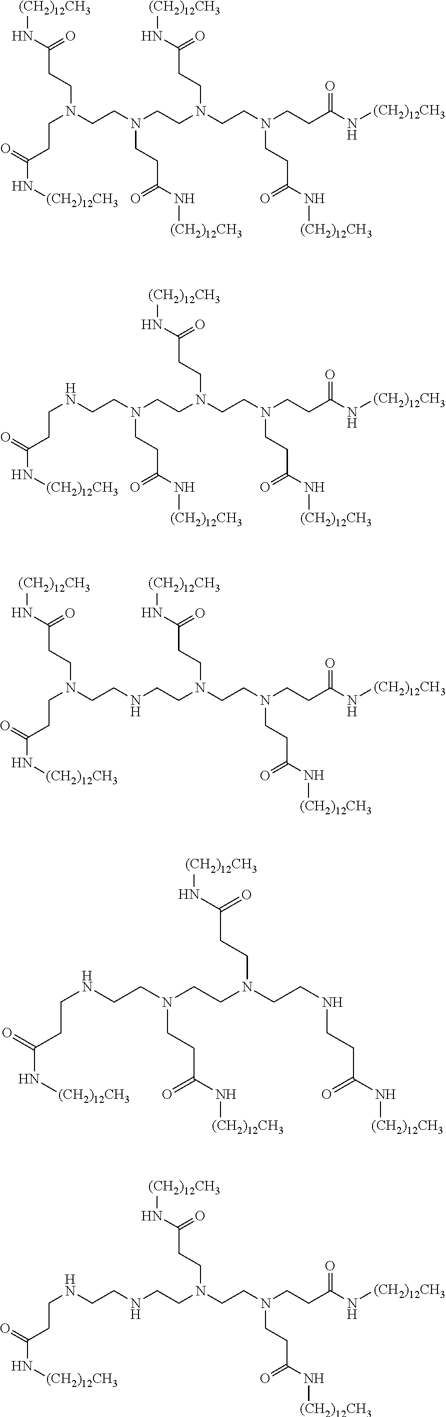

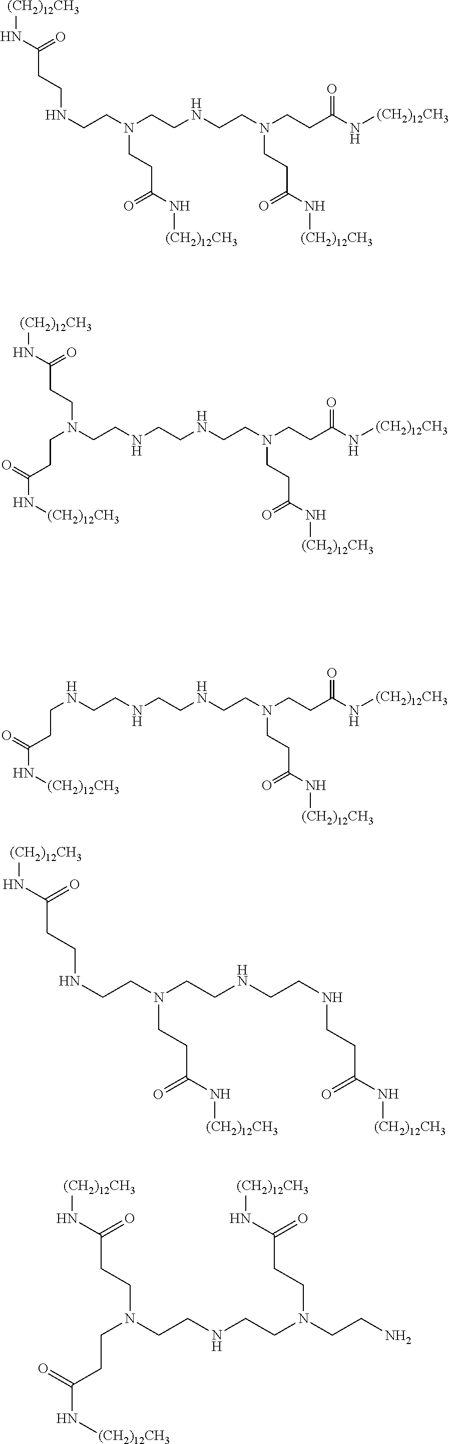

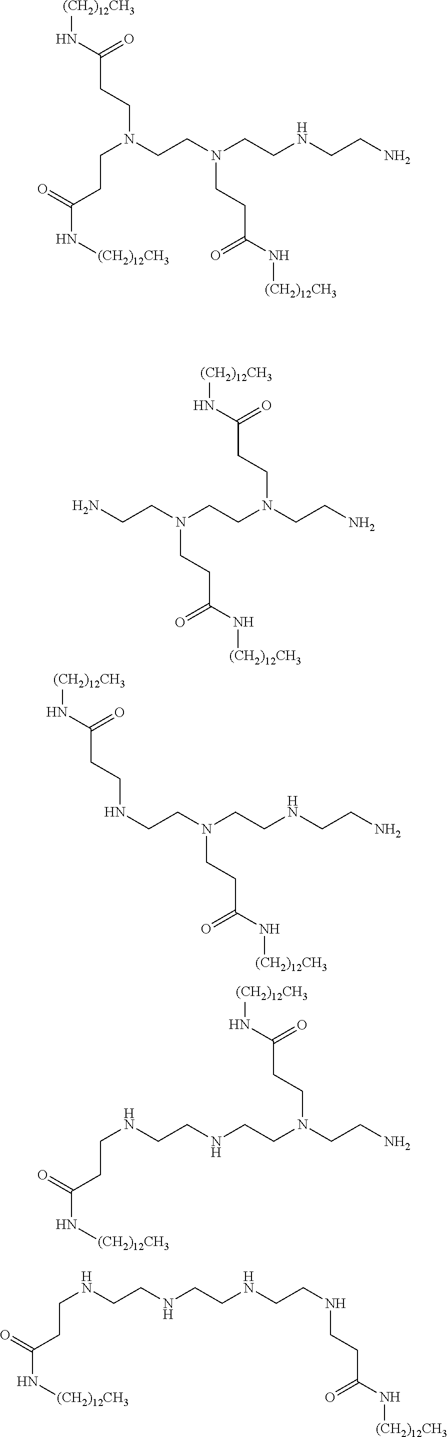

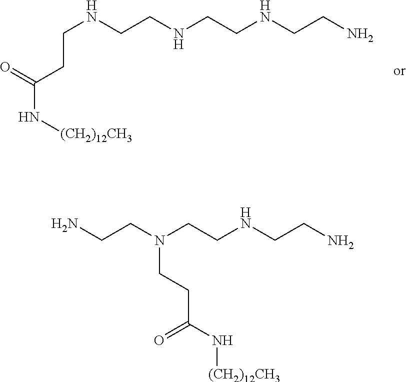

In other embodiments, the lipid is a composition of one or more of the above lipids.

In certain embodiments, the lipid is prepared by reacting amine 94 with acrylate NE to form lipid NE94. In certain embodiments, the lipid NE94 is of one of the formulae below:

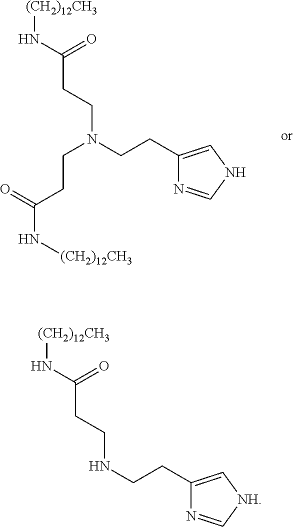

In other embodiments, the lipid is a composition of one or more of the above lipids.

In certain embodiments, the lipid is prepared by reacting amine 95 with acrylate NE to form lipid NE95. In certain embodiments, the lipid NE95 is of one of the formulae below:

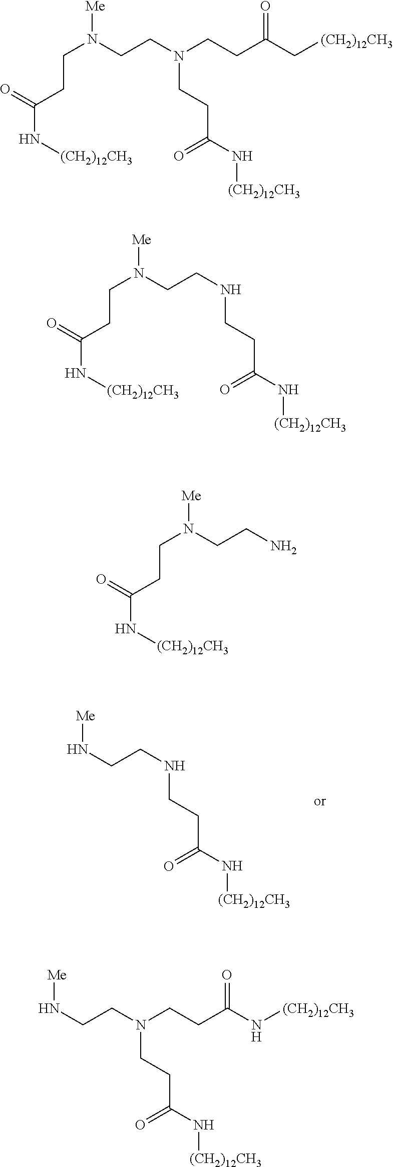

In other embodiments, the lipid is a composition of one or more of the above lipids.

In certain embodiments, the lipid is prepared by reacting amine 96 with acrylate NE to form lipid NE96. In certain embodiments, the lipid NE96 is of one of the formulae below:

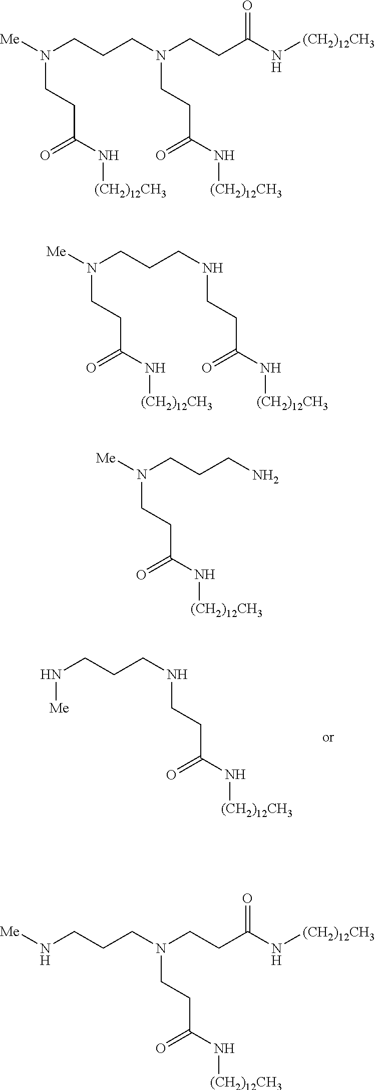

In other embodiments, the lipid is a composition of one or more of the above lipids.

In certain embodiments, the lipid is prepared by reacting amine 99 with acrylate

NE to form lipid NE99. In certain embodiments, the lipid NE99 is of one of the formulae below:

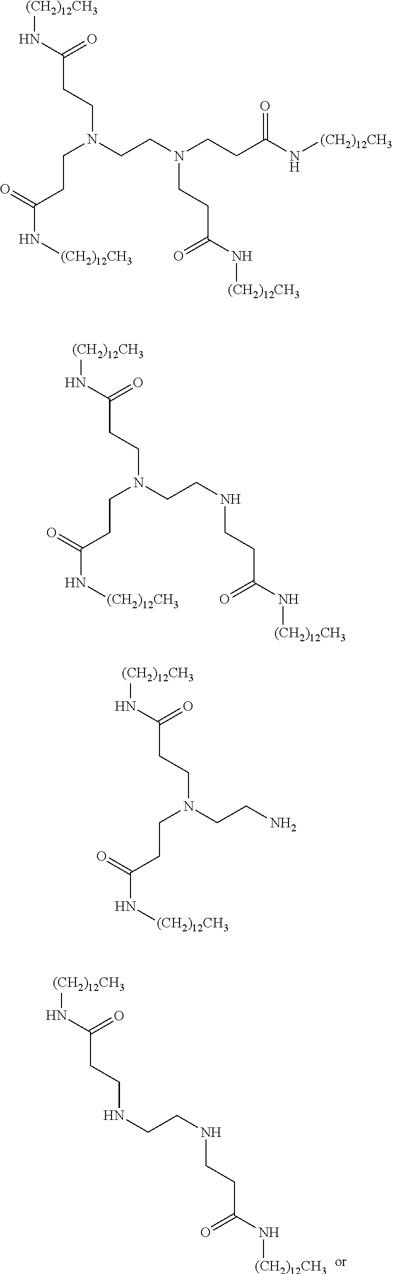

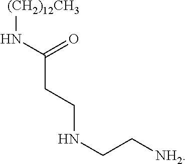

In other embodiments, the lipid is a composition of one or more of the above lipids. In certain embodiments, NE99 is treated with MeI or another alkylating agent to form lipids of the formulae:

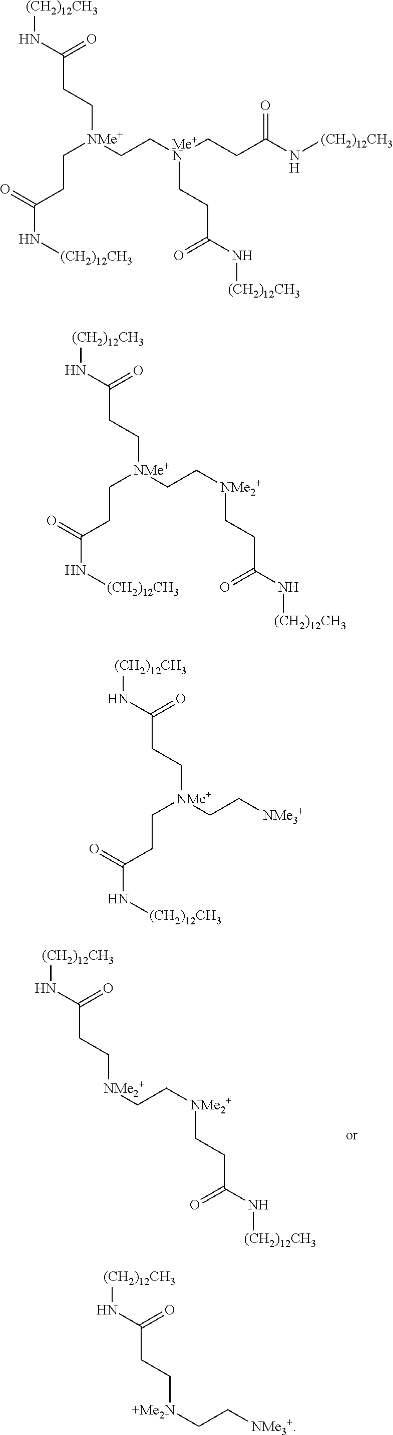

In certain embodiments, the lipid is prepared by reacting amine 100 with acrylate NE to form lipid NE100. In certain embodiments, the lipid NE100 is of one of the formulae below:

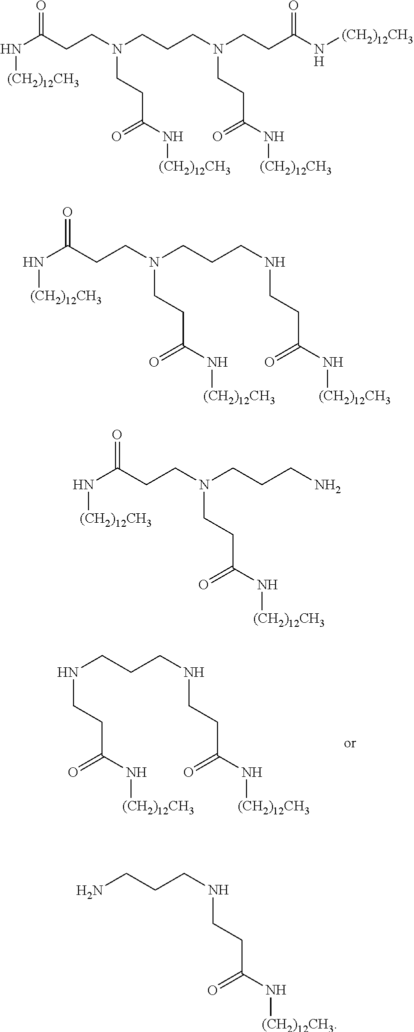

In other embodiments, the lipid is a composition of one or more of the above lipids.

In certain embodiments, the lipid is prepared by reacting amine 103 with acrylate NE to form lipid NE 103. In certain embodiments, the lipid NE 103 is of one of the formulae below:

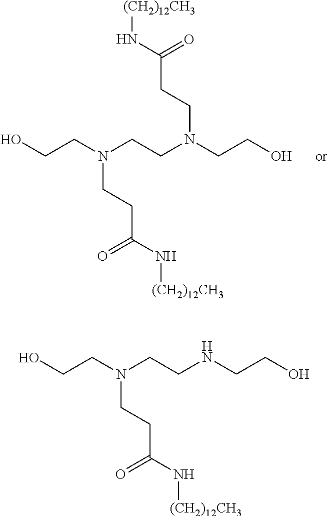

In other embodiments, the lipid is a composition of one or more of the above lipids.

In certain embodiments, the lipid is prepared by reacting amine 109 with acrylate NE to form lipid NE109. In certain embodiments, the lipid NE109 is of one of the formulae below:

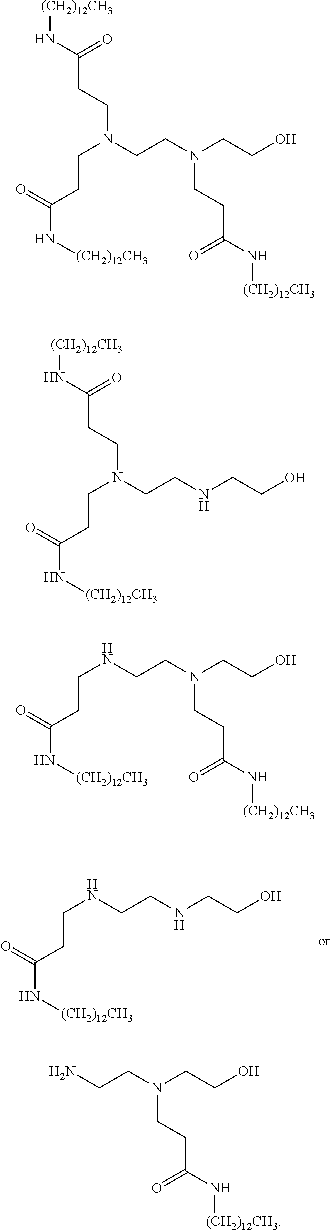

In other embodiments, the lipid is a composition of one or more of the above lipids.

In certain embodiments, the lipid is prepared by reacting amine 1 with acrylate NF to form lipid NF1. In certain embodiments, the lipid NF1 is of one of the formulae below:

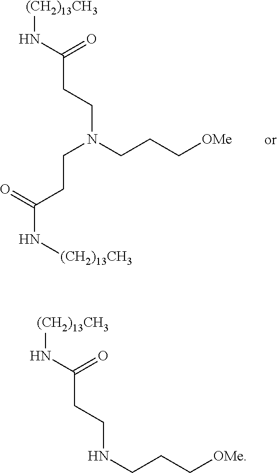

In other embodiments, the lipid is a composition of one or more of the above lipids.

In certain embodiments, the lipid is prepared by reacting amine 10 with acrylate NF to form lipid NF10. In certain embodiments, the lipid NF10 is of one of the formulae below:

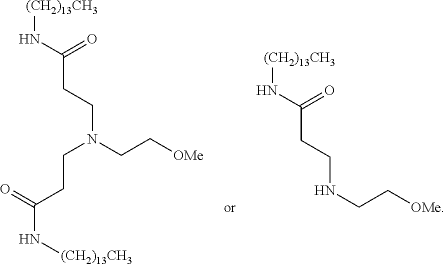

In other embodiments, the lipid is a composition of one or more of the above lipids.

In certain embodiments, the lipid is prepared by reacting amine 11 with acrylate NF to form lipid NF 11. In certain embodiments, the lipid NF 10 is of one of the formulae below:

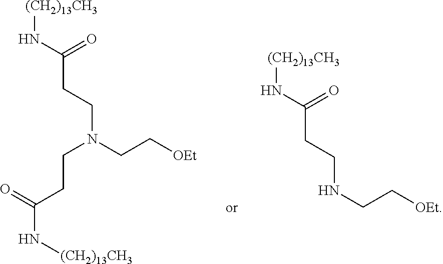

In other embodiments, the lipid is a composition of one or more of the above lipids.

In certain embodiments, the lipid is prepared by reacting amine 20 with acrylate NF to form lipid NF20. In certain embodiments, the lipid NF20 is of one of the formulae below:

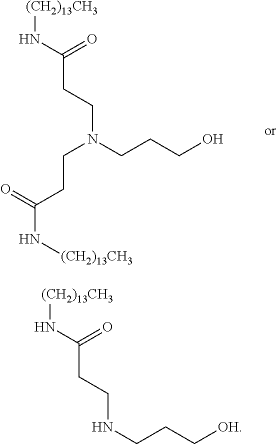

In other embodiments, the lipid is a composition of one or more of the above lipids.

In certain embodiments, the lipid is prepared by reacting amine 25 with acrylate NF to form lipid NF25. In certain embodiments, the lipid NF25 is of one of the formulae below:

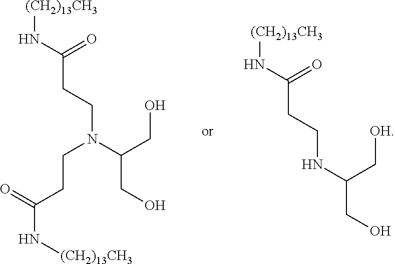

In other embodiments, the lipid is a composition of one or more of the above lipids.

In certain embodiments, the lipid is prepared by reacting amine 28 with acrylate NF to form lipid NF28. In certain embodiments, the lipid NF28 is of one of the formulae below:

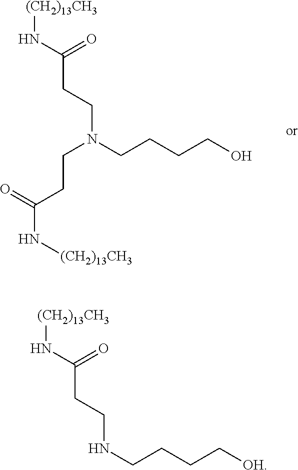

In other embodiments, the lipid is a composition of one or more of the above lipids.

In certain embodiments, the lipid is prepared by reacting amine 32 with acrylate NF to form lipid NF32. In certain embodiments, the lipid NF32 is of one of the formulae below:

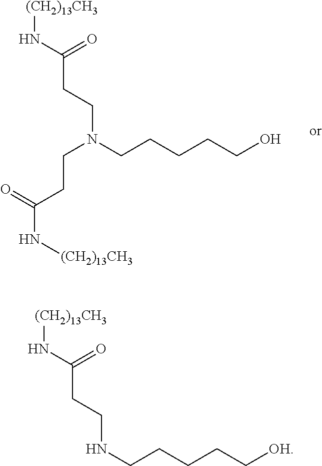

In other embodiments, the lipid is a composition of one or more of the above lipids.

In certain embodiments, the lipid is prepared by reacting amine 36 with acrylate NF to form lipid NF36. In certain embodiments, the lipid NF36 is of one of the formulae below:

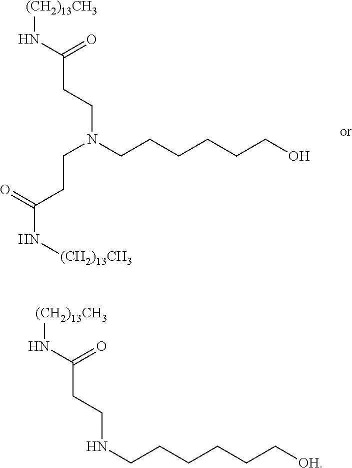

In other embodiments, the lipid is a composition of one or more of the above lipids.

In certain embodiments, the lipid is prepared by reacting amine 60 with acrylate NF to form lipid NF60. In certain embodiments, the lipid NF60 is of one of the formulae below:

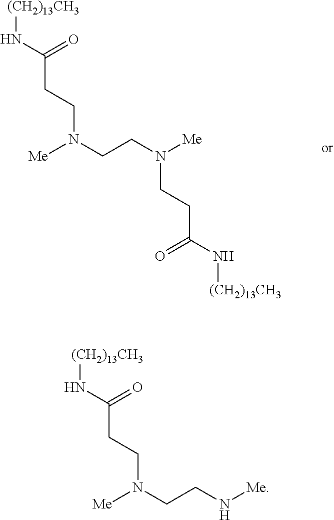

In other embodiments, the lipid is a composition of one or more of the above lipids.

In certain embodiments, the lipid is prepared by reacting amine 61 with acrylate NF to form lipid NF61. In certain embodiments, the lipid NF61 is of one of the formulae below:

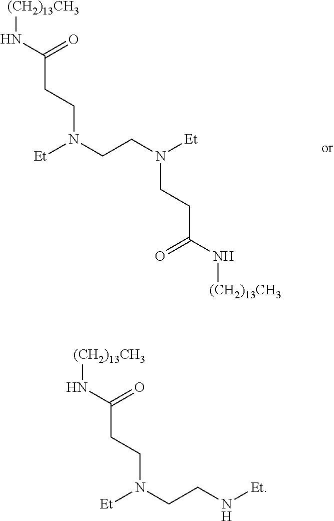

In other embodiments, the lipid is a composition of one or more of the above lipids.

In certain embodiments, the lipid is prepared by reacting amine 63 with acrylate NF to form lipid NF63. In certain embodiments, the lipid NF63 is of one of the formulae below:

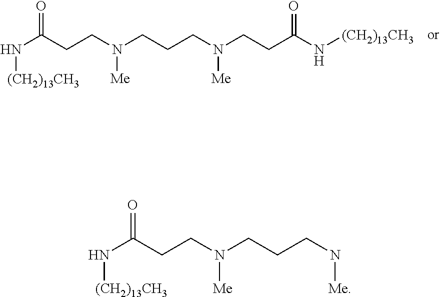

In other embodiments, the lipid is a composition of one or more of the above lipids.

In certain embodiments, the lipid is prepared by reacting amine 64 with acrylate NF to form lipid NF64. In certain embodiments, the lipid NF64 is of one of the formulae below:

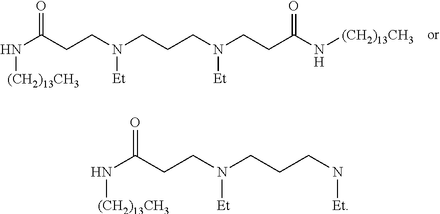

In other embodiments, the lipid is a composition of one or more of the above lipids.

In certain embodiments, the lipid is prepared by reacting amine 61 with acrylate NF to form lipid NF70. In certain embodiments, the lipid NF70 is of one of the formulae below.

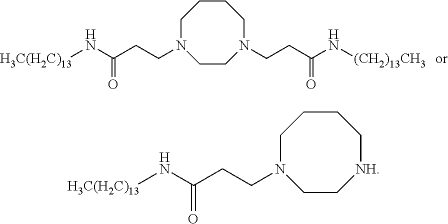

In other embodiments, the lipid is a composition of one or more of the above lipids.

In certain embodiments, the lipid is prepared by reacting amine 86 with acrylate NF to form lipid NF86. In certain embodiments, the lipid NF86 is of one of the formulae below:

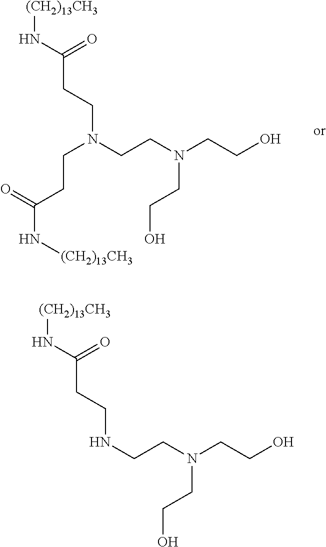

In other embodiments, the lipid is a composition of one or more of the above lipids.

In certain embodiments, the lipid is prepared by reacting amine 87 with acrylate NF to form lipid NF87. In certain embodiments, the lipid NF87 is of one of the formulae below:

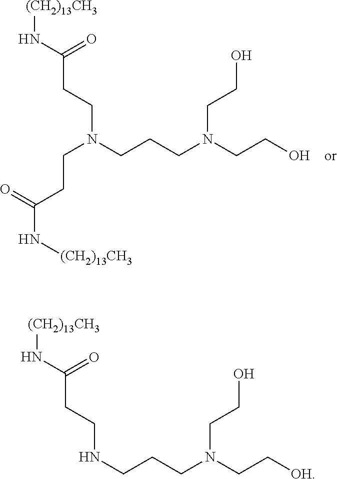

In other embodiments, the lipid is a composition of one or more of the above lipids.

In certain embodiments, the lipid is prepared by reacting amine 91 with acrylate NF to form lipid NF91. In certain embodiments, the lipid NF91 is of one of the formulae below:

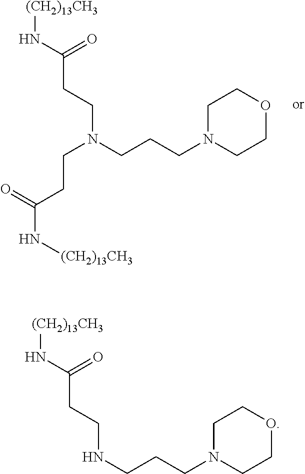

In other embodiments, the lipid is a composition of one or more of the above lipids.

In certain embodiments, the lipid is prepared by reacting amine 95 with acrylate NF to form lipid NF95. In certain embodiments, the lipid NF95 is of one of the formulae below:

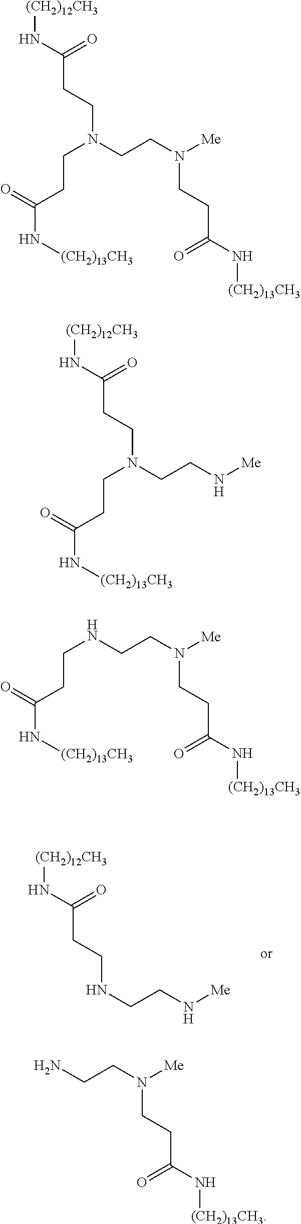

In other embodiments, the lipid is a composition of one or more of the above lipids. In certain embodiments, the lipid is prepared by reacting amine 96 with acrylate NF to form lipid NF96. In certain embodiments, the lipid NF96 is of one of the formulae below:

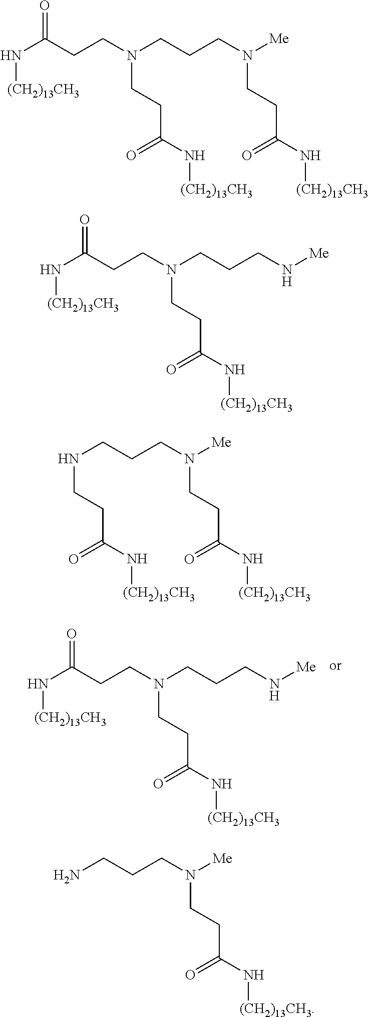

In other embodiments, the lipid is a composition of one or more of the above lipids.

In certain embodiments, the lipid is prepared by reacting amine 98 with acrylate NF to form lipid NF98. In certain embodiments, the lipid NF98 is of one of the formulae below:

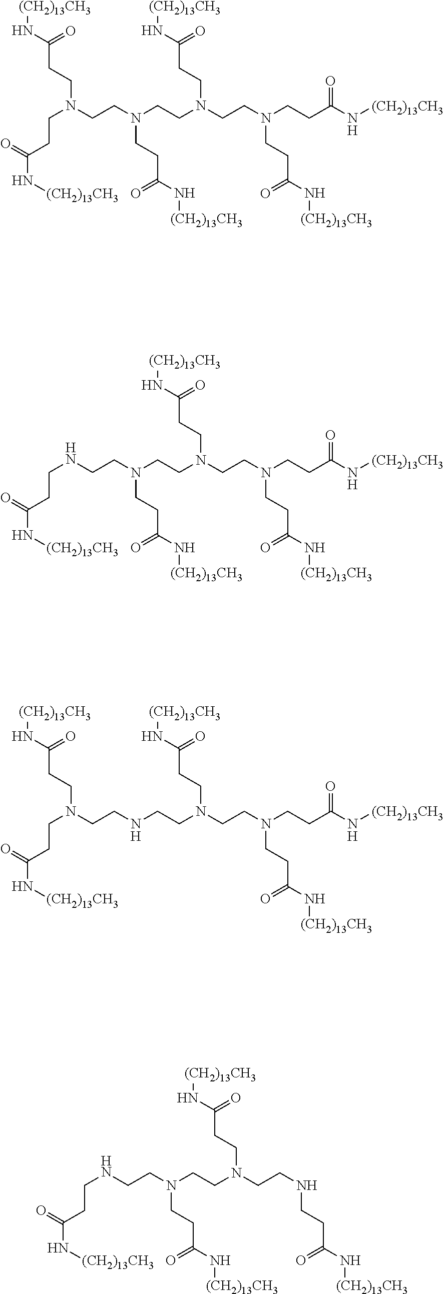

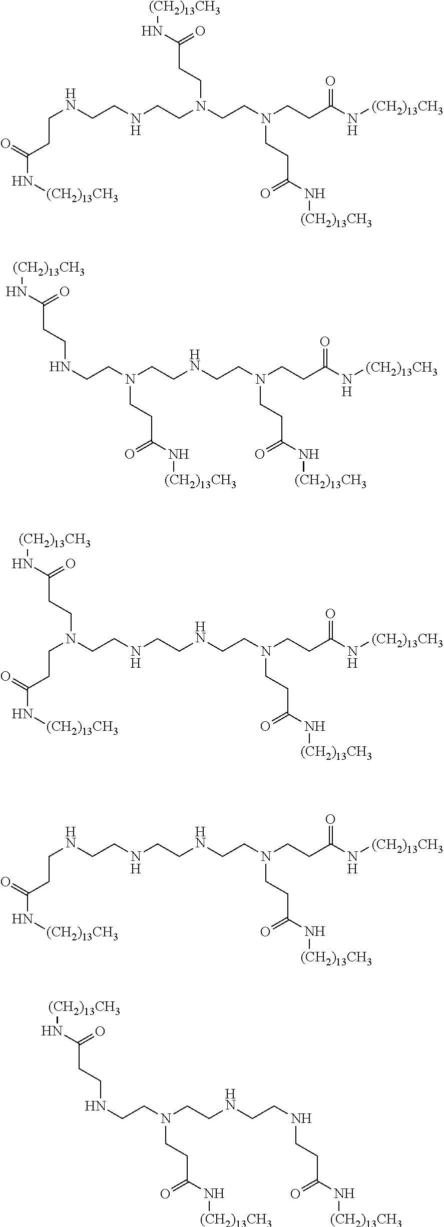

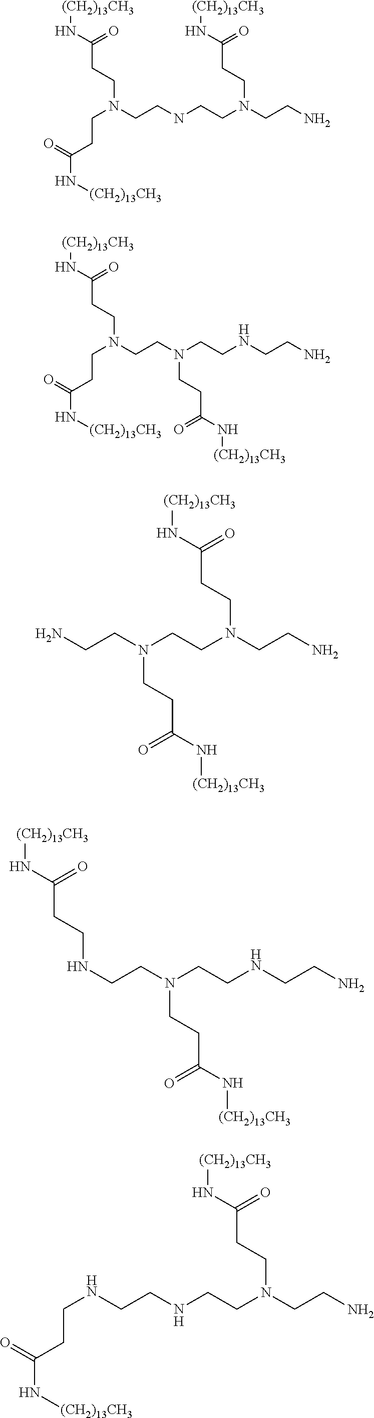

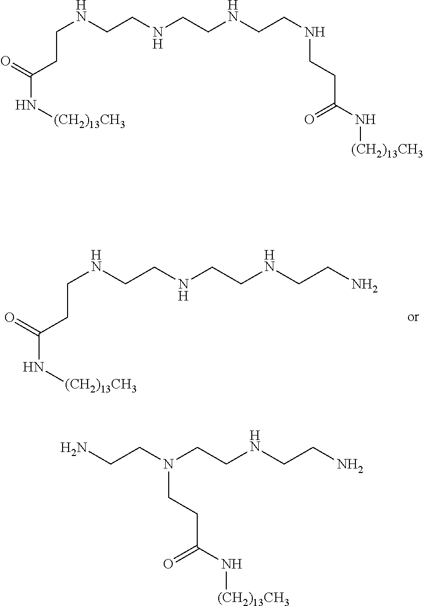

In other embodiments, the lipid is a composition of one or more of the above lipids.

In certain embodiments, the lipid is prepared by reacting amine 99 with acrylate NF to form lipid NF99. In certain embodiments, the lipid NF99 is of one of the formulae below:

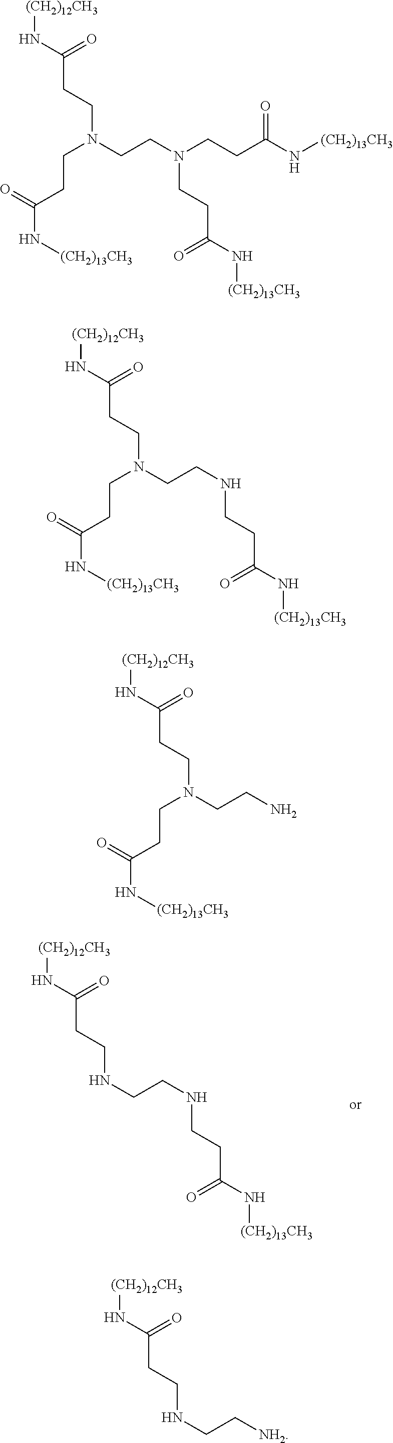

In other embodiments, the lipid is a composition of one or more of the above lipids. In certain embodiments, NF99 is treated with MeI or another alkylating agent to form lipids of the formula:

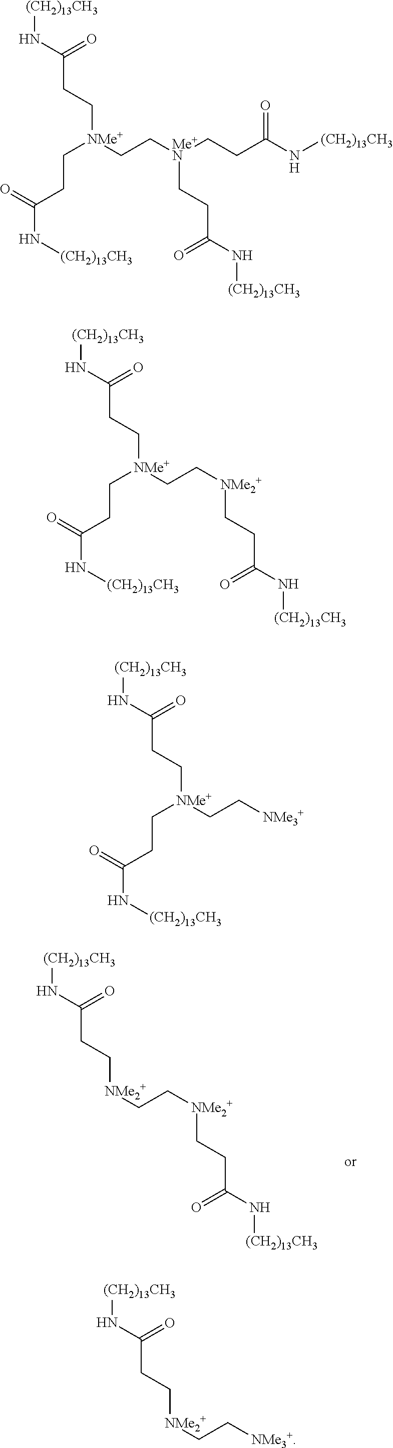

In certain embodiments, the lipid is prepared by reacting amine 100 with acrylate NF to form lipid NF100. In certain embodiments, the lipid NF100 is of one of the formulae below:

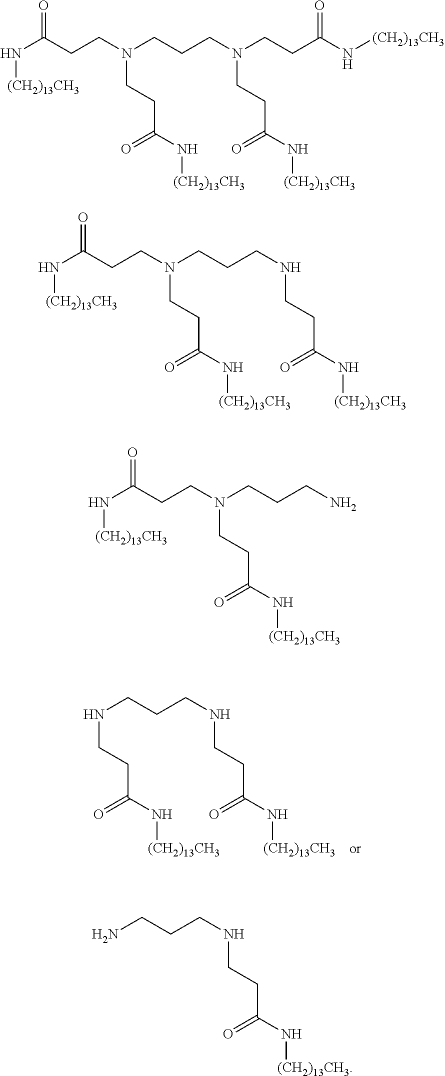

In other embodiments, the lipid is a composition of one or more of the above lipids.

In certain embodiments, the lipid is prepared by reacting amine 103 with acrylate NF to form lipid NF 103. In certain embodiments, the lipid NE 103 is of one of the formulae below:

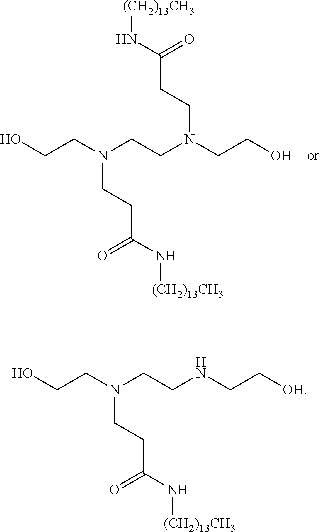

In other embodiments, the lipid is a composition of one or more of the above lipids.

In certain embodiments, the lipid is prepared by reacting amine 109 with acrylate NF to form lipid NF 109. In certain embodiments, the lipid NF 109 is of one of the formulae below:

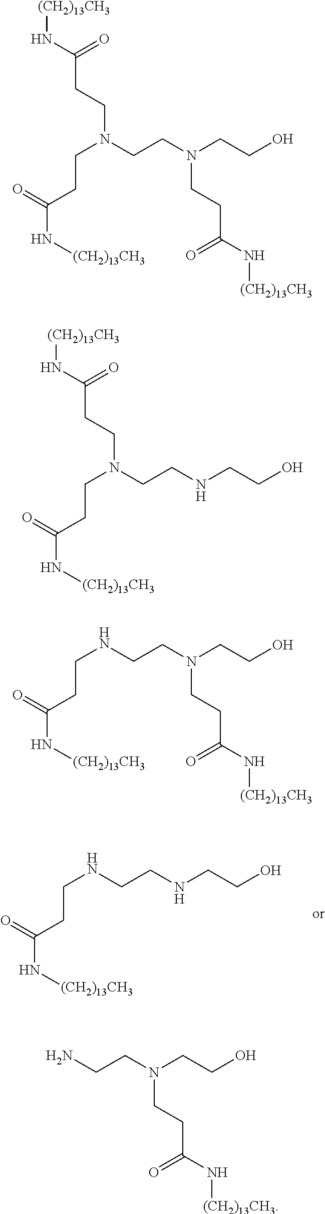

In other embodiments, the lipid is a composition of one or more of the above lipids.

In certain embodiments, the lipid is prepared by reacting amine 61 with acrylate NG to form lipid NG61. In certain embodiments, the lipid NG61 is of one of the formulae below:

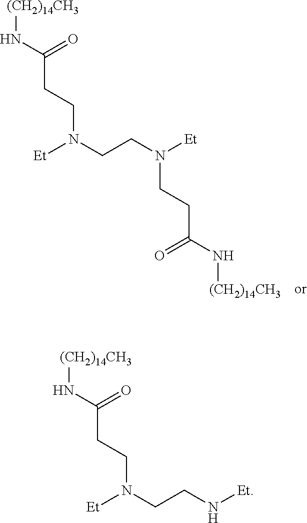

In other embodiments, the lipid is a composition of one or more of the above lipids.

In certain embodiments, the lipid is prepared by reacting amine 64 with acrylate NG to form lipid NG64. In certain embodiments, the lipid NG64 is of one of the formulae below:

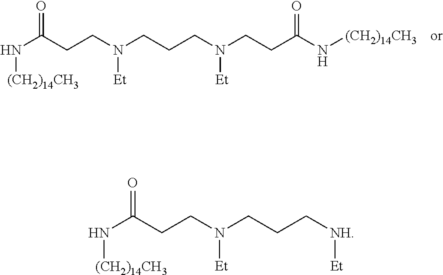

In other embodiments, the lipid is a composition of one or more of the above lipids.

In certain embodiments, the lipid is prepared by reacting amine 77 with acrylate NG to form lipid NG77. In certain embodiments, the lipid NG77 is of one of the formulae below:

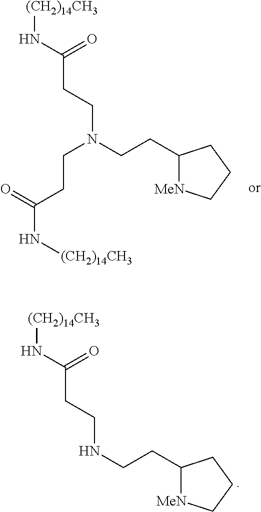

In other embodiments, the lipid is a composition of one or more of the above lipids.

In certain embodiments, the lipid is prepared by reacting amine 86 with acrylate NG to form lipid NG86. In certain embodiments, the lipid NG86 is of one of the formulae below:

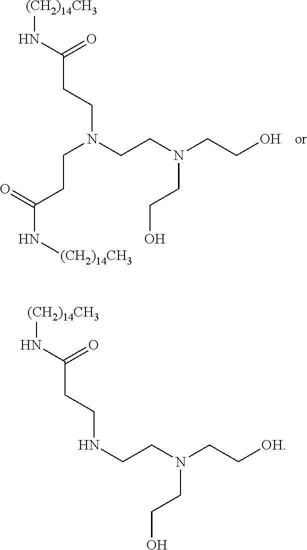

In other embodiments, the lipid is a composition of one or more of the above lipids.

In certain embodiments, the lipid is prepared by reacting amine 87 with acrylate NG to form lipid NG87. In certain embodiments, the lipid NG87 is of one of the formulae below:

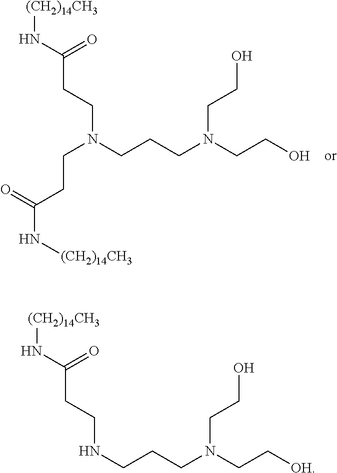

In other embodiments, the lipid is a composition of one or more of the above lipids.

In certain embodiments, the lipid is prepared by reacting amine 95 with acrylate NG to form lipid NG95. In certain embodiments, the lipid NG95 is of one of the formulae below:

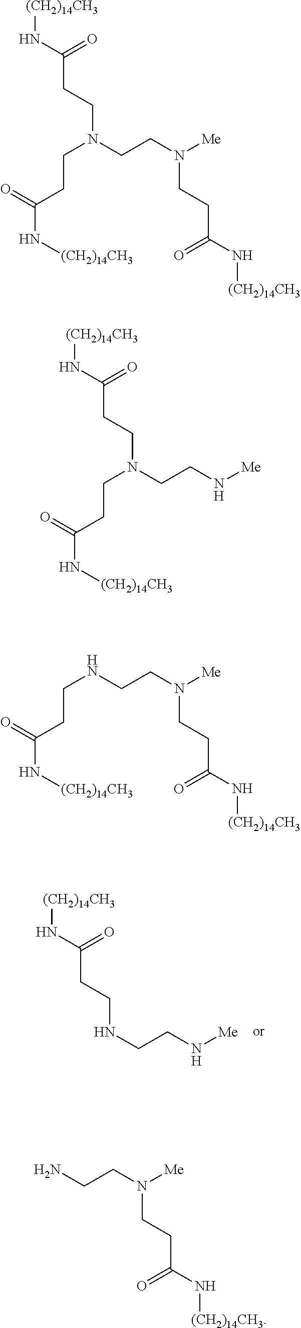

In other embodiments, the lipid is a composition of one or more of the above lipids.

In certain embodiments, the lipid is prepared by reacting amine 100 with acrylate NG to form lipid NG100. In certain embodiments, the lipid NG100 is of one of the formulae below:

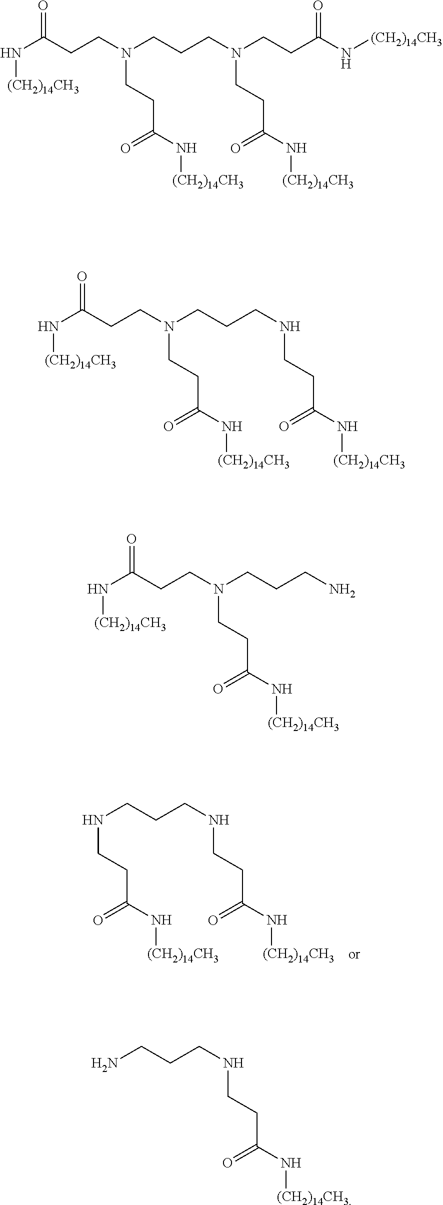

In other embodiments, the lipid is a composition of one or more of the above lipids. In certain embodiments, NG100 is alkylated with methyl iodide or another alkylating agent.

In certain embodiments, the lipid is prepared by reacting amine 62 with acrylate NP to form lipid NP62. In certain embodiments, the lipid NP62 is of one of the formulae below:

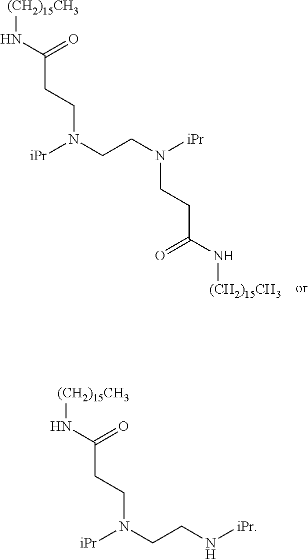

In other embodiments, the lipid is a composition of one or more of the above lipids.

In certain embodiments, the lipid is prepared by reacting amine 63 with acrylate NP to form lipid NP63. In certain embodiments, the lipid NP63 is of one of the formulae below:

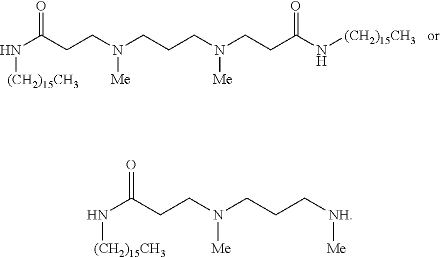

In other embodiments, the lipid is a composition of one or more of the above lipids.

In certain embodiments, the lipid is prepared by reacting amine 86 with acrylate NP to form lipid NP86. In certain embodiments, the lipid NP86 is of one of the formulae below:

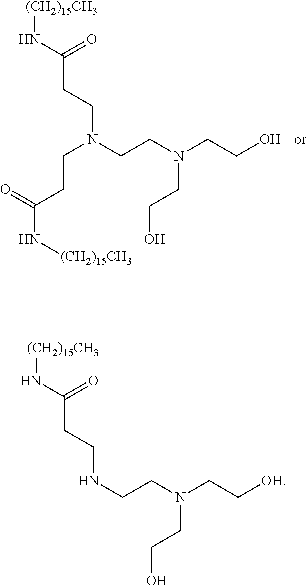

In other embodiments, the lipid is a composition of one or more of the above lipids.

In certain embodiments, the lipid is prepared by reacting amine 87 with acrylate NP to form lipid NP87. In certain embodiments, the lipid NP87 is of one of the formulae below.

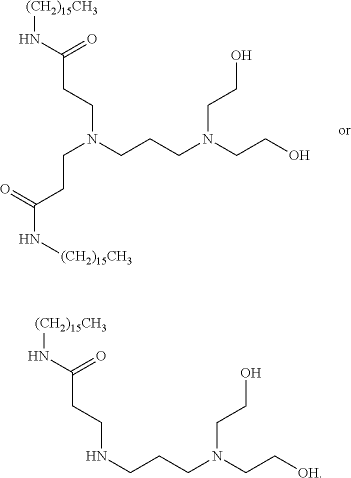

In other embodiments, the lipid is a composition of one or more of the above lipids.

In certain embodiments, the lipid is prepared by reacting amine 96 with acrylate NP to form lipid NP96. In certain embodiments, the lipid NP96 is of one of the formulae below:

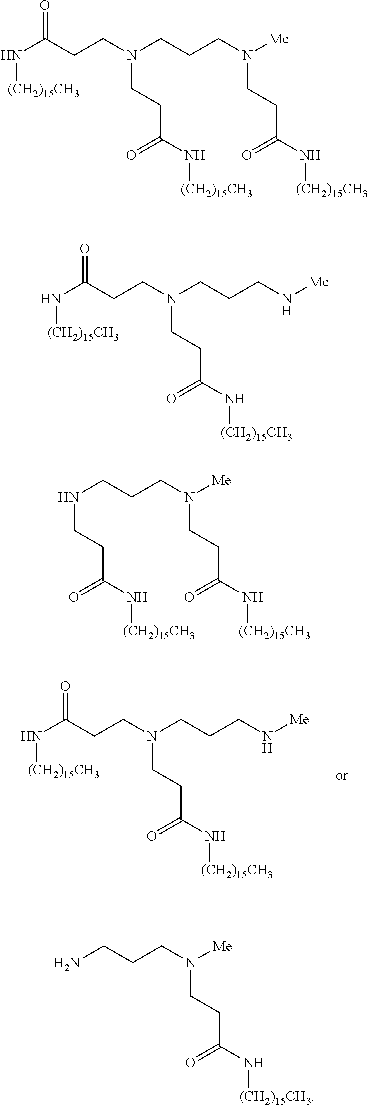

In other embodiments, the lipid is a composition of one or more of the above lipids.

In certain embodiments, the lipid is prepared by reacting amine 98 with acrylate NP to form lipid NP98. In certain embodiments, the lipid NP98 is of one of the formulae below:

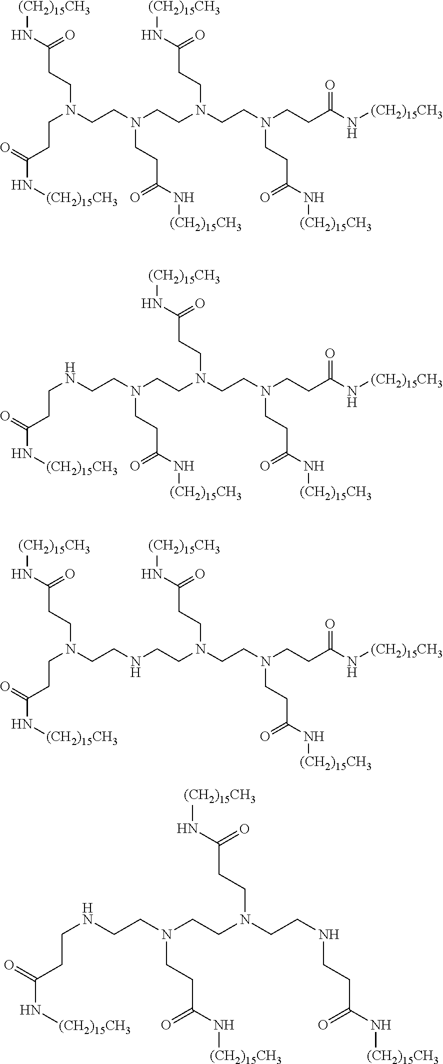

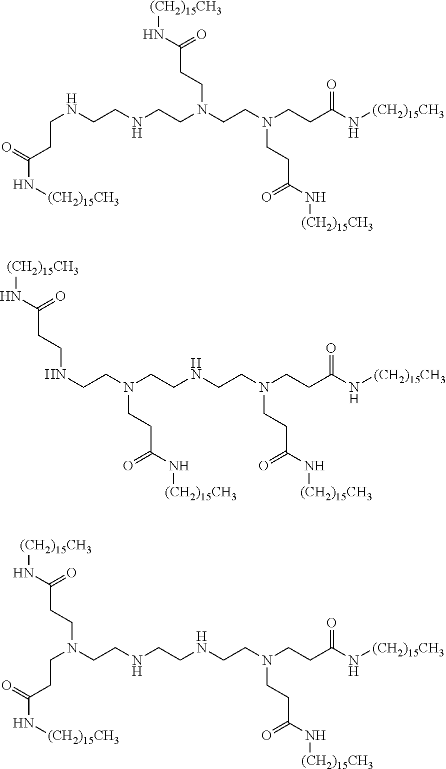

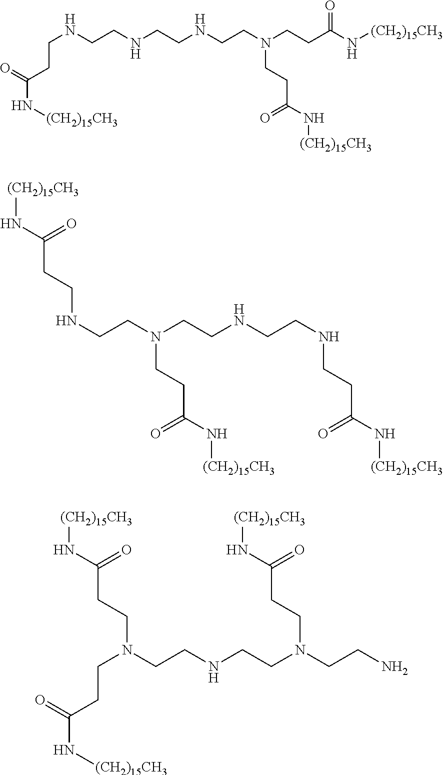

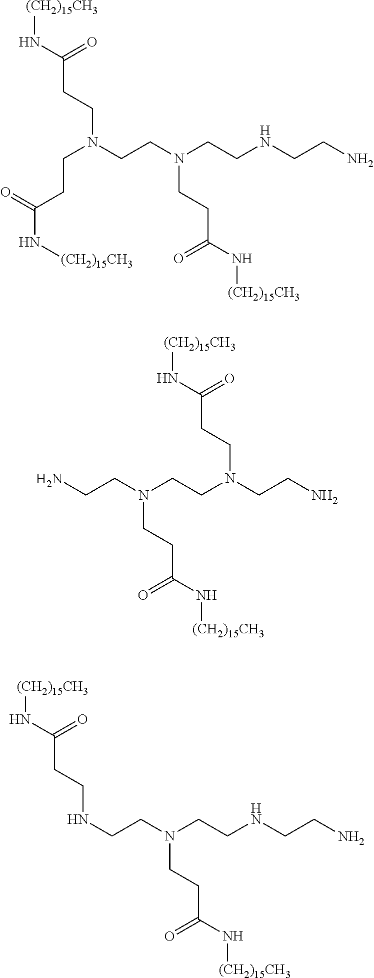

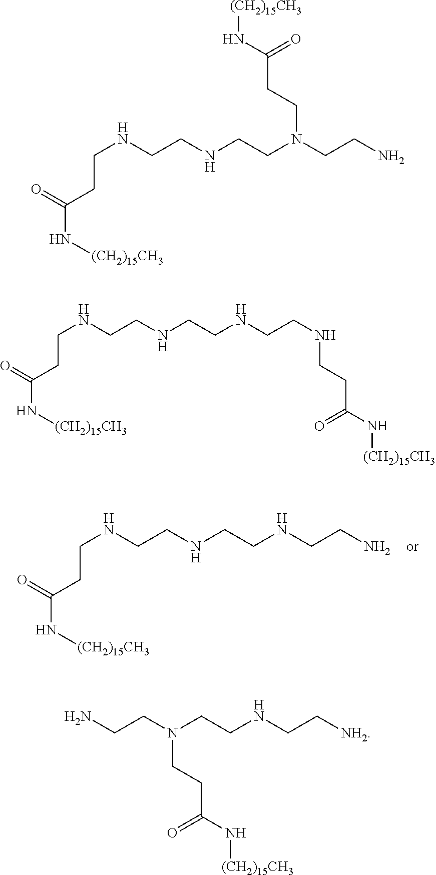

In other embodiments, the lipid is a composition of one or more of the above lipids.

In certain embodiments, the lipid is prepared by reacting amine 99 with acrylate NP to form lipid NP99. In certain embodiments, the lipid NP99 is of one of the formulae below:

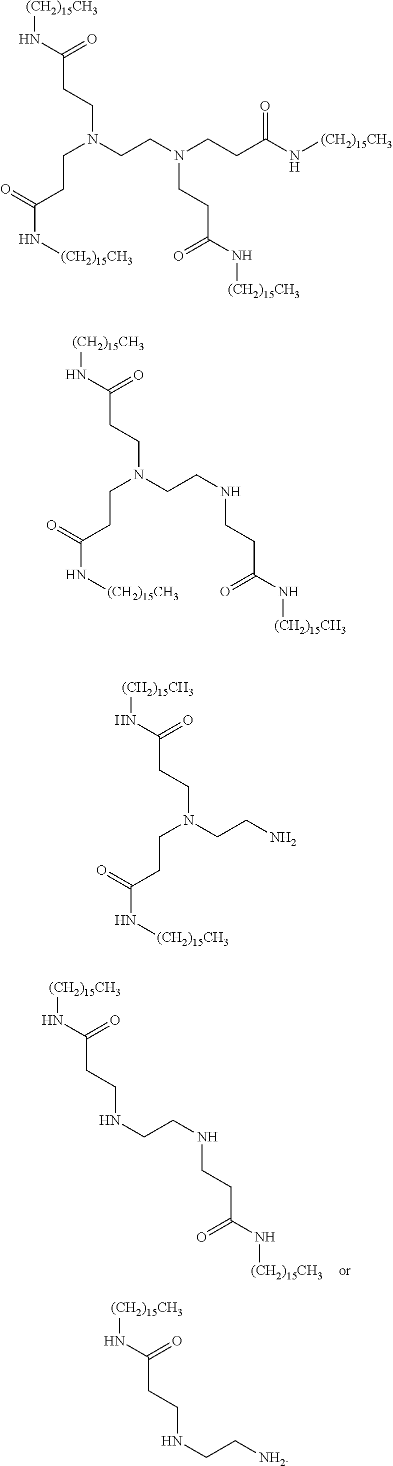

In other embodiments, the lipid is a composition of one or more of the above lipids. In certain embodiments, NF99 is treated with MeI or another alkylating agent to form lipids of the formula:

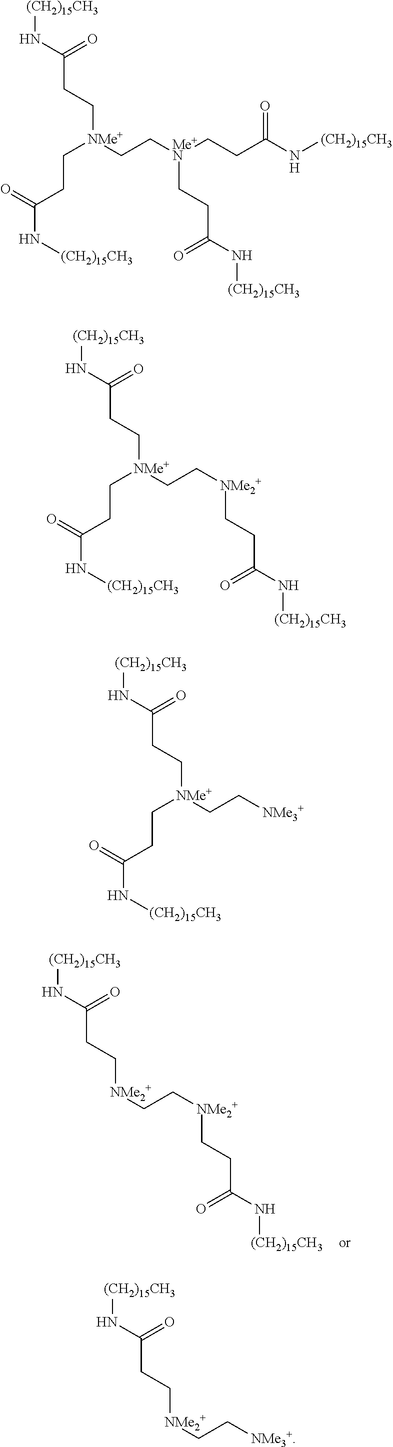

In certain embodiments, the lipid is prepared by reacting amine 100 with acrylate NP to form lipid NP100. In certain embodiments, the lipid NP100 is of one of the formulae below:

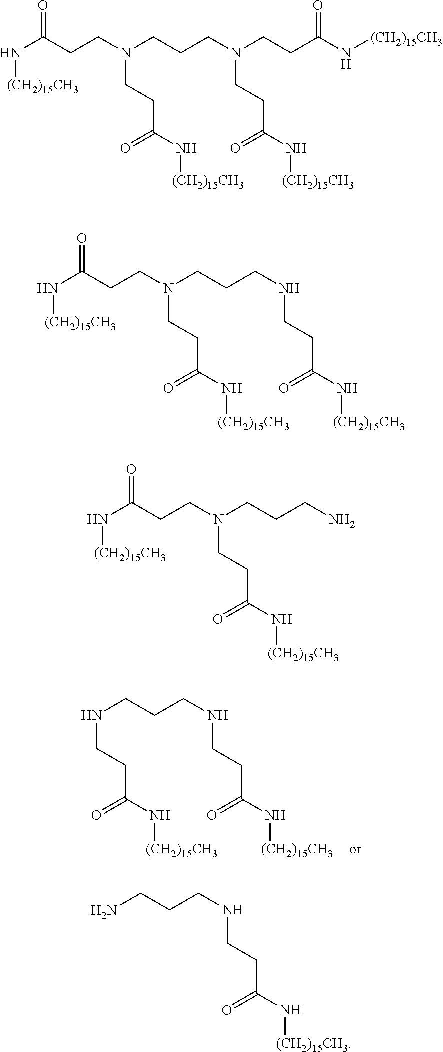

In other embodiments, the lipid is a composition of one or more of the above lipids.

In certain embodiments, the lipid is prepared by reacting amine 103 with acrylate NP to form lipid NP 103. In certain embodiments, the lipid NP 103 is of one of the formulae below:

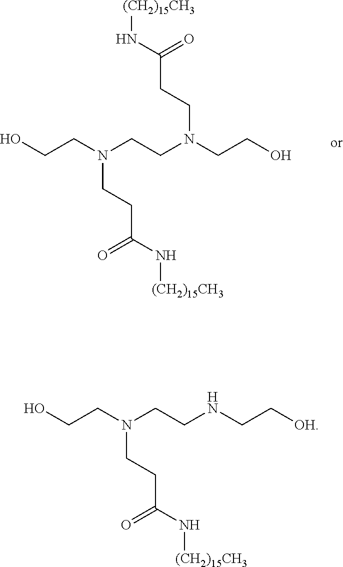

In other embodiments, the lipid is a composition of one or more of the above lipids.

In certain embodiments, the lipid is prepared by reacting amine 31 with acrylate LD to form lipid LD31. In certain embodiments, the lipid LD31 is of one of the formulae below.

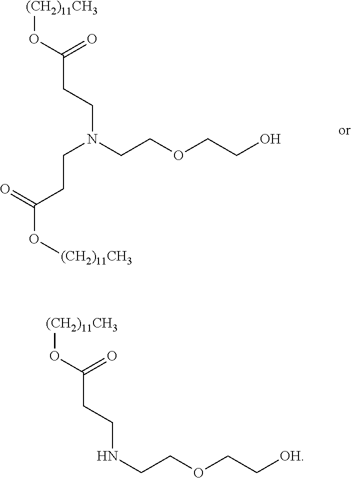

In other embodiments, the lipid is a composition of one or more of the above lipids.

In certain embodiments, the lipid is prepared by reacting amine 98 with acrylate LD to form lipid LD98. In certain embodiments, the lipid LD98 is of one of the formulae below:

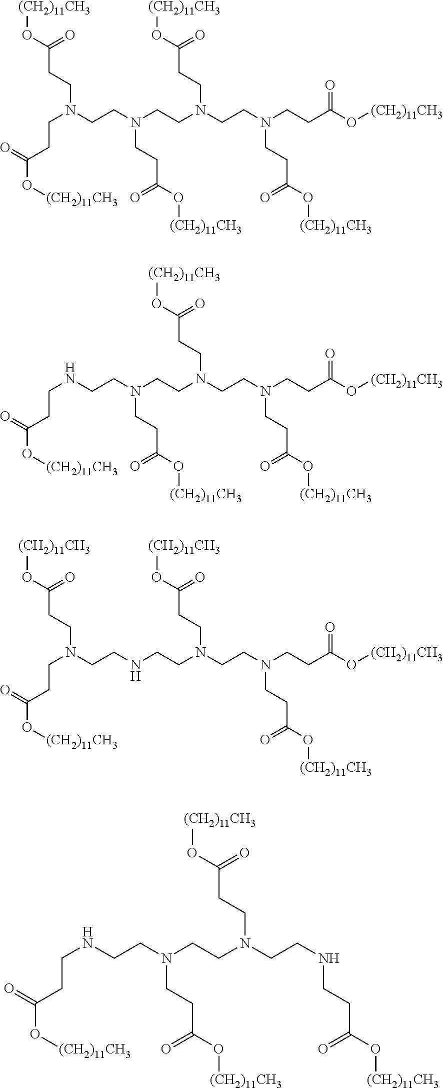

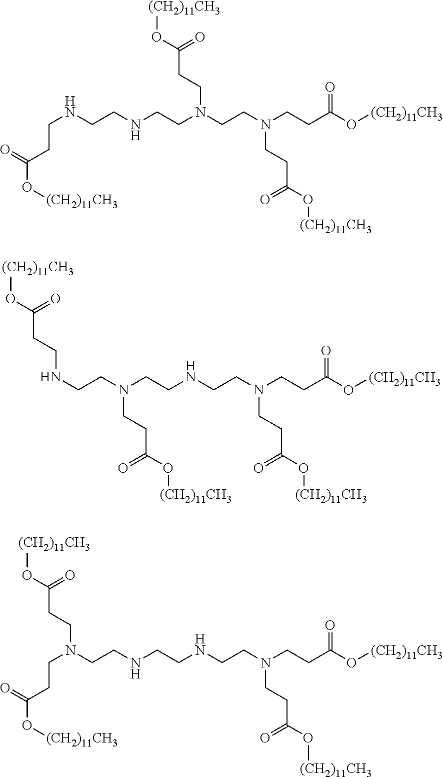

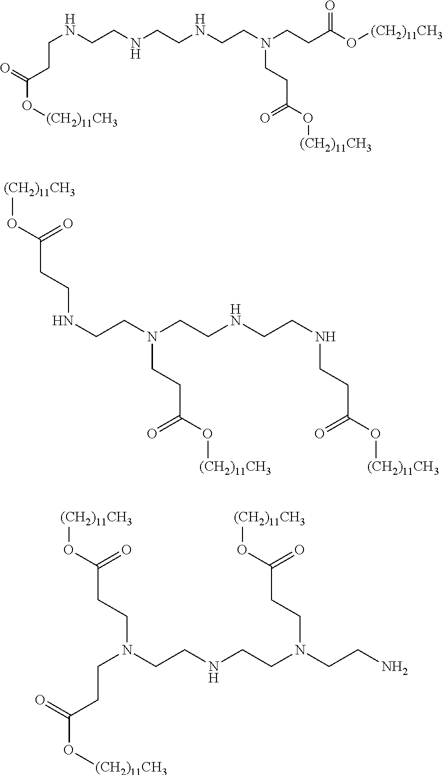

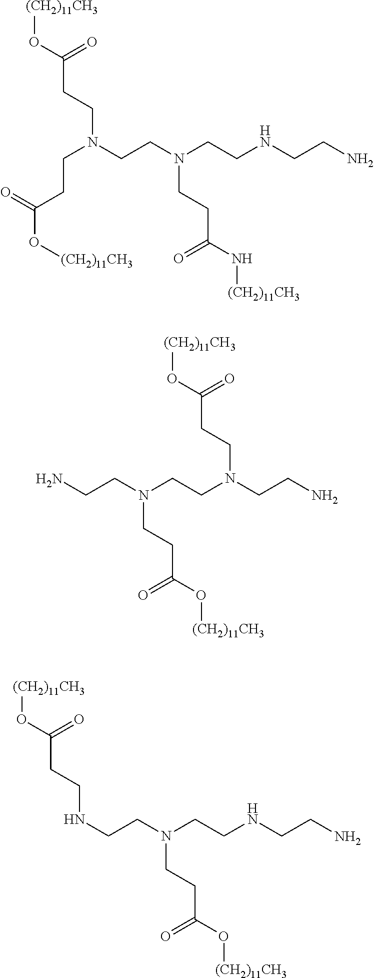

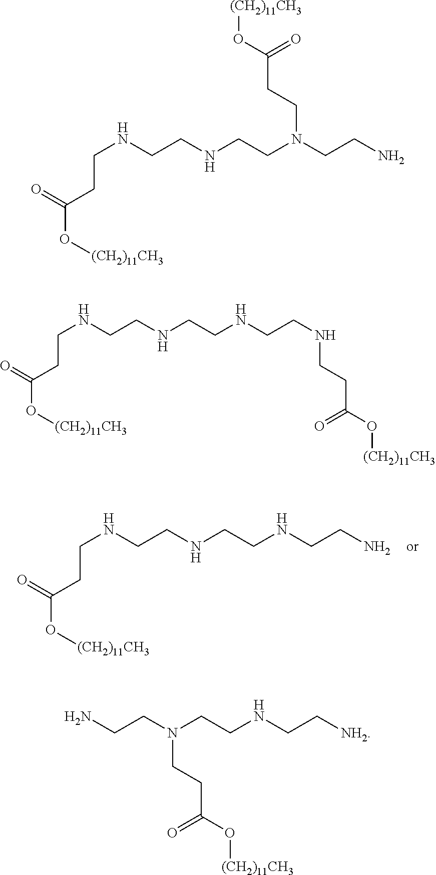

In other embodiments, the lipid is a composition of one or more of the above lipids.

In certain embodiments, the lipid is prepared by reacting amine 99 with acrylate LD to form lipid LD99. In certain embodiments, the lipid LD99 is of one of the formulae below:

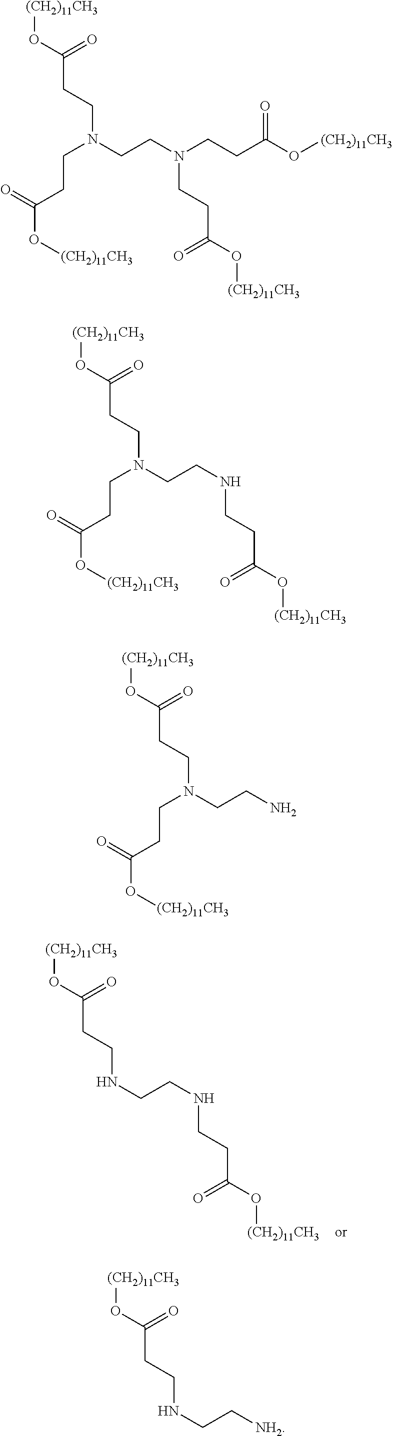

In other embodiments, the lipid is a composition of one or more of the above lipids. In certain embodiments, LD99 is treated with MeI or another alkylating agent to form lipids (QD99) of the formula:

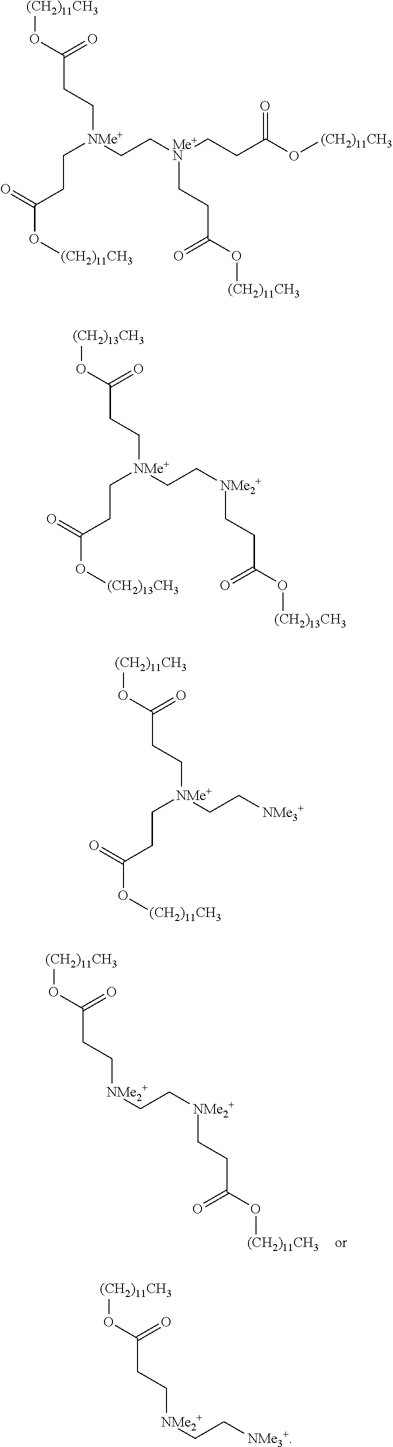

In certain embodiments, the lipid is prepared by reacting amine 100 with acrylate LD to form lipid LD100. In certain embodiments, the lipid LD100 is of one of the formulae below:

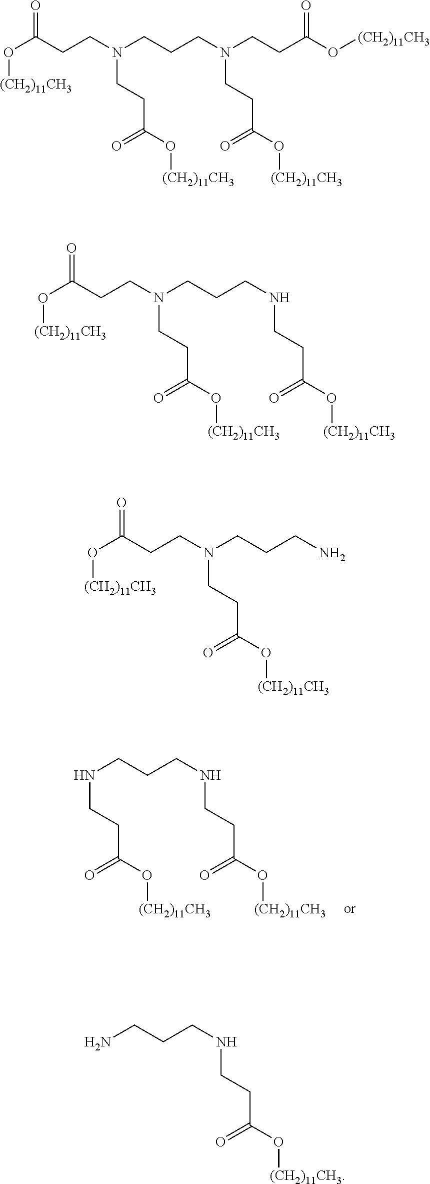

In other embodiments, the lipid is a composition of one or more of the above lipids. In certain embodiments, LD100 is treated with MeI or another alkylating agent to form lipids (QD100) of the formula:

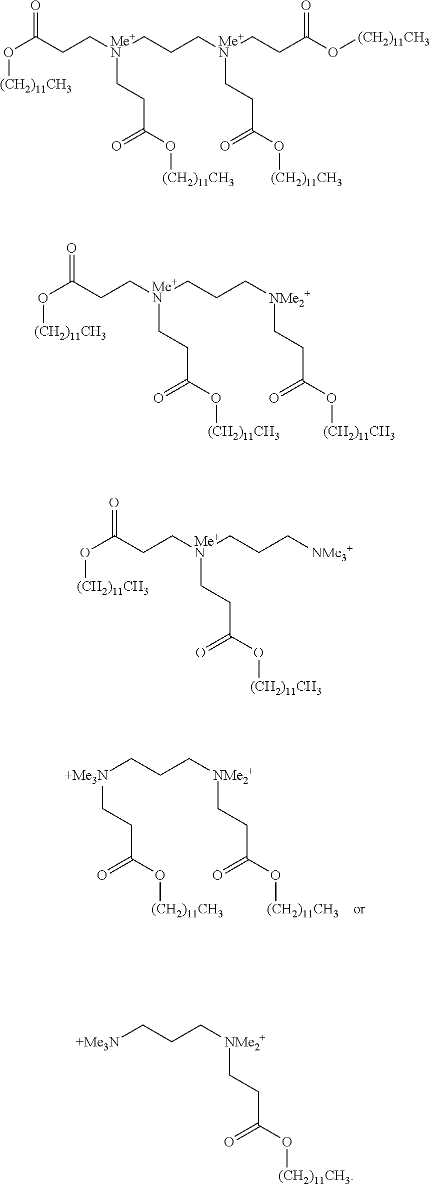

In certain embodiments, the lipid is prepared by reacting amine 87 with acrylate LE to form lipid LE87. In certain embodiments, the lipid LE87 is of one of the formulae below:

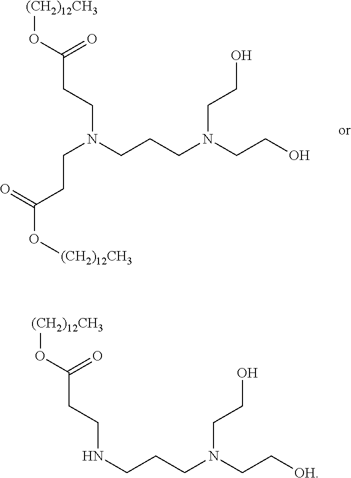

In other embodiments, the lipid is a composition of one or more of the above lipids.

In certain embodiments, the lipid is prepared by reacting amine 94 with acrylate LE to form lipid LE94. In certain embodiments, the lipid LE94 is of one of the formulae below:

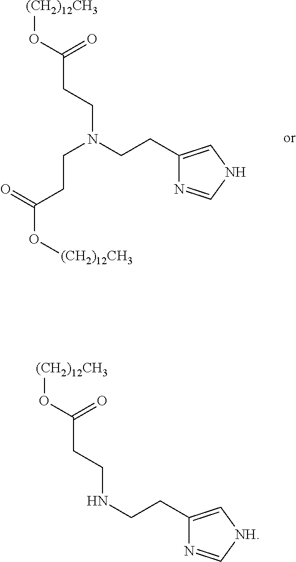

In other embodiments, the lipid is a composition of one or more of the above lipids.

In certain embodiments, the lipid is prepared by reacting amine 31 with acrylate LF to form lipid LF31. In certain embodiments, the lipid LF31 is of one of the formulae below:

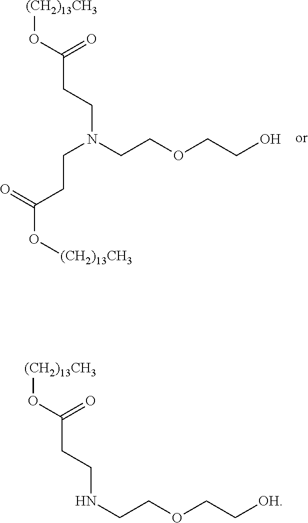

In other embodiments, the lipid is a composition of one or more of the above lipids.

In certain embodiments, the lipid is prepared by reacting amine 94 with acrylate LF to form lipid LF94. In certain embodiments, the lipid LF94 is of one of the formulae below:

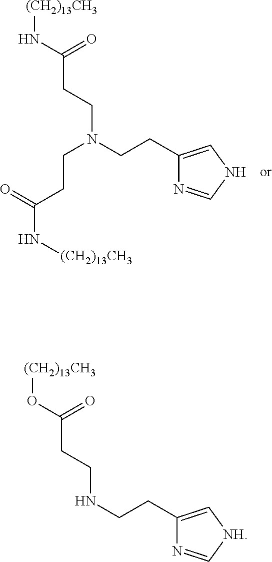

In other embodiments, the lipid is a composition of one or more of the above lipids.

In certain embodiments, the lipid is prepared by reacting amine 95 with acrylate LF to form lipid LF95. In certain embodiments, the lipid LF95 is of one of the formulae below:

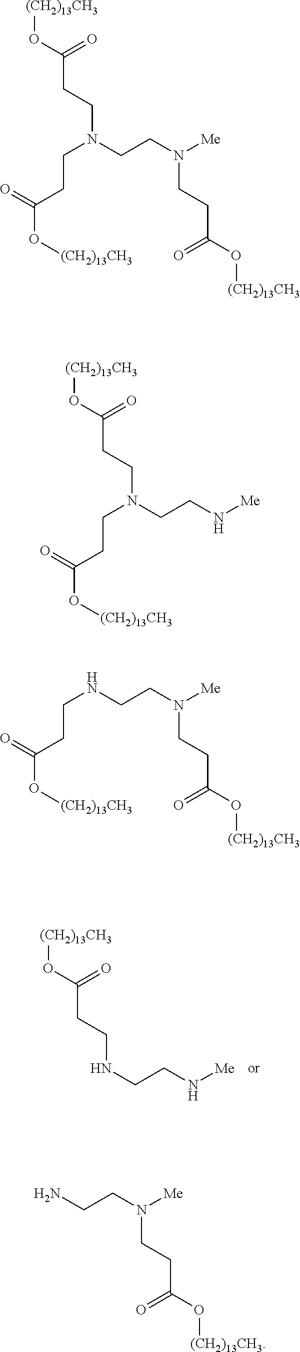

In other embodiments, the lipid is a composition of one or more of the above lipids.

In certain embodiments, the lipid is prepared by reacting amine 99 with acrylate LF to form lipid LF 99. In certain embodiments, the lipid LF99 is of one of the formulae below:

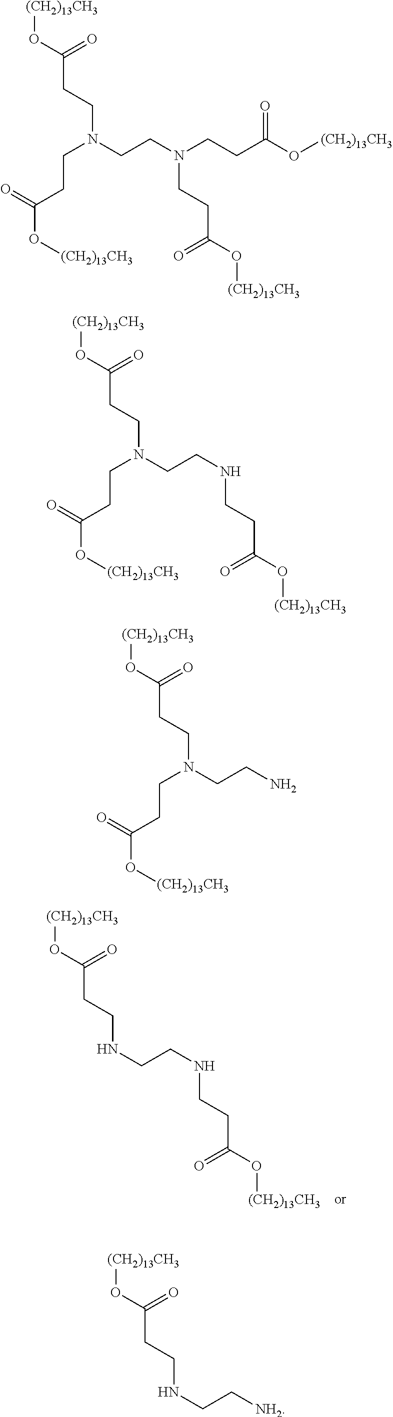

In other embodiments, the lipid is a composition of one or more of the above lipids. In certain embodiments, LF99 is treated with MeI or another alkylating agent to form lipid (QF99) of the formula:

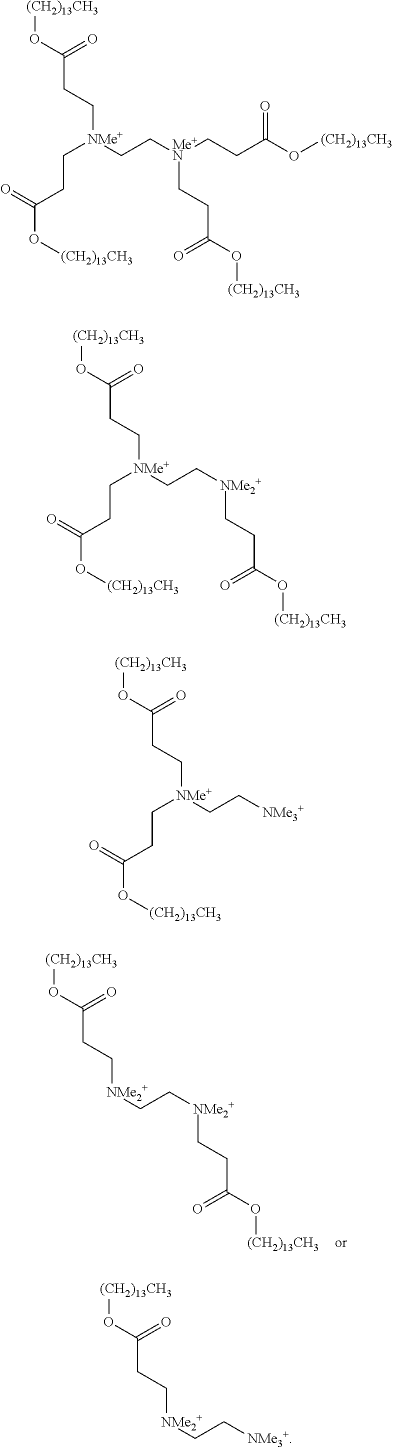

In certain embodiments, the lipid is prepared by reacting amine 32 with acrylate LG to form lipid LG32. In certain embodiments, the lipid LG32 is of one of the formulae below:

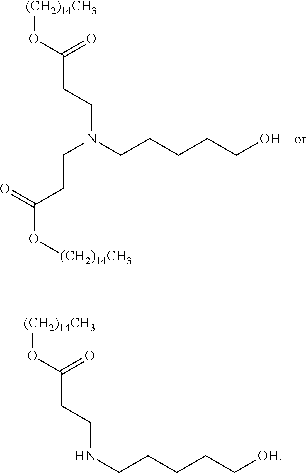

In other embodiments, the lipid is a composition of one or more of the above lipids.

In certain embodiments, the lipid is prepared by reacting amine 77 with acrylate LG to form lipid LG77. In certain embodiments, the lipid LG77 is of one of the formulae below:

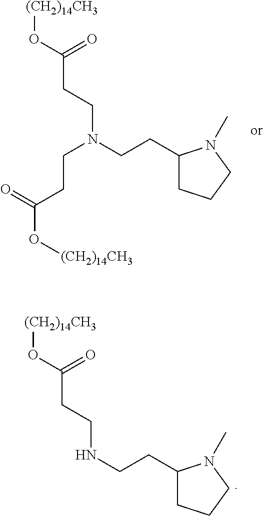

In other embodiments, the lipid is a composition of one or more of the above lipids.

In certain embodiments, the lipid is prepared by reacting amine 80 with acrylate LG to form lipid LG80. In certain embodiments, the lipid LG80 is of one of the formulae below:

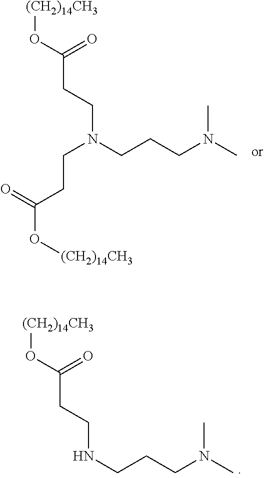

In other embodiments, the lipid is a composition of one or more of the above lipids.

In certain embodiments, the lipid is prepared by reacting amine 96 with acrylate

LG to form lipid LG96. In certain embodiments, the lipid NG96 is of one of the formulae below:

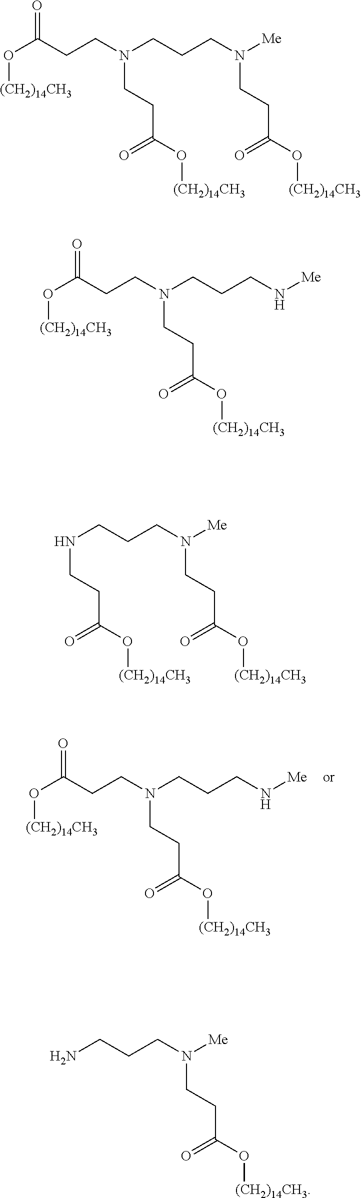

In other embodiments, the lipid is a composition of one or more of the above lipids.

In certain embodiments, the lipid is prepared by reacting amine 100 with acrylate LG to form lipid LG100. In certain embodiments, the lipid LG100 is of one of the formulae below:

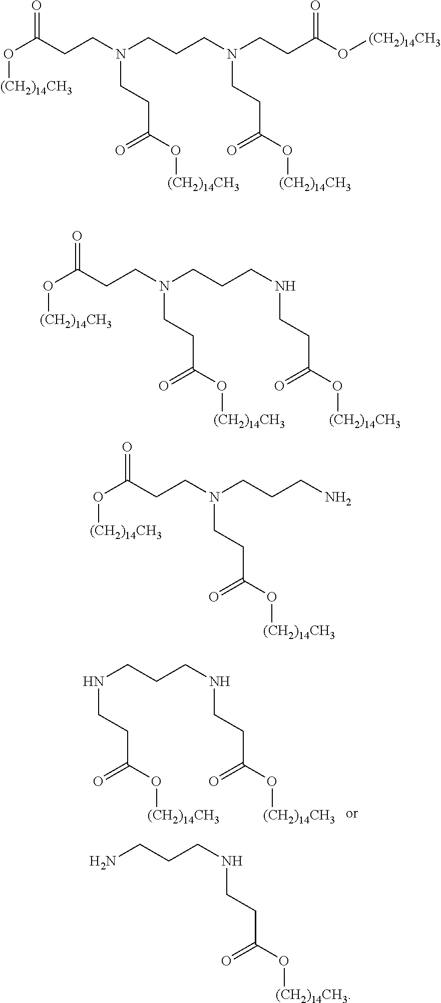

In other embodiments, the lipid is a composition of one or more of the above lipids. In certain embodiments, LG100 is treated with MeI or another alkylating agent to form lipids (QG100) of the formula:

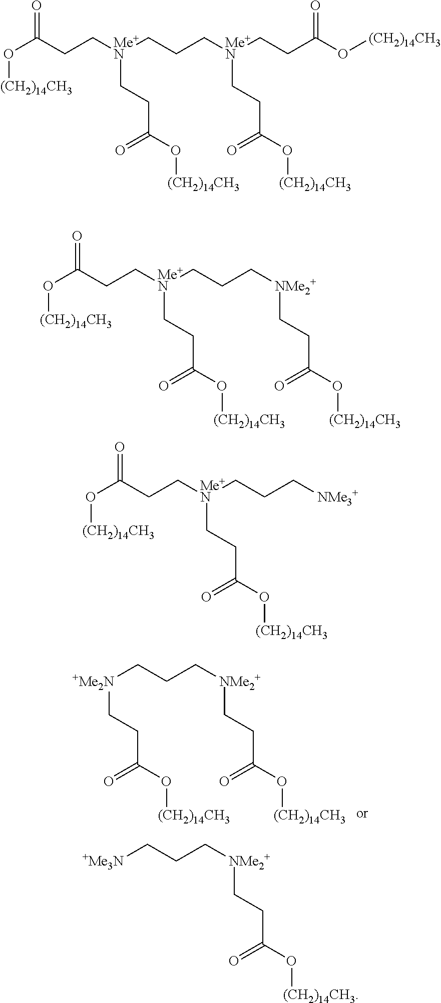

In certain embodiments, the lipid is prepared by reacting amine 109 with acrylate LG to form lipid LG109. In certain embodiments, the lipid NG 109 is of one of the formulae below:

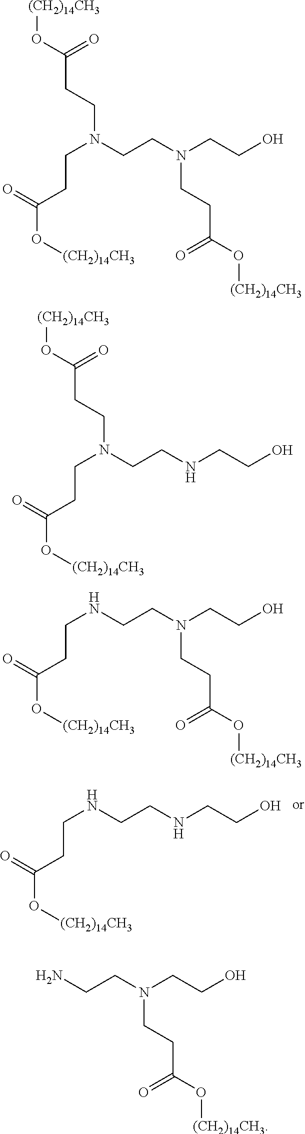

In other embodiments, the lipid is a composition of one or more of the above lipids.

In certain embodiments, the lipid is prepared by reacting amine 64 with acrylate LG to form lipid LG64. In certain embodiments, the lipid LG64 is of one of the formulae below:

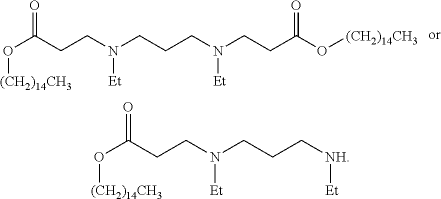

In other embodiments, the lipid is a composition of one or more of the above lipids.

In certain embodiments, the lipid is prepared by reacting amine 31 with acrylate LG to form lipid LG31. In certain embodiments, the lipid LG31 is of one of the formulae below:

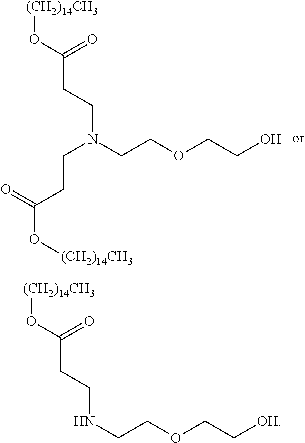

In other embodiments, the lipid is a composition of one or more of the above lipids.

In certain embodiments, the lipid is prepared by reacting amine 32 with acrylate LG to form lipid LG32. In certain embodiments, the lipid NG32 is of one of the formulae below:

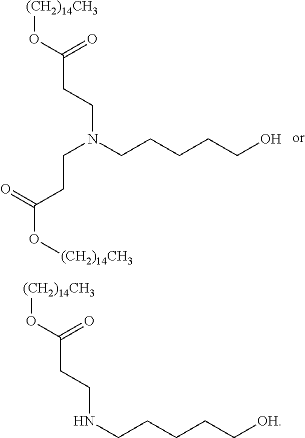

In other embodiments, the lipid is a composition of one or more of the above lipids.

Synthesis of Lipids

The lipids may be prepared by any method known in the art. Examples are provided, e.g., in WO 2006/138380.

Preferably, the lipids are prepared from commercially available starting materials, such acrylates or acrylamides, and amines. In another preferred embodiment, the lipids are prepared from easily and/or inexpensively prepared starting materials. As would be appreciated by one of skill in the art, the lipids can be prepared by total synthesis starting from commercially available starting materials. A particular lipid may be the desired final product of the synthesis, or a mixture of lipids may be the desired final product.

In a particularly preferred embodiment, the lipid is prepared via the conjugate addition of primary amines to acrylates or acrylamides. An exemplary reaction scheme is shown below:

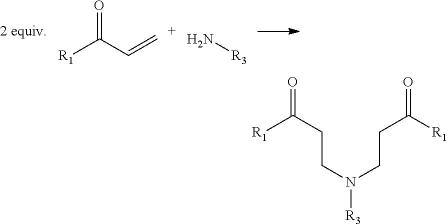

Any primary amine is useful in preparing lipids. Primary amines useful in this invention include, but are not limited to, methylamine, ethylamine, isopropylamine, aniline, substituted anilines, and ethanolamine. The primary amine may be a bis(primary amine). Preferably, the amine is commercially available. In certain embodiments, the amine used in the synthesis of the lipid is of the formula:

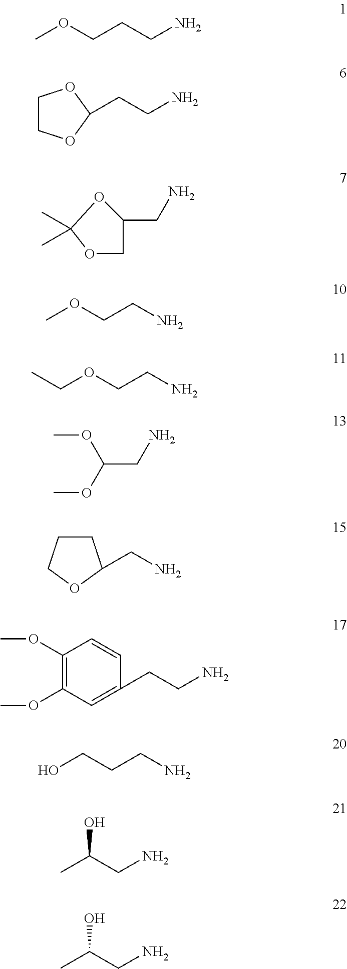

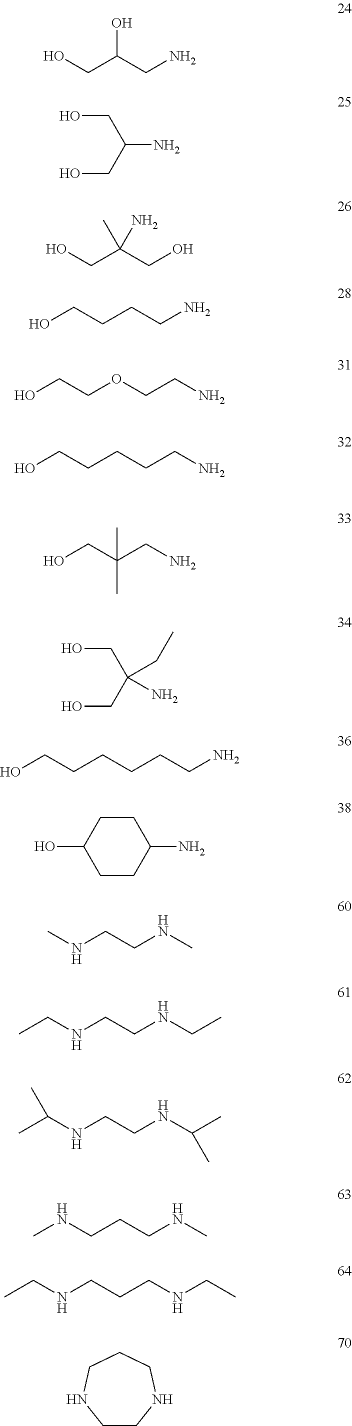

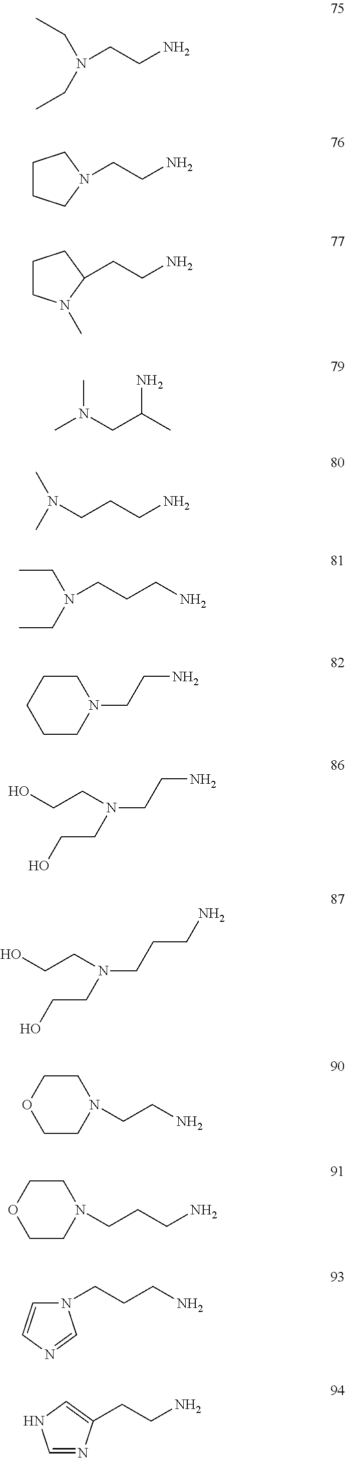

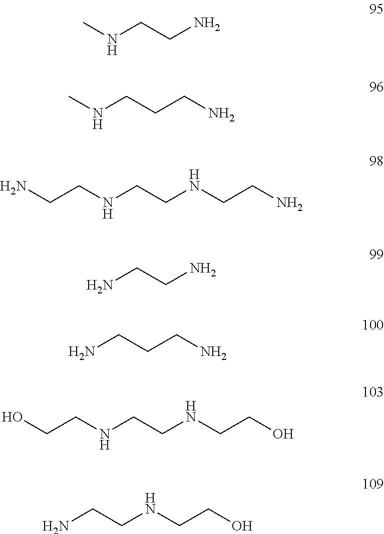

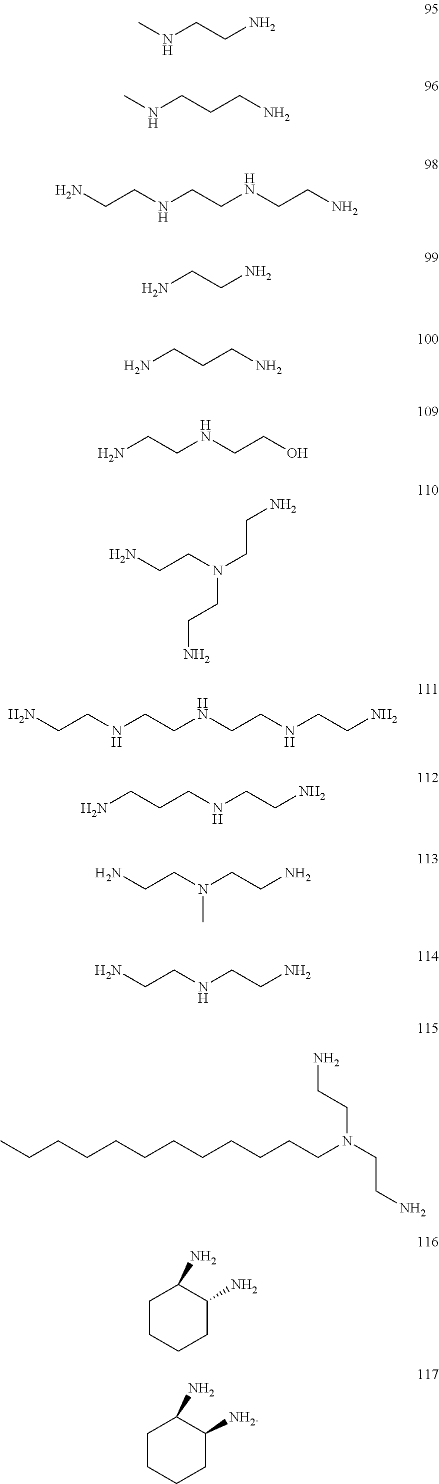

Acrylate or acrylamide monomers that are useful in the present invention include any acrylates and acrylamides. In certain embodiments, the acrylates or acrylamides are acrylates or acrylamides of straight chain alkyl groups. In certain embodiments, the acrylate or acrylamide is of the formula:

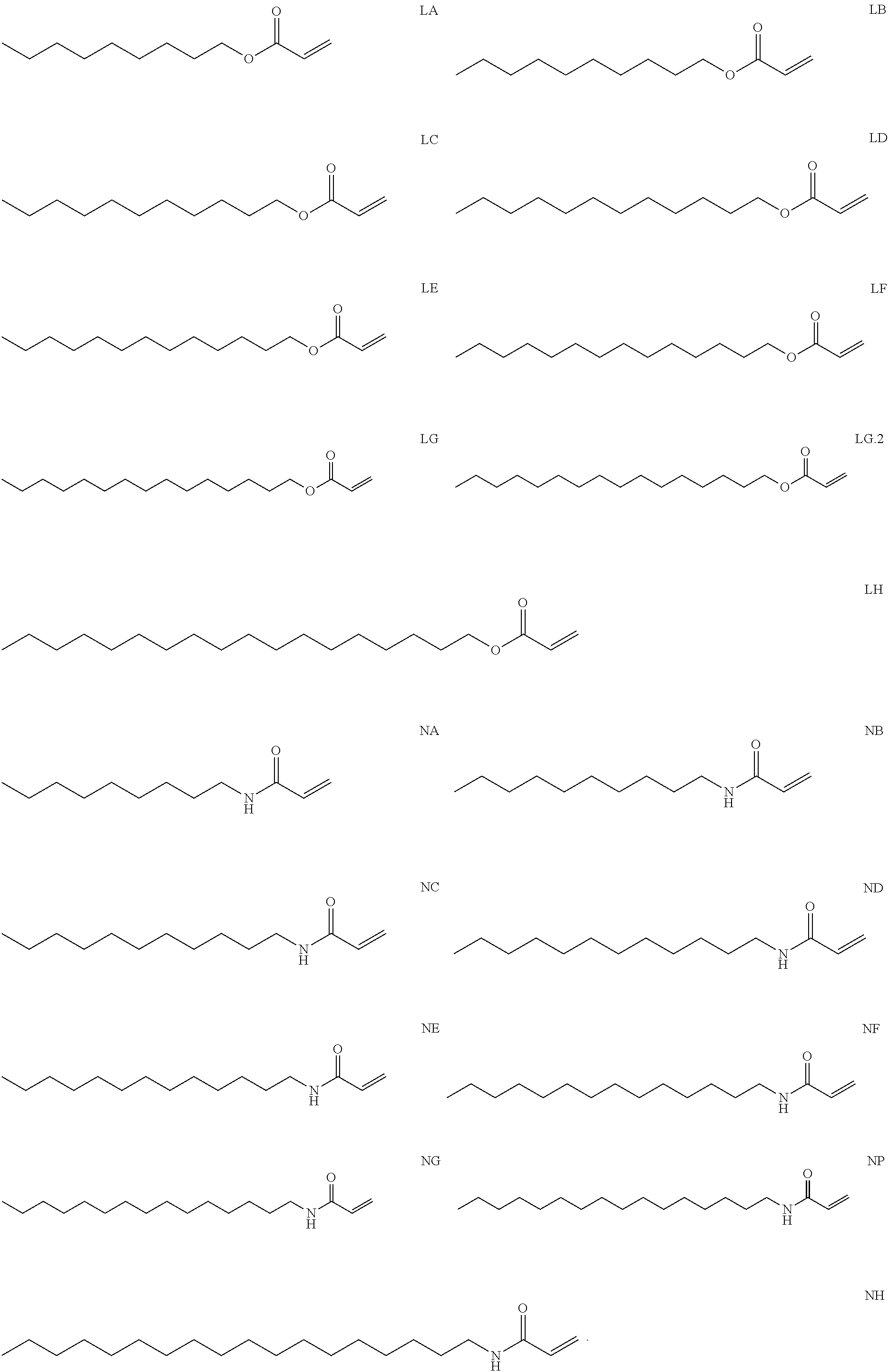

In other embodiments, the acrylate or acrylamide may include branched, substituted, or cyclic aliphatic or heteroaliphatic groups. In certain embodiments, the acrylate or acrylamide is substituted with C1-C6 alkyl group, halogens, amino groups, hydroxyl groups, alkoxy groups, etc.

In certain embodiments, the reaction is performed neat without the use of a solvent. In other embodiments, a solvent is used for the reaction. Both or one of the monomers is dissolved in an organic solvent (e.g., THF, CH2Cl2, MeOH, EtOH, CHCl3, hexanes, toluene, benzene, CCl4, glyme, diethyl ether, etc.). The resulting solutions are combined, and the reaction mixture is heated to yield the desired lipid. In a particularly preferred embodiment, the reaction mixture is heated to temperature ranging from 50-150° C. In another particularly preferred embodiment, the reaction mixture is heated to approximately 95° C. The reaction may also be catalyzed. For example, the reaction may be catalyzed by the addition of an acid, base, or metal. The reagents may be allowed to react for fours, days, or weeks. Preferably, the reaction is allowed to proceed from overnight (e.g., 8-2 hours) to 7 days.

In another particularly preferred embodiment, the lipids are prepared by the conjugate addition of a bis(amine) to an acrylate. The bis(amine) may be a bis(secondary amine) or a bis(primary amine). En exemplary reaction scheme using bis(amines) is shown below:

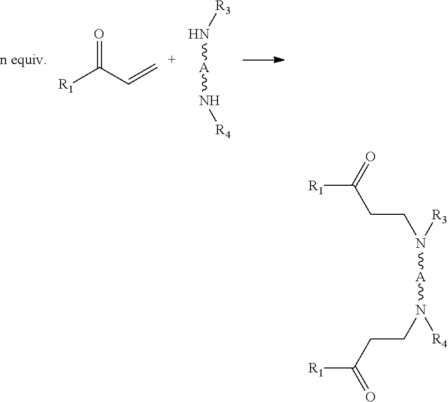

In certain embodiments, the reaction is performed neat without a solvent. In other embodiments, the reaction is performed in a solvent. One or both of the monomers are dissolved in an organic solvent (e.g., THF, CH2Cl2, MeOH, EtOH, CHCl3, hexanes, CCl4, glyme, diethyl ether, etc.). Organic solvents are preferred due to the susceptibility of polyesters to hydrolysis. The resulting solutions are combined, and the reaction mixture is heated to yield the desired lipid. In a particularly preferred embodiment, the reaction mixture is maintained at a temperature ranging from 50-150° C. In another particularly preferred embodiment, the reaction mixture is heated to approximately 95° C. The reaction may also be catalyzed. For example, the reaction may be catalyzed by the addition of an acid, base, or metal.

In yet another particularly preferred embodiment, the lipids are prepared by the conjugate addition of a poly(amine) to an acrylate or acrylamide. The poly(amine) may include primary, secondary, tertiary, or quaternary amines. In certain embodiments, the poly(amine) contains only primary and secondary amines. An exemplary reaction scheme using poly(amines) is shown below:

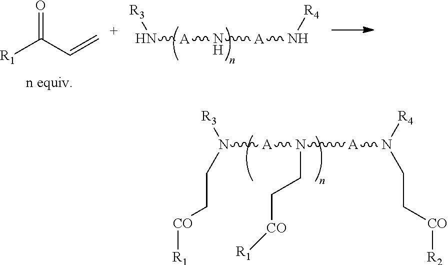

In certain embodiments, the reaction is performed with an excess of acrylate or acrylamide to fully funcationlize all amino groups of the poly(amine). In other embodiments, the equivalents of acrylate are limiting; therefore, all amino groups of the poly(amine) are not functionalized. In certain embodiments, the reaction is performed neat without a solvent. In other embodiments, the reaction is performed in a solvent. One or both of the monomers are dissolved in an organic solvent (e.g., THF, CH2Cl2, MeOH, EtOH, CHCl3, hexanes, CCl4, glyme, diethyl ether, etc.). Organic solvents are preferred due to the susceptibility of polyesters to hydrolysis. The resulting solutions are combined, and the reaction mixture is heated to yield the desired lipid. In a particularly preferred embodiment, the reaction mixture is maintained at a temperature ranging from 50-150° C. In another particularly preferred embodiment, the reaction mixture is heated to approximately 95° C. The reaction may also be catalyzed. For example, the reaction may be catalyzed by the addition of an acid, base, or metal.

The synthesized lipid may be purified by any technique known in the art including, but not limited to, precipitation, crystallization, chromatography, distillation, etc. In a particularly preferred embodiment, the lipid is purified through repeated precipitations in organic solvent (e.g., diethyl ether, hexane, etc.). In a particularly preferred embodiment, the lipid is isolated as a salt. The lipid is reacted with an acid (e.g., an organic acid or inorganic acid) to form the corresponding salt. In certain embodiments, the tertiary amine is alkylated to form a quaternary ammonium salt of the lipid. The tertiary amines may be alkylated with any alkylating agent, for example, alkyl halides such as methyl iodide may be used to from the quaternary amino groups. The anion associated with the quaternary amine may be any organic or inorganic anion. Preferably, the anion is a pharmaceutically acceptable anion.

In certain embodiments, the reaction mixture results in a mixture of isomers with varying numbers and positions of acrylate tails. Such mixtures of products may be used as is, or a single isomer may be purified from the reaction mixture. When an amine is not exhaustively alkylated, the resulting primary, secondary, or tertiary amines may be further reacted with another acrylate, acrylamide, or other electrophile. The resulting lipid may then be optionally purified.

In certain embodiments, a desired lipid is prepared by traditional total synthesis. In certain embodiments, a commercially available amine is the starting material. One or more amino groups of the amine are optionally protected. The unprotected amino groups are reacted with a acrylate or acrylamide. The product is optionally purified. Protecting groups are removed, and the free amino groups are optionally reacted with another acrylate, acrylamide, or other electrophile. Such a sequence may be repeated depending on the desired complexity of the product being prepared. The final product may then be optionally purified.

The lipids possess tertiary amines. Although these amines are hindered, they are available to interact with a therapeutic agent (e.g., a protein used in replacement therapy, e.g., a protein described herein). The therapeutic agent is contacted with the lipids under conditions suitable to form therapeutic agent/lipid complexes. In some embodiments, the lipid is preferably at least partially protonated, e.g., so as to form a complex with the negatively charged therapeutic agent. In a preferred embodiment, the therapeutic agent/lipid complexes form nanoparticles that are useful in the delivery of therapeutic agents to cells. In certain embodiments, multiple lipid molecules may be associated with a therapeutic agent molecule. The complex may include 1-100 lipid molecules, 1-1000 lipid molecules, 10-1000 lipid molecules, or 100-10,000 lipid molecules. In certain embodiments, the complex may form a nanoparticle. In a particularly preferred embodiment, the diameter of the nanoparticles ranges from 10-500 nm, more preferably the diameter of the nanoparticles ranges from 10-1200 nm, and most preferably from 50-150 nm. The nanoparticles may be associated with a targeting agent as described below.

Drug Delivery Devices

Nanoparticles are particles in the nanometer size range whereas microparticles are particles in the micrometre size range. Both types of particle can be used as drug delivery devices. The lipids described herein may also be used to form drug delivery devices (e.g., microparticles and/or nanoparticles), e.g., to deliver therapeutic agent (e.g., a therapeutic protein, e.g., a protein for use in replacement therapy, e.g., a protein described herein). The lipids may be used to encapsulate agents including therapeutic agents (e.g., therapeutic proteins, e.g., proteins used in replacement therapy, e.g., a protein described herein). The lipids have several properties that make them particularly suitable in the preparation of drug delivery devices. These include 1) the ability of the lipid to complex and “protect” labile agents; 2) the ability to buffer the pH in the endosome; 3) the ability to act as a “proton sponge” and cause endosomolysis; and 4) the ability to neutralize the charge on negatively charged agents. In a preferred embodiment, the lipids are used to form microparticles and/or nanoparticles containing the therapeutic agent to be delivered. These microparticles and/or nanoparticles may include other materials such as polynucleotides, peptides, proteins, carbohydrates, synthetic polymers (e.g., PEG, PLGA), and natural polymers. In a particularly preferred embodiment, the diameter of the microparticles ranges from between 500 nm to 50 micrometers, more preferably from 1 micrometer to 20 micrometers, and most preferably from 1 micrometer to 10 micrometers. In another particularly preferred embodiment, the microparticles range from 1-5 micrometers. Nanoparticles can be smaller in size (e.g., between 100 nanometers and 1 micron).

Methods of Preparing Microparticles

The microparticles may be prepared using any method known in this art. These include, but are not limited to, spray drying, single and double emulsion solvent evaporation, solvent extraction, phase separation, simple and complex coacervation, and other methods well known to those of ordinary skill in the art. Particularly preferred methods of preparing the particles are the double emulsion process and spray drying. Nanoparticles can be created by any technique well known in the art. They can be created in the same or similar manner as microparticles. In some embodiments, high-speed mixing or homogenization can be used to reduce the size of the polymer/emulsions to less than 2 microns (see, e.g., WO 97/04747). The conditions used in preparing the microparticles and/or nanoparticles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix.

Methods developed for making microparticles and/or nanoparticles for delivery of encapsulated agents are described in the literature (for example, Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al Reactive Polymers 6:275-283, 1987; Mathiowitz et al. J. Appl. Polymer Sci. 35:755-774, 1988).

If the particles prepared by any of the above methods have a size range outside of the desired range, the particles can be sized, for example, using a sieve. The particle may also be coated. In certain embodiments, the particles are coated with a targeting agent. In other embodiments, the particles are coated to achieve deisirable surface properties (e.g., a particular charge).

Micelles and Liposomes

The lipids described herein may be used to prepare micelles or liposomes (e.g., micelles or liposomes that contain a therapeutic agent (e.g., a protein used in replacement therapy, e.g., a protein described herein). Many techniques for preparing micelles and liposomes are known in the art, and any method may be used with the lipids to make micelles and liposomes. In addition, any agent including polynucleotides, small molecules, proteins, peptides, metals, organometallic compounds, etc. may be included in a micelle or liposome.

In certain embodiments, liposomes (lipid vesicles) are formed through spontaneous assembly. In other embodiments, liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these particles have formed, reducing the size of the particle can be modified through input of sonic energy (sonication) or mechanical energy (extrusion). See Walde, P. “Preparation of Vesicles (Liposomes)” In Encylopedia of Nanoscience and Nanotechnology; Nalwa, H. S. Ed. American Scientific Publishers: Los Angeles, 2004; Vol. 9, pp. 43-79; Szoka et al. “Comparative Properties and Methods of Preparation of Lipid Vesicles (Liposomes)” Ann Rev. Biophys. Bioeng. 9:467-508, 1980. The preparation of lipsomes involves preparing the lipid for hydration, hydrating the lipid with agitation, and sizing the vesicles to achieve a homogenous distribution of liposomes. Lipids are first dissolved in an organic solvent to assure a homogeneous mixture of lipids. The solvent is then removed to form a lipid film. This film is thoroughly dried to remove residual organic solvent by placing the vial or flask on a vaccuum pump overnight. Hydration of the lipid film/cake is accomplished by adding an aqueous medium to the container of dry lipid and agitating the mixture. Disruption of LMV suspensions using sonic energy typically produces small unilamellar vesicles (SUV) with diameters in the range of 15-50 nm Lipid extrusion is a technique in which a lipid suspension is forced through a polycarbonate filter with a defined pore size to yield particles having a diameter near the pore size of the filter used. Extrusion through filters with 100 nm pores typically yields large, unilamellar vesicles (LUV) with a mean diameter of 120-140 nm

In certain embodiments of the invention, liposomes are formed comprising a lipid, PEG-ceramide, cholesterol, and a therapeutic agent (e.g., protein used in replacement therapy, e.g., a protein described herein). In some embodiments, the lipid is ND28, ND32, LF94, ND99, ND95, NP103, NP98, ND25, ND20, ND100, NF96, NF103, NF109, NFI1, ND24, NF86, NP96, ND36, NF61, NF87, NF95, QG100, NF60, NP1005 NF1, NP99, QD99, NF63, LG109, ND103, LF95, QF99, LG100, LF31, LG32, NF109, NF64, LE87, LG77, LG96, ND96, LD31, NG64, ND 109, or LG80. In certain embodiments, the amount of lipid in the liposome ranges from 30-80 mol %, preferably 40-70 mol %, more preferably 60-70 mol %. In certain embodiments, the amount of PEG-ceramide in the liposomes ranges from 5-20 mol %, preferably 10-15 mol %, more preferably approximately 10 mol %. In certain embodiments, the amount of cholesterol in the liposome ranges from 5-25 mol %, preferably 10-20 mol %, more preferably approximately 15 mol %. In certain embodiments, the amount of cholesterol in the liposome is approximately 20 mol %. These liposomes may be prepared using any method known in the art. In certain embodiments, the liposomes are prepared by lipid extrusion.

Certain lipids can spontaneously self assemble around certain molecules to form liposomes. Use of the lipids can allow for simple assembly of liposomes without the need for additional steps or devices such as an extruder.

The following scientific papers described other methods for preparing liposomes and micelles: Narang et al. “Cationic Lipids with Increased DNA Binding Affinity for Nonviral Gene Transfer in Dividing and Nondividing Cells” Bioconjugate Chem. 16:156-68, 2005; Hofland et al. “Formation of stable cationic lipid/DNA complexes for gene transfer” Proc. Natl. Acad. Sci. USA 93:7305-7309, July 1996; Byk et al. “Synthesis, Activity, and Structure—Activity Relationship Studies of Novel Cationic Lipids for DNA Transfer” J. Med. Chem. 41(2):224-235, 1998; Wu et al. “Cationic Lipid Polymerization as a Novel Approach for Constructing New DNA Delivery Agents” Bioconjugate Chem. 12:251-57, 2001; Lukyanov et al. “Micelles from lipid derivatives of water-soluble polymers as delivery systems for poorly soluble drugs” Advanced Drug Delivery Reviews 56:1273-1289, 2004; Tranchant et al. “Physicochemical optimisation of plasmid delivery by cationic lipids” J. Gene Med. 6-.S24-S35, 2004; van Bum et al. “Liposome/Water Lipophilicity: Methods, Information Content, and Pharmaceutical Applications” Medicinal Research Rev. 24(3):299-324, 2004.

Targeting Agents

The inventive complexes, liposomes, micelles, microparticles, and nanoparticles may be modified to include targeting agents since it is often desirable to target a particular cell, collection of cells, or tissue. A variety of targeting agents that direct pharmaceutical compositions to particular cells are known in the art (see, for example, Gotten et al. Methods Enzym. 217:618, 1993). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gpl20 envelope protein of the human immunodeficiency virus (HIV), carbohydrates, receptor ligands, sialic acid, etc. If the targeting agent is included throughout the particle, the targeting agent may be included in the mixture that is used to form the particles. If the targeting agent is only on the surface, the targeting agent may be associated with (i.e., by covalent, hydrophobic, hydrogen bonding, van der Waals, or other interactions) the formed particles using standard chemical techniques. The targeting strategy may also include several different targeting moieties (Journal of Controlled Release 120 (2007) 242-249).

Targeting strategy may also involve the reductive cleavage of a sensitive bond between the protein and the polymer (e.g., strategic disulfide insertions) (Journal of Controlled Release 120 (2007) 250-258.

Pharmaceutical Compositions

Once the complexes, micelles, liposomes, microparticles, or nanoparticles (e.g., containing a lipid described herein and a therapeutic agent described herein (e.g., a therapeutic protein, e.g., a protein for use in replacement therapy, e.g., a protein described herein)) have been prepared, they may be combined with one or more pharmaceutical excipients to form a pharmaceutical composition that is suitable to administer to animals including humans. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and Tantioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., microparticles, nanoparticles, liposomes, micelles, therapeutic agent/lipid complexes), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the particles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles or nanoparticles in a proper medium. Absorption enhancers can also be used to increase the flux of the compound across the skin. The rate can be controlled by either providing a rate controlling membrane or by dispersing the particles in a polymer matrix or gel.

### EXAMPLES

For use in the preparation of therapeutic complexes containing a lipid and a therapeutic agent described herein, examples of how to prepare the lipids are as follows.

Briefly, monomers are purchased from Aldrich (Milwaukee, Wis.), TCI (Portland, Oreg.), Pfaltz & Bauer (Waterbury, Conn.), Matrix Scientific (Columbia, S.C.), Acros-Fisher (Pittsburg, Pa.), Scientific Polymer (Ontario, N.Y.), Polysciences (Warrington, Pa.), and Dajac monomer-polymer (Feasterville, Pa.). The acrylate and amine monomers are used neat to prepare the lipids. All possible pair wise combinations of amine and acrylate monomers shown in FIGS. 1A and 1B are prepared in sealed vials. The vials are then incubated overnight at approximately 95° C. with shaking. The synthesized lipids are used without further purification. The molecular weights of the synthesized lipids can be determined by mass spectroscopy and compared to predicted molecular weights to confirm synthesis of the lipid. The methods are detailed in WO2006/138380, e.g., Example 1 of WO2006/138380.

Formation of Protein/Polymer Complexes: Complexes that contain a therapeutic protein described herein and a lipid described herein are prepared. A solution containing the lipid in buffer, such as 25 mM sodium acetate buffer, is added to a solution containing the therapeutic protein in buffer. The amount of lipid added can be adjusted to provide a molar ratio of 2.5:1; 5:1, 10:1; 15:1; 20:1; or 25:1 w/w of protein to lipid. The solution containing the lipid and protein is incubated 20-30 minutes, for example, at room temperature to form the complex.

A number of embodiments of the invention have been described. Nevertheless, it will be understood that various modifications may be made without departing from the spirit and scope of the invention.

What is claimed is:

1. A therapeutic composition comprising an amine-containing lipid and a therapeutic agent, wherein the therapeutic agent comprises a protein.

2. The therapeutic composition of claim 1, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

3. A therapeutic composition comprising a therapeutic protein and a compound of the formula: 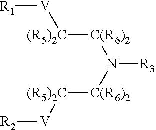
wherein V is selected from the group consisting of C═O, C═S, S═O, and SO2;
R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(−0)RA; —NRAC(═O)N(RA)2; —OC(═O)ORA; —OC(═O)RA; —OC(═O)N(RA)2; —NRAC(═O)ORA; or —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(═O)RB; —CO2RB; —CN; —SCN; —SRB; —SORB; —SO2R3; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of R3 is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;
R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORC; —C(═O)Rc; —CO2RC; —CN; —SCN; —SRO; —SORc; —SO2RC; —NO2; —N3; —N(Rc)2; —NHC(=0)Rc; —NRCC(═O)N(RC)2; —OC(═O)ORc; —OC(═O)RC; —OC(O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R5 is independently selected from the group consisting of hydrogen and C1-C6 alkyl; each occurrence of R6 is independently selected from the group consisting of hydrogen and C1-C6 alkyl; with the proviso that when all occurrences of R5 and R6 are hydrogen, V is C═O, R1 is —ORA, R2 is —ORB, and R1 and R2 are the same, then R3 is not —CH2CH2ORc′, wherein Rc′ is methyl, ethyl, propyl, isopropyl, butyl, s-butyl, isobutyl, t-butyl, pentyl, cyclopentyl, hexyl, cyclohexyl, decyl, methoxymethyl, 2-methoxyethyl, 1-ethoxyethyl, 2-ethoxyethyl, (2-methoxyethoxy)methyl, 2-tetrahydrofuranyl, 2-tetrahydropyranyl, tetrahydrofurfuryl, fo[pi]nyl, acetyl, propionyl, butyryl, isobutyryl, pivaloyl, valeryl, methoxyacetyl, ethoxyacetyl, acetoxyacetyl, 2-formyloxyethyl, 2-acetoxyethyl, 2-oxopropyl, 2-oxobutyl, 2-oxocyclopentyl, 2-oxo-3-tetrahydrofuranyl, 2-oxo-3-tetrahydropyranyl, methoxycarbonyl, ethoxycarbonyl, and t-butoxycarbonyl; and with the proviso that when all occurrences of R5 and R6 are hydrogen, V is C═O, R1 is —ORA, wherein RA is a straight-chain, branched or cyclic alkyl group of 1 to 20 carbon atoms which may contain an ether, carbonyl, or carbonyloxy group; R2 is —ORB, wherein RB is a straight-chain, branched or cyclic alkyl group of 1 to 20 carbon atoms which may contain an ether, carbonyl, or carbonyloxy group; and R1 and R2 are the same, the R3 is not —CH2CH2ORc″, wherein Rc″ is a straight-chain, branched or cyclic alkyl group of 1 to 20 carbon atoms which may contain an ether, carbonyl, or carbonyloxy group; and salts thereof.

4. The therapeutic composition of claim 3, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

5. A therapeutic composition comprising a therapeutic protein and a compound of the formula: 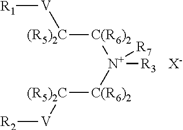
wherein V is selected from the group consisting of C═O, C═S, S═O, and SO2;
R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; or —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(═O)RB; —CO2RB; —CN; —SCN; —SRB; —SORB; —SO2RB; —NO2; —N3; —N(RB)2; —NHC(=0)RB; —NRBC(═O)N(RB)2; —OC(═O)ORB; —OC(═O)RB; —OC(═O)N(RB)2; —NRBC(=0)0RB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;
R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORC; —C(═O)RC; —CO2Rc; —CN; —SCN; —SRC; —SORC; —SO2Rc; —NO2; —N3; —N(Rc)2; —NHC(═O)Rc; —NRcC(═O)N(Rc)2; —OC(═O)0Rc; —OC(═O)Rc; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R5 is independently selected from the group consisting of hydrogen and C1-C6 alkyl; each occurrence of R6 is independently selected from the group consisting of hydrogen and C1-C6alkyl;
R7 is hydrogen or C1-C6 aliphatic; and
X is an anion; with the proviso that when all occurrences of R5 and R6 are hydrogen, V is C═O, R1 is —ORA, R2 is —ORB, and R1 and R2 are the same, then R3 is not —CH2CH2ORc′, wherein Rc′ is methyl, ethyl, propyl, isopropyl, butyl, s-butyl, isobutyl, t-butyl, pentyl, cyclopentyl, hexyl, cyclohexyl, decyl, methoxymethyl, 2-methoxyethyl, 1-ethoxyethyl, 2-ethoxyethyl, (2-methoxyethoxy)methyl, 2-tetrahydrofuranyl, 2-tetrahydropyranyl, tetrahydrofurfuryl, formyl, acetyl, propionyl, butyryl, isobutyryl, pivaloyl, valeryl, methoxyacetyl, ethoxyacetyl, acetoxyacetyl, 2-formyloxyethyl, 2-acetoxyethyl, 2-oxopropyl, 2-oxobutyl, 2-oxocyclopentyl, 2-oxo-3-tetrahydrofuranyl, 2-oxo-3-tetrahydropyranyl, methoxycarbonyl, ethoxycarbonyl, and t-butoxycarbonyl; and with the proviso that when all occurrences of R5 and R6 are hydrogen, V is C═O, R1 is —ORA, wherein RA is a straight-chain, branched or cyclic alkyl group of 1 to 20 carbon atoms which may contain an ether, carbonyl, or carbonyloxy group; R2 is —ORB, wherein RB is a straight-chain, branched or cyclic alkyl group of 1 to 20 carbon atoms which may contain an ether, carbonyl, or carbonyloxy group; and R1 and R2 are the same, the R3 is not —CH2CH2ORc″, wherein Rc″ is a straight-chain, branched or cyclic alkyl group of 1 to 20 carbon atoms which may contain an ether, carbonyl, or carbonyloxy group; and salts thereof.

6. The therapeutic composition of claim 5, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

7. A therapeutic composition comprising a therapeutic protein and a compound of the formula: 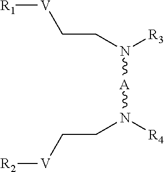
wherein A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; and substituted or unsubstituted, branched or unbranched heteroaryl;
V is selected from the group consisting of C═O, C═S, S═O, and SO2;
R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(═O)RB; —CO2RB; —CN; —SCN; —SRB; —SORB; —SO2RB; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;
R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORC; —C(═O)RC; —CO2RC; —CN; —SCN; —SRC; —SORO; —SO2RC; —NO2; —N3; —N(Rc)2; —NHC(═O)RC; —NRCC(═O)N(RC)2; —OC(═O)ORC; —OC(═O)RC; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORD; —C(═O)RD; —CO2RO; —CN; —SCN; —SRO; —SORD; —SO2RO; —NO2; —N3; —N(RD)2; —NHC(═O)RD; —NRCC(═O)N(RO)2; -0C(═O)ORD; —OC(═O)RO; —OC(═O)N(RO)2; —NRcC(═O)ORD; or —C(RO)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; and salts thereof.

8. The therapeutic composition of claim 7, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

9. A therapeutic composition comprising a therapeutic protein and a compound of the formula: 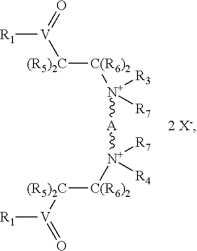
wherein A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; and substituted or unsubstituted, branched or unbranched heteroaryl;
V is selected from the group consisting of C═O, C═S, S═O, and SO2;
R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORA; —C(O)RA; —CO2RA; —CN; —SCN; —SRA; —SORA; —SO2RA; —NO2; —N3; —N(RA)2; —NHC(═O)RA; —NRAC(O)N(RA)2; —OC(═O)ORA; —OC(O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORB; —C(O)R8; —CO2RB; —CN; —SCN; —SRB; —SORB; —SO2RB; —NO2; —N3; —N(RB)2; —NHC(O)RO; —NRBC(O)N(RB)2; —OC(O)OR8; —OC(O)RB; —OC(O)N(RB)2; —NRBC(O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;
R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORO; C(O)RO; —CO2RC; —CN; —SCN; —SRC; —SORO; —SO2RC; —NO2; —N3; —N(Rc)2; —NHC(O)RO; —NROC(O)N(Rc)2; —OC(O)ORO; —OC(O)RO; —OC(O)N(Rc)2; —NRcC(O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; —ORD; —C(O)Rn; —CO2RO; —CN; —SCN; —SRD; —SORD; —SO2RO; —NO2; —N3; —N(RD)2; —NHC(O)RO; —NRCC(O)N(RD)2; —OC(O)ORO; —OC(O)RO; —OC(O)N(RD)2; —NROC(O)ORD; or —C(RD)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstitued, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R7 is C1-C6 aliphatic; X is an anion; and salts thereof.

10. The therapeutic composition of claim 9, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

11. A therapeutic composition comprising a therapeutic protein and a compound of the formula: 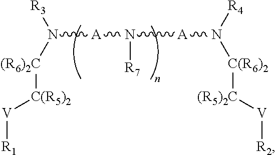
wherein
A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; and substituted or unsubstituted, branched or unbranched heteroaryl;
V is selected from the group consisting of C═O, C═S, S═O, and SO2;
R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(K))ORA; —OC(O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(═O)RB; —CO2R3; —CN; —SCN; —SRB; —SORB; —SO2RB; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;
R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORC; —C(═O)Rc; —CO2RC; —CN; —SCN; —SRC; —SORC; —SO2RC; —NO2; —N3; —N(Rc)2; —NHC(═O)Rc; —NRcC(═O)N(Rc)2; —OC(═O)ORc; —OC(═O)RO; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORD; —C(═O)RO; —CO2RO; —CN; —SCN; —SR; —SORD; —SO2RD; —NO2; —N3; —N(RD)2; —NHC(═O)RD; —NRCC(═O)N(RD)2; —OC(═O)ORD; —OC(═O)RD; —OC(═O)N(RD)2; —NRCC(═O)ORD; or —C(RD)3; wherein each occurrence of Ro is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R5 is independently selected from the group consisting of hydrogen and C1-C6 alkyl; each occurrence of R6 is independently selected from the group consisting of hydrogen and C1-C6alkyl;
R7 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORG; —CC(═O)RG; —CO2RG; —CN; —SCN; —SRG; —SORG; —SO2RG; —NO2; —N3; —N(RG)2; —NHC(═O)RG; —NRGC(═O)N(RG)2; —OC(═O)ORG; —OC(═O)RO; —OCC(═O)N(RG)2; —NRGC(═O)ORG; and —C(RG)3; wherein each occurrence of Ro is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety and salts thereof.

12. The therapeutic composition of claim 11, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

13. A therapeutic composition comprising a therapeutic protein and a compound of the formula: 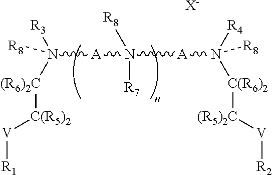
wherein
A is selected from the group consisting of cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl;
and substituted or unsubstituted, branched or unbranched heteroaryl;
V is selected from the group consisting of C═O, C═S, S═O, and SO2;
R1 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORA; —C(═O)RA; —CO2RA; —CN; —SCN; —SRA; —SORA; —SO2RA; —NO2; —N3; —N(RA)2; —NHC(═O)RA; —NRAC(═O)N(RA)2; —OC(═O)ORA; —OC(═O)RA; —OC(═O)N(RA)2; —NRAC(═O)ORA; and —C(RA)3; wherein each occurrence of RA is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R2 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORB; —C(O)R3; —CO2RB; —CN; —SCN; —SRB; —SORB; —SO2R8; —NO2; —N3; —N(RB)2; —NHC(═O)RB; —NRBC(═O)N(RB)2; —OC(═O)ORB; —OC(═O)RB; —OC(═O)N(RB)2; —NRBC(═O)ORB; or —C(RB)3; wherein each occurrence of RB is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R1 and R2 may be taken together to form a cyclic structure;
R3 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORC; —C(O)Rc; —CO2RC; —CN; —SCN; —SRO; —SORC; —SO2Rc; —NO2; —N3; —N(Rc)2; —NHC(═O)RC; —NRCC(═O)N(RC)2; —OC(═O)ORc; —OC(═O)Rc; —OC(═O)N(Rc)2; —NRcC(═O)ORc; or —C(Rc)3; wherein each occurrence of Rc is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety;
R4 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORD; —C(═O)RD; —CO2RO; —CN; —SCN; —SRD; —SORD; —SO2RO; —NO2; —N3; —N(RD)2; —NHC(═O)RD; —NRCC(═O)N(RD)2; —OC(═O)ORD; —OC(═O)RD; —OC(═O)N(RD)2; —NRCC(═O)ORD; or —C(RD)3; wherein each occurrence of RD is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; wherein R3 and R4 may be taken together to form a cyclic structure; each occurrence of R5 is independently selected from the group consisting of hydrogen and C1-C6 alkyl; each occurrence of R6 is independently selected from the group consisting of hydrogen and C1-C6alkyl;
R7 is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —ORG; —C(═O)RG; —CO2RG; —CN; —SCN; —SRG; —SORG; —SO2RG; —NO2; —N3; —N(RG)2; —NHC(═O)RG; —NRGC(═O)N(RG)2; —OC(═O)ORG; —OC(═O)RO; —OC(═O)N(RG)2; —NRGC(═O)ORG; and —C(RG)3; wherein each occurrence of RG is independently a hydrogen; a protecting group; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; an acyl moiety; alkoxy; aryloxy; alkylthio; arylthio; amino, alkylamino, dialkylamino, heteroaryloxy; or heteroarylthio moiety; each occurrence of R8 is hydrogen or C1-C6 aliphatic; each dashed line represents a bond or the absence of a bond, wherein when the dashed line represents a bond, the attached nitrogen is positively charged; and X is any anion.

14. The therapeutic composition of claim 13, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

15. A microparticle comprising the therapeutic composition of claim 1.

16. The microparticle of claim 15, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

17. A liposome comprising the therapeutic composition of claim 1.

18. The liposome of claim 17, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

19. A micelle comprising the therapeutic composition of claim 1.

20. The micelle of claim 19, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

21. A pharmaceutical composition comprising the therapeutic composition of claim 1 and a pharmaceutical agent.

22. The pharmaceutical composition of claim 21, wherein the protein comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

23. A method of making a therapeutic composition, the method comprising:
providing a lipid described herein;
providing a therapeutic agent described herein; and
combining the lipid and the therapeutic agent, thereby making a therapeutic composition.

24. The method of claim 23, wherein the therapeutic agent is a therapeutic protein that comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

25. A method of preparing microparticles, the method comprising:
contacting a therapeutic agent described herein with a lipid described herein in the presence of a solvent to form a mixture; and spray drying the mixture, thereby preparing microparticles.

26. The method of claim 25, wherein the therapeutic agent is a therapeutic protein that comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.

27. A method of treating an individual, the method comprising:
administering the therapeutic composition of claim 1 to an individual in need of such treatment.

28. The method of claim 27, wherein the individual is in need of replacement therapy.

29. The method of claim 27, wherein the therapeutic composition comprises a therapeutic agent that is a therapeutic protein that comprises Bruton's tyrosine kinase (BTK), ornithine transcarbamylase (OTC), survival motor neuron 1 protein (SMN1), galactocerebrosidase, N-sulfoglucosamine sulfohydrolase, N-acetylglucosaminadase, iduronate-2-sulfatase, alpha-glucosidase, sulfatase-modifying factor 1 (SUMF1), glucocerebrosidase (GCB), alpha galactosidase, alpha iduronidase, beta glucuronidase, or N-acetylgalactosamine-4-sulfatase.
